(19) United States
(12) Patent Application Publication
(10) Pub. No.: US 2018/0235987 A1
(43) Pub. Date: Aug. 23, 2018

(54) GLYCAN COMPOSITIONS AND USES THEREOF

(71) Applicant: KALEIDO BIOSCIENCES, INC., Cambridge, MA (US)

(72) Inventors: Geoffrey A. von Maltzahn, Somerville, MA (US); Yvonne J. Yamanaka, Cambridge, MA (US)

(21) Appl. No.: 15/754,850

(22) PCT Filed: Aug. 25, 2016
(86) PCT No.: PCT/US2016/048794

§ 371 Date: Feb. 23, 2018

Related U.S. Application Data
(60) Provisional application No. 62/209,618, filed on Aug. 25, 2015.
(60) Provisional application No. 62/209,629, filed on Aug. 25, 2015.
(60) Provisional application No. 62/209,626, filed on Aug. 25, 2015.

(51) Int. Cl. A61K 31/702; A61K 9/00; A61K 31/715; A61Q 11/00; A61K 8/60; A61K 8/73; A61K 45/06; A61K 35/741; A61K 35/742; A61K 35/744; A61K 35/745; A61K 35/747; A61K 8/99; A61P 1/02; A61P 11/02; A61P 15/02; A61P 31/04

(52) U.S. Cl. CPC A61K 31/702; A61K 2035/115; A61K 9/006; A61K 9/0034; A61K 31/715; A61Q 11/00; A61K 8/60; A61K 8/73; A61K 45/06; A61K 35/741; A61K 35/742; A61K 35/744; A61K 35/745; A61K 35/747; A61K 8/99; A61P 1/02; A61P 11/02; A61P 15/02; A61P 31/04; A61K 9/0043

(57) ABSTRACT

Compositions comprising glycan therapeutics, optionally comprising one or more oligo- or polysaccharides, probiotics, prebiotics and other agents are provided. Further, methods of generating said glycan therapeutics using polymeric catalysts are described. Also provided are methods of using said gycan therapeutics, e.g. for the modulation of vaginal microbiota.

### CLAIM OF PRIORITY

This application claims priority to U.S. Application No. 62/209,618; U.S. Application No. 62/209,626; and U.S. Application No. 62/209,629, each of which was filed on Aug. 25, 2015. The disclosure of each of the foregoing applications is incorporated herein by reference in its entirety.

### BACKGROUND OF THE INVENTION

Maintaining or restoring human health faces a large number of challenges many of which result from the lack of effective treatment options. There is a continued need for novel therapies and treatment regimens.

### SUMMARY OF THE INVENTION

Aspects of the invention relate to glycan preparations, pharmaceutical compositions, dosage forms, and methods of locally using the glycan preparations at non-gut body sites that contain mucosal tissues. In one aspect, the present invention features methods of modulating the abundance of a bacterial taxa in a non-gut body site. In some embodiments, the method comprises modulating the abundance of a bacterial taxa in a non-gut body site containing mucosal tissue of a human subject, comprising: locally administering to the non-gut body site a pharmaceutical composition comprising a glycan preparation in an amount effective to modulate the bacterial taxa in the non-gut body site containing mucosal tissue of the human subject, wherein the glycan preparation has at least one of the following properties: i) the glycan preparation comprises branched glycans that comprise glucose, galactose, arabinose, mannose, fructose, xylose, fucose, or rhamnose glycan units, ii) the average degree of branching (DB) of the branched glycans in the glycan preparation is between about 0.01 and about 0.6, iii) at least 50% of the glycans in the glycan preparation have a degree of polymerization (DP) of at least 3 and less than 30 glycan units, iv) the average DP of the glycan preparation is between about DP3 and about DP18, v) the ratio of alpha- to beta-glycosidic bonds present in the glycans of the glycan preparation is between about 0.8:1 and about 5:1, and/or optionally vi) the glycan preparation has a final solubility limit in water of at least about 60 Brix at 23° C.

In some embodiments, the non-gut body site (e.g., containing mucosal tissue) of a human subject is the nasal cavity. In some embodiments, the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity. In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site (e.g., containing mucosal tissue) of a human subject is the oral cavity. In some embodiments, the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site (e.g., containing mucosal tissue) of a human subject is the vagina. In some embodiments, the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina. In some embodiments, the abundance of at least two bacterial taxa of the species Lactobacillus crispatus, Lactobacillus gasseri, or Lactobacillus iners are modulated in the vagina.

In some embodiments, modulating comprises increasing the abundance of the bacterial taxa (e.g., by at least 5%, 10%, 25% 50%, 75%, 100%, 250%, 500%, 750%, or by at least 1000%). In some embodiments, modulating comprises decreasing the abundance of the bacterial taxa (e.g., by at least 5%, 10%, 25% 50%, 75%, 85%, 90%, 95%, 96%, 97%, 98%, 99%, or by at least 99.9%). In some embodiments, modulating comprises increasing or decreasing the relative abundance of the bacterial taxa by at least 5%, 10% or by at least 20%. In some embodiments, modulating comprises increasing or decreasing the abundance of the bacterial taxa in the non-gut body site relative to the bacterial community in the non-gut body site.

In some embodiments, modulating comprises increasing or decreasing the abundance of the bacterial taxa: i) relative to the abundance of a second bacterial taxa at the non-gut body site, or ii) relative to a reference value (e.g., a numerical or non-numerical value), optionally, i) wherein the reference value is a function of the abundance of the bacterial taxa at the non-gut body site prior to administration of the glycan preparation to the non-gut body site (e.g., in the absence of a glycan preparation), ii) wherein the reference value is a function of the abundance of the bacterial taxa at the non-gut body site in a subject having a dysbiosis of or in the non-gut body site, iii) wherein the reference value is a function of the abundance of the bacterial taxa for one or more individuals having a disease, disorder, or pathological condition (e.g. at the non-gut body site), iv) wherein the reference value is a function of the abundance of the bacterial taxa at the non-gut body site of a subject not having a disorder or a dysbiosis of or in the non-gut body site, v) wherein the reference value is a function of the value of the abundance of the bacterial taxa for one or more individuals not having a disorder a dysbiosis, and further optionally comprising comparing a value which is a function of abundance for the subject with the reverence value.

In some embodiments, modulating the abundance of a bacterial taxa in a non-gut body site containing mucosal tissue of a human subject treats a dysbiosis in the non-gut body site (e.g., treats at least one symptom of a dysbiosis in the non-gut body site).

In some embodiments, modulating the abundance of a bacterial taxa in a non-gut body site containing mucosal tissue of a human subject modulates the microbial diversity of the non-gut body site. In some embodiments, microbial diversity is decreased (e.g., by loss of a bacterial taxa or by at least 5%, 6%, 7%, 8%, 9%, or 10%, or at least 0.3 log-fold, 0.6 log-fold, or 1 log-fold, e.g., as measured by Shannon diversity index). In some embodiments, microbial diversity is increased (e.g., by gain of a bacterial taxa or by at least 55%, 6%, 7%, 8%, 9%, or 10%, or at least 0.3 log-fold, 0.6 log-fold, or 1 log-fold, e.g., as measured by Shannon diversity index).

In some embodiments, modulating the abundance of a bacterial taxa in a non-gut body site containing mucosal tissue of a human subject modulates the pH of the non-gut body site. In some embodiments, the pH becomes more basic (e.g., an increase of at least about 0.25 pH units or at least 0.5 pH units). In some embodiments, the pH becomes more acidic (e.g., a decrease of at least about 0.25 pH units or at least 0.5 pH units).

In some embodiments, modulating the abundance of a bacterial taxa in a non-gut body site containing mucosal tissue of a human subject modulates the profile of a microbial metabolite in the non-gut body site (e.g., a microbial metabolite described in Table 8). In some embodiments, modulation comprises increasing the level of a microbial metabolite in the non-gut body site (e.g., a microbial metabolite described in Table 8). In some embodiments, modulation comprises decreasing the level of a microbial metabolite in the non-gut body site (e.g., a microbial metabolite described in Table 8).

In some embodiments, modulation comprises modulating the level of a volatile fatty acid in the non-gut body site.

In some embodiments, modulating the abundance of a bacterial taxa in a non-gut body site containing mucosal tissue of a human subject modulates treats a disease, disorder or pathological condition at the non-gut body site. In some embodiments, the non-gut body site containing mucosal tissue of a human subject is the nasal cavity. In some embodiments, the disease, disorder or pathological condition at the nasal cavity is rhinosinusitis (sinus infection), chronic rhinosinusitis (CRS), S. aureus infection or carriage, nasal vestibulitis, nasal furuncles or asthma.

In some embodiments, the non-gut body site containing mucosal tissue of a human subject is the oral cavity. In some embodiments, the disease, disorder or pathological condition at the oral cavity is dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsillitis, dentoalveolar abscess, periodontal abscess, Ludwig's angina, viral infection (e.g. herpesvirus, human papilloma virus, etc.), or fungal/yeast infections (e.g. candidiasis). In some embodiments, the non-gut body site containing mucosal tissue of a human subject is the vagina. In some embodiments, the disease, disorder or pathological condition at the vagina is bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the method of modulating the abundance of a bacterial taxa in a non-gut body site containing mucosal tissue of a human subject further comprises locally or systemically administering an antimicrobial agent (e.g., an antibiotic, antifungal, or antiviral agent).

In some embodiments, the method of modulating the abundance of a bacterial taxa in a non-gut body site containing mucosal tissue of a human subject further comprises locally or systemically administering an anti-inflammatory agent or steroid.

In some embodiments, the method of modulating the abundance of a bacterial taxa in a non-gut body site containing mucosal tissue of a human subject further comprises locally administering a beneficial bacterial taxa (e.g., a commensal bacterial taxa residing in a healthy or non-dysbiotic non-gut body site described herein) to the non-gut body site. In some embodiments, the beneficial bacterial taxa is selected from the genera Streptococcus, Bifidobacterium, Lactobacillus, Escherichia, Weissella, Propionibacterium, and Bacillus. In some embodiments, the beneficial bacterial taxa is targeted to the oral cavity and is selected from Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, and Propionibacterium freudenreichii. In some embodiments, the beneficial bacterial taxa is targeted to the nasal cavity and is selected from Lactobacillus sakei, Lactobacillus reuteri, Streptococcus salivarius, Streptococcus thermophiles, Lactobacillus acidophilus, Bifidobacterium sp B420, and Lactobacillus GG. In some embodiments, the beneficial bacterial taxa is targeted to the vagina and is selected from Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, and Bacillus coagulans.

In some embodiments, the method of modulating the abundance of a bacterial taxa in a non-gut body site containing mucosal tissue of a human subject further comprises selecting a subject in need of modulating the abundance of a bacterial taxa in a non-gut body site containing mucosal tissue. In some embodiments, the selecting comprises acquiring a value representing dysbiosis at the non-gut body site (e.g. a microbial sequencing analysis of a sample of the site) and selecting the subject if a dysbiosis is present. In some embodiments, the selecting comprises acquiring a value representing the abundance of a selected bacterial taxa at the non-gut body site (e.g., a microbial sequencing analysis of a sample of the site) and selecting the subject if the abundance of the bacterial taxa at the non-gut body site differs from a predetermined value for the non-gut body site (e.g. the range of abundance for the taxa in a healthy state across a number of subjects).

In some embodiments, the method of modulating the abundance of a bacterial taxa in a non-gut body site containing mucosal tissue of a human subject comprises administering a first unit dosage form of the glycan preparation during a first or initial period. In some embodiments, the method further comprises administering a second dosage form of the glycan preparation during a second or subsequent period. In some embodiments, the first or initial period comprises conditioning or adapting the taxa to metabolize the glycan preparation and the second or subsequent period comprises modulating the abundance of the bacterial taxa at the non-gut body site of the subject. In some embodiments, the glycan preparation is administered as a unit dosage from suitable for local administration at the non-gut body site of the subject (e.g. to mucosal tissue).

In some embodiments, the glycan preparation contacts the non-gut body site before traversing the GI tract. In some embodiments, less than 90, 80, 70, 60, 50, 40, 30, 20, 10, or 5%, by weight, of the glycan preparation that is locally administered enters or passes through the GI tract, e.g., passes through the stomach. In some embodiments, the glycan preparation is introduced through the vaginal opening. In some embodiments, the glycan preparation is introduced through the nares (nostrils). In some embodiments, the glycan preparation is introduced through the mouth.

In some embodiments, modulating the abundance of a bacterial taxa in the non-gut body site containing mucosal tissue of a human subject reduces odor produced by the site (e.g., malodor).

In some embodiments, modulating the abundance of a bacterial taxa in the non-gut body site containing mucosal tissue of a human subject is determined under in vitro conditions. In some embodiments, a value for modulating the abundance of a bacterial taxa is acquired from an in vitro microbial culture propagated from a biological sample (e.g. saliva, mucus, excretion, cavity swab, etc.) taken from the non-gut body site of a human. In some embodiments, a value for modulating the abundance of a bacterial taxa is acquired from a single strain bacterium known to be associated with the non-gut body site in vivo and being propagated in vitro (e.g., strains of Staphylococcus, Lactobacillus, Propionibacterium, Corynebacterium, Rothia, Prevotella, Streptococcus, Leptotrichia, Kingella, Neisseria, Haemophilus, Oribacterium, etc.).

In some embodiments, the ratio of alpha- to beta-glycosidic bonds present in the glycans of the glycan preparation is between about 1:1 and about 5:1. In some embodiments, the average degree of branching (DB) of the branched glycans in the glycan preparation is between about 0.05 and about 0.6. In some embodiments, the average DP of the glycan preparation is one of: between about DP3 and about DP15, between about DP3 and about DP8, between about DP5 and about DP10, or between about DP6 and about DP18.

In some embodiments, at least one, at least two, at least three, at least four, or more of the glycosidic bonds independently comprise a 1→2 glycosidic bond, a 1→3 glycosidic bond, a 1→4 glycosidic bond, or a 1→6 glycosidic bond.

In another aspect, the present invention features a method of method of any of: a) modulating the abundance of a bacterial taxa in a non-gut body site containing a mucosal tissue of a subject, b) modulating microbial diversity in a non-gut body site containing a mucosal tissue of a subject, c) modulating the pH of a non-gut body site containing a mucosal tissue of a subject, d) modulating the profile of a microbial metabolite of a non-gut body site containing a mucosal tissue of a subject, e) treating a dysbiosis in a non-gut body site containing a mucosal tissue of a subject, or f) treating a disease, disorder or pathological condition of a non-gut body site containing a mucosal tissue of a subject, the method comprising: locally administering a glycan preparation to the non-gut body site containing a mucosal tissue of the subject, wherein the glycan preparation has one, two or more (e.g. 3, 4, 5 or 6) of the following properties: i) the glycan preparation comprises branched glycans that comprise glucose, galactose, arabinose, mannose, fructose, xylose, fucose, or rhamnose glycan units, ii) the average degree of branching (DB) of the branched glycans in the glycan preparation is between about 0.01 and about 0.6, iii) at least 50% of the glycans in the glycan preparation have a degree of polymerization (DP) of at least 3 and less than 30 glycan units, iv) the average DP of the glycan preparation is between about DP3 and about DP18, v) the ratio of alpha- to beta-glycosidic bonds present in the glycans of the glycan preparation is between about 0.8:1 and about 5:1, and vi) the glycan preparation has a final solubility limit in water of at least about 60 Brix at 23° C., thereby a) modulating the abundance of a bacterial taxa of, b) modulating the microbial diversity in, c) modulating the pH of, d) modulating the profile of a microbial metabolite of, e) treating a dysbiosis of, or f) treating a disorder in, a non-gut body site containing a mucosal tissue of a subject.

In some embodiments, the non-gut body site containing a mucosal tissue is the oral cavity, nasal cavity, or vagina. In some embodiments, the non-gut body site containing a mucosal tissue is the oral cavity. In some embodiments, the non-gut body site containing a mucosal tissue is the nasal cavity. In some embodiments, the non-gut body site containing a mucosal tissue is the vagina.

In some embodiments, the abundance of a bacterial taxa in the non-gut body site of the subject is independently increased by at least 5%, 10%, 25% 50%, 75%, 100%, 250%, 500%, 750%, or by at least 1000%. In some embodiments, the abundance of a bacterial taxa in the non-gut body site of the subject is independently is decreased by at least 5%, 10%, 25% 50%, 75%, 85%, 90%, 95%, 96%, 97%, 98%, 99%, or by at least 99.9%. In some embodiments, the bacterial taxa comprises a commensal bacterial taxa. In some embodiments, the bacterial taxa comprises a pathogenic bacterial taxa.

In some embodiments, modulating the abundance of a bacterial taxa in a non-gut body site containing mucosal tissue of a human subject modulates the microbial diversity of the non-gut body site. In some embodiments, microbial diversity is decreased (e.g., by loss of a bacterial taxa or by at least 5%, 6%, 7%, 8%, 9%, or 10%, or at least 0.3 log-fold, 0.6 log-fold, or 1 log-fold, e.g., as measured by Shannon diversity index). In some embodiments, microbial diversity is increased (e.g., by gain of a bacterial taxa or by at least 55%, 6%, 7%, 8%, 9%, or 10%, or at least 0.3 log-fold, 0.6 log-fold, or 1 log-fold, e.g., as measured by Shannon diversity index).

In some embodiments, modulating the abundance of a bacterial taxa in a non-gut body site containing mucosal tissue of a human subject modulates the pH of the non-gut body site. In some embodiments, the pH becomes more basic (e.g., an increase of at least about 0.25 pH units or at least 0.5 pH units). In some embodiments, the pH becomes more acidic (e.g., a decrease of at least about 0.25 pH units or at least 0.5 pH units).

In some embodiments, modulating the abundance of a bacterial taxa in a non-gut body site containing mucosal tissue of a human subject modulates the profile of a microbial metabolite in the non-gut body site (e.g., a microbial metabolite described in Table 8). In some embodiments, modulation comprises increasing the level of a microbial metabolite in the non-gut body site (e.g., a microbial metabolite described in Table 8). In some embodiments, modulation comprises decreasing the level of a microbial metabolite in the non-gut body site (e.g., a microbial metabolite described in Table 8). In some embodiments, the concentration of microbial metabolite of the non-gut body site (e.g., a microbial metabolite described in Table 8) is increased or decreased by at least about 0.5% (e.g., at least about 1%, about 5%, about 10%). In some embodiments, the concentration of the microbial metabolite of the non-gut body site (e.g., a microbial metabolite described in Table 8) is increased or decreased by at least about 0.3 log-fold (e.g., at least 0.6 log-field, 1 log-fold). In some embodiments, the microbial metabolite is selected from the group consisting of: formic acid, acetic acid, propionic acid, butryic acid, isobutyric acid, valeric acid, isovaleric acid, acorbic acid, tryptophan, serotonin, indole, succinic acid, trimethylamine (TMA), trimethylamine N-oxide (TMAO), deoxycholic acid, ethyphenyl sulfate, acetylaldehyde, lactic acid, hydrogen peroxide, and butanedione.

In some embodiments, modulation comprises modulating the level of a volatile fatty acid in the non-gut body site.

In some embodiments, modulating the abundance of a bacterial taxa in a non-gut body site containing mucosal tissue of a human subject treats a dysbiosis in the non-gut body site (e.g., treats at least one symptom of a dysbiosis in the non-gut body site). In some embodiments, modulating the abundance of a bacterial taxa in a non-gut body site containing mucosal tissue of a human subject treats a disorder of a non-gut body site (e.g., treats at least one symptom of a disorder in the non-gut body site). In some embodiments, the non-gut body site is selected from the oral cavity, the nasal cavity, and the vagina.

In some embodiments, the method comprises treating a disorder of the oral cavity, nasal cavity, or vagina in a subject in need thereof. In some embodiments, the subject experiences a reduction in at least one symptom of the disorder of the oral cavity, nasal cavity, or vagina following treatment. In some embodiments, the reduction in the severity of a symptom following treatment is about 10%, 20%, 30%, 40%, 50%, 60%, 70%, 80%, 90%, 95%, or about 100% relative to the severity of the symptom prior to treatment.

In some embodiments, the non-gut body site comprises a mucosal tissue of the oral cavity. In some embodiments, the method comprises treating a disorder of the oral cavity selected from dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, dentoalveolar abscess, periodontal abscess, Ludwig's angina, viral infection (e.g. herpesvirus, human papilloma virus, etc.), or fungal/yeast infections (e.g. candidiasis).

In some embodiments, the non-gut body site comprises a mucosal tissue of the nasal cavity. In some embodiments, the method comprises treating a disorder of the nasal cavity selected from rhinosinusitis (sinus infection), chronic rhinosinusitis (CRS), S. aureus infection or carriage, nasal vestibulitis, nasal furuncles, and asthma.

In some embodiments, the non-gut body site comprises a mucosal tissue of the vagina. In some embodiments, the method comprises treating a disorder of the vagina selected from bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the non-gut body site containing mucosal tissue of a human subject is the nasal cavity. In some embodiments, the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity. In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site containing mucosal tissue of a human subject is the oral cavity. In some embodiments, the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site containing mucosal tissue of a human subject is the vagina. In some embodiments, the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina.

In some embodiments, the abundance of at least two bacterial taxa of the species Lactobacillus crispatus, Lactobacillus gasseri, or Lactobacillus iners are modulated in the vagina.

In some embodiments, the ratio of alpha- to beta-glycosidic bonds present in the glycans of the glycan preparation is between about 1:1 and about 5:1. In some embodiments, the average degree of branching (DB) of the branched glycans in the glycan preparation is between about 0.05 and about 0.6. In some embodiments, the average DP of the glycan preparation is one of: between about DP3 and about DP15, between about DP3 and about DP8, between about DP5 and about DP10, or between about DP6 and about DP18.

In some embodiments, at least one, at least two, at least three, at least four, or more of the glycosidic bonds independently comprise a 1→2 glycosidic bond, a 1→3 glycosidic bond, a 1→4 glycosidic bond, or a 1→6 glycosidic bond.

In another aspect, the present invention features a formulation of a glycan preparation for local administration to a non-gut body site containing a mucosal tissue of a subject, wherein the glycan preparation has one, two, or more (e.g. 3, 4, 5 or 6) of the following properties: i) the glycan preparation comprises branched glycans that comprise glucose, galactose, arabinose, mannose, fructose, xylose, fucose, or rhamnose glycan units, ii) the average degree of branching (DB) of the branched glycans in the glycan preparation is between about 0.01 and about 0.6, iii) at least 50% of the glycans in the glycan preparation have a degree of polymerization (DP) of at least 3 and less than 30 glycan units, iv) the average DP of the glycan preparation is between about DP3 and about DP18, v) the ratio of alpha- to beta-glycosidic bonds present in the glycans of the glycan preparation is between about 0.8:1 and about 5:1, and/or vi) the glycan preparation has a final solubility limit in water of at least about 60 Brix at 23° C.

In some embodiments, the non-gut body site containing a mucosal tissue is the oral cavity, nasal cavity, or vagina. In some embodiments, the non-gut body site containing a mucosal tissue is the oral cavity. In some embodiments, the non-gut body site containing a mucosal tissue is the nasal cavity. In some embodiments, the non-gut body site containing a mucosal tissue is the vagina.

In some embodiments, the non-gut body site comprises a mucosal tissue of the oral cavity. In some embodiments, the formulation is administered to treat a disorder of the oral cavity selected from dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, dentoalveolar abscess, periodontal abscess, Ludwig's angina, viral infection (e.g. herpesvirus, human papilloma virus, etc.), or fungal/yeast infections (e.g. candidiasis).

In some embodiments, the non-gut body site comprises a mucosal tissue of the nasal cavity. In some embodiments, the formulation is administered to treat a disorder of the nasal cavity selected from rhinosinusitis (sinus infection), chronic rhinosinusitis (CRS), S. aureus infection or carriage, nasal vestibulitis, nasal furuncles, and asthma.

In some embodiments, the non-gut body site comprises a mucosal tissue of the vagina. In some embodiments, the formulation is administered to treat a disorder of the vagina selected from bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the non-gut body site containing mucosal tissue of a human subject is the nasal cavity. In some embodiments, the formulation is administered to modulate the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity.

In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site containing mucosal tissue of a human subject is the oral cavity. In some embodiments, the formulation is administered to modulate the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site containing mucosal tissue of a human subject is the vagina. In some embodiments, the formulation is administered to modulate the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina. In some embodiments, the abundance of at least two bacterial taxa of the species Lactobacillus crispatus, Lactobacillus gasseri, or Lactobacillus iners are modulated in the vagina.

In some embodiments, the formulation is provided as a unit dosage form.

In some embodiments, the formulation further comprises a sugar, a sugar alcohol, an amino acid, a peptide, a micronutrient, a fatty acid, or a polyphenol. In some embodiments, the formulation further comprises a sugar or sugar alcohol. In some embodiments, the sugar or sugar alcohol comprises glucose, galactose, fructose, fucose, mannose, xylose, arabinose, rhamnose, ribose, sucrose, sorbose, lactose, sorbitol, maltose, mannitol, lactulose, lactitol, erythritol, tagatose, kojibiose, nigerose, isomaltose, trehalose, sophorose, laminaribiose, gentiobiose, turanose, maltulose, palatinose, gentiobiulose, mannobiose, melibiulose, rutinulose, or xylobiose. In some embodiments, the formulation further comprises a micronutrient. In some embodiments, the micronutrient comprises a vitamin, an element, or a mineral. In some embodiments, the formulation further comprises a fatty acid. In some embodiments, the fatty acid comprises a short-chain fatty acid (SCFA), a medium-chain fatty acid (MCFA), a long-chain fatty acid (LCFA), or a very long chain fatty acid (VLCFA). In some embodiments, the formulation further comprises a polyphenol. In some embodiments, the polyphenol comprises a catechin, ellagitannin, isoflavone, flavonol, flavanone, anthocyanin, or lignin.

In some embodiments, the formulation further comprises a therapeutic agent (e.g., standard care therapeutic agent). In some embodiments, the therapeutic agent comprises an antibiotic, antifungal, antiviral, a fluoride treatment, a steroid, silver nitrate, a sugar or sugar alcohol (e.g., lactulose, xylitol), an oil (e.g., coconut oil, MCT oil, tea tree oil), zinc, iodine, an isoflavone (e.g., soy), an acid (e.g., acetic acid, boric acid), a natural extract (e.g., elderberry, milk thistle, lavender), an antioxidant (e.g., vitamin C), or garlic. In some embodiments, the formulation further comprises an antimicrobial agent (e.g., an antibiotic, antifungal, or antiviral agent). In some embodiments, the formulation further comprises an anti-inflammatory agent or steroid.

In some embodiments, the formulation further comprises a beneficial bacterial taxa (e.g., a commensal bacterial taxa residing in a healthy or non-dysbiotic non-gut body site described herein). In some embodiments, the beneficial bacterial taxa is from the genera Streptococcus, Bifidobacterium, Lactobacillus, Escherichia, Weissella, Propionibacterium, or Bacillus. In some embodiments, the beneficial bacterial taxa is targeted to the oral cavity. In some embodiments, the beneficial bacterial taxa targeted to the oral cavity is selected from Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, and Propionibacterium freudenreichii. In some embodiments, the beneficial bacterial taxa is targeted to the nasal cavity. In some embodiments, the beneficial bacterial taxa targeted to the nasal cavity is selected from Lactobacillus sakei, Lactobacillus reuteri, Streptococcus salivarius, Streptococcus thermophiles, Lactobacillus acidophilus, Bifidobacterium sp B420, and Lactobacillus GG. In some embodiments, the beneficial bacterial taxa is targeted to the vagina. In some embodiments, the beneficial bacterial taxa targeted to the vagina is selected from Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, and Bacillus coagulans.

In some embodiments, the formulation is prepared as a unit dosage form. In some embodiments, the unit dosage form is prepared for administration to the oral cavity, nasal cavity, or vagina. In some embodiments, the unit dosage form for administration to the oral cavity comprises a solid that rapidly dissolves in the mouth (e.g. dissolving strip, film, fast melt), a liquid (e.g., mouthwash, spray, tincture, drop) or a gel (e.g., a toothpaste, cream or ointment). In some embodiments, the unit dosage form for administration to the vagina comprises a suppository (e.g., pessary), cream, ointment, solution, suspension, emulsion, vaginal ring, tampon, pad, douche, sponge, strip, spray, foam, applicator, or adhesive. In some embodiments, the unit dosage form for administration to the oral cavity comprises a mist (e.g. aqueous mist), dry powder, spray, foam, applicator, cream, ointment, solution, suspension, emulsion.

In some embodiments, the ratio of alpha- to beta-glycosidic bonds present in the glycans of the glycan preparation is between about 1:1 and about 5:1. In some embodiments, the average degree of branching (DB) of the branched glycans in the glycan preparation is between about 0.05 and about 0.6. In some embodiments, the average DP of the glycan preparation is one of: between about DP3 and about DP15, between about DP3 and about DP8, between about DP5 and about DP10, or between about DP6 and about DP18.

In some embodiments, at least one, at least two, at least three, at least four, or more of the glycosidic bonds independently comprise a 1→2 glycosidic bond, a 1→3 glycosidic bond, a 1→4 glycosidic bond, or a 1→6 glycosidic bond.

In another aspect, the present invention features a container comprising a plurality of unit dosage forms of a glycan preparation suitable for local administration to a non-gut body site. In some embodiments, the container comprises a first compartment comprising a first unit dosage for and a second compartment comprising a second dosage form. In some embodiments, the first and second dosage forms are the same. In some embodiments, the first and second dosage forms are different from one another, e.g., they have different amounts of glycan preparation, have different release properties, comprise different excipients, or comprise different or different amounts of a drug. In some embodiments, the container comprises a first unit dosage form which is administered to the subject during a first or initial period and a second unit dosage form which is administered to the subject in a second or subsequent period. In some embodiments, the first period is an adaption period and the second period is a maintenance period.

In some embodiments, the non-gut body site containing a mucosal tissue is the oral cavity, nasal cavity, or vagina. In some embodiments, the non-gut body site containing a mucosal tissue is the oral cavity. In some embodiments, the non-gut body site containing a mucosal tissue is the nasal cavity. In some embodiments, the non-gut body site containing a mucosal tissue is the vagina.

In some embodiments, the non-gut body site comprises a mucosal tissue of the oral cavity. In some embodiments, the container comprises a glycan preparation to treat a disorder of the oral cavity selected from dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, dentoalveolar abscess, periodontal abscess, Ludwig's angina, viral infection (e.g. herpesvirus, human papilloma virus, etc.), or fungal/yeast infections (e.g. candidiasis).

In some embodiments, the non-gut body site comprises a mucosal tissue of the nasal cavity. In some embodiments, the container comprises a glycan preparation to treat a disorder of the nasal cavity selected from rhinosinusitis (sinus infection), chronic rhinosinusitis (CRS), S. aureus infection or carriage, nasal vestibulitis, nasal furuncles, and asthma.

In some embodiments, the non-gut body site comprises a mucosal tissue of the vagina. In some embodiments, the container comprises a glycan preparation to treat a disorder of the vagina selected from bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the non-gut body site containing mucosal tissue of a human subject is the nasal cavity. In some embodiments, the container comprises a glycan preparation to modulate the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity. In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site containing mucosal tissue of a human subject is the oral cavity. In some embodiments, the container comprises a glycan preparation to modulate the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site containing mucosal tissue of a human subject is the vagina. In some embodiments, the container comprises a glycan preparation to modulate the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina. In some embodiments, the abundance of at least two bacterial taxa of the species Lactobacillus crispatus, Lactobacillus gasseri, or Lactobacillus iners are modulated in the vagina.

In some embodiments, the container comprises a glycan preparation provided as a unit dosage form. In some embodiments, the container further comprises a sugar, a sugar alcohol, an amino acid, a peptide, a micronutrient, a fatty acid, or a polyphenol. In some embodiments, the container further comprises a sugar or sugar alcohol. In some embodiments, the sugar or sugar alcohol comprises glucose, galactose, fructose, fucose, mannose, xylose, arabinose, rhamnose, ribose, sucrose, sorbose, lactose, sorbitol, maltose, mannitol, lactulose, lactitol, erythritol, tagatose, kojibiose, nigerose, isomaltose, trehalose, sophorose, laminaribiose, gentiobiose, turanose, maltulose, palatinose, gentiobiulose, mannobiose, melibiulose, rutinulose, or xylobiose.

In some embodiments, the container further comprises a micronutrient. In some embodiments, the micronutrient comprises a vitamin, an element, or a mineral. In some embodiments, the container further comprises a fatty acid. In some embodiments, the fatty acid comprises a short-chain fatty acid (SCFA), a medium-chain fatty acid (MCFA), a long-chain fatty acid (LCFA), or a very long chain fatty acid (VLCFA). In some embodiments, the container further comprises a polyphenol. In some embodiments, the polyphenol comprises a catechin, ellagitannin, isoflavone, flavonol, flavanone, anthocyanin, or lignin.

In some embodiments, the container further comprises a therapeutic agent (e.g., standard care therapeutic agent). In some embodiments, the therapeutic agent comprises an antibiotic, antifungal, antiviral, a fluoride treatment, a steroid, silver nitrate, a sugar or sugar alcohol (e.g., lactulose, xylitol), an oil (e.g., coconut oil, MCT oil, tea tree oil), zinc, iodine, an isoflavone (e.g., soy), an acid (e.g., acetic acid, boric acid), a natural extract (e.g., elderberry, milk thistle, lavender), an antioxidant (e.g., vitamin C), or garlic. In some embodiments, the container further comprises an antimicrobial agent (e.g., an antibiotic, antifungal, or antiviral agent). In some embodiments, the container further comprises an anti-inflammatory agent or steroid.

In some embodiments, the container further comprises a beneficial bacterial taxa (e.g., a commensal bacterial taxa residing in a healthy or non-dysbiotic non-gut body site described herein). In some embodiments, the beneficial bacterial taxa is from the genera Streptococcus, Bifidobacterium, Lactobacillus, Escherichia, Weissella, Propionibacterium, or Bacillus. In some embodiments, the beneficial bacterial taxa is targeted to the oral cavity. In some embodiments, the beneficial bacterial taxa targeted to the oral cavity is selected from Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, and Propionibacterium freudenreichii. In some embodiments, the beneficial bacterial taxa is targeted to the nasal cavity. In some embodiments, the beneficial bacterial taxa targeted to the nasal cavity is selected from Lactobacillus sakei, Lactobacillus reuteri, Streptococcus salivarius, Streptococcus thermophiles, Lactobacillus acidophilus, Bifidobacterium sp B420, and Lactobacillus GG. In some embodiments, the beneficial bacterial taxa is targeted to the vagina. In some embodiments, the beneficial bacterial taxa targeted to the vagina is selected from Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, and Bacillus coagulans.

In some embodiments, the container comprises a glycan preparation formulated as a unit dosage form. In some embodiments, the unit dosage form is prepared for administration to the oral cavity, nasal cavity, or vagina. In some embodiments, the unit dosage form for administration to the oral cavity comprises a solid that rapidly dissolves in the mouth (e.g. dissolving strip, film, fast melt), a liquid (e.g., mouthwash, spray, tincture, drop) or a gel (e.g., a toothpaste, cream or ointment). In some embodiments, the unit dosage form for administration to the vagina comprises a suppository (e.g., pessary), cream, ointment, solution, suspension, emulsion, vaginal ring, tampon, pad, douche, sponge, strip, spray, foam, applicator, or adhesive. In some embodiments, the unit dosage form for administration to the oral cavity comprises a mist (e.g. aqueous mist), dry powder, spray, foam, applicator, cream, ointment, solution, suspension, emulsion.

In some embodiments, the ratio of alpha- to beta-glycosidic bonds present in the glycans of the glycan preparation is between about 1:1 and about 5:1. In some embodiments, the average degree of branching (DB) of the branched glycans in the glycan preparation is between about 0.05 and about 0.6. In some embodiments, the average DP of the glycan preparation is one of: between about DP3 and about DP15, between about DP3 and about DP8, between about DP5 and about DP10, or between about DP6 and about DP18.

In some embodiments, at least one, at least two, at least three, at least four, or more of the glycosidic bonds independently comprise a 1→2 glycosidic bond, a 1→3 glycosidic bond, a 1→4 glycosidic bond, or a 1→6 glycosidic bond.

In another aspect, the present invention comprises a kit comprising a glycan preparation for local administration to a non-gut body site containing a mucosal tissue. In some embodiments, the glycan preparation has two or more (e.g. 3, 4, 5 or 6) of the following properties: i) the glycan preparation comprises branched glycans that comprise glucose, galactose, arabinose, mannose, fructose, xylose, fucose, or rhamnose glycan units, ii) the average degree of branching (DB) of the branched glycans in the glycan preparation is between about 0.01 and about 0.6 or between 0.05 and about 0.5, iii) at least 50% of the glycans in the glycan preparation have a degree of polymerization (DP) of at least 3 and less than 30 glycan units, iv) the average DP of the glycan preparation is between about DP2 and about DP20, between about DP3 and about DP15, between about DP3 and about DP8, between about DP5 and about DP10, or between about DP6 and about DP18, v) the ratio of alpha- to beta-glycosidic bonds present in the glycans of the glycan preparation is between about 1:1 and about 5:1 or between about 0.8:1 and about 5:1, and/or vi) the glycan preparation has a final solubility limit in water of at least about 60 Brix at 23° C.

In some embodiments, the ratio of alpha- to beta-glycosidic bonds present in the glycans of the glycan preparation is between about 1:1 and about 5:1. In some embodiments, the average degree of branching (DB) of the branched glycans in the glycan preparation is between about 0.05 and about 0.6. In some embodiments, the average DP of the glycan preparation is one of: between about DP3 and about DP15, between about DP3 and about DP8, between about DP5 and about DP10, or between about DP6 and about DP18.

In some embodiments, at least one, at least two, at least three, at least four, or more of the glycosidic bonds independently comprise a 1→2 glycosidic bond, a 1→3 glycosidic bond, a 1→4 glycosidic bond, or a 1→6 glycosidic bond.

In some embodiments, the non-gut body site containing a mucosal tissue is the oral cavity, nasal cavity, or vagina. In some embodiments, the non-gut body site containing a mucosal tissue is the oral cavity. In some embodiments, the non-gut body site containing a mucosal tissue is the nasal cavity. In some embodiments, the non-gut body site containing a mucosal tissue is the vagina.

In some embodiments, the non-gut body site comprises a mucosal tissue of the oral cavity. In some embodiments, the kit comprises a glycan preparation to treat a disorder of the oral cavity selected from dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, dentoalveolar abscess, periodontal abscess, Ludwig's angina, viral infection (e.g. herpesvirus, human papilloma virus, etc.), or fungal/yeast infections (e.g. candidiasis).

In some embodiments, the non-gut body site comprises a mucosal tissue of the nasal cavity. In some embodiments, the kit comprises a glycan preparation to treat a disorder of the nasal cavity selected from rhinosinusitis (sinus infection), chronic rhinosinusitis (CRS), S. aureus infection or carriage, nasal vestibulitis, nasal furuncles, and asthma.

In some embodiments, the non-gut body site comprises a mucosal tissue of the vagina. In some embodiments, the kit comprises a glycan preparation to treat a disorder of the vagina selected from bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the non-gut body site containing mucosal tissue of a human subject is the nasal cavity. In some embodiments, the kit comprises a glycan preparation to modulate the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity. In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site containing mucosal tissue of a human subject is the oral cavity. In some embodiments, the kit comprises a glycan preparation to modulate the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site containing mucosal tissue of a human subject is the vagina. In some embodiments, the kit comprises a glycan preparation to modulate the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina. In some embodiments, the abundance of at least two bacterial taxa of the species Lactobacillus crispatus, Lactobacillus gasseri, or Lactobacillus iners are modulated in the vagina.

In some embodiments, the kit comprises a glycan preparation provided as a unit dosage form. In some embodiments, the kit further comprises a sugar, a sugar alcohol, an amino acid, a peptide, a micronutrient, a fatty acid, or a polyphenol. In some embodiments, the kit further comprises a sugar or sugar alcohol. In some embodiments, the sugar or sugar alcohol comprises glucose, galactose, fructose, fucose, mannose, xylose, arabinose, rhamnose, ribose, sucrose, sorbose, lactose, sorbitol, maltose, mannitol, lactulose, lactitol, erythritol, tagatose, kojibiose, nigerose, isomaltose, trehalose, sophorose, laminaribiose, gentiobiose, turanose, maltulose, palatinose, gentiobiulose, mannobiose, melibiulose, rutinulose, or xylobiose. In some embodiments, the kit further comprises a micronutrient. In some embodiments, the micronutrient comprises a vitamin, an element, or a mineral. In some embodiments, the kit further comprises a fatty acid. In some embodiments, the fatty acid comprises a short-chain fatty acid (SCFA), a medium-chain fatty acid (MCFA), a long-chain fatty acid (LCFA), or a very long chain fatty acid (VLCFA). In some embodiments, the kit further comprises a polyphenol. In some embodiments, the polyphenol comprises a catechin, ellagitannin, isoflavone, flavonol, flavanone, anthocyanin, or lignin.

In some embodiments, the kit further comprises a therapeutic agent (e.g., standard care therapeutic agent). In some embodiments, the therapeutic agent comprises an antibiotic, antifungal, antiviral, a fluoride treatment, a steroid, silver nitrate, a sugar or sugar alcohol (e.g., lactulose, xylitol), an oil (e.g., coconut oil, MCT oil, tea tree oil), zinc, iodine, an isoflavone (e.g., soy), an acid (e.g., acetic acid, boric acid), a natural extract (e.g., elderberry, milk thistle, lavender), an antioxidant (e.g., vitamin C), or garlic. In some embodiments, the kit further comprises an antimicrobial agent (e.g., an antibiotic, antifungal, or antiviral agent). In some embodiments, the kit further comprises an anti-inflammatory agent or steroid.

In some embodiments, the kit further comprises a beneficial bacterial taxa (e.g., a commensal bacterial taxa residing in a healthy or non-dysbiotic non-gut body site described herein). In some embodiments, the beneficial bacterial taxa is from the genera Streptococcus, Bifidobacterium, Lactobacillus, Escherichia, Weissella, Propionibacterium, or Bacillus. In some embodiments, the beneficial bacterial taxa is targeted to the oral cavity. In some embodiments, the beneficial bacterial taxa targeted to the oral cavity is selected from Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, and Propionibacterium freudenreichii. In some embodiments, the beneficial bacterial taxa is targeted to the nasal cavity. In some embodiments, the beneficial bacterial taxa targeted to the nasal cavity is selected from Lactobacillus sakei, Lactobacillus reuteri, Streptococcus salivarius, Streptococcus thermophiles, Lactobacillus acidophilus, Bifidobacterium sp B420, and Lactobacillus GG. In some embodiments, the beneficial bacterial taxa is targeted to the vagina. In some embodiments, the beneficial bacterial taxa targeted to the vagina is selected from Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, and Bacillus coagulans.

In some embodiments, the kit comprises a glycan preparation formulated as a unit dosage form.

In some embodiments, the unit dosage form is prepared for administration to the oral cavity, nasal cavity, or vagina. In some embodiments, the unit dosage form for administration to the oral cavity comprises a solid that rapidly dissolves in the mouth (e.g. dissolving strip, film, fast melt), a liquid (e.g., mouthwash, spray, tincture, drop) or a gel (e.g., a toothpaste, cream or ointment). In some embodiments, the unit dosage form for administration to the vagina comprises a suppository (e.g., pessary), cream, ointment, solution, suspension, emulsion, vaginal ring, tampon, pad, douche, sponge, strip, spray, foam, applicator, or adhesive. In some embodiments, the unit dosage form for administration to the oral cavity comprises a mist (e.g. aqueous mist), dry powder, spray, foam, applicator, cream, ointment, solution, suspension, emulsion.

In another aspect, the present invention features a method of manufacturing a glycan preparation unit dosage form suitable for local administration to a non-gut body site of a subject comprising: providing a first amount of the glycan preparation; dividing the first amount of the glycan preparation into a plurality of unit dosage forms suitable for local administration to a non-gut body site of a subject, thereby manufacturing a glycan preparation unit dosage form suitable for administration to a non-gut body site of a subject.

In another aspect, the present invention features a method of manufacturing a glycan preparation unit dosage form suitable for local administration to a non-gut body site of a subject comprising: (a) providing a glycan preparation; (b) acquiring a value for one or more of the following characteristics of the glycan preparation: (i) the degree of polymerization (DP), (ii) the average degree of branching (DB), or (iii) the ratio of alpha-glycosidic to beta-glycosidic bonds, and (c) formulating the preparation as a unit dosage form suitable for local administration to a non-gut body site of a subject if one or more of the following criteria are met: (i) at least 50% of the glycans in the preparation have a DP of at least 3 and less than 30 glycan units, (ii) the average degree of branching (DB) of the glycans in the preparation is at least 0.01, (iii) the ratio of alpha- to beta-glycosidic bonds present in the glycans of the preparation is between about 0.8:1 to about 5:1, thereby manufacturing a glycan preparation unit dosage form suitable for local administration to a non-gut body site of a subject.

In some embodiments, the method of manufacturing a glycan preparation further comprises: acquiring a value for any one or both additional characteristics of the preparation: (iv) the identity of the glycan units, (v) the ratio of glycan units, and formulating the preparation as a pharmaceutical composition if: (vi) the glycan unit ratio in the preparation is about the same as the ratio of the glycan unit input.

In some embodiments, the method of manufacturing further comprises: b) acquiring a value for any one or both additional characteristics of the preparation: (iv) the level of bacterial growth, in media supplemented with the glycan preparation, of at least one commensal bacterial taxa (e.g. a bacterial strain) known to be associated with (or to reside in) the non-gut body site, and c) formulating the preparation as a pharmaceutical composition if the glycan preparation modulates (e.g. increases) the growth of the bacterial taxa i) relative to a predetermined level (e.g. that of a control carbon source, such as e.g., a sugar monomer or dimer, e.g., glucose) or ii) relative to another predetermined bacterial taxa (e.g. a pathogen or pathobiont).

In some embodiments, the bacterial taxa is a lactobacillus, e.g., L. crispatus, L. iners, L. gasseri, and L. jensenii and the non-gut body site is the vagina. In some embodiments, the bacterial taxa is Neisseria (e.g. Neisseria mucosa, Neisseria sicca, and Neisseria subflava), Rothia (e.g. Rothia mucilaginosa), Streptococcus (e.g. Streptococcus salivarius), or Veillonella (e.g. Veillonella parvula) and the non-gut body site is the oral cavity. In some embodiments, the bacterial taxa is Streptococcus mutans and its growth is reduced relative to another predetermined bacterial taxa (e.g. Neisseria (e.g. Neisseria mucosa, Neisseria sicca, and Neisseria subflava), Rothia (e.g. Rothia mucilaginosa), Streptococcus (e.g. Streptococcus salivarius), or Veillonella (e.g. Veillonella parvula) and the non-gut body site is the oral cavity. In some embodiments, the bacterial taxa is C. pseudodiphtheriticum or S. epidermidis and the non-gut body site is the nasal cavity. In some embodiments, the bacterial taxa is Staphylococcus aureus or Corynebacterium accolens and its growth is reduced relative to another predetermined bacterial taxa (e.g. C. pseudodiphtheriticum or S. epidermidis) and the non-gut body site is the nasal cavity.

In some embodiments, the step of formulating the preparation as a pharmaceutical composition comprises one or more of: i) removing unwanted constituents from the preparation, ii) reducing the volume of the preparation, iii) sterilizing the preparation, iv) admixing the preparation with a pharmaceutically acceptable excipient or carrier, v) admixing the preparation with a second drug or pharmaceutical agent, and vi) formulating the preparation into a dosage form suitable for the non-gut body site.

In some embodiments, the step of formulating the preparation as a pharmaceutical composition comprises one or more of: (i) packaging the preparation, (ii) labeling the packaged preparation, and (iii) selling or offering for sale the packaged and labeled preparation.

In another aspect, the present invention features a method of making a pharmaceutical composition, the method comprising: (i) providing a glycan preparation (e.g., a therapeutic glycan preparation) comprising at least one glycan unit selected from the group consisting of glucose, galactose, fucose, xylose, arabinose, rhamnose, and mannose, (ii) determining if a preselected NMR peak or group of NMR peaks is associated with the glycan preparation, and (iii) if the preselected peak or group of peaks is present, formulating the preparation as a pharmaceutical composition.

In another aspect, the present invention features a pharmaceutical composition comprising glycan preparation unit dosage form suitable for local administration to a non-gut body site of a subject, comprising a mixture of branched glycans, wherein the average degree of branching (DB) of the glycans in the preparation is at least 0.01, and wherein i) at least 50% of the glycans in the preparation have a degree of polymerization (DP) of at least 3 and less than 30 glycan units, ii) the glycan preparation comprises both alpha- and beta-glycosidic bonds, iii) at least one of the glycosidic bonds present in the glycans of the preparation comprise a 1→2 glycosidic bond, a 1→3 glycosidic bond, a 1→4 glycosidic bond, or a 1→6 glycosidic bond, and/or iv) the ratio of alpha- to beta-glycosidic bonds present in the glycans of the preparation is between about 1:1 to about 5:1.

In some embodiments, at least two of the glycosidic bonds independently comprise a 1→2 glycosidic bond, a 1→3 glycosidic bond, a 1→4 glycosidic bond, or a 1→6 glycosidic bond. In some embodiments, at least three of the glycosidic bonds independently comprise a 1→2 glycosidic bond, a 1→3 glycosidic bond, a 1→4 glycosidic bond, or a 1→6 glycosidic bond.

In some embodiments, the glycan unit comprises at least one of a monosaccharide selected from the group of glucose, galactose, arabinose, mannose, fructose, xylose, fucose, and rhamnose. In some embodiments, at least a 20% (by weight or number) of the glycans in the preparation, do not comprise more than a preselected reference level, of a repeating unit of 2 glycan units. In some embodiments, the glycan preparation is synthetic and not isolated from a natural oligosaccharide or polysaccharide source.

In some embodiments, the pharmaceutical composition further comprises a pharmaceutically acceptable excipient. In some embodiments, the composition is formulated as a unit-dosage form. In some embodiments, the unit-dosage form is formulated as a delayed release or time controlled system.

In some embodiments, the composition is locally administered to a non-gut body site containing mucosal tissue comprises local administration to a mucosal tissue of the non-gut body site.

In some embodiments, the non-gut body site (e.g., containing mucosal tissue) of a human subject is the nasal cavity. In some embodiments, the abundance of a bacterial taxa of the genus Corynebacterium, Alloiococcus, or Staphylococcus is modulated in the nasal cavity. In some embodiments, the abundance of a bacterial taxa of the genus Corynebacterium or Staphylococcus is modulated in the nasal cavity. In some embodiments, the abundance of a bacterial taxa of the genus Corynebacterium and Staphylococcus is modulated in the nasal cavity. In some embodiments, the abundance of a bacterial taxa of the species Staphylococcus epidermidis, Staphylococcus hominis, Staphylococcus aureus, or Propionibacterium acnes is modulated in the nasal cavity. In some embodiments, the abundance of at least two bacterial taxa of the species Staphylococcus epidermidis, Staphylococcus hominis, Staphylococcus aureus, or Propionibacterium acnes are modulated in the nasal cavity. In some embodiments, the abundance of at least three bacterial taxa of the species Staphylococcus epidermidis, Staphylococcus hominis, Staphylococcus aureus, or Propionibacterium acnes are modulated in the nasal cavity.

In some embodiments, the non-gut body site (e.g., containing mucosal tissue) of a human subject is the oral cavity. In some embodiments, the abundance of a bacterial taxa of the genus Prevotella, Oribacterium, Bifidobacterium, or Moryella is modulated in the oral cavity. In some embodiments, the abundance of a bacterial taxa of the genus Bifidobacterium, Abiotrophia, Clostridiales, Catonella, Moryella, Leptotrichia, Eikenella, Aggregatibacter, Prevotella, Oribacterium, Neisseria or Haemophilus is modulated in the oral cavity. In some embodiments, the abundance of a bacterial taxa of the genus Prevotella, Oribacterium, Neisseria or Haemophilus is modulated in the oral cavity. In some embodiments, the abundance of at least two bacterial taxa of the genera Prevotella, Oribacterium, Neisseria or Haemophilus are modulated in the oral cavity. In some embodiments, the abundance of at least three bacterial taxa of the genera Prevotella, Oribacterium, Neisseria or Haemophilus are modulated in the oral cavity. In some embodiments, the abundance of a bacterial taxa of the species Neisseria subflava or Streptococcus oralis is modulated in the oral cavity. In some embodiments, the abundance of a bacterial taxa of the species Neisseria subflava and Streptococcus oralis is modulated in the oral cavity.

In some embodiments, the non-gut body site (e.g., containing mucosal tissue) of a human subject is the vagina. In some embodiments, the abundance of a bacterial taxa of the genus lactobacillus is modulated in the vagina. In some embodiments, the abundance of a bacterial taxa of the species Lactobacillus crispatus, Lactobacillus gasseri, or Lactobacillus iners is modulated in the vagina. In some embodiments, the abundance of at least two bacterial taxa of the species Lactobacillus crispatus, Lactobacillus gasseri, or Lactobacillus iners are modulated in the vagina.

In some embodiments, modulating comprises increasing the abundance of the bacterial taxa (e.g., by at least 5%, 10%, 25% 50%, 75%, 100%, 250%, 500%, 750%, or by at least 1000%). In some embodiments, modulating comprises decreasing the abundance of the bacterial taxa (e.g., by at least 5%, 10%, 25% 50%, 75%, 85%, 90%, 95%, 96%, 97%, 98%, 99%, or by at least 99.9%). In some embodiments, modulating comprises increasing or decreasing the relative abundance of the bacterial taxa by at least 5%, 10% or by at least 20%. In some embodiments, modulating comprises increasing or decreasing the abundance of the bacterial taxa in the non-gut body site relative to the bacterial community in the non-gut body site.

In some embodiments, modulating comprises increasing or decreasing the abundance of the bacterial taxa: i) relative to the abundance of a second bacterial taxa at the non-gut body site, or ii) relative to a reference value (e.g., a numerical or non-numerical value), optionally, i) wherein the reference value is a function of the abundance of the bacterial taxa at the non-gut body site prior to administration of the glycan preparation to the non-gut body site (e.g., in the absence of a glycan preparation), ii) wherein the reference value is a function of the abundance of the bacterial taxa at the non-gut body site in a subject having a dysbiosis of or in the non-gut body site, iii) wherein the reference value is a function of the abundance of the bacterial taxa for one or more individuals having a disease, disorder, or pathological condition (e.g. at the non-gut body site), iv) wherein the reference value is a function of the abundance of the bacterial taxa at the non-gut body site of a subject not having a disorder or a dysbiosis of or in the non-gut body site, v) wherein the reference value is a function of the value of the abundance of the bacterial taxa for one or more individuals not having a disorder a dysbiosis, and further optionally comprising comparing a value which is a function of abundance for the subject with the reverence value.

In some embodiments, modulating the abundance of a bacterial taxa in a non-gut body site containing mucosal tissue of a human subject treats a dysbiosis in the non-gut body site (e.g., treats at least one symptom of a dysbiosis in the non-gut body site).

In some embodiments, modulating the abundance of a bacterial taxa in a non-gut body site containing mucosal tissue of a human subject modulates the microbial diversity of the non-gut body site. In some embodiments, microbial diversity is decreased (e.g., by loss of a bacterial taxa or by at least 5%, 6%, 7%, 8%, 9%, or 10%, or at least 0.3 log-fold, 0.6 log-fold, or 1 log-fold, e.g., as measured by Shannon diversity index). In some embodiments, microbial diversity is increased (e.g., by gain of a bacterial taxa or by at least 55%, 6%, 7%, 8%, 9%, or 10%, or at least 0.3 log-fold, 0.6 log-fold, or 1 log-fold, e.g., as measured by Shannon diversity index).

In some embodiments, modulating the abundance of a bacterial taxa in a non-gut body site containing mucosal tissue of a human subject modulates the pH of the non-gut body site. In some embodiments, the pH becomes more basic (e.g., an increase of at least about 0.25 pH units or at least 0.5 pH units). In some embodiments, the pH becomes more acidic (e.g., a decrease of at least about 0.25 pH units or at least 0.5 pH units).

In some embodiments, modulating the abundance of a bacterial taxa in a non-gut body site containing mucosal tissue of a human subject modulates the profile of a microbial metabolite in the non-gut body site (e.g., a microbial metabolite described in Table 8). In some embodiments, modulation comprises increasing the level of a microbial metabolite in the non-gut body site (e.g., a microbial metabolite described in Table 8). In some embodiments, modulation comprises decreasing the level of a microbial metabolite in the non-gut body site (e.g., a microbial metabolite described in Table 8).

In some embodiments, modulation comprises modulating the level of a volatile fatty acid in the non-gut body site.

In some embodiments, modulating the abundance of a bacterial taxa in a non-gut body site containing mucosal tissue of a human subject modulates treats a disease, disorder or pathological condition at the non-gut body site. In some embodiments, the non-gut body site containing mucosal tissue of a human subject is the nasal cavity. In some embodiments, the disease, disorder or pathological condition at the nasal cavity is rhinosinusitis (sinus infection), chronic rhinosinusitis (CRS), S. aureus infection or carriage, nasal vestibulitis, nasal furuncles or asthma.

In some embodiments, the non-gut body site containing mucosal tissue of a human subject is the oral cavity. In some embodiments, the disease, disorder or pathological condition at the oral cavity is dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsillitis, dentoalveolar abscess, periodontal abscess, Ludwig's angina, viral infection (e.g. herpesvirus, human papilloma virus, etc.), or fungal/yeast infections (e.g. candidiasis). In some embodiments, the non-gut body site containing mucosal tissue of a human subject is the vagina. In some embodiments, the disease, disorder or pathological condition at the vagina is bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the pharmaceutical composition modulates the abundance of a bacterial taxa in a non-gut body site containing mucosal tissue of a human subject further comprises locally or systemically administering an antimicrobial agent (e.g., an antibiotic, antifungal, or antiviral agent).

In some embodiments, the pharmaceutical composition modulates the abundance of a bacterial taxa in a non-gut body site containing mucosal tissue of a human subject further comprises locally or systemically administering an anti-inflammatory agent or steroid.

In some embodiments, the pharmaceutical composition modulates the abundance of a bacterial taxa in a non-gut body site containing mucosal tissue of a human subject further comprises locally administering a beneficial bacterial taxa (e.g., a commensal bacterial taxa residing in a healthy or non-dysbiotic non-gut body site described herein) to the non-gut body site. In some embodiments, the beneficial bacterial taxa is selected from the genera Streptococcus, Bifidobacterium, Lactobacillus, Escherichia, Weissella, Propionibacterium, and Bacillus. In some embodiments, the beneficial bacterial taxa is targeted to the oral cavity and is selected from Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, and Propionibacterium freudenreichii. In some embodiments, the beneficial bacterial taxa is targeted to the nasal cavity and is selected from Lactobacillus sakei, Lactobacillus reuteri, Streptococcus salivarius, Streptococcus thermophiles, Lactobacillus acidophilus, Bifidobacterium sp B420, and Lactobacillus GG. In some embodiments, the beneficial bacterial taxa is targeted to the vagina and is selected from Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, and Bacillus coagulans.

In some embodiments, the glycan preparation contacts the non-gut body site before traversing the GI tract. In some embodiments, less than 90, 80, 70, 60, 50, 40, 30, 20, 10, or 5%, by weight, of the glycan preparation that is locally administered enters or passes through the GI tract, e.g., passes through the stomach. In some embodiments, the glycan preparation is introduced through the vaginal opening. In some embodiments, the glycan preparation is introduced through the nares (nostrils). In some embodiments, the glycan preparation is introduced through the mouth.

### BRIEF DESCRIPTION OF THE FIGURES

FIG. 1: A portion of an exemplary catalyst with a polymeric backbone and side chains is illustrated in FIG. 1A. A portion of an exemplary catalyst, in which a side chain with the acidic group is connected to the polymeric backbone by a linker and in which a side chain with the cationic group is connected directly to the polymeric backbone is illustrated in FIG. 1B.

FIG. 2: An exemplary SEC curve between 16 and 20.5 minutes of a medium molecular weight (MW) glu100 sample showing the average MW and the MW at 10% of maximum absorption on both the leading and trailing edges of the curve.

FIG. 3: A graph comparing the degree of polymerization (DP)+ yield (bars) and the average DP (line), demonstrating that the two properties move together and can be controlled.

FIG. 4: A graph comparing the average DP and alpha-/beta-ratio of two preparations each of three different glycans demonstrates that the average DP and alpha-/beta-ratio are unrelated properties, but they can be controlled independently.

FIG. 5: A graph comparing the degree of branching (DB) and average DP of two preparations each of three different glycans demonstrates that the two properties move in tandem and can be controlled.

FIG. 6: A chart showing the relative abundance of key beneficial bacterial genera from two human oral microbiomes grown ex vivo for 20 hours with exemplary glycan preparations.

FIG. 7: An illustration depicting the increase of oral ex vivo microbiome bacteria from two subjects treated with 9 glycan preparations. All boxes represent significant fold change of the indicated genera over FOS or glucose when normalized to growth (adj. P<0.05, t-test).

### DETAILED DESCRIPTION OF THE INVENTION

Described herein are glycan preparations and pharmaceutical compositions, dosage forms suitable for local administration, and related methods, which have been found to be effective to, e.g., modulate bacterial taxa, modulate bacterial abundance, modulate pH, modulate bacterial metabolites, treat dysbioses, and/or a number of diseases, disorders or pathological conditions in various non-gut body sites that contain mucosa, such as, e.g. the oral cavity, the nasal cavity and the vagina.

### Definitions

As used herein, the term “abundance” as it relates to a microbial taxa refers to the presence of one microbial taxa as compared to another microbial taxa in a defined microbial niche at a body site, such as the oral cavity, nasal cavity, or vagina.

“Acquire” or “acquiring” as the terms are used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing a synthetic or analytical method or protocol) to obtain the value or physical entity. “Indirectly acquiring” refers to receiving the value or physical entity from another party or source (e.g., a third party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., an NMR spectrometer to obtain an NMR spectrum.

As used herein, “colonization” of a host organism refers to the non-transitory residence of a bacterium or other microbial organism in a niche.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each agent can be effected by any appropriate route of administration, including local and systemic routes. The agents can be administered by the same route or by different routes. For example, a first agent of the combination may be administered by local administration while a second agent of the combination may be administered systemically. “Diversity of a microbial community” or “microbial diversity” as used herein refers to the diversity found in the microbiota of a within a given niche or host subject. Diversity can relate to the number of distinct microbial taxa and/or richness of the microbial taxa within the niche or host and can be expressed, e.g. using the Shannon Diversity index (Shannon entropy), alpha-beta diversity, total number of observed OTUs, or Chaol index, as described herein. In some embodiments, a microbiome regulator described herein modulates diversity within a microbial community, which may be expressed using Shannon entropy as a measure. For example, the more unequal the abundances of the bacterial taxa, the larger the weighted geometric mean of the pᵢ values in Shannon's formula, and the smaller the corresponding Shannon entropy. If practically all abundance is concentrated to one taxa, and the other taxa are very rare (even if there are many of them), Shannon entropy approaches zero. When there is only one taxa Shannon entropy exactly equals zero.

As used herein, a “dosage regimen”, “dosing regimen”, or “treatment regimen” is a modality of drug administration that achieves a therapeutic objective. A dosage regimen includes definition of one, two, three, or four of: a route of administration, a unit dose, a frequency of dosage, or a length of treatment.

As used herein, a “dysbiosis” refers to the state of the microbiota under conditions of host disease, predisposition to host disease, or other unwanted condition or symptom of the host, including within a distinct microbial niche or body site, such as, e.g. the nasal cavity, the oral cavity and the vagina. In an embodiment, dysbiosis refers to the state of the microbiota under conditions of disease. Dysbiosis can be contrasted with eubiosis, which refers to the state of the microbiota under healthy conditions of the host. The state of the microbiota may include the characteristics relating to either the structure or function of the microbiota. In an embodiment, a dysbiosis includes an imbalance in the state of the microbiota, wherein the normal diversity or relative abundance of a microbial taxa is affected, e.g., relative to a second bacterial taxa or relative to the abundance of said taxa under conditions of health. In an embodiment, a dysbiosis comprises an imbalance in the function of the microbiota, e.g., a change in level of gene expression, level of a gene product, or metabolic output (e.g., an immune function such as immune surveillance or the inflammation response). In some embodiments, a dysbiosis is an undesired, e.g., unhealthy, state associated with unwanted symptoms in the host including within a distinct microbial niche or body site, such as, e.g. the nasal cavity, the oral cavity and the vagina, and that no longer promotes health, e.g., in the niche or body site.

As used herein, “ecological niche” or simply “niche” refers to the ecological space in which an organism or group of organisms occupies (such as a non-gut body site, e.g. a non-gut body site containing mucosal tissue, such as the oral cavity, nasal cavity and vagina). In some embodiments, niche specifically refers to a space that microorganisms occupy in a non-gut body site. Niche may describe how an organism or population of organisms responds to the distribution of resources, physical parameters (e.g., host tissue space, such as mucosal tissue) and competitors (e.g., by growing when resources are abundant, and when predators, parasites and pathogens are scarce) and how it in turn alters those same factors (e.g., limiting access to resources by other organisms, acting as a food source for predators and a consumer of prey).

An “effective amount” and “therapeutically effective amount” as used herein refers to an amount of a pharmaceutical composition or a drug agent that is sufficient to provide a desired effect. In some embodiments, a physician or other health professional decides the appropriate amount and dosage regimen. An effective amount also refers to an amount of a pharmaceutical composition or a drug agent that prevents the development or relapse of a medical condition.

As used herein, a “glycan preparation” is a preparation comprising glycans that exhibits a therapeutic effect. A glycan preparation comprises a synthetic mixture of a plurality of mono-, di-, oligomeric and/or polymeric glycan species (e.g. oligo- and/or polysaccharides, referred to as “oligosaccharides”), wherein the oligomeric and/or polymeric glycan species comprise glycan units that are linked by glycosidic bonds. In some embodiments, a glycan preparation may be formulated into a pharmaceutical composition for human use, e.g. for local application to a non-gut body site. In some embodiments, a glycan preparation may be formulated in any suitable dosage form including a kit. In some embodiments, glycan preparations do not contain one or more naturally occurring or synthetic oligo- or polysaccharide, including: glucooligosaccharide, mannanoligosaccharide, inulin, lychnose, maltotretraose, nigerotetraose, nystose, sesemose, stachyose, isomaltotriose, nigerotriose, maltotriose, melezitose, maltotriulose, raffinose, kestose, fructooligosaccharide, 2′-fucosyllactose, galactooligosaccharide, glycosyl, idraparinux, isomaltooligosaccharide, maltodextrin, xylooligosaccharide, agar, agarose, alginic acid, alguronic acid, alpha glucan, amylopectin, amylose, arabioxylan, beta-glucan, callose, capsulan, carrageenan, cellodextrin, cellulin, cellulose, chitin, chitin nanofibril, chitin-glucan complex, chitosan, chrysolaminarin, curdlan, cyclodextrin, alpha-cylcodextrin, dextran, dextrin, dialdehyde starch, ficoll, fructan, fucoidan, galactoglucomannan, galactomannan, galactosamineogalactan, gellan gum, glucan, glucomannan, glucoronoxyland, glycocalyx, glycogen, hemicellulose, hypromellose, icodextrin, kefiran, laminarin, lentinan, levan polysaccharide, lichenin, mannan, mucilage, natural gum, paramylon, pectic acid, pectin, pentastarch, phytoglycogen, pleuran, poligeenan, polydextrose, porphyran, pullulan, schizophyllan, sepharose, sinistrin, sizofiran, sugammadex, welan gum, xantham gum, xylan, xyloglucan, zymosan, and the like. In some embodiments, a glycan exists as a salt, e.g., a pharmaceutically acceptable salt.

A “glycan unit” as used herein refers to the individual unit of a glycan disclosed herein, e.g., the building blocks from which the glycan is made. In an embodiment, a glycan unit is a monomer.

In an embodiment, a glycan unit is a dimer. In an embodiment a glycan unit is a monosaccharide.

In an embodiment, a glycan unit is a disaccharide. In some embodiments, the glycan unit is a carbohydrate and may be selected from a sugar alcohol, a short-chain fatty acid, a sugar acid, an imino sugar, a deoxy sugar, and an amino sugar. In some embodiments, the glycan unit is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, and the like. In some embodiments, the glycan unit is glucose, galactose, arabinose, mannose, fructose, xylose, fucose, or rhamnose. In embodiments, a glycan comprises distinct glycan units, e.g., a first and a second monosaccharide, or a first and a second disaccharide, or a monosaccaride and a disaccharide. In embodiments, a glycan comprises distinct glycan units, e.g., a first, a second, a third, a fourth, and/or a fifth distinct glycan unit.

As used herein, an “isolated” or “purified” glycan preparation is substantially pure and free of contaminants, e.g. pathogens or otherwise unwanted biological material, or toxic or otherwise unwanted organic or inorganic compounds. In some embodiments, pure or isolated compounds, compositions or preparations may contain traces of solvents and/or salts (such as less than 10%, 9%, 8%, 7%, 6%, 5%, 4%, 3%, 2%, 1%, less than 0.5% or 0.1% by w/w, w/v, v/v or molar %). Purified compounds are or preparations contain at least about 60% (by w/w, w/v, v/v or molar %), at least about 75%, at least about 90%, at least about 95%, at least about 97%, at least about 98%, or at least about 99% by w/w, w/v, v/v or molar % the compound(s) of interest. For example, a purified (substantially pure) or isolated glycan preparation is one that is at least 80%, 85%, 90%, 91%, 92%, 93%, 94%, 95%, 98%, 99%, 99.5%, 99.8%, 99.9% or 100% of the glycan preparation by w/w, w/v, v/v or molar % (i.e. not including any solvent, such as e.g. water, in which the glycan preparation may be dissolved) and separated from the components that accompany it, e.g. during manufacture, extraction/purification and/or processing (e.g. such that the glycan preparation is substantially free from undesired compounds). Purity may be measured by any appropriate standard method, for example, by column chromatography (e.g., size-exclusion chromatography (SEC)), thin layer chromatography (TLC), gas chromatography (GC), high-performance liquid chromatography (HPLC) or nuclear magnatic resonance (NMR) spectroscopy. Purified or purity may also define a degree of sterility that is safe for administration to a human subject, e.g., lacking viable infectious or toxic agents.

As used herein, the terms “locally” or “local administration” mean administration at a particular site of the body intended for a substantially local effect at that site. Examples of local administration include epicutaneous, inhalational, intraarticular, intrathecal, intravaginal, intravitreal, intrauterine, intralesional, lymph node administration, intratumoral, topical administration, and administration to a mucous membrane of the subject, in each case wherein the administration is intended to have a substantially local effect. In some embodiments, the glycan preparation is applied, e.g., by spraying or droplets, instillation of a liquid, or other direct contact with the glycan preparation or a composition or dosage form comprising the glycan preparation, to the non-gut body site (e.g., a tissue or mucosa thereof). “Substantially” local means that the primary effect of the agent is concentrated at the local site (e.g., a non-gut body site containing mucosal tissue) and is not systemic (e.g. does not have a substantially systemic effect). In one embodiment, the agent (e.g. the glycan preparation) is not substantially absorbed into the blood. In one embodiment, the agent (e.g. the glycan preparation) is not substantially absorbed into the lymph system. In one embodiment, the agent (e.g. the glycan preparation) is not substantially absorbed in the gut (e.g. including stomach, colon and intestines). In one embodiment, less than 50%, 40%, 30, 20, 10, or 5%, by weight, of the glycan preparation locally administered to the non-gut body site enters or passes through the GI tract, e.g., the stomach or downstream of the stomach.

As used herein, “microbiome” refers to the genetic content of the communities of microbes that live in and on a subject (e.g. a human subject), both sustainably and transiently, including eukaryotes, archaea, bacteria, and viruses (including bacterial viruses (e.g., phage)), wherein “genetic content” includes genomic DNA, RNA such as ribosomal RNA and messenger RNA, the epigenome, plasmids, and all other types of genetic information. In some embodiments, microbiome specifically refers to genetic content of the communities of microorganisms in a niche.

“Microbiota” as used herein refers to the community of microorganisms that occur (sustainably or transiently) in and on a subject (e.g. a human subject), including eukaryotes, archaea, bacteria, and viruses (including bacterial viruses, e.g. phage). In some embodiments, microbiota specifically refers to the microbial community in a niche.

“Modulate the microbiota” or “modulating the microbiota” as used herein refers to changing the state of the microbiota. Changing the state of the microbiota may include changing the structure and/or function of the microbiota. A change in the structure of the microbiota is, e.g., a change in the relative composition of a taxa, e.g., in a non-gut body site, e.g., the oral cavity, the nasal cavity, or the vagina or a specific mucosal tissue thereof. In an embodiment, a change in the structure of the microbiota, e.g., at the non-gut body site, comprises a change in the abundance of a taxa, e.g., relative to another taxa or relative to what would be observed in the absence of the modulation. Modulation of the microbiota may also, or in addition, include a change in a function of the microbiota, such as a change in microbiota gene expression, level of a gene product (e.g., RNA or protein), or metabolic output of the microbiota. Functions of the microbiota may also include host pathogen protection and host immune modulation. Modulation of the structure or function of the microbiota may additionally induce a change in one or more functional pathway of the host (e.g., a change in gene expression, level of a gene product, and/or output of a host cell or host process) as a result of a change in the microbiota or its function. The term “nasal cavity” as used herein refers to any region or subsection of the nose and nasal passages, including the nostril/nares, nasopharynx, nasal conchae (e.g., inferior conchae), vestibule, maxilla, palatine bone, medial pterygoid plate, labyrinth of ethmoid, sinuses (e.g., paranasal sinus, frontal sinus, maxillary sinus, sphenoid sinus, ethmoid sinus), ostia, nasal wall (e.g., lateral nasal wall), infundibulum, palate, nasopharynx, olfactory epithelium, respiratory epithelium, and vomeronasal organ, including the mucosal tissues thereof.

The term “non-gut body site” as used herein refers to a body site (e.g. a site of microbial growth) other than the stomach or any portion of the GI tract after (e.g. downstream of) the stomach, e.g., the duodenum, jejunum, large intestine, duodenum, small intestine, colon, ileum, cecum and rectum. In some embodiments, a non-gut body site includes the oral cavity, the nasal cavity and the vagina. In some embodiments, non-gut mucosal tissue refers to mucosal tissue that is other than that of the stomach and any portion of the GI tract thereafter (e.g. downstream of the stomach). In some embodiments, the non-gut site comprises mucosal tissue(s).

As used herein, the term “oligosaccharide” refers to a molecule consisting of multiple (i.e., two or more) individual glycan units linked covalently. Each glycan unit may be linked through a glycosidic bond (e.g., a 1→2 glycosidic bond, a 1→3 glycosidic bond, a 1→4 glycosidic bond, a 1→5 glycosidic bond or a 1→6 glycosidic bond) present in either the alpha or beta configuration.

As used herein, the term “pathogenic” (e.g. “pathogenic bacteria”) refers to a substance, microorganism or condition that has the capability to cause a disease. In certain contexts, pathogens also include microbes (e.g. bacteria) that are associated with a disease or condition but for which a causative relationship (e.g., a direct causative relationship) has not been established or has yet to be established. In some embodiments, microbes which are not pathogens and may be commensals may cause or be associated with a disease or a dysbiosis depending on various factors (e.g. immune state of the site, abundance of the microbial taxa, etc.). Such microbes are referred to as “pathobionts.”

The term “oral cavity” as used herein refers to region or subsection of the mouth or throat, such as the lips, gums, tongue, cheek, palate (e.g., lingual palate), tonsils, salivary gland, jaw, pharynx, oropharynx, laryngopharynx, epiglottis, larynx, trachea, and esophagus, including the mucosal tissues thereof.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions meet the microbiological specifications and criteria for non-sterile pharmaceutical products as described in the U.S. Pharmacopeia (USP) or European Pharmacopoeia (EP). Pharmaceutical compositions may further comprise or may be co-administered with additional active agents, such as, e.g. additional therapeutic agents. Pharmaceutical compositions may also comprise pharmaceutically acceptable excipients, solvents, carriers, fillers, or any combination thereof.

The term “phenotype” refers to a set of observable characteristics of an individual entity. For example, an individual subject may have a phenotype of “healthy” or “diseased.” A phenotype may describe the state of an entity, wherein all entities within a phenotype share the same set of characteristics that describe the phenotype. The phenotype of an individual results in part, or in whole, from the interaction of the entities genome and/or microbiome with the environment.

As used herein, the term “polysaccharide” refers to a polymeric molecule consisting of multiple individual glycan units linked covalently. In some embodiments, a polysaccharide comprises at least 10 or more glycan units (e.g., at least 10, at least 15, at least 20, at least 25, or at least 50, at least 100, at least 250, at least 500, or at least 1000 glycan units). Each glycan unit may be linked through a glycosidic bond (e.g., a 1→2 glycosidic bond, a 1→3 glycosidic bond, a 1→4 glycosidic bond, a 1→5 glycosidic bond and a 1→6 glycosidic bond) present in either the alpha or beta confirguation. In some embodiments, a polysaccharide is a homogenous polymer comprising identical repeating units. In other embodiments, a polysaccharide is a heterogenous polymer comprised of varied repeating units. Polysaccharides may further be characterized by a degree of branching (DB, branching points per residue) or a degree of polymerization (DP).

As used herein, the term “subject” or “patient” generally refers to any human subject. The term does not denote a particular age or gender. Subjects may include pregnant women. Subjects may include a newborn (a preterm newborn, a full term newborn), an infant up to one year of age, young children (e.g., 1 yr to 12 yrs), teenagers, (e.g., 13-19 yrs), adults (e.g., 20-64 yrs), and elderly adults (65 yrs and older). A subject does not include an agricultural animal, e.g., farm animals or livestock, e.g., cattle, horses, sheep, swine, chickens, etc. In general, a subject comprises a host and its corresponding microbiota.

A “substantial decrease” as used herein is a decrease of 5%, 10%, 20%, 30%, 40%, 50%, 60%, 70%, 80%, 90%, 95%, 97%, 98%, 99%, 99.9%, or 100%.

A “substantial increase” as used herein is an increase of 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000%, or more than 1000%.

“Synthetic” as used herein refers to a man-made compound or preparation, such as a glycan preparation, that is not naturally occurring. In one embodiment, the polymeric catalyst described herein is used to synthesize the glycans of the preparation under suitable reaction conditions, e.g. by a polymerization (or condensation) reaction that creates oligomers and polymers from individual glycan units that are added to the reaction. In some embodiments, the polymeric catalyst acts as a hydrolysis agent and can break glycosidic bonds. In other embodiments, the polymer catalyst can form glycosidic bonds (hydrolysis). Synthetic glycan preparations may also include glycan preparations that are not isolated from a natural oligo- or polysaccharide source (e.g. N-linked or O-linked glycans from polypeptides). It is to be understood that while the glycan preparation is not isolated from a natural oligo- or polysaccharide source, the glycan units making up the glycan preparation can be and often are isolated from natural oligo- or polysaccharide sources, including those listed herein, or are synthesized de novo.

The terms “treating” and “treatment” as used herein refer to the administration of an agent or composition to a subject (e.g., a symptomatic subject afflicted with an adverse condition, disorder, or disease) so as to affect a reduction in severity and/or frequency of a symptom, eliminate a symptom and/or its underlying cause, and/or facilitate improvement or remediation of damage, and/or preventing an adverse condition, disorder, or disease in an asymptomatic subject who is susceptible to a particular adverse condition, disorder, or disease, or who is suspected of developing or at risk of developing the condition, disorder, or disease.

The term “vagina” as used herein refers to region or subsection of the vagina or surrounding area, including the labia, vulva, cervix, uterus, fallopian tube, ovary, urethra, and bladder, including the mucosal tissues thereof.

### Generation of Glycan Preparations

Preparations comprising a plurality of glycans such as, e.g., oligosaccharide mixtures can be generated using a non-enzymatic catalyst, e.g., the polymeric catalyst described in U.S. Pat. No. 8,466,242, “POLYMERIC ACID CATALYSTS AND USES THEREOF” or by other suitable methods. Methods to prepare the polymeric and solid-supported catalysts described herein can be found in WO 2014/031956, “POLYMERIC AND SOLID-SUPPORTED CATALYSTS, AND METHODS OF DIGESTING CELLULOSIC MATERIALS USING SUCH CATALYSTS.” The glycans generated, e.g., by using the catalyst, for example as described in WO 2016/007778, “OLIGOSACCHARIDE COMPOSITIONS AND METHODS FOR PRODUCING THEREOF” and WO/2016/122889 “GLYCAN THERAPEUTICS AND RELATED METHODS THEREOF” can be structurally much more diverse glycans than those produced by enzymatic reactions. All patent applications are incorporated herein by reference. Provided are also methods for generating the preparations of glycans (e.g. oligosaccharides) described herein, for example by: a) providing one or more mono- or disaccharide glycan unit, or a combination thereof, b) contacting the mono- or disaccharides with any of the polymeric catalysts described herein and a suitable solvent (such as, e.g. water or a non-aqueous solvent) for a period of time sufficient to produce a polymerized species population (with a desired average degree of polymerization); and c) isolating and/or recovering at least a portion of the polymerized glycan preparation.

In some embodiments, preparations of glycans (e.g. oligosaccharides) are polymolecular. In some embodiments, preparations of glycans (e.g. oligosaccharides) are polymolecular and polydisperse. For example, the glycan preparations comprise a mixture of distinct oligosaccharide species (e.g. of different degree of polymerization and degree of branching and different alpha-to-beta glycosidic bond ratios). In some embodiments, the glycan preparations comprise a plurality of distinct species (e.g. oligosaccharides) and may consist of 1×10³, 1×10⁴, 1×10⁵, 1×10⁶, 1×10⁷, 1×10, 1×10⁹, 1×10¹⁰, 1×10¹¹, 1×10¹², 1×10¹³, 1×10¹⁴, or more species in various proportions to each other. Herein described are the average properties of the glycan preparations, such as degree of polymerization, degree of branching, alpha- and beta-glycosidic bond ratios, etc.

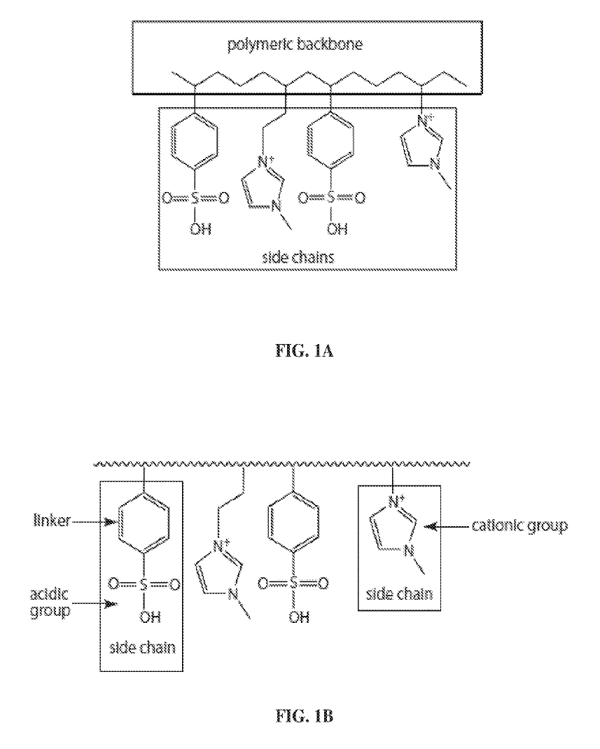

In certain embodiments, the starting material (comprising the glycan units) is contacted with a polymer catalyst under conditions that promote the formation of one or more glycosidic bond between glycan units, thereby producing a preparation of glycans. In one embodiment, the glycan unit is a monosaccharide. In one embodiment, the glycan unit is a disaccharide. Suitable polymer catalysts comprise acidic monomers and ionic monomers that are connected to form a polymeric backbone, wherein each acidic monomer has at least one Bronsted-Lowry acid, and each ionic monomer independently has at least one nitrogen-containing cationic group or phosphorous-containing cationic group. In some embodiments, each acidic monomer of the polymer catalyst may have one Bronsted-Lowry acid, and optionally the Bronsted-Lowry acids are distinct. In some embodiments, each ionic monomer of the polymer catalyst has one nitrogen-containing cationic group or phosphorous-containing cationic group. In some embodiments, at least one ionic monomer of the polymer catalyst has two nitrogen-containing cationic groups or phosphorous-containing cationic groups. A schematic outlining the general functional groups is shown in FIGS. 1a and 1b.

Generally, the polymeric catalyst and the glycan units are introduced into an interior chamber of a reactor, either concurrently or sequentially. Glycan (e.g. oligosaccharides) synthesis can be performed in a batch process or a continuous process. For example, in one embodiment, glycan synthesis is performed in a batch process, where the contents of the reactor are continuously mixed or blended, and all or a substantial amount of the products of the reaction are removed (e.g. isolated and/or recovered). In one variation, glycan synthesis is performed in a batch process, where the contents of the reactor are initially intermingled or mixed but no further physical mixing is performed. In another variation, glycan synthesis is performed in a batch process, wherein once further mixing of the contents, or periodic mixing of the contents of the reactor, is performed (e.g., at one or more times per hour), all or a substantial amount of the products of the reaction are removed (e.g. isolated and/or recovered) after a certain period of time.

In other embodiments, glycan (e.g. oligosaccharide) synthesis is performed in a continuous process, where the contents flow through the reactor with an average continuous flow rate but with no explicit mixing. After introduction of the polymeric catalyst and glycan units into the reactor, the contents of the reactor are continuously or periodically mixed or blended, and after a period of time, less than all of the products of the reaction are removed (e.g. isolated and/or recovered). In one variation, glycan synthesis is performed in a continuous process, where the mixture containing the catalyst and glycan units is not actively mixed. Additionally, mixing of catalyst and the glycan units may occur as a result of the redistribution of polymeric catalysts settling by gravity, or the non-active mixing that occurs as the material flows through a continuous reactor.

In some embodiments of the method, the starting material for the polymerization reaction is one or more glycan unit selected from one or more monosaccharides, one or more disaccharides, or a combination thereof. In some embodiments of the method, the starting material for the polymerization reaction is one or more glycan unit selected from a furanose sugar and a pyranose sugar. In some embodiments of the method, the starting material for the polymerization reaction is one or more glycan unit selected from a tetrose, a pentose, a hexose, or a heptose. In some embodiments of the method, the starting material for the polymerization reaction is one or more glycan unit selected from a glucose, a galactose, an arabinose, a mannose, a fructose, a xylose, a fucose, and a rhamnose, all optionally in either their L- or D-form, in alpha or beta configuration (for dimers), and/or a deoxy-form, where applicable, and any combination thereof. In some embodiments, the glycan units are substituted or derivatized with one or more of an acetate ester, sulfate half-ester, phosphate ester, or a pyruvyl cyclic acetal group, or have been otherwise derivatized at, e.g., at one or more hydroxyl groups.

The glycan units used in the methods described herein may include one or more sugars. In some embodiments, the one or more sugars are selected from monosaccharides, disaccharides, and trisaccharides, or any mixtures thereof. In some embodiments, the one or more sugars are monosaccharides, such as one or more C5 or C6 monosaccharides. In some embodiments, the one or more sugars are C5 monosaccharides. In other embodiments, the one or more sugars are C6 monosaccharides.

In some embodiments, the starting material for the polymerization reaction is one or more glycan unit selected from monosaccharides and other carbohydrates including glycolaldehyde, glyceraldehyde, dihydroxyacetone, erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, neuraminic acid, N-acetylneuraminic acid, N-acetylgalactosamine, N-acetylglucosamine, fructosamine, galactosamine, glucosamine, sorbitol, glycerol, erythritol, threitol, arabitol, xylitol, mannitol, sorbitol, galactitol, fucitol, and lactic acid.

In some embodiments, the starting material for the polymerization reaction is one or more glycan unit selected from a monosaccharide. In some embodiments, the monosaccharide is glucose, galactose, fructose, fucose, mannose, arabinose, rhamnose, and xylose. In one embodiment, the glycan unit is not glucose. In one embodiment, the glycan unit is not galactose. In one embodiment, the glycan unit is not fructose. In one embodiment, the glycan unit is not fucose. In one embodiment, the glycan unit is not mannose. In one embodiment, the glycan unit is not arabinose. In one embodiment, the glycan unit is not rhamnose. In one embodiment, the glycan unit is not xylose.

In some embodiments, the starting material for the polymerization reaction is one or more glycan unit selected from disaccharides and other carbohydrates including acarviosin, N-acetyllactosamine, allolactose, cellobiose, chitobiose, glactose-alpha-1,3-galactose, gentiobiose, isomalt, isomaltose, isomaltulose, kojibiose, lactitol, lactobionic acid, lactose, lactulose, laminaribiose, maltitol, maltose, mannobiose, melibiose, melibiulose, neohesperidose, nigerose, robinose, rutinose, sambubiose, sophorose, sucralose, sucrose, sucrose acetate isobutyrate, sucrose octaacetate, trehalose, turanose, vicianose, and xylobiose.

In some embodiments, the starting material for the polymerization reaction is one or more glycan unit selected from an amino sugar, a deoxy sugar, an imino sugar, a sugar acid, a short-chain fatty acid, and a sugar alcohol.

Suitable glycan units include amino sugars, such as, e.g. acarbose, N-acetylemannosamine, N-acetylmuramic acid, N-acetylneuraminic acid, N-acetyletalosaminuronic acid, arabinopyranosyl-N-methyl-N-nitrosourea, D-fructose-L-histidine, N-glycolyneuraminic acid, ketosamine, kidamycin, mannosamine, 1B-methylseleno-N-acetyl-D-galactosamine, muramic acid, muramyl dipeptide, phosphoribosylamine, PUGNAc, sialyl-Lewis A, sialyl-Lewis X, validamycin, voglibose, N-acetylgalactosamine, N-acetylglucosamine, aspartylglucosamine, bacillithiol, daunosamine, desosamine, fructosamine, galactosamine, glucosamine, meglumine, and perosamine.

Suitable glycan units include deoxy sugars, such as, e.g. 1-5-ahydroglucitol, cladinose, colitose, 2-deoxy-D-glucose, 3-deoxyglucasone, deoxyribose, dideoxynucleotide, digitalose, fludeooxyglucose, sarmentose, and sulfoquinovose.

Suitable glycan units include imino sugars, such as, e.g. castanospermine, 1-deoxynojirimycin, iminosugar, miglitol, miglustat, and swainsonine.

Suitable glycan units include sugar acids, such as, e.g. N-acetylneuraminic acid, N-acetyltalosamnuronic acid, aldaric acid, aldonic acid, 3-deoxy-D-manno-oct-2-ulosonic acid, glucuronic acid, glucosaminuronic acid, glyceric acid, N-glycolylneuraminic acid, iduronic acid, isosaccharinic acid, pangamic acid, sialic acid, threonic acid, ulosonic acid, uronic acid, xylonic acid, gluconic acid, ascorbic acid, ketodeoxyoctulosonic acid, galacturonic acid, galactosaminuronic acid, mannuronic acid, mannosaminuronic acid, tartaric acid, mucic acid, saccharic acid, lactic acid, oxalic acid, succinic acid, hexanoic acid, fumaric acid, maleic acid, butyric acid, citric acid, glucosaminic acid, malic acid, succinamic acid, sebacic acid, and capric acid.

Suitable glycan units include short-chain fatty acids, such as, e.g., formic acid, acetic acid, propionic acid, butryic acid, isobutyric acid, valeric acid, and isovaleric acid.

Suitable glycan units include sugar alcohols, such as, e.g., methanol, ethylene glycol, glycerol, erythritol, threitol, arabitol, ribitol, xylitol, mannitol, sorbitol, galactitol, iditol, volemitol, fucitol, inositol, maltotritol, maltotetraitol, and polyglycitol.

In some embodiments, the glycan unit may exist as a salt (e.g., a pharmaceutically acceptable salt), such as, e.g., a hydrochlorate, hydroiodate, hydrobromate, phosphate, sulfate, methanesulfate, acetate, formate, tartrate, malate, citrate, succinate, lactate, gluconate, pyruvate, fumarate, propionate, aspartate, glutamate, benzoate, ascorbate salt.

The glycan units used in the methods described herein may be obtained from any commercially known sources, or produced according to any methods known in the art.

In some embodiments, the glycan preparation is synthetic and not isolated from a natural product (e.g., a natural oligosaccharide or natural polysaccharide). In some embodiments, the glycan preparation is not derived or prepared from an N-linked glycan or an O-linked glycan. In some embodiments, the glycan preparation is not derived or prepared from a mucin.

Reaction Conditions

In some embodiments, the glycan units and catalyst (e.g., polymeric catalyst or solid-supported catalyst) are allowed to react for at least 1 hour, at least 2 hours, at least 3 hours, at least 4 hours, at least 6 hours, at least 8 hours, at least 16 hours, at least 24 hours, at least 36 hours, or at least 48 hours; or between 1-24 hours, between 2-12 hours, between 3-6 hours, between 1-96 hours, between 12-72 hours, or between 12-48 hours.

In some embodiments, the degree of polymerization (DP) of the glycan preparation produced according to the methods described herein can be regulated by the reaction time. For example, in some embodiments, the degree of polymerization of the glycan preparation is increased by increasing the reaction time, while in other embodiments, the degree of polymerization of the glycan preparation is decreased by decreasing the reaction time.

### Reaction Temperature

In some embodiments, the reaction temperature is maintained in the range of about 25° C. to about 150° C. In certain embodiments, the temperature is from about 30° C. to about 125° C., about 60° C. to about 120° C., about 80° C. to about 115° C., about 90° C. to about 110° C., about 95° C. to about 105° C., or about 100° C. to 110° C.

### Amount of Glycan Units

The amount of the glycan unit used in the methods described herein relative to the amount solvent used may affect the rate of reaction and yield. The amount of the glycan unit used may be characterized by the dry solids content. In certain embodiments, dry solids content refers to the total solids of a slurry as a percentage on a dry weight basis. In some embodiments, the dry solids content of the glycan unit is between about 5 wt % to about 95 wt %, between about 10 wt % to about 80 wt %, between about 15 wt %, to about 75 wt %, or between about 15 wt %, to about 50 wt %.

### Amount of Catalyst

The amount of the catalyst used in the methods described herein may depend on several factors including, for example, the selection of the type(s) of glycan unit, the concentration of the glycan unit, and the reaction conditions (e.g., temperature, time, and pH). In some embodiments, the weight ratio of the catalyst to the glycan unit(s) is about 0.01 g/g to about 50 g/g, about 0.01 g/g to about 5 g/g, about 0.05 g/g to about 1.0 g/g, about 0.05 g/g to about 0.5 g/g, about 0.05 g/g to about 0.2 g/g, or about 0.1 g/g to about 0.2 g/g.

### Solvent

In certain embodiments, synthesis of the glycans (e.g. oligosaccharides) using the polymeric catalyst is carried out in an aqueous environment. One suitable aqueous solvent is water. Generally, water with lower concentrations of ionic species is preferable, as such ionic species may reduce the effectiveness of the polymeric catalyst. In some embodiments where the aqueous solvent is water, the water has less than 10% of ionic species (e.g., salts of sodium, phosphorous, ammonium, magnesium). In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

### Water Content

In some embodiments, water is produced with each glycosidic bond formed between the one or more glycan units (dehydration reaction). In certain embodiments, the methods described herein may further include monitoring the amount of water present in the reaction mixture and/or the ratio of water to glycan unit or catalyst over a period of time. In some embodiments, the method further includes removing at least a portion of water produced in the reaction mixture (e.g., by removing at least about any of 10%, 20%, 30%, 40%, 50%, 60%, 70%, 80%, 90%, 95%, 97%, 99%, or 100%, such as by vacuum filtration). It should be understood, however, that the amount of water to glycan unit may be adjusted based on the reaction conditions and specific catalyst used.

Any method known in the art may be used to remove water in the reaction mixture, including, for example, by vacuum filtration, vacuum distillation, heating, and/or evaporation. In some embodiments, the method comprises including water in the reaction mixture.

In some aspects, provided herein are methods of producing a glycan preparation, by: combining a glycan unit and a catalyst having acidic and ionic moieties to form a reaction mixture, wherein water is produced in the reaction mixture; and removing at least a portion of the water produced in the reaction mixture. In certain variations, at least a portion of water is removed to maintain a water content in the reaction mixture of less than 99%, less than 90%, less than 80%, less than 70%, less than 60%, less than 50%, less than 40%, less than 30%, less than 20%, less than 10%, less than 5%, or less than 1% by weight.

In some embodiments, the degree of polymerization of the glycan preparation produced can be regulated by adjusting or controlling the concentration of water present in the reaction mixture.

For example, in some embodiments, the degree of polymerization of the glycan preparation is increased by decreasing the water concentration, while in other embodiments, the degree of polymerization of the glycan preparation is decreased by increasing the water concentration. In some embodiments, the water content of the reaction is adjusted during the reaction to regulate the degree of polymerization of the glycan preparation produced.

For example, a majority, e.g. about 55%, 60%, 65%, 70%, 75%, 80%, 85%, 90%, 95%, or about 97% of the glycan preparation has a DP of between 2 and 25, between 3 and 25, between 4 and 25, between 5 and 25, between 6 and 25, between 7 and 25, between 8 and 25, between 9 and 25, between 10 and 25, between 2 and 30, between 3 and 30, between 4 and 30, between 5 and 30, between 6 and 30, between 7 and 30, between 8 and 30, between 9 and 30, or between 10 and 30.

In one example, to a round bottom flask equipped with an overhead stirrer and a jacketed short-path condenser one or more glycan units may be added along with 1-50% (1-10%, 1-20%, 1-30%, 1-40%, 1-60%, 1-70%) by dry weight of one or more of the catalysts described herein.

Water or another compatible solvent (0.1-5 equiv, 1-5 equiv, 1-4 equiv, 0.1-4 equiv) may be added to the dry mixture and the slurry can be combined at slow speed (e.g. 10-100 rpm, 50-200 rpm, 100-200 rpm) using a paddle sized to match the contours of the selected round bottom flask as closely as possible. The mixture is heated to 70-180° C. (70-160° C., 75-165° C., 80-160° C.) under 10-1000 mbar vacuum pressure. The reaction may be stirred for 30 minutes to 6 hours, constantly removing water from the reaction. Reaction progress can be monitored by HPLC.

The yield of conversion for the one or more glycan units in the methods described herein can be determined by any suitable method known in the art, including, for example, high performance liquid chromatography (HPLC). In some embodiments, the yield of conversion to a glycan preparation with DP>1 after combining the one or more glycan units with the catalyst (e.g., at 2, 3, 4, 8, 12, 24, or 48 hours after combining the one or more glycan units with the catalyst) is greater than about 50% (e.g., greater than about 55%, 60%, 65%, 70%, 75%, 80%, 85%, 90%, 95%, or 98%). In some embodiments, the yield of conversion to a glycan preparation with >DP2 after combining the one or more glycan units with the catalyst (e.g., at 2, 3, 4, 8, 12, 24, or 48 hours after combining the one or more glycan units with the catalyst) is greater than 30% (e.g., greater than 35%, 40%, 45%, 50%, 55%, 60%, 65%, 70%, 75%, 80%, 85%, 90%, 95%, or 98%).

In some embodiments, the yield of conversion to a glycan preparation with >DP3 after combining the one or more glycan units with the catalyst (e.g., at 2, 3, 4, 8, 12, 24, or 48 hours after combining the one or more glycan units with the catalyst) is greater than 30% (e.g., greater than 35%, 40%, 45%, 50%, 55%, 60%, 65%, 70%, 75%, 80%, 85%, 90%, 95%, or 98%).

In some embodiments, the glycan preparation has a degree of polymerization (DP) distribution after combining the one or more glycan units with the polymeric catalyst (e.g., at 2, 3, 4, 8, 12, 24, or 48 hours after combining the one or more glycan units with the catalyst) is: DP2=0%-40%, such as less than 40%, less than 30%, less than 20%, less than 10%, less than 5%, or less than 2%; or 10%-30% or 15%-25%; DP3=0%-20%, such as less than 15%, less than 10%, less than 5%; or 5%-15%; and DP4+=greater than 15%, greater than 20%, greater than 30%, greater than 40%, greater than 50%; or 15%-75%, 20%-40% or 25%-35%.

The solid mass obtained by the process can be dissolved in a volume of water sufficient to create a solution of approximately 50 Brix (grams sugar per 100 g solution). Once dissolution is complete, the solid catalyst can be removed by filtration. The solution comprising therapeutic glycans can be concentrated to about 50-75 Brix, e.g., by rotary evaporation. In some embodiments, the solution comprising therapeutic glycans can be concentrated to about 50-60 Brix, 60-70 Brix, 70-80 Brix, 55-65 Brix, 65-75 Brix, or 75-85 Brix. In some embodiments, the solution comprising therapeutic glycans can be concentrated to about 50, 55, 60, 65, 70, 75, 80, or about 85 Brix. Optionally, an organic solvent can be used and water immiscible solvents can be removed by biphasic extraction and water miscible solvents can be removed, e.g., by rotary evaporation concomitant to the concentration step.

Additional processing steps Optionally, the glycan preparation produced may undergo additional processing steps.

Additional processing steps may include, for example, purification steps. Purification steps may include, for example, separation, dilution, concentration, filtration, desalting or ion-exchange, chromatographic separation, or decolorization, or any combination thereof.

### Decolorization

In some embodiments, the methods described herein further include a decolorization step. The glycan preparation produced may undergo a decolorization step using any method known in the art, including, for example, treatment with an absorbent, activated carbon, chromatography (e.g., using ion exchange resin), hydrogenation, and/or filtration (e.g., microfiltration).

In certain embodiments, the glycan preparations produced are contacted with a color-absorbing material at a particular temperature, at a particular concentration, and/or for a particular duration of time. In some embodiments, the mass of the color absorbing species contacted with the glycan preparation is less than 50% of the mass of the glycan preparation, less than 35% of the mass of the glycan preparation, less than 20% of the mass of the glycan preparation, less than 10% of the mass of the glycan preparation, less than 5% of the mass of the glycan preparation, less than 2% of the mass of the glycan preparation, or less than 1% of the mass of the glycan preparation.

In some embodiments, the glycan preparations are contacted with a color absorbing material. In certain embodiments, the glycan preparations are contacted with a color absorbing material for less than 10 hours, less than 5 hours, less than 1 hour, or less than 30 minutes. In a particular embodiment, the glycan preparations are contacted with a color absorbing material for 1 hour.

In certain embodiments, the glycan preparations are contacted with a color absorbing material at a temperature from about 20 to 100 degrees Celsius, about 30 to 80 degrees Celsius, about 40 to 80 degrees Celsius, or about 40 to 65 degrees Celsius. In a particular embodiment, the glycan preparations are contacted with a color absorbing material at a temperature of about 50 degrees Celsius.

In certain embodiments, the color absorbing material is activated carbon. In one embodiment, the color absorbing material is powdered activated carbon. In other embodiments, the color absorbing material is an ion exchange resin. In one embodiment, the color absorbing material is a strong base cationic exchange resin in a chloride form. In another embodiment, the color absorbing material is cross-linked polystyrene. In yet another embodiment, the color absorbing material is cross-linked polyacrylate. In certain embodiments, the color absorbing material is Amberlite FPA91, Amberlite FPA98, Dowex 22, Dowex Marathon MSA, or Dowex Optipore SD-2.

### Ion-Exchange/De-Salting (Demineralization)

In some embodiments, the glycan preparations are contacted with a material to remove salts, minerals, and/or other ionic species. In certain embodiments, the glycan preparations are flowed through an anionic/cationic exchange column pair. In one embodiment, the anionic exchange column contains a weak base exchange resin in a hydroxide form and the cationic exchange column contains a strong acid exchange resin in a protonated form.

### Separation and Concentration

In some embodiments, the methods described herein further include isolating the glycan preparation produced. In certain variations, isolating the glycan preparation comprises separating at least a portion of the glycan preparation from at least a portion of the catalyst, using any method known in the art, including, for example, centrifugation, filtration (e.g., vacuum filtration, membrane filtration), and gravity settling. In some embodiments, isolating the glycan preparation comprises separating at least a portion of the glycan preparation from at least a portion of any unreacted glycan units, using any method known in the art, including, for example, filtration (e.g., membrane filtration), chromatography (e.g., chromatographic fractionation), differential solubility, and centrifugation (e.g., differential centrifugation). In some embodiments, the methods further include a concentration step. For example, the isolated glycan preparations undergo evaporation (e.g., vacuum evaporation) to produce a concentrated glycan preparation. In other embodiments, the isolated glycan preparations undergo a spray drying step to produce a powdered glycan preparation. In certain embodiments, the isolated glycan preparations undergo both an evaporation step and a spray drying step.

### Fractionation

In some embodiments, glycan preparations (e.g. oligosaccharides) are created that are polydisperse, exhibiting a range of degrees of polymerization. In some embodiments, the methods described herein further include a fractionation step. Glycan species (e.g., oligosaccharides) may be separated by molecular weight using any method known in the art, including, for example, high-performance liquid chromatography, adsorption/desorption (e.g. low-pressure activated carbon chromatography), or filtration (for example, ultrafiltration or diafiltration). In certain embodiments, glycan species are separated into pools representing 60%, 65%, 70%, 75%, 80%, 85%, 90%, 95%, 97%, 98%, or greater than 98% short (about DP1-2), medium (about DP3-10), long (about DP11-18), or very long (about DP>18) species.

In certain embodiments, glycan species are fractionated by adsorption onto a carbonaceous material and subsequent desorption of fractions by washing the material with mixtures of an organic solvent in water at a concentration of 1%, 5%, 10%, 20%, 50%, or 100%. In one embodiment, the adsorption material is activated charcoal. In another embodiment, the adsorption material is a mixture of activated charcoal and a bulking agent such as diatomaceous earth or Celite 545 in 5%, 10%, 20%, 30%, 40%, or 50% portion by volume or weight.

In further embodiments, glycan species are separated by passage through a high-performance liquid chromatography system. In certain variations, glycan species are separated by ion-affinity chromatography, hydrophilic interaction chromatography, or size-exclusion chromatography including gel-permeation and gel-filtration.

In other embodiments, low molecular weight materials are removed by filtration methods. In certain variations, low molecular weight materials may be removed by dialysis, ultrafiltration, diafiltration, or tangential flow filtration. In certain embodiments, the filtration is performed in static dialysis tube apparatus. In other embodiments, the filtration is performed in a dynamic flow filtration system. In other embodiments, the filtration is performed in centrifugal force-driven filtration cartridges.

### Characteristics of Glycan Preparations

The glycan preparations described herein may comprise oligosaccharides and/or polysaccharides (referred to herein as “oligosaccharides”). In some embodiments, the glycan preparations comprise homo-oligo- or polymers (e.g., homoglycans), wherein all the glycan units in the oligomer or polymer are of the same type. Glycan preparations comprising homopolymers can include monosaccharides bonded together via a single or multiple glycosidic bond types.

In some embodiments, the glycan preparations comprise hetero-oligo- or polymers (e.g., heteroglycans), wherein more than one type of glycan unit is present. Glycan preparations comprising heteropolymers can include distinct types of monosaccharides bonded together via a single or multiple glycosidic bond types.

In some embodiments, hydrolysis may be used to generate the constituent glycan units that are suitable to produce the glycans described herein. In one embodiment, the glycan unit is a monosaccharide. Monosaccharides may exist in many different forms, for example, conformers, cyclic forms, acyclic forms, stereoisomers, tautomers, anomers, and isomers.

### Degree of Polymerization

In some embodiments, about 55%, 60%, 65%, 70%, 75%, 80%, 85%, 90%, 95%, or about 97% of the glycan preparation has a DP of at least 5 and less than 30 glycan units. In some embodiments, about 55%, 60%, 65%, 70%, 75%, 80%, 85%, 90%, 95%, or about 97% of the glycan preparation has a DP of at least 3 and less than 30 glycan units. In some embodiments, about 55%, 60%, 65%, 70%, 75%, 80%, 85%, 90%, 95%, or about 97% of the glycan preparation has a DP of at least 3 and less than 25 glycan units. In some embodiments, about 55%, 60%, 65%, 70%, 75%, 80%, 85%, 90%, 95%, or about 97% of the glycan preparation has a DP of at least 8 and less than 30 glycan units. In some embodiments, about 55%, 60%, 65%, 70%, 75%, 80%, 85%, 90%, 95%, or about 97% of the glycan preparation has a DP of at least 10 and less than 30 glycan units. In some embodiments, about 55%, 60%, 65%, 70%, 75%, 80%, 85%, 90%, 95%, or about 97% of the glycan preparation has a DP of between 3, 4, 5, 6, 7, 8 and 10, 11, 12, 13, 14, 15, 16, 17, 18, 19, 20 glycan units. In some embodiments, about 55%, 60%, 65%, 70%, 75%, 80%, 85%, 90%, 95%, or about 97% of the glycan preparation has a DP of between 10, 11, 12, 13, 14, 15, 16, 17, 18, 19 and 20, 21, 22, 23, 24, 25, 26, 27, 28, 29, 30 glycan units. In some embodiments, about 55%, 60%, 65%, 70%, 75%, 80%, 85%, 90%, 95%, or about 97% of the glycan preparation has a DP of between 3, 4, 5, 6, 7, 8, 9, 10 and 20, 21, 22, 23, 24, 25, 26, 27, 28 glycan units.

In one embodiment, the glycan preparation has a degree of polymerization (DP) of at least 3 and less than 30 glycan units. In one embodiment, the glycan preparation has a degree of polymerization (DP) of at least 5 and less than 30 glycan units. In one embodiment, the glycan preparation has a degree of polymerization (DP) of at least 3 and less than 25 glycan units.

In one embodiment, about 55%, 60%, 65%, 70%, 75%, 80%, 85%, 90%, 95%, or about 97% of the glycan preparation has a DP of at least 2. In one embodiment, about 55%, 60%, 65%, 70%, 75%, 80%, 85%, 90%, 95%, or about 97% of the glycan preparation has a DP of at least 3.

In some embodiments, glycan preparations are provided, wherein at least 5%, 10%, 15%, 20%, 25%, 30%, 35%, 40%, 45%, 50%, 55%, 60%, 65%, 70%, 75%, 80%, 85%, 90%, 95%, 97%, 98%, 99%, 99.8%, or at least 99.9% or even 100% of the glycan preparation has a degree of polymerization (DP) of at least 2, 3, 4, 5, 6, 7, 8, 9, 10, 11, or at least 12 glycan units and less than 75, 70, 65, 60, 55, 50, 45, 40, 35, 30, 25, 20, 19, 18, 17, 16, or less than 15 glycan units.

In some embodiments, glycan preparations are provided, wherein at least 5%, 10%, 15%, 20%, 25%, 30%, 35%, 40%, 45%, 50%, 55%, 60%, 65%, 70%, 75%, 80%, 85%, 90%, 95%, 97%, 98%, 99%, 99.8%, or at least 99.9% or even 100% of the glycan preparation has a degree of polymerization (DP) of at least 3 and less than 30 glycan units, at least 5 and less than 30 glycan units, or at least 8 and less than 30 glycan units.

In some embodiments, about 55%, 60%, 65%, 70%, 75%, 80%, 85%, 90%, 95%, or about 97% of the glycan preparation has an average degree of polymerization (DP) of about DP5, DP6, DP7, DP8, DP9, DP10, DP11, DP12, DP13, DP14, or DP15.

In some embodiments, glycan preparations are provided wherein at least 50%, 60%, 70%, or 80% of the glycan preparation has a degree of polymerization of at least 3 and less than 30 glycan units, or of at least 5 and less than 25 glycan units. In some embodiments, the average DP of the glycan preparation is between about DP7 and DP9 or between about DP6 and DP10. In some embodiments, these glycan preparations comprise an alpha- to beta-glycosidic bond ratio from 0.8:1 to 5:1 or from 1:1 to 4:1. In some embodiments, the fractionated preparations have an average degree of branching of between about 0.01 and about 0.2 or between about 0.05 and 0.1.

In one embodiment, a polydisperse, fractionated glycan preparation is provided comprising at least 85%, 90%, or at least 95% medium-length species with a DP of about 3-10. In one embodiment, a polydisperse, fractionated glycan preparation is provided comprising at least 85%, 90%, or at least 95% long-length species with a DP of about 11-18. In one embodiment, a polydisperse, fractionated glycan preparation is provided comprising at least 85%, 90%, or at least 95% very long-length species with a DP of about 18-30. In some embodiments, the medium, long and very long fractionated preparations comprise an alpha- to beta-glycosidic bond ratio from 0.8:1 to 5:1 or from 1:1 to 4:1. In some embodiments, the fractionated preparations have an average degree of branching of between about 0.01 and about 0.2 or between about 0.05 and 0.1.

In some embodiments, about 55%, 60%, 65%, 70%, 75%, 80%, 85%, 90%, 95%, or about 97% of the glycan preparation has an average molecular weight of about 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800 g/mol and less than 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, and 5000 g/mol.

### Degree of Branching

In some embodiments, the glycan preparations (e.g. oligosaccharides) range in structure from linear to highly branched. Unbranched glycans may contain only alpha linkages or only beta linkages. Unbranched glycans may contain at least one alpha and at least one beta linkage. Branched glycans may contain at least one glycan unit being linked via an alpha or a beta glycosidic bond so as to form a branch. The branching rate or degree of branching (DB) may vary, such that about every 2^{nd}, 3^{rd}, 4^{th}, 5^{th}, 6^{th}, 7^{th}, 8^{th}, 9^{th}, 10^{th}, 15^{th}, 20^{th}, 25^{th}, 30^{th}, 35^{th}, 40^{th}, 45^{th}, 50^{th}, 60^{th}, or 70^{th} unit comprises at least one branching point. For example, animal glycogen contains a branching point approximately every 10 units.

In some embodiments, glycan preparations are provided, wherein the preparation comprises a mixture of branched glycans, wherein the average degree of branching (DB, branching points per residue) is 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.95, 0.99, 1, or 2. In some embodiments, glycan preparations are provided, wherein the average degree of branching is at least 0.01, 0.05, 0.1, 0.2, 0.3, or at least 0.4. In some embodiments, glycan preparations are provided, wherein the average degree of branching is between about 0.01 and 0.1, 0.01 and 0.2, 0.01 and 0.3, 0.01 and 0.4, or 0.01 and 0.5. In some embodiments, glycan preparations are provided, wherein the average degree of branching is between about 0.05 and 0.1, 0.05 and 0.2, 0.05 and 0.3, 0.05 and 0.4, or 0.05 and 0.5. In some embodiments, glycan preparations are provided, wherein the average degree of branching is between about 0.1 and 0.2, 0.1 and 0.3, 0.1 and 0.4, or 0.1 and 0.5. In some embodiments, glycan preparations are provided, wherein the average degree of branching is not 0. In some embodiments, glycan preparations are provided, wherein the average degree of branching is not between at least 0.1 and less than 0.4 or at least 0.2 and less than 0.4. In some embodiments, the glycan preparations comprise linear glycans. In some embodiments, the glycan preparations comprise glycans that exhibit a branched or branch-on-branch structure, e.g., branched glycans (such as, e.g., branched oligosaccharides and/or branched polysaccharides).

In some embodiments, glycan preparations are provided wherein the average degree of branching (DB) is not 0, but is at least 0.01, 0.05, 0.1, or at least 0.2, or ranges between about 0.01 and about 0.2 or between about 0.05 and 0.1.

### Glycosidic Linkages

The linkage or bonds between two glycan units can be expressed, for example, as 1,4, 1→4, or (1-4), used interchangeably and are referred to herein as glycosidic linkages or bonds for compounds comprising one or more sugars (e.g. monosaccharides, disaccharides and the like). Monosaccharides can be in the cyclic form (e.g. pyranose or furanose form). For example, lactose is a disaccharide composed of cyclic forms of galactose and glucose joined by a beta (1-4) linkage where the acetal oxygen bridge is in the beta orientation.

Linkages or bonds between the individual glycan units found in glycan preparations may include one or more (e.g., two or more, three or more, four or more, five or more, six or more, etc.) of alpha 1→2, alpha 1→3, alpha 1→4, alpha 1→6, alpha 2→1, alpha 2→3, alpha 2→4, alpha 2→6, beta 1→2, beta 1→3, beta 1→4, beta 1→6, beta 2→1, beta 2→3, beta 2→4, and beta 2→6.

In some embodiments, the glycan preparation comprises both alpha- and beta-glycosidic bonds selected from the group consisting of 1→2 glycosidic bond, a 1→3 glycosidic bond, a 1→4 glycosidic bond, a 1→5 glycosidic bond and a 1→6 glycosidic bond. In some embodiments, the glycan preparation comprises at least two or at least three alpha and beta 1→2 glycosidic bonds, alpha and beta 1→3 glycosidic bonds, alpha and beta 1→4 glycosidic bonds, alpha and beta 1→5 glycosidic bonds, and/or alpha and beta 1→6 glycosidic bonds.

In some embodiments, the glycan preparations comprise only alpha linkages. In some embodiments, the glycan comprise only beta linkages. In some embodiments, the glycan preparations comprise mixtures of alpha and beta linkages.

In some embodiments, the alpha:beta glycosidic bond ratio in a preparation is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or about 10:1.

In some embodiments, the glycan preparations comprise and alpha:beta glycosidic bond ratio in a preparation of about 0.8:1, 1:1, 2:1, 3:1, 4:1 or 5:1, or it ranges from about 0.8:1 to about 5:1 or from about 1:1 to about 4:1.

In some embodiments, the preparations of glycan preparations (e.g. oligosaccharides) comprises substantially all alpha- or beta configured glycan units, optionally comprising about 1%, 2%, 3%, 4% 5%, 6%, 7%, 8%, 9%, 10%, 11%, 12%, 13%, 14%, 15%, 16%, 17%, 18%, 19%, or 20% of the respective other configuration.

In some embodiments, the preparations of glycan preparations comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% glycans with alpha glycosidic bonds. In some embodiments, the glycan preparations comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% glycans with beta glycosidic bonds. In some embodiments, glycan preparations are provided, wherein at least 10%, 15%, 20%, 25%, 30%, 35%, 40%, 45%, 50%, 55%, 60%, 65%, 70%, 75%, 80%, or at least 85% of glycans with glycosidic bonds that are alpha glycosidic bonds, at least 10%, 15%, 20%, 25%, 30%, 35%, 40%, 45%, 50%, 55%, 60%, 65%, 70%, 75%, 80%, or at least 85% of glycans with glycosidic bonds that are beta glycosidic bonds, and wherein the percentage of alpha and beta glycosidic bonds does not exceed 100%.

In some embodiments, glycan preparations are provided, wherein at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% of glycan glycosidic bonds are one or more of: 1→2 glycosidic bonds, 1→3 glycosidic bonds, 1→4 glycosidic bonds, and 1→6 glycosidic bonds. In some embodiments, glycan preparations are provided, wherein at least 1%, 2%, 3%, 4%, 5%, 6%, 7%, 8%, 9%, 10%, 15%, at least 20%, or 25% each of glycan glycosidic bonds are 1→2, 1→3, 1→4, and 1→6 glycosidic bonds. Optionally, glycan preparations further comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, or at least 85% of glycan glycosidic bonds that are selected from the group consisting of: alpha 2→1, alpha 2→3, alpha 2→4, alpha 2→6, beta 2→1, beta 2→3, beta 2→4, and beta 2→6, glycosidic bonds.

In some embodiments, the glycan preparations comprise glycans with at least two glycosidic bonds selected from the group consisting of alpha 1→2 and alpha 1→3, alpha 1→2 and alpha 1→4, alpha 1→2 and alpha 1→6, alpha 1→2 and beta 1→2, alpha 1→2 and beta 1→3, alpha 1→2 and beta 1→4, alpha 1→2 and beta 1→6, alpha 1→3 and alpha 1→4, alpha 1→3 and alpha 1→6, alpha 1→3 and beta 1→2, alpha 1→3 and beta 1→3, alpha 1→3 and beta 1→4, alpha 1→3 and beta 1→6, alpha 1→4 and alpha 1→6, alpha 1→4 and beta 1→2, alpha 1→4 and beta 1→3, alpha 1→4 and beta 1→4, alpha 1→4 and beta 1→6, alpha 1→6 and beta 1→2, alpha 1→6 and beta 1→3, alpha 1→6 and beta 1→4, alpha 1→6 and beta 1→6, beta 1→2 and beta 1→3, beta 1→2 and beta 1→4, beta 1→2 and beta 1→6, beta 1→3 and beta 1→4, beta 1→3 and beta 1→6, and beta 1→4 and beta 1→6.

For preparations comprising branched glycan preparations (e.g. those with a DB of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 0.95, 0.99, 1, or 2) comprising a side chain, which can be the same or a different side chain, the side chain may be attached via one or more beta and alpha linkages, such as (1-2), (1-3), (1-4), (1-6), (2-3), (2-6) or other suitable linkages to the main chain.

### Glycan Units

In some embodiments, glycan preparation are provided, wherein at least one glycan unit is a sugar in L-form. In some embodiments, preparations of glycans are provided, wherein at least one glycan unit is a sugar in D-form. In some embodiments, preparations of glycans are provided, wherein the glycan units are sugars in L- or D-form as they naturally occur or are more common (e.g. D-glucose, D-xylose, L-arabinose).

In some embodiments, the glycan preparation (e.g. oligosaccharides) comprises a desired mixture of L- and D-forms of glycan units, e.g. of a desired ratio, such as: 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, 1:100, 1:150 L- to D-forms or D- to L-forms.

In some embodiments, the glycan preparation comprises glycans with substantially all L- or D-forms of glycan units, optionally comprising about 1%, 2%, 3%, 4% 5%, 6%, 7%, 8%, 9%, 10%, 11%, 12%, 13%, 14%, 15%, 16%, 17%, 18%, 19%, 20%, 25%, 30%, 35%, 40%, 45%, or 50% of the respective other form.

In some embodiments, glycan preparations are provided, wherein at least one glycan unit is a diose, triose, tetrose, a pentose, a hexose, or a heptose. Optionally, the glycan units involved in the formation of the glycans are varied. Examples of monosaccharide glycan units include hexoses, such as glucose, galactose, and fructose, and pentoses, such as xylose. The monosaccharide glycan units may exist in an acyclic (open-chain) form. Open-chain monosaccharides with same molecular graph may exist as two or more stereoisomers. The monosaccharides may also exist in a cyclic form through a nucleophilic addition reaction between the carbonyl group and one of the hydroxyls of the same molecule. The reaction creates a ring of carbon atoms closed by one bridging oxygen atom. In these cyclic forms, the ring usually has 5 (furanoses) or 6 atoms (pyranoses).

In some embodiments, the glycan preparation (e.g. oligosaccharides) comprises a desired mixture of different monosaccharide glycan units, such as a mixture of a diose, a triose, tetrose, pentose, hexose, or heptose, including any mixtures of two or more pentoses (e.g., arabinose and xylose) and mixtures of two or more hexoses (e.g., glucose and galactose), in any desired ratio, e.g. for any two glycan units: 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, 1:100, 1:150, etc., for any three glycan units: 1:1:1, 1:2:1, 1:3:1, 1:4:1, 1:5:1, 1:6:1, 1:7:1, 1:8:1, 1:9:1, 1:10:1, 1:12:1, 1:14:1, 1:16:1, 1:18:1, 1:20:1, 1:1:2, 1:2:2, 1:3:2, 1:4:2, 1:5:2, 1:6:2, 1:7:2, 1:8:2, 1:9:2, 1:10:2, 1:1:3, 1:2:3, 1:3:3, 1:4:3, 1:5:3, 1:6:3, 1:7:3, 1:8:3, 1:9:3, 1:10:3, 1:1:4, 1:2:4, 1:3:4, 1:4:4, 1:5:4, 1:6:4, 1:7:4, 1:8:4, 1:9:4, 1:10:4, 1:1:5, 1:2:5, 1:3:5, 1:4:5, 1:5:5, 1:6:5, 1:7:5, 1:8:5, 1:9:5, 1:10:5, etc., for any four glycan units: 1:1:1:1, 1:2:2:1, 1:3:2:1, 1:4:2:1, 1:5:2:1, 1:6:2:1, 1:7:2:1, 1:8:2:1, 1:9:2:1, 1:10:2:1, 1:1:1:2, 1:2:2:2, 1:3:2:2, 1:4:2:2, 1:5:2:2, 1:6:2:2, 1:7:2:2, 1:8:2:2, 1:9:2:2, 1:10:2:2, etc., for any five glycan units: 1:1:1:1:1, 1:2:2:1:1, etc., for any six glycan units: 1:1:1:1:1:1, 1:1:1:1:1:2, etc., for any seven glycan units: 1:1:1:1:1:1:1, 1:1:1:1:1:1:2, etc., and so on.

In some embodiments, the glycan preparation comprises a desired mixture of two, three, four or five different glycan units, such as a mixture of, e.g., i) one or more glycan units selected from monosaccharides, selected from glucose, galactose, arabinose, mannose, fructose, xylose, fucose, and rhamnose; ii) one or more glycan units selected from disaccharides selected from acarviosin, n-acetyllactosamine, allolactose, cellobiose, chitobiose, galactose-alpha-1,3-galactose, gentiobiose, isomalt, isomaltose, isomaltulose, kojibiose, lactitol, lactobionic acid, lactose, lactulose, laminaribiose, maltitol, maltose, mannobiose, melibiose, melibiulose, neohesperidose, nigerose, robinose, rutinose, sambubiose, sophorose, sucralose, sucrose, sucrose acetate isobutyrate, sucrose octaacetate, trehalose, turanose, vicianose, and xylobiose; iii) one or more glycan units selected from amino sugars selected from acarbose, N-acetylemannosamine, N-acetylmuramic acid, N-acetylnueraminic acid, N-acetyletalosaminuronic acid, arabinopyranosyl-N-methyl-N-nitrosourea, D-fructose-L-histidine, N-glycolyneuraminic acid, ketosamine, kidamycin, mannosamine, 1B-methylseleno-N-acetyl-D-galactosamine, muramic acid, muramyl dipeptide, phosphoribosylamine, PUGNAc, sialyl-Lewis A, sialyl-Lewis X, validamycin, voglibose, N-acetylgalactosamine, N-acetylglucosamine, aspartylglucosamine, bacillithiol, daunosamine, desosamine, fructosamine, galactosamine, glucosamine, meglumine, and perosamine; iv) one or more glycan units selected from deoxy sugars selected from 1-5-ahydroglucitol, cladinose, colitose, 2-deoxy-D-glucose, 3-deoxyglucasone, deoxyribose, dideoxynucleotide, digitalose, fludeooxyglucose, sarmentose, and sulfoquinovose; v) one or more glycan units selected from imino sugars selected from castanospermine, 1-deoxynojirimycin, iminosugar, miglitol, miglustat, and swainsonine; one or more glycan units selected from sugar acids selected from N-acetylneuraminic acid, N-acetyltalosamnuronic acid, aldaric acid, aldonic acid, 3-deoxy-D-manno-oct-2-ulosonic acid, glucuronic acid, glucosaminuronic acid, glyceric acid, N-glycolylneuraminic acid, iduronic acid, isosaccharinic acid, pangamic acid, sialic acid, threonic acid, ulosonic acid, uronic acid, xylonic acid, gluconic acid, ascorbic acid, ketodeoxyoctulosonic acid, galacturonic acid, galactosaminuronic acid, mannuronic acid, mannosaminuronic acid, tartaric acid, mucic acid, saccharic acid, lactic acid, oxalic acid, succinic acid, hexanoic acid, fumaric acid, maleic acid, butyric acid, citric acid, glucosaminic acid, malic acid, succinamic acid, sebacic acid, and capric acid; vi) one or more glycan units selected from short-chain fatty acids selected from formic acid, acetic acid, propionic acid, butryic acid, isobutyric acid, valeric acid, and isovaleric acid; and vii) one or more glycan units selected from sugar alcohols selected from methanol, ethylene glycol, glycerol, erythritol, threitol, arabitol, ribitol, xylitol, mannitol, sorbitol, galactitol, iditol, volemitol, fucitol, inositol, maltotritol, maltotetraitol, and polyglycitol.

In some embodiments, the glycan preparation does not comprise polydextrose.

In some embodiments, the glycan preparation comprises a glycan unit or plurality of glycan units present in a salt form (e.g., a pharmaceutically acceptable salt form), such as, e.g., a hydrochlorate, hydroiodate, hydrobromate, phosphate, sulfate, methanesulfate, acetate, formate, tartrate, malate, citrate, succinate, lactate, gluconate, pyruvate, fumarate, propionate, aspartate, glutamate, benzoate, ascorbate salt.

Exemplary glycans are described by a three-letter code representing the monomeric sugar component followed by a number out of one hundred reflecting the percentage of the material that monomer constitutes. Thus, ‘glu100’ is ascribed to a glycan generated from a 100% D-glucose (glycan unit) input and ‘glu50gal50’ is ascribed to a glycan generated from 50% D-glucose and 50% D-galactose (glycan units) input or, alternatively from a lactose dimer (glycan unit) input. As used herein: xyl=D-xylose; ara=L-arabinose; gal=D-galactose; glu=D-glucose; rha=L-rhamnose; fuc=L-fucose; man=D-mannose; sor=D-sorbitol; gly=D-glycerol; neu=NAc-neuraminic acid.

In some embodiments, the glycan preparation comprises one glycan unit A selected from i) to vii) above, wherein glycan unit A comprises 100% of the glycan unit input. For example, in some embodiments, the glycan preparation is selected from the homo-glycans xyl100, rha100, ara100, gal100, glu100, and man100. In some embodiments, the glycan preparation is selected from the homo-glycans fuc100 and fru100. In some embodiments, the glycan preparation comprises man100.

In some embodiments, the glycan preparation comprises a mixture of two glycan units A and B selected independently from i) to vii) above, wherein A and B may be selected from the same or a different group i) to vii) and wherein A and B may be selected in any desired ratio (e.g. anywhere from 1-99% A and 99-1% B, not exceeding 100%).

For example, in some embodiments, the glycan therapeutic preparation is selected from the hetero-glycans ara50gal50, xyl75ga125, ara80xyl20, ara60xyl40, ara50xyl50, glu80man20, glu60man40, man60glu40, man80glu20, ga175xyl25, glu50gal50, man62glu38, and the hybrid glycans glu90sor10 and glu90gly10.

In some embodiments, the glycan preparation comprises a mixture of three glycan units A, B and C selected independently from i) to vii) above, wherein A, B and C may be selected from the same or a different group i) to vii) and wherein A, B and C may be selected in any desired ratio (e.g. anywhere from 1-99% A, 1-99% B, 1-99% C, not exceeding 100%).

For example, in some embodiments, the glycan therapeutic preparation is selected from the hetero-glycans xyl75glu12gal12, xyl33glu33ga133, glu33gal33fuc33, man52glu29gal19, and the hybrid glycan glu33gal33neu33.

In some embodiments, the glycan preparation comprises a mixture of four glycan units A, B, C and D selected independently from i) to vii) above, wherein A, B, C and D may be selected from the same or a different group i) to vii) and wherein A, B, C and D may be selected in any desired ratio (e.g. anywhere from 1-99% A, 1-99% B, 1-99% C, 1-99% D, not exceeding 100%).

In some embodiments, the glycan preparation comprises a mixture of five glycan units A, B, C, D and E selected independently from i) to vii) above, wherein A, B, C, D and E may be selected from the same or a different group i) to vii) and wherein A, B, C, D and E may be selected in any desired ratio (e.g. anywhere from 1-99% A, 1-99% B, 1-99% C, 1-99% D, 1-99% E, not exceeding 100%).

In some embodiments, preparations of glycan are provided, wherein at least one glycan unit is selected from the group consisting of a glucose, a galactose, an arabinose, a mannose, a fructose, a xylose, a fucose, and a rhamnose. In one embodiment, the glycan unit is not glucose. In one embodiment, the glycan unit is not galactose. In one embodiment, the glycan unit is not fructose.

In one embodiment, the glycan unit is not fucose. In one embodiment, the glycan unit is not mannose. In one embodiment, the glycan unit is not arabinose. In one embodiment, the glycan unit is not rhamnose. In one embodiment, the glycan unit is not xylose.

In some embodiments, the glycan preparation comprises a desired mixture of two different monosaccharide glycan units, such as a mixture of, e.g., glucose and galactose, glucose and arabinose, glucose and mannose, glucose and fructose, glucose and xylose, glucose and fucose, glucose and rhamnose, galactose and arabinose, galactose and mannose, galactose and fructose, galactose and xylose, galactose and fucose, and galactose and rhamnose, arabinose and mannose, arabinose and fructose, arabinose and xylose, arabinose and fucose, and arabinose and rhamnose, mannose and fructose, mannose and xylose, mannose and fucose, and mannose and rhamnose, fructose and xylose, fructose and fucose, and fructose and rhamnose, xylose and fucose, xylose and rhamnose, and fucose and rhamnose, etc., e.g. a in a ratio of 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, or 1:100 or the reverse ratio thereof.

In some embodiments, the glycan preparation (e.g. oligosaccharides) comprises a desired mixture of three different monosaccharide glycan units, such as a mixture of, e.g. for glucose-containing glycan-therapeutic preparations, glucose, galactose and arabinose; glucose, galactose and mannose; glucose, galactose and fructose; glucose, galactose and xylose; glucose, galactose and fucose, glucose, galactose and rhamnose; glucose, arabinose, and mannose; glucose, arabinose and fructose; glucose, arabinose and xylose; glucose, arabinose and fucose; glucose, arabinose and rhamnose; glucose, mannose and fructose; glucose, mannose and xylose; glucose, mannose and fucose; glucose, mannose rhamnose; glucose, fructose and xylose; glucose, fructose and fucose; glucose, fructose and rhamnose; glucose, fucose and rhamnose, etc., e.g. a in a ratio of 1:1:1, 1:2:1, 1:3:1, 1:4:1, 1:5:1, 1:6:1, 1:7:1, 1:8:1, 1:9:1, 1:10:1, 1:12:1, 1:14:1, 1:16:1, 1:18:1, 1:20:1, 1:1:2, 1:2:2, 1:3:2, 1:4:2, 1:5:2, 1:6:2, 1:7:2, 1:8:2, 1:9:2, 1:10:2, 1:1:3, 1:2:3, 1:3:3, 1:4:3, 1:5:3, 1:6:3, 1:7:3, 1:8:3, 1:9:3, 1:10:3, 1:1:4, 1:2:4, 1:3:4, 1:4:4, 1:5:4, 1:6:4, 1:7:4, 1:8:4, 1:9:4, 1:10:4, 1:1:5, 1:2:5, 1:3:5, 1:4:5, 1:5:5, 1:6:5, 1:7:5, 1:8:5, 1:9:5, 1:10:5, etc.

In some embodiments, preparations of glycan therapeutics are provided, wherein at least one glycan unit is a furanose sugar. In some embodiments, preparations of glycans are provided, wherein at least one glycan unit is a pyranose sugar. In some embodiments, glycan therapeutics comprise mixtures of furanose and pyranose sugars. In some embodiments, the furanose: pyranose sugar ratio in a preparation is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or about 10:1.

In some embodiments, the glycan preparation (e.g. oligosaccharides) comprises a desired mixture of furanose and pyranose sugars, e.g. of a desired ratio, such as: 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, 1:100, 1:150 furanose to and pyranose or pyranose to furanose.

In some embodiments, the glycan preparation comprises substantially all furanose or pyranose sugar, optionally comprising 1%, 2%, 3%, 4% 5%, 6%, 7%, 8%, 9%, 10%, 11%, 12%, 13%, 14%, 15%, 16%, 17%, 18%, 19%, or 20% of the respective other sugar.

In some embodiments, the glycan preparation comprises substantially all pyranose sugar and no more than about 0.1%, 02%, 0.5%, 1%, 2%, 3%, 4%, or no more than 5% of monomeric glycan units in the preparation in furanose form. In some embodiments, no more than 3%, 2% or no more than 1% of monomeric glycan units in the preparation are in furanose form.

In some embodiments, the glycan preparation does not comprise N-acetylgalactosamine or N-acetylglucosamine. In some embodiments, the glycan preparation does not comprise neuraminic acid. In some embodiments, the preparation of glycans does not comprise sialic acid. In some embodiments, the glycan preparation does not comprise a lipid and fatty acid. In some embodiments, the glycan preparation does not comprise an amino acid. In some embodiments, the glycan preparation does not comprise sorbitol. In some embodiments, the glycan preparation does not comprise glucose, galactose, mannose, arabinose, fructose, xylose, fucose, or rhamnose.

In some embodiments, the glycan preparation does not comprise a detectable repeating unit. In some embodiments, the glycan preparation does not comprise a statistically significant amount of a repeating unit. In some embodiments, the repeating unit has a DP of at least 2, 3, 4, 5, or at least 6 glycan units. For example, hyaluronan is a glycosaminoglycan with a repeating disaccharide unit consisting of two glucose derivatives, glucuronate (glucuronic acid) and N-acetylglucosamine. The glycosidic linkages are beta (1→3) and beta (1→4). Cellulose is a polymer made with repeated glucose units linked together by beta-linkages. The presence and amount of repeating units can be determined, e.g. using by total hydrolysis (e.g. to determine the proportion of glycan units), methylation analysis (e.g. to determine the distribution of bond types), and HSQC (e.g. to determine the distribution of alpha- and beta-glycosides). Statistical methods to determine significance are known by one of skill in the art.

If desired, the monosaccharide or oligosaccharide glycan units of the glycans are further substituted or derivatized, e.g., hydroxyl groups can be etherified or esterified. For example, the glycans (e.g. oligosaccharide) can contain modified saccharide units, such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). The degree of substitution (DS, average number of hydroxyl groups per glycosyl unit) can be 1, 2, or 3, or another suitable DS. In some embodiments, 1%, 2%, 3%, 4% 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 80%, 85%, 90%, 95%, 96%, 97%, 98%, 99%, or 100% of glycan units are substituted or derivatized. In some embodiments, the degree of substitution varies between subunits, e.g., a certain percentage is not derivatized, exhibits a DS of 1, exhibits a DS of 2, or exhibits a DS of 3. Any desired mixture can be generated, e.g. 0-99% of subunits are not derivatized, 0-99% of subunits exhibit a DS of 1, 0-99% of subunits exhibit a DS of 2, and 0-99% of subunits exhibit a DS of 3, with the total making up 100%. The degree of substitution can be controlled by adjusting the average number of moles of substituent added to a glycosyl moiety (molar substitution (MS)). The distribution of substituents along the length of the glycan oligo- and polysaccharide chain can be controlled by adjusting the reaction conditions, reagent type, and extent of substitution. In some embodiments, the monomeric subunits are substituted with one or more of an acetate ester, sulfate half-ester, phosphate ester, or a pyruvyl cyclic acetal group.

### Solubility

In some embodiments, the glycan therapeutic preparations are highly banched, e.g. have an average DB of at least 0.01, 0.05, or 0.1. In some embodiments, the glycan therapeutic preparations have an average DB of about 0.01 to about 0.05, 0.01 to 0.1, 0.05 to 0.1, or about 0.1 to about 0.2. In some embodiments, the glycan therapeutic preparations comprising branched oligosaccharide are highly soluble. In some embodiments, glycan therapeutic preparations can be concentrated to at least to 55 Brix, 65 Brix, 60 Brix, 65 Brix, 70 Brix, 75 Brix, 80 Brix, or at least 85 Brix without obvious solidification or crystallization at 23° C. (final solubility limit). In some embodiments, glycan therapeutic preparations can be concentrated to about 50-60 Brix, 60-70 Brix, 70-80 Brix, 55-65 Brix, 65-75 Brix, or to about 75-85 Brix. In some embodiments, glycan therapeutic preparations can be concentrated to about 50, 55, 60, 65, 70, 75, 80, or about 85 Brix without obvious solidification or crystallization at 23° C. (final solubility limit).

In some embodiments, glycan therapeutic preparations are concentrated to at least about 0.5 g/ml, 1 g/ml, 1.5 g/ml, 2 g/ml, 2.5 g/ml, 3 g/ml, 3.5 g/ml or at least 4 g/ml without obvious solidification or crystallization at 23° C. (final solubility limit).

In some embodiments, the glycan therapeutic preparations (e.g. oligosaccharides) are branched, e.g. have an average DB of at least 0.01, 0.05, or 0.1 and has a final solubility limit in water of at least about 70 Brix, 75 Brix, 80 Brix, or at least about 85 Brix at 23° C. or is at least about 1 g/ml, 2 g/ml or at least about 3 g/ml.

In some embodiments, the glycan preparation has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L in deionized water, or in a suitable buffer such as, e.g., phosphate-buffered saline, pH 7.4 or similar physiological pH and at 20° C.

In some embodiments, the glycan preparation is greater than 50%, greater than 60%, greater than 70%, greater than 80%, greater than 90%, greater than 95%, greater than 96%, greater than 97%, greater than 98%, greater than 99%, or greater than 99.5% soluble with no precipitation observed at a concentration of greater than 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L in deionized water, or in a suitable buffer such as, e.g., phosphate-buffered saline, pH 7.4 or similar physiological pH and at 20° C.

### Sweetness

In some embodiments, the glycan preparation has a desired degree of sweetness. For example, sucrose (table sugar) is the prototype of a sweet substance. Sucrose in solution has a sweetness perception rating of 1, and other substances are rated relative to this (e.g., fructose, is rated at 1.7 times the sweetness of sucrose). In some embodiments, the sweetness of the glycan preparation ranges from 0.1 to 500,000 relative to sucrose. In some embodiments, the relative sweetness is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 25000, 50000, 75000, 100000, 150000, 200000, 250000, 300000, 350000, 40000, 450000, 500000, or more than 500,000 relative to sucrose (with sucrose scored as one). In some embodiments, the glycan preparation is mildly sweet, or both sweet and bitter.

In some embodiments, the glycan preparation, e.g. a preparation that is substantially DP2+ or DP3+ (e.g. at least 80%, 90%, or at least 95%, or a fractionated preparation of DP2+ or DP3+), is substantially imperceptible as sweet and the relative sweetness is about 0, 0.0001, 0.001, 0.005, 0.01, 0.05, 0.1, 0.2, 0.3, 0.4, 0.5, 0.6, 0.7, 0.8, or about 0.9 relative to sucrose (with sucrose scored as one).

In some embodiments, the glycan preparation has one or more (e.g., 2, 3, 4, 5, or 6) of the following (bulk) properties:
i) the glycan preparation comprises branched glycans that comprise glucose, galactose, arabinose, mannose, fructose, xylose, fucose, or rhamnose glycan units,
ii) the average degree of branching (DB) of the branched glycans in the glycan preparation is between about 0.01 and about 0.6,
iii) at least 50% of the glycans in the glycan preparation have a degree of polymerization (DP) of at least 3 and less than 30 glycan units,
iv) the average DP of the glycan preparation is between about DP3 and about DP18,
v) the ratio of alpha- to beta-glycosidic bonds present in the glycans of the glycan preparation is between about 0.8:1 and about 5:1, and/or
vi) the glycan preparation has a final solubility limit in water of at least about 60 Brix at 23° C.

In some embodiments, the glycan preparation has an average degree of branching (DB) of the branched glycans in the glycan preparation is between about 0.05 and about 0.6.

In some embodiments, the glycan preparation has an average DP of the glycan preparation is one of: between about DP3 and about DP15, between about DP3 and about DP8, between about DP5 and about DP10, or between about DP6 and about DP18.

In some embodiments, the glycan preparation has a ratio of alpha- to beta-glycosidic bonds present in the glycans of the glycan preparation is between about 1:1 and about 5:1.

### Identification and Characterization of Glycan Therapeutic Preparations

If desired, the glycan therapeutic preparations can be characterized by any method known in the art and by the methods described herein.

The molar percentage of species with a degree of polymerization (DP) of n (denoted here as DP(n)) in a population is determined by high performance liquid chromatography (HPLC), e.g., on an Agilent 1260 BioInert series instrument equipped with a refractive index (RI) detector and a variety of columns familiar to those skilled in the art using water as the mobile phase. The columns are selected from chemistries including HILIC, metal coordination, and aqueous size-exclusion chromatography that best isolate the species of interest. Molar % DP(n) is determined by the formula:

% DP(n)=100*AUC[DP(n)]/AUC[DP(total)],

where AUC is defined as the area under the curve for the species of interest as determined by calibration to known standards. The molar percentage of glycosidic bond isomers (% alpha and % beta) are determined by nuclear magnetic resonance (NMR) spectroscopy using a variety of 2D techniques familiar to those skilled in the art. Alpha- and beta-isomers may be distinguished, e.g., by their distinct shift on the NMR spectrum and the molar percentage is determined by the formula:

% (glycosidic isomer n) of glycosidic bonds=100*AUC[shift(isomer n)]/AUC[shift(isomer alpha+isomer beta)],

where AUC is defined as the area under the curve at a specific shift value known to represent the desired isomer n. The molar percentage of regiochemical isomers is determined in an analogous fashion using the formula:

% (regioisomer n) of regioisomers=100*AUC[shift(regioisomer n)]/AUC[shift(all regioisomers)].

The relative percentage of monomeric sugars making up the oligomeric population is determined, e.g., by total acidic digestion of the oligomeric sample followed by conversion to the alditol acetate followed by gas chromatographic (GC) analysis of the resultant monomeric solutions compared against GC of known standards. The molar percentage of monomer (n), where n can be any sugar, is determined by the formula:

% (sugar n)=100*AUC[sugar n]/AUC[total of all monomeric sugars].

In some embodiments, the solubility of the glycan preparation can be controlled, e.g. by selecting the charge, structure (e.g. DP, degree of branching), and/or derivatization of the glycan units.

For glycan therapeutic preparations, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infra red)/Raman spectroscopy, and NMR (Nuclear magnetic resonance) spectroscopy techniques. For polymers of crystalline consistency, the crystal structure can be solved using, e.g., solid-state NMR, FT-IR (Fourier transform infrared spectroscopy), and WAXS (wide-angle X-ray scattering). The DP, DP distribution, and polydispersity can be determined by, e.g., viscosimetry and SEC (SEC-HPLC, high performance size-exclusion chromatography). Alien groups, end groups and substituents can be identified, e.g., using SEC with labeling, aqueous analytics, MALDI-MS, FT-IR, and NMR. To identify the monomeric components of the glycans methods such as, e.g. acid-catalyzed hydrolysis, HPLC (high performance liquid chromatography) or GLC (gas-liquid chromatography) (after conversion to alditol acetates) may be used. To determine the linkages present in the glycans, in one example, the polysaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). In some embodiments, to determine the polysaccharide sequence a partial depolymerization is carried out using an acid or enzymes to determine the structures. Possible structures of the polysaccharide are compared to those of the hydrolytic oligomers, and it is determined which one of the possible structures could produce the oligomers. To identify the anomeric configuration, in one example, the intact polysaccharide or a preparation of oligosaccharides are subjected to enzymatic analysis, e.g. they are contacted with an enzyme that is specific for a particular type of linkage, e.g., β-galactosidase, or α-glucosidase, etc., and NMR may be used to analyze the products.

For example, the distribution of (or average) degree of polymerization (DP) of a glycan therapeutic preparation may be measured by injecting a sample with a concentration of, e.g., 10-100 mg/mL onto an Agilent 1260 BioPure HPLC (or similar) equipped with a 7.8×300 mm BioRad Aminex HPX-42A column (or similar) and RI detector as described, e.g., in Gómez et al. (Purification, Characterization, and Prebiotic Properties of Pectic Oligosaccharides from Orange Peel Wastes, J Agric Food Chem, 2014, 62:9769). Alternatively, a sample with a concentration may be injected into a Dionex ICS5000 HPLC (or similar) equipped with a 4×250 mm Dionex CarboPac PA1 column (or similar) and PAD detector as described, e.g., in Holck et al., (Feruloylated and nonferuloylated arabino-oligosaccharides from sugar beet pectin selectively stimulate the growth of bifidobacterium spp. in human fecal in vitro fermentations, Journal of Agricultural and Food Chemistry, 2011, 59(12), 6511-6519). Integration of the resulting spectrum compared against a standard solution of oligomers allows determination of the average DP.

Distribution of molecular weights can be measured, e.g, by MALDI mass spectrometry.

Oligosaccharide concentration can be measured with a Mettler-Toledo sugar refractometer (or similar) with the final value adjusted against a standardized curve to account for refractive differences between monomers and oligomers.

Distribution of glycoside regiochemistry can be characterized, e.g., by a variety of 2D-NMR techniques including COSY, HMBC, HSQC, DEPT, and TOCSY analysis using standard pulse sequences and a Bruker 500 MHz spectrometer. Peaks can be assigned by correlation to the spectra of naturally occurring polysaccharides with known regiochemistry.

In some embodiments, the relative peak assignment of a sample is dependent on a number of factors including the concentration and purity of the sample, the identity and quality of the solvent (e.g., the isotopically labeled solvent), and the pulse sequence utilized. As such, in embodiments, the relative peak assignment of, for example, a glycan comprising glucose may vary (e.g., by about 0.01 ppm, about 0.02 ppm, or about 0.05 ppm) when the NMR spectrum is obtained in similar conditions due to said factors. In these instances as used herein, the terms “corresponding peak” or “corresponding peaks” refer to NMR peaks associated with the same sample but that vary (e.g., by about 0.01 ppm, about 0.02 ppm, or about 0.05 ppm) due to factors including, for example, the concentration and purity of the sample, the identity and quality of the isotopically labeled solvent, and the pulse sequence utilized.

Monomeric compositions of oligomers may be measured, e.g., by the complete hydrolysis method in which a known amount of oligomer is dissolved into a strong acid at elevated temperature and allowed sufficient time for total hydrolysis to occur. The concentration of individual monomers may then be measured by the HPLC or GC methods described herein and known in the art to achieve relative abundance measurements as in Holck et al. Absolute amounts can be measured by spiking the HPLC sample with a known amount of detector active standard selected to prevent overlap with any of the critical signals.

The degree of branching in any given population may be measured by the methylation analysis method established, e.g, by Hakomori (J. Biochem. (Tokyo), 1964, 55, 205). With these data, identification of potential repeat units may be established by combining data from the total hydrolysis, average DP, and methylation analysis and comparing them against the DEPT NMR spectrum. Correlation of the number of anomeric carbon signals to these data indicates if a regular repeat unit is required to satisfy the collected data as demonstrated, e.g., in Harding, et al. (Carbohydr. Res. 2005, 340, 1107).

Glycan preparation (e.g. those comprising monosaccharide or disaccharide glycan units such as glucose, galactose, fucose, xylose, arabinose, rhamnose, and mannose) may be identified using one, two, three, or four of the following parameters: a) the presence of 2, 3, 4, 5, 6, 7 or more (e.g. at least 4 or 5) diagnostic anomeric NMR peaks each representing a different glycosidic bond type, b) an alpha- to beta-bond ratio between about 0.8 to 1 and about 5 to 1 (e.g. between about 1:1 and 4:1, commonly favoring the alpha bond type), c) at least 2 or at least 3 different glycoside regiochemistries from the list of 1,2-; 1,3-; 1,4-; and 1,6-substituted and at least 2 or at least 3 different glycoside regiochemistries from list of 1,2,3-; 1,2,4-; 1,2,6-; 1,3,4-; 1,3,6-; and 1,4,6-substituted, and d) a DP distribution in which at least 50%, 60%, 70% or at least 80% of the individual species have a DP of at least 2, at least 3, between 3 and 30 or between 5 and 25.

In some embodiments, glycan therapeutic preparations have average properties (e.g., DP, DB, alpha:beta glycosidic bond ratio) that are distinct from naturally occurring preparations of oligosaccharides. These structural features may be analyzed and optionally quantified by any suitable method known in the art and those described herein. The glycan therapeutic preparations described herein have at least one, two, three, four, or at least five of the following characteristics:
(i) a distribution of molecular weights ranging, e.g. from about DP3 to about DP30, about DP2 to about DP30, about DP2 to about 20, about DP2 to about DP10, about DP3 to about DP20, about DP3 to about DP10, or from about DP5 to about DP25 that may be identified by quantitative mass spectrometry measurements, SEC-HPLC, IAC-HPLC, or IEC-HPLC;
(ii) a significant proportion of both alpha and beta bonds, with bond ratios, e.g., ranging from 0.8:1, 1:1, 2:1, 3:1, 4:1, to 5:1 (genrally favoring the alpha stereochemistry) that may be identified by a variety of NMR techniques including the HSQC pulse sequence which allows explicit discrimination and quantitation of signals from alpha and beta glycosides. The presence of both alpha- and beta-glycosidic bonds in the observed ratios in glycan therapeutic preparation of some embodiments, is distinct from preparations of naturally occurring oligo- or polysaccharides which genrally favor one primary glycosidic stereochemistry and optionally comprise only a relatively small portion of the opposing stereochemistry;
(iii) presence of at least one, two, three or four glycoside regiochemistries that may be identified either by a fingerprint NMR process or by the permethylation branching identification developed by Hakomori, et al. In some embodiments, glycan therapeutic preparations have at least 0.1%, 0.2%, 0.5%, 1%, 2%, 3%, 4%, 5%, 6%, 7%, 8%, 9%, or at least 10% of one, two, three or four of the 1,2-; 1,3-; 1,4-, and 1,6-glycoside bond types. In some embodiments, glycan therapeutic preparations have at least 0.1%, 0.2%, 0.5%, 1%, 2%, 3%, 4%, 5%, 6%, 7%, 8%, 9%, or at least 10% of two of the 1,2-; 1,3-; 1,4-, and 1,6-glycoside bond types. In some embodiments, glycan therapeutic preparations have at least 0.1%, 0.2%, 0.5%, 1%, 2%, 3%, 4%, 5%, 6%, 7%, 8%, 9%, or at least 10% of three of the 1,2-; 1,3-; 1,4-, and 1,6-glycoside bond types. In some embodiments, glycan therapeutic preparations have at least 0.1%, 0.2%, 0.5%, 1%, 2%, 3%, 4%, 5%, 6%, 7%, 8%, 9%, or at least 10% of all four of the 1,2-; 1,3-; 1,4-, and 1,6-glycoside bond types. In some embodiments, the glycan therapeutic preparation additionally comprises at least 0.1%, 0.2%, 0.5%, 1%, 2%, 3%, 4% or at least 5% of branched bond types. In some embodiments, the glycan therapeutic preparation comprises at least 0.1%, 0.2%, 0.5%, 1%, 2%, 3%, 4% or at least 5% of at least one, two, or at least three branched bond types including 1,3,6-; 1,4,6-; or 1,2,4-glycosides. In some embodiments, the glycan therapeutic preparation comprises at least two branched bond types of 1,3,6-; 1,4,6-; or 1,2,4-glycosides. In some embodiments, the glycan therapeutic preparation comprises at least 0.1%, 0.2%, 0.5%, 1%, 2%, 3%, 4% or at least 5% of three branched bond types of 1,3,6-; 1,4,6-; or 1,2,4-glycosides. Sugars that do not have a hydroxyl group at a given position X will not will not have the 1,X-bond type, e.g. fucose (6-dehydroxy-galactose) will not have 1,6-glycosidic bonds but will have 1,2-; 1,3-; and 1,4-glycosidic bonds. In some embodiments, the glycan therapeutic preparation comprises at least 0.1%, 02%, 0.5%, 1%, 2%, or at least 3% of monomeric glycan units in furanose form. The presence of a large number of glycoside regiochemistries and branching in glycan therapeutic preparation of some embodiments, is distinct from preparations of naturally occurring oligo- or polysaccharides which genrally favor specific bond architectures. Although all of these regiochemistries are known to occur in oligosaccharides of natural sources, preparations of naturally sourced oligosaccharide do not comprise the number and complexity of regiochemistries that are exhibited by glycan therapeutic preparations of some embodiments,
(iv) a distribution of glycosidic bonds that represents at least 50%, 60%, 70%, 80% or at least 90% of all possible combinations of regio- and stereochemistries. Individually, the regiochemical distribution can be determined by branching analysis and the stereochemical distribution can be determined by NMR. The HSQC-NMR. In some embodiments, the glycan therapeutic preparations exhibit a diversity of peaks in the anomeric region that are associated with a multiplicative combination of both regiochemistry and stereochemistry. In some embodiments, the glycan therapeutic preparation comprises at least two or at least three of alpha-1,2-; alpha-1,3-; alpha-1,4-; and alpha-1,6-glycosides and at least two, or at least three of beta-1,2-; beta-1,3-; beta-1,4-; and beta-1,6-glycosides. In some embodiments, the glycan therapeutic preparation comprises all four of alpha-1,2-; alpha-1,3-; alpha-1,4-; and alpha-1,6-glycosides and all four of beta-1,2-; beta-1,3-; beta-1,4-; and beta-1,6-glycosides. As an exemplar, HSQC of a glu100 preparation shows that the preparation contains all alpha-1,2-; alpha-1,3-; alpha-1,4-; and alpha-1,6-glycosides as well as all beta-1,2-; beta-1,3-; beta-1,4-; and beta-1,6-glycosides. Sugars that do not have a hydroxyl group at a given position X will not will not have the 1,X-bond type, e.g. fucose (6-dehydroxy-galactose) will not have 1,6-glycosidic bonds but will have 1,2-; 1,3-; and 1,4-glycosidic bonds.

### Methods of Modulating Bacterial Taxa and Microbial Diversity

Provided herein are methods of modulating the abundance of a bacterial taxa in a non-gut site containing mucosal tissue of a human subject. Also provided herein are methods of modulating microbial diversity in a non-gut site containing mucosal tissue. The methods comprise locally (e.g. directly) administering to the non-gut site (e.g. to the mucosal tissue) a glycan preparation described herein in an amount and for a time effective to modulate the bacterial taxa and/or microbial diversity in the site. In some embodiments, the non-gut site is the oral cavity, the nasal cavity, or the vagina.

### Vagina

In some embodiments, methods of modulating the abundance of a bacterial taxa in the vagina of a human subject are provided. The methods comprise locally (e.g. directly) administering to the vagina a glycan preparation described herein in an amount and for a time effective to modulate the bacterial taxa.

In some embodiments, the glycan preparation modulates (e.g. increasing or decreasing) the growth or relative abundance of one or more (e.g. two, three, four, five or more) bacterial taxa, such as, e.g., the most abundant bacterial taxa. In some embodiments, the bacterial taxa in the vagina that are being modulated by administration of the glycan preparations described herein are one or more (e.g. two, three, four, five or more) of the bacterial genera Actinomyces, Corynebacterium, Bacteroides, Prevotella, Staphylococcus, Lactobacillus, Streptococcus, Anaerococcus, Finegoldia, Peptoniphilus, and Dialister which are common vaginal bacterial taxa.

In some embodiments, methods of modulating one or more lactobacilli in the vagina are provided comprising administering, e.g., locally to the vagina the glycan preparations described herein. In some embodiments, the one or more lactobacilli are thought to be associated with vaginal health, and include one or more (e.g., two, three, four, or more) of Lactobacillus coleohominis, Lactobacillus crispatus, Lactobacillus gasseri, Lactobacillus iners, Lactobacillus jensenii, and Lactobacillus vaginalis.

In some embodiments, the glycan therapeutic drives selective changes in both the composition and activity of the vaginal microbiota, thereby conferring health benefits upon the human host.

In some embodiments, the health benefit includes the reduction of symptoms for a disease, disorder or pathological condition, such as, e.g. bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, risk for a preterm birth or miscarriage. In some embodiments, the disease, disorder or pathological condition is bacterial vaginosis (BV). In some embodiments, the disease, disorder or pathological condition is infection with vancomycin-resistant enterococci (VRE) or Group B Streptococcus infection.

Under certain conditions, pathogenic species and pathobionts that are capable of causing disease, e.g. by inducing an infection and/or inflammation and/or bacteria associated with a disease state without necessarily being a causative agent are present in the niche. In some embodiments, methods are provided for modulating (e.g. decreasing) the abundance of vaginal disease-associated bacteria, pathobionts or pathogens by administering to the vagina the glycan preparations described herein.

In some embodiments, the disease-associated bacteria, pathobionts or pathogens include one or more of Gardnerella vaginalis, Prevotella species, Porphyromonas species, Peptostreptococcus species, Mycoplasma hominis, and Mobiluncus species, Fusobacterium species, Atopobium vaginae, and Enterococcus faecium.

In some embodiments, the disease-associated bacteria, pathobionts or pathogens include one or more of the genera Actinomyces, Aerococcus, Atopobium, Bacteroides, Corynebacterium, Dialister, Eggerthella, Escherichia, Gardnerella, Haemophilus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, and the order Clostridiales (e.g. bacterial vaginosis-associated bacterium-1 (BVAB-1), BVAB-2, and BVAB-3).

In some embodiments, the disease-associated bacteria, pathobionts or pathogens include one or more of the species Aerococcus christensenii Atopobium vaginae, Bacteroides urealyticus, Corynebacterium vaginale, Dialister micraerophilus, Escherichia coli, Gardnerella vaginalis, Haemophilus influenza, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

In some embodiments, the methods for modulating (e.g. decreasing) the abundance of vaginal disease-associated bacteria, pathobionts or pathogens by administering to the vagina the glycan preparations described herein include modulating (e.g. increasing) the abundance of one or more bacterial taxa associated with vaginal health, e.g. one or more lactobacilli.

In some embodiments, methods for modulating (e.g. decreasing) bacterial diversity in the vagina are provided by locally administering to the vagina the glycan preparations described herein and modulating (e.g. increasing) the abundance of one or more bacterial taxa associated with vaginal health, e.g. one or more lactobacilli, and decreasing the bacterial diversity in the site.

### Nasal Cavity

In some embodiments, methods of modulating the abundance of a bacterial taxa in the nasal cavity of a human subject are provided. The methods comprise locally (e.g. directly) administering to the nasal cavity a glycan preparation described herein in an amount and for a time effective to modulate the bacterial taxa.

In some embodiments, the glycan preparation modulates (e.g. increasing or decreasing) the growth or relative abundance of one or more (e.g. two, three, four, five or more) bacterial taxa, such as, e.g., the most abundant bacterial taxa. In some embodiments, the bacterial taxa in the nasal cavity that are being modulated by administration of the glycan preparations described herein are one or more (e.g. two, three, four, five or more) of the bacterial species Propionibacterium acnes, Staphylococcus epidermidis, Staphylococcus aureus, Corynebacterium accolens, Corynebacterium tuberculostearicum, Corynebacterium pseudodiphtericum, Mycobacterium fallax, Corynebacterium mucifaciens, Dolosigranulum pigrum, Finegoldia magna, and Moraxella catarrhalis, which are common nasal taxa. In some embodiments, the bacterial taxa in the nasal cavity that are being modulated by administration of the glycan preparations described herein are one or more of the bacterial genera Tomitella, Peptoniphilus, Anaerococcus, which are common nasal taxa.

In some embodiments, methods of modulating one or both of Lactobacillus (e.g. Lactobacillus sakei) and Staphylococcus (e.g. Staphylococcus epidermidis) bacterial taxa in the nasal cavity are provided comprising administering, e.g., locally to the nasal cavity the glycan preparations described herein. These bacterial taxa are thought to be associated with a healthy nasal cavity.

In some embodiments, the glycan preparation drives selective changes in both the composition and activity of the nasal microbiota, thereby conferring health benefits upon the human host.

In some embodiments, the health benefit includes the reduction of symptoms for a disease, disorder or pathological condition, such as, e.g., rhinosinusitis (sinus infection), chronic rhinosinusitis (CRS), S. aureus infection or carriage, nasal vestibulitis, nasal furuncles and asthma.

Under certain conditions, pathogenic species and pathobionts that are capable of causing disease, e.g. by inducing an infection and/or inflammation and/or bacteria associated with a disease state without necessarily being a causative agent are present in the niche. In some embodiments, methods are provided for modulating (e.g. decreasing) the abundance of nasal disease-associated bacteria, pathobionts or pathogens by administering to the nasal cavity the glycan preparations described herein.

In some embodiments, the disease-associated bacteria, pathobionts or pathogens include one or more of the genus Corynebacterium, Dolosigranulum, Haemophilus, Moraxella, Peptoniphilus, Propionibacterium, Pseudomonas, Staphylococcus, and Streptococcus.

In some embodiments, the disease-associated bacteria, pathobionts or pathogens include one or more of the species Corynebacterium accolens, Corynebacterium pseudodiphtericum, Corynebacterium tuberculostearicum, Dolosigranulum pigrum, Haemophilus influenza, Moraxella catarrhalis, Peptoniphilus rhinitidis, Propionibacterium acnes, Pseudomonas aeruginosa, Staphylococcus aureus, and Streptococcus pneumonia.

In some embodiments, the methods for modulating (e.g. decreasing) the abundance of nasal disease-associated bacteria, pathobionts or pathogens by administering to the nasal cavity the glycan preparations described herein include modulating (e.g. increasing) the abundance of one or more bacterial taxa associated with nasal health, e.g. Lactobacillus (e.g. Lactobacillus sakei) and Staphylococcus (e.g. Staphylococcus epidermidis).

In some embodiments, methods for modulating (e.g. decreasing) bacterial diversity in the nasal cavity are provided by locally administering to the nasal cavity the glycan preparations described herein and modulating (e.g. increasing) the abundance of one or more bacterial taxa associated with nasal health, e.g. Lactobacillus sakei and/or Staphylococcus, and decreasing the bacterial diversity in the site.

### Oral Cavity

In some embodiments, methods of modulating the abundance of a bacterial taxa in the oral cavity of a human subject are provided. The methods comprise locally (e.g. directly) administering to the oral cavity a glycan preparation described herein in an amount and for a time effective to modulate the bacterial taxa.

In some embodiments, the glycan preparation modulates (e.g. increasing or decreasing) the growth or relative abundance of one or more (e.g. two, three, four, five or more) bacterial taxa, such as, e.g., the most abundant bacterial taxa. In some embodiments, the bacterial taxa in the oral cavity that are being modulated by administration of the glycan preparations described herein are one or more (e.g. two, three, four, five or more) of the bacterial genera Actinomyces, Corynebacterium, Rothia, Porphyromonas, Prevotella, Capnocytophaga, Gemella, Granulicatella, Streptococcus, Selenomonas, Veillonella, Fusobacterium, Leptotrichia, Kingella, Neisseria, Haemophilus, and/or Oribacterium which are common in the oral cavity. Common oral bacterial taxa in the oral cavity, specifically the teeth, include genera Actinomyces, Corynebacterium, Rothia, Porphyromonas, Prevotella, Capnocytophaga, Gemella, Granulicatella, Streptococcus, Selenomonas, Veillonella, Fusobacterium, Leptotrichia, Kingella, Neisseria, and Haemophilus.

Common oral bacterial taxa in the oral cavity, specifically the mouth include genera Actinomyces, Prevotella, Porphyromonas, Capnocytophaga, Streptococcus, Veillonella, Gemella, Oribacterium, Selenomonas, Granulicatella, Fusobacterium, Leptotrichia, Haemophilus, and Neisseria.

In some embodiments, methods of modulating one or more of Neisseria (including, e.g., Neisseria mucosa, Neisseria sicca, and Neisseria subflava), Rothia (e.g. Rothia mucilaginosa), Streptococcus (e.g. Streptococcus salivarius), and Veillonella (e.g. Veillonella parvula) bacterial taxa in the oral cavity are provided comprising administering, e.g., locally to the oral cavity the glycan preparations described herein. These bacterial taxa are thought to be associated with a healthy oral cavity.

In some embodiments, the glycan preparation drives selective changes in both the composition and activity of the oral microbiota, thereby conferring health benefits upon the human host.

In some embodiments, the health benefit includes the reduction of symptoms for a disease, disorder or pathological condition, such as, e.g. dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, tonsillitis, dentoalveolar abscess, periodontal abscess, Ludwig's angina, viral infection (e.g. herpesvirus, human papilloma virus, etc.), or fungal/yeast infections (e.g. candidiasis).

Under certain conditions, pathogenic species and pathobionts that are capable of causing disease, e.g. by inducing an infection and/or inflammation and/or bacteria associated with a disease state without necessarily being a causative agent are present in the niche. In some embodiments, methods are provided for modulating (e.g. decreasing) the abundance of oral disease-associated bacteria, pathobionts or pathogens by administering to the oral cavity the glycan preparations described herein.

In some embodiments, the disease-associated bacteria, pathobionts or pathogens include one or more of the species Streptococcus mutans; Streptococcus sobrinus.

In some embodiments, the disease-associated bacteria, pathobionts or pathogens include one or more of the species Aggregatibacter actinomycetemcomitans, Porphyromonas gingivalis, Campylobacter rectus, Treponema denticola, Fusobacterium nucleatum, Tannerella forsythia, and Prevotella intermedia.

In some embodiments, the disease-associated bacteria, pathobionts or pathogens include one or more of the species Actinomyces gerencseriae, Aggregatibacter actinomycetemcomitans, Atopobium minitum, Atopobium parvulum, Atopobium rimae, Bacteroides forsythus, Campylobacter rectus, Fusobacterium animalis, Fusobacterium nucleatum, Gemella morbillorum, Kingella oralis, Lactobacillus crispatus, Lactobacillus fermentum, Lactobacillus rhamnosus, Peptostreptococcus micros, Peptostreptococcus prevotii, Prevotella intermedia, Porphyromonas gingivalis, Selenomonas sputigena, Selenomonas noxia, Streptococcus anginosus, Streptococcus constellatus, Streptococcus mitis, Streptococcus mutans, Streptococcus oralis, Streptococcus salivarius, Streptococcus sanguinis, Streptococcus sobrinus, Tannerella forsythia, and Treponema denticola.

In some embodiments, the disease-associated bacteria, pathobionts or pathogens include one or more of the genera Veillonella, Actinomyces, Granulicatella, Leptotrichia, Thiomonas, Bifidobacterium, Prevotella, Atopobium, Olsenella, Pseudoramibacter, Propionibacterium, and Selenemonas.

In some embodiments, the disease-associated bacteria, pathobionts or pathogens include one or more of the genera Actinomyces, Aggregatibacter, Atopobium, Bacteroides, Bifidobacterium, Campylobacter, Capnocytophaga, Corynebacterium, Dialister, Eubacterium, Fusobacterium, Gemella, Granulicatella, Kingella, Lactobacillus, Leptotrichia, Olsenella, Parascardovia, Peptostreptococcus, Prevotella, Porphyromonas, Propionibacterium, Pseudoramibacter, Selenemonas, Sphingomonas, Streptococcus, Tannerella, Thiomonas, Treponema, and Veillonella.

In some embodiments, the methods for modulating (e.g. decreasing) the abundance of oral disease-associated bacteria, pathobionts or pathogens by administering to the oral cavity the glycan preparations described herein include modulating (e.g. increasing) the abundance of one or more bacterial taxa associated with oral health, e.g. one or more of Neisseria (including, e.g., Neisseria mucosa, Neisseria sicca, and Neisseria subflava), Rothia (e.g. Rothia mucilaginosa), Streptococcus (e.g. Streptococcus salivarius), and Veillonella (e.g. Veillonella parvula).

In some embodiments, methods for modulating (e.g. decreasing) bacterial diversity in the oral cavity are provided by locally administering to the oral cavity the glycan preparations described herein and modulating (e.g. increasing) the abundance of one or more bacterial taxa associated with nasal health, e.g. Neisseria (including, e.g., Neisseria mucosa, Neisseria sicca, and Neisseria subflava), Rothia (e.g. Rothia mucilaginosa), Streptococcus (e.g. Streptococcus salivarius), and Veillonella (e.g. Veillonella parvula), and decreasing the bacterial diversity in the site.

The thickness of the mucosal tissue may vary depending on the anatomical site. In some embodiments, the thickness of the mucosal tissue is between 0.5 μm to about 1 cm (e.g., between about 1 μm and about 5 mm, about 10 μm to about 1 mm, about 50 μm to about 500 μm, or about 100 μm to about 500 μm).

In some embodiments, provided herein are glycan preparations that are substrates substantially only for a selected group bacteria that are capable of utilizing the glycan preparation as a food source. The breakdown of the glycan preparation may then exert beneficial effects on the health of the host. In some embodiments, the beneficial health effects are due to a selective stimulation of the growth and/or biological activity of a selected number of microbial taxa (e.g., genera, species, or strains) in the microbiota resident at the non-gut site (e.g., nasal cavity, oral cavity and vagina) that are capable of utilizing the glycan preparation as a food source and confer health benefits to the host. The effects of the glycan preparation, in certain embodiments, are due to selective stimulation of the growth of the beneficial bacteria in the non-gut site. In some embodiments, the beneficial bacteria modulate metabolites, signaling factors, stimulants, etc. at the site and/or outcompete a pathogen or undesired bacteria in the niche. Such increases and decreases in the abundance of certain taxa may be sufficient to “normalize” the resident microbiota, e.g. to reinstate a healthy state or equilibrium. In certain embodiments, the ratio of certain bacteria or their relative abundance may be shifted. Such shifts may be measured with respect to the ratio present in the subject's non-gut site prior to, e.g., local administration of the glycan preparation, or to a control group not administering the glycan preparation to the site. The composition of the microbiota at the non-gut site can be determined on the level phylum, class, family, genus and/or species by methods known in the art, including sequencing 16S rDNA gene, FISH, real time PCR and micro-arrays, using specific probes and/or primers known in the art.

In some embodiments, the glycan preparation is a selective substrate for one or a limited number of potentially beneficial bacteria that reside in the non-gut site, stimulating their growth and/or metabolic activity. In some embodiments, the glycan preparation alters the microbial composition of the non-gut site to a composition richer or poorer in specific bacteria. In some embodiments, the glycan therapeutic selectively stimulates the growth and/or selective activity of one or more bacteria associated with health (e.g. of the site) and well-being (e.g. of the subject).

In some embodiments, the glycan preparations described herein modulate (e.g. stimulate/increase or suppress/decrease) the growth of one or more (e.g. 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 150, 200, or more than 200) endogenous commensal microbes, resident pathogens or pathobionts, or exogenously administered beneficial bacteria of suitable genera or species for the site. Exogenously administered beneficial bacteria may include those thought to be associated with a healthy (e.g. non-dysbiotic) non-gut site as described elsewhere herein.

In some embodiments, the glycan preparations described herein modulate (e.g. substantially increase or substantially decrease) the growth (and the total number) of (or substantially increase or substantially decrease the relative representation in the total bacterial community) of (or substantially increase or substantially decrease the relative abundance of a taxa in the bacterial community) of one or more of (e.g. 1, 2, 3, 4, 5, 6, 7, 8, 9, 10, 11, 12, 13, 14, 15, 16, 17, 18, 19, 20, 25, 30, 35, 40, 45, 50, or more than 50) of the taxa (e.g., the genus, species, or phylogenetic clade) listed in Tables 4-7 for the respective non-gut site.

In some embodiments, the glycan preparations described herein increase the growth (and the total number) of (or substantially increase or substantially decrease the relative representation in the total bacterial community) of (or substantially increase or substantially decrease the relative abundance of a taxa in the bacterial community) of one or more of (e.g. 1, 2, 3, 4, 5, 6, 7, 8, 9, 10, 11, 12, 13, 14, 15, 16, 17, 18, 19, 20, 25, 30, 35, 40, 45, 50, or more than 50) of the taxa (e.g., the genus, species, or phylogenetic clade) listed in Tables 4-7 for the respective non-gut site.

In some embodiments, the glycan preparations described herein decrease the growth (and the total number) of (or substantially increase or substantially decrease the relative representation in the total bacterial community) of (or substantially increase or substantially decrease the relative abundance of a taxa in the bacterial community) of one or more of (e.g. 1, 2, 3, 4, 5, 6, 7, 8, 9, 10, 11, 12, 13, 14, 15, 16, 17, 18, 19, 20, 25, 30, 35, 40, 45, 50, or more than 50) of the taxa (e.g., the genus, species, or phylogenetic clade) listed in Tables 4-7 for the respective non-gut site.

In some embodiments, the glycan preparations described herein increase and decrease the growth (and the total number) of (or substantially increase or substantially decrease the relative representation in the total bacterial community) of (or substantially increase or substantially decrease the relative abundance of a taxa in the bacterial community) of one or more of (e.g. 1, 2, 3, 4, 5, 6, 7, 8, 9, 10, 11, 12, 13, 14, 15, 16, 17, 18, 19, 20, 25, 30, 35, 40, 45, 50, or more than 50) of the taxa (e.g., the genus, species, or phylogenetic clade) listed in Tables 4-7 for the respective non-gut site.

Table 4 lists of bacterial taxa of the nasal cavity. Tables 5-6 lists of bacterial taxa of the oral cavity, e.g., teeth and the mouth, respectively. Table 7 lists of bacterial taxa of the vagina. Modulation of the composition and metabolic activity of the non-gut bacterial community (e.g. the bacterial community of the nasal cavity, oral cavity or the vagina) may be achieved, e.g., through the administration (e.g., local administration) of i) a glycan preparation alone (such as in the absence of exogenously administered bacteria), ii) a glycan preparation and one or more beneficial microorganism, or iii) a combination of a glycan preparation, a beneficial microorganism, and another agent, such as, e.g. a therapeutic agent, such as, e.g. an antibacterial agent (e.g. antibiotic), an anti-inflammatory agent, and the like. In some embodiments, to maximize the beneficial effect of endogenous commensal microbes or exogenously administered microorganisms, glycan preparations described herein are administered to stimulate the growth and/or activity of advantageous bacteria in the non-gut site. Aspects of the invention relate to glycan therapeutics that selectively improve the survival, growth, and/or effectiveness (e.g. provision of microbial metabolites or other agents (such as, e.g., bacteriocins) that support a healthy bacterial community in the non-gut site), of exogenously administered beneficial microorganism and/or resident, commensal or beneficial bacteria.

A healthy microbial community is thought to protect the host, e.g., by providing an increased barrier, e.g., by competitive exclusion of potential pathogens or disease-associated bacteria, and by growth inhibition of bacterial pathogens and disease-associated bacteria. A healthy bacterial community may exert direct antibacterial effects on pathogens and disease-associated bacteria through production of antibacterial substances, including bacteriocins and acid (e.g., a lower pH at the non-gut site that acts antiseptic) (Cotter P D, et al. 2005 Nat Rev, 3:777-788; Servin A L, 2004 FEMS Microbiol Rev, 28: 405-440). The antibacterial substances exert their effects alone or synergistically to inhibit the growth of pathogens or disease-associated bacteria. A healthy bacterial community may decrease adhesion of both pathogens and their toxins to surfaces of non-gut sites, such as, e.g., mucosal surfaces. Some methods described herein include the administration of both glycan therapeutics and exogenous beneficial bacteria to a subject's non-gut site.

Provided herein are compositions comprising a glycan preparation and compositions comprising a glycan preparation and a beneficial bacterium—or combinations of beneficial bacteria—that modulate (e.g. increasing or decreasing) the growth of bacterial constituents of the non-gut site (e.g., the nasal cavity, the oral cavity and the vagina) and/or inhibit or displace a pathogen residing at the non-gut site.

In some embodiments, glycan preparations are administered that are less efficiently metabolized by a target pathogenic bacterium than commensals. In such embodiments, glycan preparations are selected to be generally more efficiently metabolized by common commensals of the non-gut site. In such embodiments, glycan preparations are administered that stimulate/increase the growth of more than 2, more than 3, more than 4, more than 5, more than 6, more than 8, more than 12, more than 20, more than 30, or more desired beneficial bacterial taxa. In some such embodiments, glycan preparations are administered that suppress/decrease the growth of more than 2, more than 3, more than 4, more than 8, more than 12, more than 20, more than 30, or more undesired, disease-associated or harmful bacterial taxa.

In some embodiments, administration of the glycan preparations reduces inflammation. In some embodiments, administration of the glycan preparations reduces infection. Some methods described herein include the administration of both glycan preparations and exogenous beneficial bacteria to a subject's non-gut site. In some embodiments, glycan preparations and beneficial exogenous bacteria are administered to one of the oral cavity, the nasal cavity or the vagina. Alternatively or in addition, beneficial exogenous bacteria optionally together with a glycan therapeutic may be administered, e.g., orally to the gut of the subject, e.g., to modulate (e.g. upregulate/increase activity of, or downregulate/decrease activity of) immune functions. Upregulation of immune function improves, e.g., the ability to fight infections, while downregulation of immune function prevents inflammation. Optionally, glycan preparations are administered to the gut to stimulate intestinal epithelial cell responses, including restitution of damaged epithelial barrier, production of antibacterial substances and cell-protective proteins, and blocking of cytokine-induced intestinal epithelial cell apoptosis. Many of these responses result from stimulation of specific intracellular signaling pathways in the intestinal epithelial cells. Alternatively or in addition, the glycan preparations are administered locally to the non-gut site to modulate local inflammation.

Bacteria can elicit both pro- and anti-inflammatory responses from host (mammalian) cells, and different bacterial species can elicit different host responses. In one embodiment, glycan preparations are used to alter the bacterial population to elicit a desired host response. The host response may be modulated via direct interactions with the bacterial population or via indirect interactions via secreted or shed bacterial products (e.g., short-chain fatty acids). Glycan preparations may alter the bacterial population such that the bacterial population, upon either direct or indirect interaction with host cells, stimulates the production of antimicrobial peptides (AMPs), or modulates (i.e., increases or decreases the production of) inflammatory and immunomodulatory cytokines including interleukin-1α (IL-1α), IL-1β, IL-2, IL-4, IL-6, IL-8, IL-10, IL-12, IL-13, IL-17A, IL-17F, IL-22, IL-23, tumor necrosis factor (TNF), chemokine (C—C motif) ligand 5 (CCL5, also known as RANTES), transforming growth factor beta (TGF-β), interferon gamma (IFN-γ), or modulates other innate or adaptive immune responses.

In some embodiments, modulation of the non-gut site microbiota via local administration of glycan preparation to the non-gut site cavity reduces the inflammatory state of the non-gut site (e.g. the nasal cavity, oral cavity, or the vagina).

In one example, in subjects exhibiting chronic rhinosinusitis the disease-associated nasal microbiota promotes inflammation (Chalermwatanachai et al., The microbiome of the upper airways: focus on chronic rhinosinusitis, World Allergy Organ J, 2015, 8:3).

In another example, in subjects exhibiting gingivitis and periodontitis the disease-associated oral microbiota promotes both local and systemic inflammation (Seymour et al., Relationship between periodontal infections and systemic disease, Clin Microbiol Infect, 2007, Suppl 4:3).

In yet another example, in subjects exhibiting bacterial vaginosis (BV) the disease-associated vaginal microbiota promotes inflammation. Vaginal inflammation increases the susceptibility to sexually transmitted infections and the risk of preterm birth or miscarriage (Anahtar et al., Cervicovaginal bacteria are a major modulator of host inflammatory responses in the female genital tract, Immunity, 2015, 42:965; Lamont et al., The vaginal microbiome: new information about genital tract flora using molecular based techniques, BJOG, 2011, 118:533).

In some embodiments, the inflammatory state of the non-gut site is modulated by oral administration of a glycan preparation. In some embodiments, bacterial fermentation of glycan preparations in the gut produces short-chain fatty acids (SCFAs). SCFAs produced by the gut microbiota serve as energy sources for colonic epithelial cells and are thought to contribute to the maintenance of gut barrier function, which in turn limits plasma endotoxin levels and prevents systemic inflammation (Cani et al., Changes in gut microbiota control inflammation in obese mice through a mechanism involving GLP-2-driven improvement of gut permeability, Gut, 2009, 58:1091). In addition, SCFAs promote the generation of regulatory T (Treg) cells, and are thought to play a role in limiting inflammatory responses (Arpaia et al., Metabolites produced by commensal bacteria promote peripheral regulatory T-cell generation, Nature, 2013, 504:451). In some embodiments, glycan preparations are orally administered, optionally in combination with administration of a glycan preparation to the non-gut site to induce systemic effects, e.g. of SCFAs and other microbially produced immunomodulatory molecules or metabolites to modulate the inflammatory state of distal sites such as the non-gut site, e.g., the nasal cavity, oral cavity and vagina.

In some embodiments, modulation of resident bacterial taxa may be assessed by measuring one or more markers. These markers include, e.g.: i) changes in microbiota, ii) the overall metabolism of the environment, such as the production of certain metabolites, and iii) modulation of the immune system, assessing inflammatory and immune globulins.

Provided herein are methods for modulating (e.g. increasing or decreasing) microbial diversity in a non-gut site containing mucosal tissue, such as, e.g., the oral cavity, nasal cavity and the vagina. The methods can comprise administering to a subject in need thereof locally to the site a glycan preparation in an amount and for a time period effective to modulate microbial diversity in the non-gut site.

Microbial diversity can be measured by any suitable method known in the art, including analysis of 16S rDNA sequences described herein. Diversity can be expressed, e.g. using the Shannon Diversity index (Shannon entropy), number of observed OTUs, Chaol index, etc. In some embodiments, the glycan preparations modulate (e.g. increase or decrease) diversity within a microbial community, e.g. that of the non-gut, mucosal site (e.g., oral cavity, nasal cavity, or vagina), which may be expressed using Shannon entropy as a measure.

In some embodiments, the glycan therapeutics described herein increase microbial diversity and associated Shannon entropy by 0.0001%, 0.0005%, 0.001%, 0.005%, 0.01%, 0.05%, 0.1%, 0.5%, 1%, 5%, 10%, 50%, 100%, 500%, 1000%, 5000%, or 10000%. In some embodiments, the glycan therapeutics described herein increase microbial diversity and associated Shannon entropy by 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or more.

In some embodiments, the glycan therapeutics described herein decrease microbial diversity and associated Shannon entropy by 0.0001%, 0.0005%, 0.001%, 0.005%, 0.01%, 0.05%, 0.1%, 0.5%, 1%, 5%, 10%, 20%, 30%, 40%, 50%, 60%, 70%, 80%, 90%, or 99% or more. In some embodiments, the glycan therapeutics described herein decrease microbial diversity and associated Shannon entropy by 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or more.

In some embodiments, a state of lower microbial diversity is desired at the non-gut, mucosal site and methods of decreasing microbial diversity are provided. In some embodiments, high bacterial diversity is associated with dysbiosis.

### Pharmaceutical Compositions and Unit Dosage Forms

Provided herein are pharmaceutical compositions and dosage forms suitable for local administration to a non-gut site containing mucosal tissue, such as, e.g. the nasal cavity, the oral cavity and the vagina. The pharmaceutical compositions and dosage forms include a glycan preparation described herein and optionally further comprise a second (or third, fourth, etc.) therapeutic agent or active compound, such as a pharmaceutical agent, a beneficial bacterium, another active agent, etc. and/or an excipient, such as pharmaceutically acceptable excipients. In one embodiment, the agent or compound is a micronutrient, such as a vitamin, mineral or polyphenol compound. In one embodiment, the agent or compound is a therapeutic drug.

The pharmaceutical compositions and dosage forms described herein are useful to, e.g., modulate the abundance of a bacterial taxa in a non-gut tissue of a subject, modulate microbial diversity in a non-gut tissue of a subject, modulate the pH of a non-gut tissue of a subject, modulate the profile of a microbial metabolite (e.g., a volatile fatty acid) of a non-gut tissue of a subject, and/or treating a dysbiosis in a non-gut tissue of a subject.

The pharmaceutical compositions and dosage forms described herein are useful to for treatment of non-gut site diseases, disorders, or pathological conditions.

Such diseases, disorders, or pathological conditions include, e.g., for the oral cavity: dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, tonsillitis, dentoalveolar abscess, periodontal abscess, Ludwig's angina, viral infection (e.g. herpesvirus, human papilloma virus, etc.), and fungal/yeast infections (e.g. candidiasis).

Such diseases, disorders, or pathological conditions include, e.g., for the nasal cavity: rhinosinusitis (sinus infection), chronic rhinosinusitis (CRS), S. aureus infection or carriage, nasal vestibulitis, nasal furuncles and asthma.

Such diseases, disorders, or pathological conditions include, e.g., for the vagina: bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, and risk for a preterm birth or miscarriage.

In some embodiments, the pharmaceutical compositions comprising glycan preparations do not contain a prebiotic substance. In some embodiments, the pharmaceutical compositions comprising glycan preparations do not contain a beneficial bacterium.

In some embodiments, the pharmaceutical compositions comprise a glycan preparation of xyl100, rha100, ara100, ga1100, glu100, fuc100, fru100 or man100.

In some embodiments, the pharmaceutical compositions comprise a glycan preparation of ara50gal50, xyl75ga125, ara80xyl20, ara60xyl40, ara50xyl50, glu80man20, glu60man40, man60glu40, man80glu20, ga175xyl25, glu50gal50, man62glu38, and the hybrid glycans glu90sor10 or glu90gly10.

In some embodiments, the pharmaceutical compositions comprise a glycan preparation of xyl75glu12gal12, xyl33glu33ga133, glu33gal33fuc33, man52glu29gal19, and the hybrid glycan glu33gal33neu33.

In some embodiments, the pharmaceutical compositions comprise a glycan preparation of xyl100, ara100, ga1100, glu100, and man100.

In some embodiments, the pharmaceutical compositions comprise a glycan preparation of xyl75ara25, glu80man20, glu60man40, man60glu40, man80glu20, man80gal20, man66gal33, and glu50gal50.

In some embodiments, the pharmaceutical compositions comprise a glycan preparation of glu33gal33fuc33 and man52glu29gal19.

In some embodiments, pharmaceutical compositions comprising glycan preparations (and kits comprising the same) comprise one or more fatty acids. In some embodiments, the fatty acid comprises a short-chain fatty acid (SCFA), a medium-chain fatty acid (MCFA), a long-chain fatty acid (LCFA), or a very long chain fatty acid (VLCFA). In some embodiments, the short chain fatty acid comprises acetic acid, propionic acid, butryic acid, isobutyric acid, valeric acid, isovaleric acid, hexanoic acid, or octanoic acid. In some embodiments, the fatty acid comprises a saturated or unsaturated fatty acid.

In some embodiments, pharmaceutical compositions comprising glycan preparations (and kits comprising the same) comprise one or more peptides, e.g., a dipeptide, tripeptide, tetrapeptide, or pentapeptide, hexapeptide, or other length of peptide.

In some embodiments, pharmaceutical compositions comprising glycan preparations (and kits comprising same) comprise one or more micronutrient. In some embodiments, the micronutrient is selected from the group consisting of a trace mineral, choline, a vitamin, and a polyphenol.

In some embodiments, the micronutrient is a trace metal. Trace minerals suitable as a micronutrient include boron, cobalt, chromium, calcium, copper, fluoride, iodine, iron, magnesium, manganese, molybdenum, selenium, and zinc.

In some embodiments, the micronutrient is a vitamin. Vitamins suitable as a micronutrient include Vitamin B complex, Vitamin B1 (thiamin), Vitamin B2 (riboflavin), Vitamin B3 (niacin), Vitamin B5 (pantothenic acid), Vitamin B6 group (pyridoxine, pyridoxal, pyridoxamine), Vitamin B7 (biotin), Vitamin B8 (ergadenylic acid), Vitamin B9 (folic acid), Vitamin B12 (cyanocobalamin), Choline, Vitamin A (retinol), Vitamin C (ascorbic acid), Vitamin D, Vitamin E (tocopherol), Vitamin K, carotenoids (alpha carotene, beta carotene, cryptoxanthin, lutein, lycopene) and zeaxanthin.

In some embodiments, the micronutrient is a polyphenol. Polyphenols are chemical compounds or molecules that are characterized by having at least one aromatic ring with one or more hydroxyl groups. In some embodiments, the polyphenol is a synthetic polyphenol or a naturally occurring polyphenol. In some embodiments, the polyphenol is a naturally occurring polyphenol and is derived from plant source material.

In some embodiments, the polyphenol is a flavonoid or catechin. In some embodiments, the flavonoid or catechin is selected from anthocyanins, chalcones, dihydrochalcones, dihydroflavonols, flavanols, flavanones, flavones, flavonols and isoflavonoids. In some embodiments, the polyphenol is a lignan.

In some embodiments, the polyphenol is selected from alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, methoxyphenols, naphtoquinones, phenolic terpenes, and tyrosols. In some embodiments, the polyphenol is a tannin or tannic acid.

In some embodiments, the polyphenol is selected from hydroxybenzoic acids, hydroxycinnamic acids, hydroxyphenylacetic acids, hydroxyphenylpropanoic acids, and hydroxyphenylpentanoic acids. In some embodiments, the polyphenol is a stilbene.

In some embodiments, the pharmaceutical compositions comprising glycan preparations described herein further comprise a prebiotic substance or preparation thereof.

Prebiotics include various galactans and carbohydrate based gums, such as psyllium, guar, carrageen, gellan, lactulose, and konjac. In some embodiments, the prebiotic is one or more of galactooligosaccharides (GOS), lactulose, raffinose, stachyose, lactosucrose, fructo-oligosaccharides (FOS, e.g. oligofructose or oligofructan), inulin, isomalto-oligosaccharide, xylo-oligosaccharides (XOS), paratinose oligosaccharide, isomaltose oligosaccharides (IMOS), transgalactosylated oligosaccharides (e.g. transgalacto-oligosaccharides), transgalactosylate disaccharides, soybean oligosaccharides (e.g. soyoligosaccharides), chitosan oligosaccharide (chioses), gentiooligosaccharides, soy- and pectin-oligosaccharides, glucooligosaccharides, pecticoligosaccharides, palatinose polycondensates, difructose anhydride III, sorbitol, maltitol, lactitol, polyols, polydextrose, linear and branched dextrans, pullalan, hemicelluloses, reduced paratinose, cellulose, beta-glucose, beta-galactose, beta-fructose, verbascose, galactinol, xylan, inulin, chitosan, beta-glucan, guar gum, gum arabic, pectin, high sodium alginate, and lambda carrageenan, or mixtures thereof.

In some embodiments, the pharmaceutical compositions comprising glycan preparations further comprise a beneficial bacterium or preparation thereof, e.g., derived from bacterial cultures that are generally recognized as safe (GRAS) or known commensal or beneficial microbes.

Examples of suitable beneficial bacteria include:

Oral cavity: Streptococcus oralis, Streptococcus uberis, Streptococcus rattus, Bifidobacterium dentium, Bifidobacterium longum, Bifidobacterium bifidum, Lactobacillus salivarius, Lactobacillus rhamnosus, Lactobacillus plantarum, Lactobacillus salivarius, Lactobacillus paracasei, Bacillus subtilis, Lactobacillus acidophilus, Lactobacillus brevis, Lactobacillus casei, Lactobacillus reuteri, E. coli Nisle, Streptococcus salivarius, Weissella confuse, Propionibacterium freudenreichii
Vagina: Lactobacillus rhamnosus, Lactobacillus paracasei, Lactobacillus plantarum, Lactobacillus fermentum, Lactobacillus iners, Lactobacillus crispatus, Lactobacillus gasseri, Lactobacillus acidophilus, Lactobacillus jenesenii, Lactobacillus brevis, Lactobacillus casei, Lactobacillus vaginalis, Lactobacillus delbrueckii, Lactobacillus salivarius, Lactobacillus reuteri, Lactobacillus rahmnosus, Lactobacillus pentosus, Bacillus coagulans.
Nasal cavity: Lactobacillus sakei, Lactobacillus reuteri, Streptococcus salivarius, Streptococcus thermophiles, Lactobacillus acidophilus, Bifidobacterium sp B420, and Lactobacillus GG.

In some embodiments, the beneficial or commensal bacteria include one or more of the bacteria listed in Tables 4-7.

The prebiotic substances and beneficial strains that may be combined with glycan preparations described herein to produce a composition may be isolated at any level of purity by standard methods and purification can be achieved by conventional means known to those skilled in the art, such as distillation, recrystallization and chromatography. If desired, the cultivated bacteria may be used in the composition. The bacteria may be separated from the culture broth by any method including, without limitations, centrifugation, filtration or decantation. The cells separated from the fermentation broth are optionally washed by water, saline (0.9% NaCl) or with any suitable buffer. The wet cell mass obtained may be dried by any suitable method, e.g., by lyophilization.

In some embodiments, the beneficial bacteria are lyophilized vegetative cells. In some embodiments, preparations of spores from sporulating beneficial bacteria are used.

In one embodiment, the pharmaceutical compositions comprise a glycan preparation and beneficial bacteria whose viability has been partially attenuated (e.g. a mixture comprising 10%, 20%, 30%, 40%, 50% or more non-viable bacteria), or beneficial bacteria consisting primarily of non-viable microbes (e.g. 95%, 96%, 97%, 98%, 99%, 99.9% or 100%). The compositions may further comprise microbial membranes and/or cell walls that have been isolated and purified from microbes or microbial vesicles. If desired, the beneficial microbial organism(s) can be incorporated into the pharmaceutical glycan composition as a culture in water or another liquid or semisolid medium in which the beneficial bacterium remains viable. In another technique, a freeze-dried powder containing the beneficial bacterium may be incorporated into a particulate material or liquid or semisolid material comprising the glycan preparation by mixing or blending.

In some embodiments, the pharmaceutical compositions comprising glycan preparations further comprise a second therapeutic agent or preparation thereof, such as a drug.

For example, the second therapeutic agent is a steroid, such as, e.g. prednisone or dexamethasone.

In some embodiments, the therapeutic agent is an anti-inflammatory agent, such as, e.g., an NSAID, including ibuprofen, naproxen sodium, aspirin, celecoxib, sulindac, oxaprozin, salsalate, diflunisal, piroxicam, indomethacin, etodolac, meloxicam, nabumetone, ketorolac tromethamine, naproxen/esomeprazole, or diclofenac.

In some embodiments, the second therapeutic agent is an antimicrobial agent, such as an antibiotic, an antifungal agent, or an antiviral. Antibiotics include aminoglycosides, such as amikacin, gentamicin, kanamycin, neomycin, streptomycin, and tobramycin; cephalosporins, such as cefamandole, cefazolin, cephalexin, cephaloglycin, cephaloridine, cephalothin, cephapirin, and cephradine; macrolides, such as erythromycin and troleandomycin; penicillins, such as penicillin G, amoxicillin, ampicillin, carbenicillin, cloxacillin, dicloxacillin, methicillin, nafcillin, oxacillin, phenethicillin, and ticarcillin; polypeptide antibiotics, such as bacitracin, colistimethate, colistin, polymyxin B; tetracyclines, such as chlortetracycline, demeclocycline, doxycycline, methacycline, minocycline, tetracycline, and oxytetracycline; and miscellaneous antibiotics such as chloramphenicol, clindamycin, cycloserine, lincomycin, rifampin, spectinomycin, vancomycin, viomycin and metronidazole.

For example, the second therapeutic agent is a pain-management drug. In some embodiments, the pain-management drug is an opioid, such as, e.g., codeine, fentanyl, hydrocodone, hydrocodone/acetaminophen, hydromorphone, meperidine, methadone, morphine, oxycodone, oxycodone and acetaminophen, or oxycodone and naloxone. In other embodiments, the pain-management drug is a non-opioid, such as, e.g., acetaminophen or nonsteroidal anti-inflammatory drugs (NSAIDs), such as aspirin and ibuprofen.

The glycan preparations described herein and the therapeutic agent or active compound may be comingled or mixed in a single pharmaceutical composition. In other embodiments, they may be contained in separate containers (and/or in various suitable unit dosage forms) but packaged together in one or more kits. In some embodiments, the preparations or compositions are not packaged or placed together.

In some embodiments, a pharmaceutical composition comprises between 0.1% and 100% glycan preparation by w/w, w/v, v/v or molar %. In another embodiment, a pharmaceutical composition comprises about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 712%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% of glycan preparation by w/w, w/v, v/v or molar %. In one embodiment, a pharmaceutical composition comprises about 1-90%, about 10-90%, about 20-90%, about 30-90%, about 40-90%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-80%, about 70-90%, about 70-80%, about 80-90%, about 90-96%, about 93-96%, about 93-95%, about 94-98%, about 93-99%, or about 90-100% of glycan preparation by w/w, w/v, v/v or molar %.

Optionally, the pharmaceutical compositions comprising glycan preparations comprise one or more excipients or carriers, including diluents, binders, disintegrants, dispersants, lubricants, glidants, stabilizers, surfactants, flavoring agents, and colorants. The pharmaceutical composition can comprise from about 1% to about 90% of the one or more excipients or carriers by w/w, w/v, v/v or molar %. For example, the pharmaceutical composition can comprise about 1-90%, 1-75%, 1-60%, 1-55%, 1-50%, 1-45%, 1-40%, 1-25%, 1-15%, 1-10%, 10-90%, 10-75%, 10-60%, 10-55%, 10-50%, 10-45%, 10-40%, 10-25%, 10-15%, 15-90%, 15-75%, 15-60%, 15-55%, 15-50%, 15-45%, 15-40%, 15-25%, 25-90%, 25-75%, 25-60%, 25-55%, 25-50%, 25-45%, 25-40%, 40-90%, 40-75%, 40-60%, 40-55%, 40-50%, 40-45%, 45-90%, 45-75%, 45-60%, 45-55%, 45-50%, 50-90%, 50-75%, 50-60%, 50-55%, 55-90%, 55-75%, 55-60%, 60-90%, 60-75%, 75-90% of the one or more excipients or carriers by w/w, w/v, v/v or molar %.

Pharmaceutical carriers or vehicles suitable for administration, e.g., local administration of the pharmaceutical glycan compositions provided herein to a non-gut site include all such carriers known to those skilled in the art to be suitable for the particular mode of administration. In addition, the compositions can one or more components that do not impair the desired action, or with components that supplement the desired action, or have another action.

### Dosage Forms

The glycan compositions described herein may be formulated into any suitable dosage form including semi-solids, such as, e.g., gels, creams, ointments, mists, aerosols, liquids, and solids, such as, e.g. powders or coatings, as well as in suitable devices and applicators, such as patches, films, syringes, vaginal rings, brushes, spray bottles, squirt bottles, dispensers, etc. or may be formulated as capsules, tablets, packet, sachet, canister, ampoule, ramekin, cans, soft packs, and the like. Kits or packages may comprise the compositions packaged in bulk (e.g., in a container containing sufficient glycan preparation or other substances for a subject to follow for an entire course of treatment or a defined portion of a course of treatment), or as individual packets (e.g., packets containing a single dose of glycan preparation optionally plus other components, or packets containing the dose of glycan preparation and other components needed for a particular day of a glycan preparation treatment regimen).

### Vagina

Vaginal delivery may involve introduction of the glycan therapeutic composition onto or into any region or subsection of the vagina or surrounding area, including the labia, vulva, cervix, uterus, fallopian tube, ovary, urethra, bladder, anus, and rectum. In some embodiments, vaginal delivery occurs through transvaginal absorption into the muscosal tissue. Exemplary dosage forms for vaginal delivery include a suppository (e.g., pessary), cream, ointment, solution, suspension, emulsion, vaginal ring, tampon, pad, douche, sponge, cup, intrauterine device (IUD), intravesical infusion, strip, spray, foam, tablet, capsule, pill, patch, pellet, cap, membrane, fiber, applicator, adhesive, shield (e.g., condom), or extra-amniotic infusion. In some embodiments, the dosage form suitable for vaginal delivery is capable of maintaining a particular shape or consistency upon administration. In some embodiments, the dosage form suitable for vaginal delivery dissolves or changes form upon administration.

Exemplary vaginal applications include topical, sublabial, intradermal, intramuscular, intracavity, subcutaneous, or insufflation, or may occur via direct injection or by spray. Vaginal administration of the glycan therapeutic composition may involve a single dosage or may occur in multiple dosage, e.g., over a selected period of time.

In some embodiments, the dosage form suitable for vaginal delivery is capable of delivering the glycan therapeutic composition to a specific site in a controlled manner. In some embodiments, the vaginal dosage form is formulated in a timed-release or dissolvable fashion, and may release the glycan therapeutic composition immediately or after about 2 seconds, about 5 seconds, about 10 seconds, about 20 seconds, about 30 seconds, about 45 seconds, about 1 minute, about 2 minutes, about 5 minutes, about 10 minutes, about 15 minutes, about 30 minutes, about 1 hour, about 1.5 hours, about 2 hours, about 4 hours, about 8 hours, about 12 hours, about 16 hours, about 20 hours, about 1 day, about 2 days, about 3 days, about 4 days, about 5 days, about 6 days, about 1 week, about 2 weeks, about 4 weeks, or longer.

In some embodiments, the dosage form for vaginal delivery of an exemplary glycan therapeutic composition comprises two or more components. In some embodiments, the first component of the dosage form comprises a device of a solid and flexible material that houses a second component, which comprises a medium, e.g., a gel or liquid, in which the glycan therapeutic composition is dissolved or mixed. Such a two component system may entail applying the first component into or onto the vagina, before or after placement of the second component, which allows for controlled delivery of the dosage form. For example, the dosage form for vaginal delivery may entail a semi-solid cream or liquid comprising the glycan therapeutic composition and a syringe or other injection device for administration into the vaginal cavity.

In some embodiments, the dosage form for vaginal delivery further comprises a contraceptive, e.g., a spermicide, an intrauterine device, a hormonal contraceptive, or rubber latex shield (e.g., condom). In other embodiments, the dosage form for vaginal delivery further comprises an agent to prevent or combat a sexually transmitted disease (e.g., a viral, fungal, or bacterial disease or infection), e.g., nonoxynol-9, azithromycin, penicillin, ceftriaxone, ciprofloxacin, or metronidazole. In other embodiments, the dosage form for vaginal delivery further comprises an agent to prevent or combat a urogenital infection, e.g., a urinary tract infection, bacterial vaginosis, or other dysbiosis associated with the vaginal cavity (e.g., miconazole, terconazole).

In some embodiments, the glycan compositions are formulated for local vaginal administration, such as intravaginal administration. The dosage forms include, e.g., a vaginal tablet, vaginal cream or gel, douche, vaginal suppository, intravaginal implant or pessary, tampon, or a vaginal ring.

### Oral Cavity

In some embodiments, the dosage form is formulated for oral delivery. Oral delivery may involve introduction of the glycan therapeutic composition onto or into any region or subsection of the oral cavity, such as the mouth, lips, gums, tongue, cheek, palate, salivary gland, jaw, pharynx, epiglottis, nasal cavity, respiratory cavity (e.g., upper lung cavity or lower lung cavity), larynx, and esophagus. Oral delivery further comprises delivery to the skin or mucosal surfaces, e.g., of the mouth (e.g., masticatory and lining musosa), throat, nasal passages, and respiratory cavity. Exemplary oral dosage forms include a solid (e.g., a tablet, pill, capsule, pastille, granule, candy, drop, lozenge, gum, powder, paste, troche, crystal, chew, dissolving strip, film, fast melt, foodstuff, or semi-solid formulation), liquid (e.g., a beverage, suspension, syrup, elixir, solution, linctus, syrup, mouthwash, spray, tincture, drop, infusion, or emulsion), or gel (e.g., a toothpaste or ointment). In some embodiments, the oral dosage form is formulated as a food item, e.g., a nutritional supplement, baked good, bar, beverage, spread, candy, confection, or as a powder for dilution.

Exemplary oral applications include topical, buccal, sublingual, intradermal, intramuscular, subcutaneous, insufflation, or inhalational administration, or may occur via a gastric feeding tube. Oral administration of the glycan therapeutic composition may involve a single dosage or may occur in multiple dosages, e.g., over a selected period of time. In some embodiments, the dosage form for oral delivery further comprises an agent to prevent tooth caries, periodontitis, and/or gingivitis, e.g., fluoride or an antibacterial agent. In other embodiments, the dosage form for oral delivery further comprises an agent to prevent or combat halitosis, e.g., an antibacterial agent, zinc, or triclosan. In other embodiments, the dosage form for oral delivery further comprises an agent to prevent or combat an oral sore, e.g., a cold sore or canker sore (e.g., an antiviral agent (e.g., acyclovir, famiciclovir, valacyclovir), lysine, lemon balm, aloe vera, zinc, dexamethasone, fluocinonide, hydrogen peroxide, colchicine, sucralfate, silver nitrate, or debacterol).

In some embodiments, the dosage form suitable for oral delivery is capable of delivering the glycan therapeutic composition to a specific site in a controlled manner. In some embodiments, the oral dosage form is formulated in a timed-release or dissolvable fashion, and may release the glycan therapeutic composition immediately or after about 2 seconds, about 5 seconds, about 10 seconds, about 20 seconds, about 30 seconds, about 45 seconds, about 1 minute, about 2 minutes, about 5 minutes, about 10 minutes, about 15 minutes, about 30 minutes, about 1 hour, about 1.5 hours, about 2 hours, about 4 hours, about 8 hours, about 12 hours, about 16 hours, about 20 hours, about 1 day, or longer. In some embodiments, the oral dosage form is administered to the oral cavity through the aid of a device, such as a syringe, feeding tube, retainer, inhaler, spray, or bioadhesive patch.

In some embodiments, oral delivery comprises delivery to the gastrointestinal tract. In other embodiments, oral delivery is confined to the oral cavity (e.g., mouth, lips, gums, tongue, cheek, nasal cavity, palate, salivary gland, jaw, pharynx, epiglottis, larynx, and esophagus) and does not enter the gastrointestinal tract and/or has minimal systemic exposure. In some embodiments, the subject holds the oral dosage form in the mouth without swallowing. In some embodiments, the subject activates the oral dosage form in the mouth by swirling or gargling. In some embodiments, the oral dosage form has a residence time of greater than about 5 seconds in the oral cavity of a subject, e.g., greater than about 10 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the oral dosage form has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

In some embodiments, the glycan compositions are formulated for local oral administration. In one embodiment, the dosage forms include, e.g., a spray, a mist, a gel, a film, a gum, a rinse (mouthwash), a lollipop, a tablet, a capsule, a lozenge.

### Nasal Cavity

In some embodiments, the dosage form is formulated for nasal delivery. Nasal delivery may involve introduction of the glycan therapeutic composition onto or into any region or subsection of the nasal cavity, such as the nose, nasal conchae (e.g., inferior conchae), vestibule, maxilla, palatine bone, medial pterygoid plate, labyrinth of ethmoid, sinuses (e.g., paranasal sinus, frontal sinus, maxillary sinus, sphenoid sinus, ethmoid sinus), ostia, nasal wall (e.g., lateral nasal wall), infundibulum, palate, nasopharynx, olfactory epithelium, respiratory epithelium, and vomeronasal organ. In some embodiments, the dosage form is targeted to the olfactory segment and/or the respiratory segment of the nasal cavity. Nasal delivery further comprises delivery to the skin or mucosal surfaces, e.g., of the nose, sinuses, nasal passages, and respiratory cavity. Exemplary nasal dosage forms include a solid (e.g., a tablet, pill, capsule, pastille, granule, powder, paste, crystal, dissolving strip, film, or semi-solid formulation), liquid (e.g., a spray, mist, drop, suspension, solution, tincture, infusion, aerosol, or emulsion), or gel (e.g., an ointment). In some embodiments, the nasal dosage form is administered by an inhaler (e.g., metered dose inhaler, dry-powder inhaler), a nebulizer, a syringe, neti pot, dropper, bottle, pump (e.g., atomized pump, atomizer), or pressurized aerosol. The nasal dosage form may be administered as a particle with a discrete size. In some embodiments, the particle size of the nasal dosage form is between about 1 μm and about 50 μm (e.g., about 5 μm and about 30 μm, about 10 μm and about 20 μm). In some embodiments, the dosage form for administration to the nasal cavity comprises a nanoparticle (e.g., a mucus-penetrating particle).

Exemplary nasal applications include topical, intradermal, subcutaneous, insufflation, or inhalational administration, or may occur via a nasal tube. In some embodiments, the dosage form for nasal delivery further comprises an agent to treat or prevent rhinosinusitis (sinus infection, e.g., acute sinusitis), chronic rhinosinusitis (CRS), S. aureus infection or carriage, nasal vestibulitis, or nasal furuncles, e.g., an antibiotic (e.g., amoxicillin, amoxicillin-clavulante, azithromycin, cefprozil, moxifloxacin, erythromycin, ampicillin), a decongestant (e.g., pseudoephedrine, phenylephrine, ephedrine, levomethamphetamine, naphazoline, oxymetazoline, phenylpropanolamine, propylhexedrine, synephrine, tetrahydrozoline, xylometazoline, tramazoline), a corticosteroid (e.g., fluticasone propionate, triamcinolone acetonide), or a mucolytic (e.g., acetylcysteine, ambroxol, bromhexine, carbocisteine, domiodol, domase alfa, eprazinone, erdosteine, letosteine, mannitol, mesna, neltenexine, sorberol, stepronin, tiopronin). In some embodiments, the dosage form for nasal delivery further comprises an agent to treat or prevent asthma, e.g., a corticosteroid (e.g., beclomethasone), a long-acting beta agonist (e.g., salmeterol, formoterol), a short-acting beta agonist (e.g., salbutamol), an anticholinergic agent (e.g., ipratropium bromide), an antileukotriene agent (e.g., montelukast, zafirlukast), a mast cell stabilizer (e.g., cromolyn sodium), or magnesium sulfate.

Nasal administration of the glycan therapeutic composition may involve a single dosage or may occur in multiple dosages, e.g., over a selected period of time. In some embodiments, the dosage form suitable for nasal delivery is capable of delivering the glycan therapeutic composition to a specific site in a controlled manner. In some embodiments, the nasal dosage form is formulated in a timed-release or dissolvable fashion, and may release the glycan therapeutic composition immediately or after about 2 seconds, about 5 seconds, about 10 seconds, about 20 seconds, about 30 seconds, about 45 seconds, about 1 minute, about 2 minutes, about 5 minutes, about 10 minutes, about 15 minutes, about 30 minutes, about 1 hour, about 1.5 hours, about 2 hours, about 4 hours, about 8 hours, about 12 hours, about 16 hours, about 20 hours, about 1 day, or longer.

In some embodiments, the glycan compositions are formulated for local nasal administration. In one embodiment, the dosage forms include a spray, a mist, a gel, an ointment (e.g., applied to the nares), a swab, a dropper, a nebulizer, a dry powder inhaler, a tablet, a capsule, and a lozenge.

The dosage forms described herein can be manufactured using processes that are known to those of skill in the art. The dosage form may be suitable for any route of administration, including local administration, e.g., administration to the mucosal or non-mucosal tissues of the non-gut sites. In some embodiments, the local administration is topical administration.

The dosage form may be a packet, such as any individual container that contains a pharmaceutical glycan therapeutic composition in the form of, e.g., a liquid (wash/rinse), a gel, a cream, an ointment, a powder, a tablet, a pill, a capsule, a depository, a single-use applicator or medical device (e.g. a syringe). For example, provided is also an article of manufacture, such as a container comprising a unit dosage form of the pharmaceutical glycan composition, and a label containing instructions for use of such glycan therapeutic.

The pharmaceutical compositions provided herein can be in unit-dosage forms or multiple-dosage forms. A unit-dosage form, as used herein, refers to physically discrete unit suitable for administration to a human (e.g. locally to the non-gut site) in need thereof. In an embodiment, the unit-dosage form is provided in a package. Each unit-dose can contain a predetermined quantity of an active ingredient(s) sufficient to produce the desired therapeutic effect, in association with other pharmaceutical carriers or excipients. Examples of unit-dosage forms include ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include vials, bottles of tablets or capsules, or bottles of pints or gallons. In another embodiment the multiple dosage forms comprise different pharmaceutically active agents. For example, a multiple dosage form can be provided which comprises a first dosage element comprising a composition comprising a glycan preparation and a second dosage element comprising a second active compound, e.g., a second glycan preparation or a therapeutic agent (e.g. a drug) or beneficial bacterium. The dosage elements can be in a modified release form. In this example, a pair of dosage elements can make a single unit dosage. In one embodiment, a kit is provided comprising multiple unit dosages, wherein each unit comprises a first dosage element comprising a composition comprising a glycan preparation and a second dosage element comprising a second active compound, e.g., a second glycan preparation, a therapeutic agent (e.g., a pharmaceutical agent), a beneficial bacterium, a micronutrient, etc. or a combination thereof).

In certain embodiments, the unit-dosage form may comprise about 1 g to about 5 g, about 1 g to about 10 g, about 1 g to about 15 g, about 1 g to about 20 g, about 1 g to about 25 g, about 1 g to about 30 g, about 1 g to about 40 g, about 1 g to about 50 g, about 5 g to about 10 g, about 5 g to about 15 g, about 5 g to about 20 g, about 5 g to about 25 g, about 5 g to about 30 g, about 10 g to about 20 g, or about 10 g to about 30 g, about 10 g to about 40 g, about 10 g to about 50 g of the glycan preparation.

In certain embodiments, the unit-dosage form comprises about 0.001 mg to about 100 mg, about 0.005 mg to about 75 mg, about 0.01 mg to about 50 mg, about 0.05 mg to about 25 mg, about 0.1 mg to about 10 mg, about 0.5 mg to about 7.5 mg, or about 1 mg to about 5 mg of the glycan preparation. In other embodiments, the unit-dosage form comprises about 1 mg to about 100 mg, about 2.5 mg to about 75 mg, about 5 mg to about 50 mg, or about 10 mg to about 25 mg of the glycan therapeutic. In other embodiments, the unit-dosage form comprises about 100 mg to about 10 g, about 250 mg to about 7.5 g, about 500 mg to about 5 g, about 750 mg to about 2.5 g, or about 1 g to about 2 g of the glycan preparation.

In other embodiments, the unit-dosage form comprises between about 0.001 mL to about 1000 mL of the glycan preparation. For example, the unit-dosage form may comprise about 0.001 mL to about 950 mL, about 0.005 mL to about 900 mL, about 0.01 mL to about 850 mL, about 0.05 mL to about 800 mL, about 0.075 mL to about 750 mL, about 0.1 mL to about 700 mL, about 0.25 mL to about 650 mL, about 0.5 mL to about 600 mL, about 0.75 mL to about 550 mL, about 1 mL to about 500 mL, about 2.5 mL to about 450 mL, about 5 mL to about 400 mL, about 7.5 mL to about 350 mL, about 10 mL to about 300 mL, about 12.5 mL to about 250 mL, about 15 mL to about 200 mL, about 17.5 mL to about 150 mL, about 20 mL to about 100 mL, or about 25 mL to about 75 mL of the glycan preparation.

In some embodiments, the unit-dosage form has a body length of between about 0.1 inches to about 1.5 inches (e.g., about 0.5 inches and about 1 inch), or about 5 mm to about 50 mm (e.g., about 10 mm to about 25 mm). In some embodiments, the unit-dosage form. e.g., a tablet, capsule (e.g., a hard capsule, push-fit capsule, or soft capsule), or softgel, has an external diameter of about 0.05 inches to about 1 inch (e.g., about 0.1 inches to about 0.5 inches), or about 1 mm to about 25 mm (e.g., about 5 mm to about 10 mm).

The dosage forms described herein can be manufactured using processes that are known to those of skill in the art.

Excipients and additives include diluents, binders, disintegrants, dispersants, lubricants, glidants, stabilizers, surfactants, antiadherents, sorbents, sweeteners, and colorants, or a combination thereof. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Non-limiting examples of suitable binders include starch (including corn starch and pregelatinized starch), gelatin, sugars (e.g., glucose, dextrose, sucrose, lactose and sorbitol), celluloses, polyethylene glycol, alginic acid, dextrin, casein, methyl cellulose, waxes, natural and synthetic gums, e.g., acacia, tragacanth, sodium alginate, gum arabic, xantan gum, and synthetic polymers such as polymethacrylates, polyvinyl alcohols, hydroxypropylcellulose, and polyvinylpyrrolidone. Non-limiting examples of lubricans include magnesium stearate, calcium stearate, stearic acid, glyceryl behenate, and polyethylene glycol. Non-limiting examples of disintegrants include starches, alginic acid, crosslinked polymers such as, e.g., crosslinked polyvinylpyrrolidone, croscarmellose sodium, potassium or sodium starch glycolate, clays, celluloses (e.g., carboxymethylcelluloses (e.g., carboxymethylcellulose (CMC), CMC-Na, CMC-Ca)), starches, gums and the like. Non-limiting examples of suitable glidants include silicon dioxide, talc, and the like. Stabilizers can inhibit or retard drug decomposition reactions, including oxidative reactions. Surfactants can also include and can be anionic, cationic, amphoteric or nonionic. Exemplary sweeteners may include stevia extract, aspartame, sucrose, alitame, saccharin, and the like. If desired, the compositions can also comprise nontoxic auxiliary substances such as pH buffering agents, preservatives, e.g., antioxidants, wetting or emulsifying agents, solubilizing agents, coating agents, flavoring agents (e.g., mint, cherry, anise, peach, apricot, licorice, raspberry, vanilla), and the like. Additional excipients and additives may include aluminum acetate, benzyl alcohol, butyl paraben, butylated hydroxy toluene, calcium disodium EDTA, calcium hydrogen phosphate dihydrate, dibasic calcium phosphate, tribasic calcium phosphate, candelilla wax, carnuba wax, castor oil hydrogenated, cetylpyridine chloride, citric acid, colloidal silicone dioxide, copolyvidone, corn starch, cysteine HCl, dimethicone, disodium hydrogen phosphate, erythrosine sodium, ethyl cellulose, gelatin, glycerin, glyceryl monooleate, glyceryl monostearate, glycine, HPMC pthalate, hydroxypropylcellulose, hydroxyl propyl methyl cellulose, hypromellose, iron oxide red or ferric oxide, iron oxide yellow, iron oxide or ferric oxide, magnesium carbonate, magnesium oxide, magnesium stearate, methionine, methacrylic acid copolymer, methyl paraben, silicified microcrystalline cellulose, mineral oil, phosphoric acid, plain calcium phosphate, anhydrous calcium phosphate, polaxamer 407, polaxamer 188, plain polaxamer, polyethylene oxide, polyoxyl40 stearate, polysorbate 80, potassium bicarbonate, potassium sorbate, potato starch, povidone, propylene glycol, propylene paraben, propyl paraben, retinyl palmitate, saccharin sodium, selenium, silica, silica gel, fumed silica, sodium benzoate, sodium carbonate, sodium citrate dihydrate, sodium crossmellose, sodium lauryl sulfate, sodium metabisulfite, sodium propionate, sodium starch, sodium starch glycolate, sodium stearyl fumarate, sorbic acid, sorbitol, sorbitan monooleate, pregelatinized starch, succinic acid, triacetin, triethyl citrate, vegetable stearin, vitamin A, vitamin E, vitamin C, or a combination thereof. The amounts of these excipients and additives can be properly selected based on their relation to other components and properties of the preparation and production method.

In some embodiments, the formulations described herein comprise an excipient specific for mucosal delivery. Examples of such excipients include microcrystalline cellulose, carboxymethylcellulose sodium, dextrose, benzalkonium chloride (e.g., at a concentration of about 0.01-0.05%, e.g., 0.02% w/w), polysorbate 80, phenylethyl alcohol (e.g., at a concentration of about 0.1-0.5%, e.g., about 0.25% w/w), or edetate disodium. In other embodiments, the formulations described herein comprise a mucosal penetrating agent, which may increase the permeability of the active agent through the mucosa. Exemplary permeation enhances include surfactants, bile salts, non-surfactants (e.g., cyclodextrins, chitosan, and Azones), and/or fatty acids. Other exemplary excipients that can be used for mucosal delivery are described in Expert Opin Drug Deliv. 2012 Jun. 9(6):615-28, incorporated herein by reference.

In embodiments, the composition is formulated for mucosal delivery, e.g., nasal mucosal delivery or oral mucosal delivery. In embodiments, the composition is in/on/within/incorporated with a polymer, e.g., mucoadhesive polymers, e.g., hydrogel. Without wishing to be bound by theory, it is believed that the inclusion of a mucoadhesive polymer in the formulation can increase the contact time of the active agent with the mucosa, e.g., thereby increasing the duration time for absorption. Exemplary mucoadhesive polymers include Carbopol 934P, hydroxy propyl cellulose, poly(vinyl pyrrolidone), sodium carboxymethyl cellulose, hydroxy propyl methyl cellulose, hydroxy ethyl cellulose, poly(vinyl alcohol), poly(isobutylene), poly(isoprene), xanthum gum, locust bean gum, chitosan, pectin, polycarbophil, hyaluronic acid benzyl esters, poly(acrylic acid), poly(methacrylic acid), poly(acrylic acid-co-acrylamide), poly(acrylic acid-co-methyl methacrylate), poly(acrylic acid-co-butylacrylate), HEMA copolymerized with Polymeg® (polytetramethylene glycol), Cydot® by 3M (bioadhesive polymeric blend of CP and PIB), Carbopol EX-55, polyethylene oxide, polymethylvinylether/maleic anhydride (PME/MA), tragacanth, poly(acrylic acid-co-poly ethyleneglycol) copolymer of acrylic acid and poly ethyleneglycol monomethylether monomethacryalte, polyethylene glycol, drum dried waxy maize starch (DDWM), and sodium stearylfumarate. Immediate-release formulations of an effective amount of a glycan composition can comprise one or more combinations of excipients that allow for a rapid release of a pharmaceutically active agent (such as from 1 minute to 1 hour after administration). Controlled-release formulations (also referred to as sustained release (SR), extended-release (ER, XR, or XL), time-release or timed-release, controlled-release (CR), or continuous-release) refer to the release of a glycan composition from a dosage form at a particular desired point in time after the dosage form is administered to a subject (e.g., locally to the non-gut site).

In one embodiment a controlled release dosage form begins its release and continues that release over an extended period of time. Release can occur beginning almost immediately or can be sustained. Release can be constant, can increase or decrease over time, can be pulsed, can be continuous or intermittent, and the like. In one embodiment, a controlled release dosage refers to the release of an agent from a composition or dosage form in which the agent is released according to a desired profile over an extended period of time. In one aspect, controlled-release refers to delayed release of an agent from a composition or dosage form in which the agent is released according to a desired profile in which the release occurs after a period of time.

In some embodiments, the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva.

In another embodiment, the dosage form can be in a candy form (e.g., matrix), such as a lollipop or lozenge. In one embodiment an effective amount of a glycan preparation is dispersed within a candy matrix. In one embodiment the candy matrix comprises one or more sugars (such as dextrose or sucrose). In another embodiment the candy matrix is a sugar-free matrix. The choice of a particular candy matrix is subject to wide variation. Conventional sweeteners (e.g., sucrose), sugar alcohols suitable for use with diabetic patients (e.g., sorbitol or mannitol), or other sweeteners (e.g., sweeteners described herein) may be employed. The candy base can be very soft and fast dissolving, or can be hard and slower dissolving. Various forms will have advantages in different situations.

A candy mass composition comprising an effective amount of the glycan preparation can be orally administered to a subject in need thereof so that an effective amount of the glycan preparation will be released locally into the subject's mouth as the candy mass dissolves.

The dosage forms described herein can also take the form of pharmaceutical particles manufactured by a variety of methods, including high-pressure homogenization, wet or dry ball milling, or small particle precipitation. Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In one embodiment, the pharmaceutical particles have a final size of 3-1000 microns, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 microns. In another embodiment the pharmaceutical particles have a final size of 10-500 microns. In another embodiment the pharmaceutical particles have a final size of 50-600 microns. In another embodiment the pharmaceutical particles have a final size of 100-800 microns.

In another embodiment, an oral dosage form is provided comprising a glycan composition, wherein the oral dosage form is a syrup. The syrup can comprise about 1%, 5%, 10%, 15%, 20%, 25%, 30%, 35%, 40%, 45%, 50%, 55%, 60%, 65%, 70%, 75%, 80%, or 85% solid. The syrup can comprise about 15%, 20%, 25%, 30%, 35%, 40%, 45%, or 50% liquid, for example, water. The solid can comprise a glycan composition. The solid can be, for example, about 1-96%, 10-96%, 20-96%, 30-96%, 40-96%, 50-96%, 60-96%, 70-96%, 80-96%, or 90-96% glycan composition. In another embodiment, a glycan composition is formulated as a viscous fluid.

In one embodiment, the composition comprises a foaming component or a neutralizing component. A foaming component can be at least one member selected from the group consisting of sodium hydrogencarbonate, sodium carbonate, and calcium carbonate. In one embodiment a neutralizing component can be at least one member selected from the group consisting of citric acid, L-tartaric acid, fumaric acid, L-ascorbic acid, DL-malic acid, acetic acid, lactic acid, and anhydrous citric acid. The formulation can contain a sucrose fatty acid ester, powder sugar, fruit juice powder, and/or flavoring material.

In some embodiments, the dosage form for the pharmaceutical glycan compositions described herein is a mucoadhesive delivery system that adheres to the mucosal surfaces, such as those of a non-gut site, such as, e.g., the oral cavity, the nasal cavity and the vagina. They are typically composed of polymers with numerous hydrogen-bonding groups, e.g., cross-linked polyacrylic acids, sodium carboxymethyl cellulose, sodium alginate, carrageenan, Carbopol 934P, or thiolated polycarbophil.

In some embodiments, the dosage form for the pharmaceutical glycan compositions described herein are suppositories. Suppositories are solid dosage forms that melt or dissolve, e.g, when inserted into the vagina, releasing the glycan preparation. Typical excipients for suppository formulations include cocoa butter, polyethylene glycols, and agar.

Further provided herein are methods of making a unit-dosage form described herein, comprising providing a glycan preparation; formulating the glycan preparation into a unit-dosage form, packaging the unit-dosage form, labelling the packaged unit-dosage form, and/or selling or offering for sale the packaged and labeled unit-dosage form.

The unit-dosage forms described herein may also be processed. In one embodiment, the processing comprises one or more of: processing the dosage form into a pharmaceutical composition, e.g., formulating, combining with a second component, e.g., an excipient or buffer or a second active compound or therapeutic agent; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a pharmaceutical composition, shipping, moving to a different location, formulating, labeling, packaging, releasing into commerce, or selling or offering for sale, depending on whether the predetermined threshold is met. In some embodiments, the processed dosage forms comprise a glycan preparation described herein.

### Kits

Kits are also contemplated. For example, a kit can comprise unit dosage forms of the pharmaceutical glycan composition, and a package insert containing instructions for use of the glycan preparation in treatment of a disease, disorder or pathological condition. The kits include a pharmaceutical glycan composition in suitable packaging for use by a subject in need thereof.

Any of the compositions described herein can be packaged in the form of a kit. A kit can contain an amount of a pharmaceutical glycan composition (optionally additionally comprising a beneficial bacterium, a micronutrient, and/or a second therapeutic agent, such as a drug) sufficient for an entire course of treatment, or for a portion of a course of treatment. Doses of a pharmaceutical glycan composition can be individually packaged, or the pharmaceutical glycan therapeutic composition can be provided in bulk, or combinations thereof. Thus, in one embodiment, a kit provides, in suitable packaging, individual doses of a glycan composition that correspond to dosing points in a treatment regimen, wherein the doses are packaged in one or more packets.

In one embodiment, the pharmaceutical glycan composition can be provided in bulk in a single container, or in two, three, four, five, or more than five containers. For example, each container may contain enough of a pharmaceutical glycan composition for a particular week of a treatment program that runs for a month. If more than one bulk container is provided, the bulk containers can be suitably packaged together to provide sufficient pharmaceutical glycan composition for all or a portion of a treatment period. The container or containers can be labeled with a label indicating information useful to the subject in need thereof or the physician performing the treatment protocol, such as, e.g. dosing schedules.

The pharmaceutical glycan composition can be packaged with other suitable substances, e.g., a second active compound or therapeutic agent or a buffer/carrier. The other substance or substances can be packaged separately from the pharmaceutical glycan composition, or mixed with the pharmaceutical glycan composition, or combinations thereof. Thus, in one embodiment, kits include a dosage form containing all the ingredients intended to be used in a course of treatment or a portion of a course of treatment, e.g., a pharmaceutical glycan composition and optionally a second active compound or therapeutic agent or a buffer/carrier. In one embodiment, a pharmaceutical glycan composition is packaged in one package or set of packages, and additional components, such as beneficial bacteria and therapeutic agents (e.g., drugs) are packaged separately from the pharmaceutical glycan composition.

Kits can further include written materials, such as instructions, expected results, testimonials, explanations, warnings, clinical data, information for health professionals, and the like. In one embodiment, the kits contain a label or other information indicating that the kit is only for use under the direction of a health professional. The container can further include scoops, syringes, bottles, cups, applicators or other measuring or serving devices.

### Administration to a Subject

The glycan preparations, pharmaceutical compositions and therapeutic agents described herein can be administered to a subject in need thereof by various routes. For example, the glycan preparations can be administered locally to the non-gut site. In some embodiments, the glycan preparation is locally administered to the oral cavity, the nasal cavity or the vagina. In one embodiment, the glycan preparation is administered to a mucosal tissue. If desired, a second agent may be administered, e.g. a drug. The drug may be administered locally, e.g., to the non-gut site or systemically (e.g. orally or intravenously or by any other suitable route).

Active compounds and pharmaceutical agents, e.g., beneficial bacteria or drugs, may be administered separately, e.g., prior to, concurrent with or after administration of the glycan preparation and not as a part of the pharmaceutical glycan composition (e.g. as a co-formulation). In some embodiments, pharmaceutical glycan compositions are administered in combination with a recommended or prescribed diet, e.g. a diet that is rich in probiotic, prebiotic, and/or micronutrient-containing foods, such as it may be determined by a physician or other healthcare professional.

Additional Substances can be Given in Conjunction with a Glycan Composition. These Substances can enhance the action or efficacy of glycan preparations. These substances can be given prior to treatment with glycan preparations, during treatment with glycan preparations, after treatment with glycan preparations, or any combination thereof. If administered during glycan preparation treatment, they can be administered with the dose of glycan preparation being given, or before or after the dose of glycan preparation, or any combination thereof.

### Methods of Treating

Provided herein are methods of treating a dysbiosis in a non-gut tissue of a subject. Further provided herein are methods of treating a disease, disorder or pathological condition of a non-gut tissue of a subject. The methods may include modulating the pH of a non-gut tissue of a subject.

The methods may further include modulating the metabolic profile (e.g., of volatile fatty acid) of a non-gut tissue. Further provided are methods of preventing, treating, or reducing or eliminating one or more symptoms of a non-gut site associated disease, disorder or pathological condition.

The methods comprise locally (e.g. directly) administering to the non-gut site (e.g. to the mucosal tissue) a glycan preparation described herein in an amount and for a time effective to: treat the dysbiosis, treat the disease, disorder or pathological condition, modulate the pH, modulate the metabolic profile of the non-gut site of the subject, prevent or treat the disease, disorder or condition, or reduce or eliminate one or more symptoms of the non-gut site associated disease, disorder or condition. In one embodiment, methods are provided to prevent, treat, ameliorate symptoms of, and/or prevent relapse of pathogen colonization at a non-gut site.

In some embodiments, the non-gut site is the oral cavity, the nasal cavity, or the vagina.

In some embodiments, the subject experiences a reduction in at least one symptom following treatment. In some embodiments, a reduction in the severity of a symptom following treatment can be determined (e.g. by measuring a known biomarker) and is in the order of about 3%, 5%, 7%, 10%, 20%, 30%, 40%, 50%, 60%, 70%, 80%, 90%, 95%, or about 100%. In some embodiments, the symptoms, measured as described herein, are decreased by an average of about 10%, 20%, 30%, 40%, 50%, 60%, 70%, 80%, 90%, 95%, or about 100% when compared to symptoms prior to the administration of a pharmaceutical glycan composition. In some embodiments, the reduction in the severity of the symptom persists for at least about a day, two days, three days, four days, five days, a week, two weeks, three weeks, a month, 3 months, 6 months, 9 months, a year, two years, five years, ten years after treatment or the reduction is permanent.

In one embodiment, a symptom remains partially, substantially, or completely eliminated or decreased in severity in a subject for at least about 1 day, 1 week, 1 month, 2 months, 3 months, 4 months, 5 months, 6 months, 9 months, one year, 18 months, two years, three years, four years, five years, ten years, or more than ten years after the termination of treatment. In another embodiment a symptom is permanently eliminated or decreased in severity in a subject after the termination of treatment.

In certain embodiments, the subject is a human subject having one or more symptoms of a dysbiosis at a non-gut site, such as the nasal cavity, the oral cavity and the vagina. Symptoms of dysbioses include overgrowth of an undesired pathogen or an undesired bacterial taxa, reduced representation of key health-associated bacterial taxa, reduced or increased diversity of microbial taxa compared to a healthy individual, and/or reduced overall abundance of beneficial bacterial taxa.

Further provided are methods for the (re-) colonization of the non-gut site with beneficial, commensal taxa. In one embodiment, the relative abundance of the beneficial taxa is increased by administration of a glycan preparation described herein to treat disease, disorder or pathological condition or prevent a relapse of a non-gut site-associated disease, disorder or pathological condition. In one embodiment, the a beneficial taxa is co-administered with a glycan preparation described herein to treat disease, disorder or pathological condition or prevent a relapse of a non-gut site-associated disease, disorder or pathological condition.

In some embodiments, microbial metabolite profiles of patient samples or microbial cultures from subject samples are used to identify risk factors for developing a non-gut disease, disorder or condition. Exemplary metabolites for the purposes of diagnosis, prognostic risk assessment, or treatment assessment purposes include those listed in Table 8. In some embodiments, microbial metabolite profiles are taken at different time points during a subject's disease and treatment in order to better evaluate the subject's disease state including recovery or relapse events. Such monitoring is also important to lower the risk of a subject developing a new disease, disorder or pathological condition, e.g., at the non-gut site. In some embodiments, metabolite profiles inform subsequent treatment.

In some embodiments, the glycan composition may also be combined with an antibiotic that disrupts normal microbial growth at the non-gut site. During a course of antibiotic treatment, the glycan composition may be provided at the initiation of antibiotic treatment; shortly following antibiotic treatment, e.g. 1, 2, 3, 4, 5, 6, 7, or more days following treatment; or may be administered upon diagnosis of undesirable pathogen growth at the non-gut site.

Further, if determined useful by a treating physician or other healthcare provider, the glycan compositions described herein can be administered in combination with various other standard of care therapies. The glycan compositions may be administered prior to, concurrent with, or post treatment with standard of care therapies. In some instances, the therapies disrupt the composition and health of the normal microbiota at the non-gut site, which may lead to the undesirable proliferation of harmful bacteria or pathogens, which may cause one or more of the symptoms described herein. In some embodiments, administration of the glycan compositions described herein is useful for alleviating those symptoms and restoring a normal microbial community at the non-gut site.

Nasal cavity Provided herein are methods to treat a nasal disease, disorder or pathological condition, the method comprising administering (e.g. locally) to the nasal cavity a glycan preparation described herein in an amount (e.g. dose) and for a time (e.g., treatment interval) effective to treat the nasal disease, disorder or pathological condition. In some embodiments, the nasal disease, disorder or pathological condition, is rhinosinusitis (sinus infection), chronic rhinosinusitis (CRS), S. aureus infection or carriage, nasal vestibulitis, nasal furuncles and asthma.

In some embodiments, the glycan compositions described herein is administered in combination with a standard of care therapy. In one embodiment, the therapy is directed to the elimination of nasal S. aureus. In some embodiments, the therapy includes the administration of a topical mupirocin application or oral antibiotics, such as rifampin and doxycycline.

Provided herein are methods to treat a nasal dysbiosis, the method comprising administering (e.g. locally) to the nasal cavity a glycan preparation described herein in an amount (e.g. dose) and for a time (e.g., treatment interval) effective to treat the dysbiosis.

In some embodiments, the dysbiosis includes an increased abundance (e.g., relative to a non-dysbiotic state) of a disease-associated bacterium, pathobiont or pathogen, for example of the genera Corynebacterium, Dolosigranulum, Haemophilus, Moraxella, Peptoniphilus, Propionibacterium, Pseudomonas, Staphylococcus, and Streptococcus.

In some embodiments, the dysbiosis includes an increased abundance (e.g., relative to a non-dysbiotic state) of a disease-associated bacterium, pathobiont or pathogen, for example of the species Corynebacterium accolens, Corynebacterium pseudodiphtericum, Corynebacterium tuberculostearicum, Dolosigranulum pigrum, Haemophilus influenza, Moraxella catarrhalis, Peptoniphilus rhinitidis, Propionibacterium acnes, Pseudomonas aeruginosa, Staphylococcus aureus, and Streptococcus pneumonia.

In some embodiments, the dysbiosis includes a modulated (e.g., increased or decreased) abundance (e.g., relative to a non-dysbiotic state) of one or more of the species Propionibacterium acnes, Corynebacterium accolens, Corynebacterium tuberculostearicum, Corynebacterium pseudodiphtericum, Mycobacterium fallax, Corynebacterium mucifaciens, Staphylococcus epidermidis, Staphylococcus aureus, Dolosigranulum pigrum, Finegoldia magna, and Moraxella catarrhalis, and the genera Peptoniphilus, Anaerococcus, Tomitella.

Dysbiosis can give rise to a disease, disorder or condition, such as, e.g., rhinosinusitis (sinus infection), chronic rhinosinusitis (CRS), S. aureus infection or carriage, nasal vestibulitis, nasal furuncles and asthma.

At the genus level, 457 bacteria have been characterized by 16S rRNA sequencing in the anterior nares (Zhou et al., 2013). At the phylum level, the nasal microbiome is dominated by Actinobacteria, Firmicutes, and Proteobacteria (Bassis et al., 2014); at the genus level, Corynebacterium, Propionibacterium, Staphylococcus, and Moraxella are prevalent members (Zhou et al., 2013).

The nasal cavity serves as a reservoir for species Staphylococcus aureus, and carriage of S. aureus is a significant risk factor for nosocomial S. aureus bacteraemia (Wertheim et al., 2004).

The presence or absence of other bacterial species are also correlated with S. aureus carriage. For example, Corynebacterium accolens species is more common in carriers, whereas C. pseudodiphtheriticum is more common in non-carriers (Yan et al., 2013), and the presence of S. epidermidis is correlated with the absence of S. aureus (Iwase et al., 2010). The nasal microbiome is also thought to play a role in the pathogenesis of chronic rhinosinusitis (CRS). Although the total bacterial burden is similar in CRS patients and healthy controls, CRS patients tend to have less diverse microbiomes and higher prevalence of certain bacteria (e.g., S. aureus) than controls (Wilson and Hamilos, 2014). (Zhou, Y. et al. (2013). Biogeography of the ecosystems of the healthy human body. Genome Biol. 14, R1; Bassis, C. M., et al. (2014). The nasal cavity microbiota of healthy adults. Microbiome 2, 27; Wertheim, H. F. L., et al. (2004). Risk and outcome of nosocomial Staphylococcus aureus bacteraemia in nasal carriers versus non-carriers. Lancet Lond. Engl. 364, 703-705; Yan, M. et al. (2013). Nasal microenvironments and interspecific interactions influence nasal microbiota complexity and S. aureus carriage. Cell Host Microbe 14, 631-640; Iwase, T. et al. (2010). Staphylococcus epidermidis Esp inhibits Staphylococcus aureus biofilm formation and nasal colonization. Nature 465, 346-349; Wilson, M. T., and Hamilos, D. L. (2014). The nasal and sinus microbiome in health and disease. Curr. Allergy Asthma Rep. 14, 485.)

### Oral Cavity

Provided herein are methods to treat an oral disease, disorder or pathological condition, the method comprising administering (e.g. locally) to the oral cavity a glycan preparation described herein in an amount (e.g. dose) and for a time (e.g., treatment interval) effective to treat the oral disease, disorder or pathological condition. In some embodiments, the oral disease, disorder or pathological condition, is dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, tonsiloliths, dentoalveolar abscess, periodontal abscess, Ludwig's angina, viral infection (e.g. herpesvirus, human papilloma virus, etc.), or fungal/yeast infections (e.g. candidiasis).

In some embodiments, the glycan compositions described herein is administered in combination with a standard of care therapy. In one embodiment, the therapy includes, e.g., antibiotic treatment, a physical method to remove plaque, or administration of a beneficial bacterium.

Provided herein are methods to treat an oral dysbiosis, the method comprising administering (e.g. locally) to the oral cavity a glycan preparation described herein in an amount (e.g. dose) and for a time (e.g., treatment interval) effective to treat the dysbiosis.

In some embodiments, the dysbiosis includes an increased abundance (e.g., relative to a non-dysbiotic state) of a disease-associated bacterium, pathobiont or pathogen, for example of the genera Actinomyces, Aggregatibacter, Atopobium, Bacteroides, Bifidobacterium, Campylobacter, Capnocytophaga, Corynebacterium, Dialister, Eubacterium, Fusobacterium, Gemella, Granulicatella, Kingella, Lactobacillus, Leptotrichia, Olsenella, Parascardovia, Peptostreptococcus, Prevotella, Porphyromonas, Propionibacterium, Pseudoramibacter, Selenemonas, Sphingomonas, Streptococcus, Tannerella, Thiomonas, Treponema, and Veillonella.

In some embodiments, the dysbiosis includes an increased abundance (e.g., relative to a non-dysbiotic state) of a disease-associated bacterium, pathobiont or pathogen, for example of the species Streptococcus mutans; Streptococcus sobrinus.

In some embodiments, the dysbiosis includes an increased abundance (e.g., relative to a non-dysbiotic state) of a disease-associated bacterium, pathobiont or pathogen, for example of the species Aggregatibacter actinomycetemcomitans, Porphyromonas gingivalis, Campylobacter rectus, Treponema denticola, Fusobacterium nucleatum, Tannerella forsythia, and Prevotella intermedia.

In some embodiments, the dysbiosis includes an increased abundance (e.g., relative to a non-dysbiotic state) of a disease-associated bacterium, pathobiont or pathogen, for example of the species genera Veillonella, Actinomyces, Granulicatella, Leptotrichia, Thiomonas, Bifidobacterium, Prevotella, Atopobium, Olsenella, Pseudoramibacter, Propionibacterium, and Selenemonas.

In some embodiments, the dysbiosis includes an increased abundance (e.g., relative to a non-dysbiotic state) of a disease-associated bacterium, pathobiont or pathogen, for example of the species Actinomyces gerencseriae, Aggregatibacter actinomycetemcomitans, Atopobium minitum, Atopobium parvulum, Atopobium rimae, Bacteroides forsythus, Campylobacter rectus, Fusobacterium animalis, Fusobacterium nucleatum, Gemella morbillorum, Kingella oralis, Lactobacillus crispatus, Lactobacillus fermentum, Lactobacillus rhamnosus, Peptostreptococcus micros, Peptostreptococcus prevotii, Prevotella intermedia, Porphyromonas gingivalis, Selenomonas sputigena, Selenomonas noxia, Streptococcus anginosus, Streptococcus constellatus, Streptococcus mitis, Streptococcus mutans, Streptococcus oralis, Streptococcus salivarius, Streptococcus sanguinis, Streptococcus sobrinus, Tannerella forsythia, and Treponema denticola.

Dysbiosis can give rise to a disease, disorder or condition, such as, e.g., dental caries (cavities), periodontal disease, gingivitis, periodontitis, periapical periodontitis, halitosis (bad breath), severe early childhood caries (S-ECC), root caries (RC), oral squamous cell carcinoma (OSCC), tonsiloliths, tonsiloliths, dentoalveolar abscess, periodontal abscess, Ludwig's angina, viral infection (e.g. herpesvirus, human papilloma virus, etc.), or fungal/yeast infections (e.g. candidiasis).

The oral cavity contains a diverse but relatively stable community of bacterial species (Zhou et al., 2013). Over 600 bacterial species in the oral cavity have been characterized by 16S rRNA sequencing (Dewhirst et al., 2010). The main phyla represented in the oral microbiome are Actinobacteria, Bacteroidetes, Chlamydiae, Chloroflexi, Euryarchaeota, Firmicutes, Fusobacteria, Proteobacteria, Spirochaetes, SRi, Synergistetes, Tenericutes, and TM7 (Dewhirst et al., 2010). Although there is some overlap in the constituents of each oral site (e.g., teeth vs. cheek), distinct sites have distinct characteristic populations (Zhou et al., 2013). For example, saliva contains ˜1.4×108 CFU/mL of bacteria primarily from phyla Actinobacteria, Bacteroidetes, Firmicutes, Fusobacteria, Proteobacteria, Spirochaetes, and TM7. The oral microbiome has been directly implicated in the pathogenesis of oral diseases such as dental caries (cavities) and periodontal disease, and has also been indirectly implicated in a range of other diseases (reviewed in He et al., 2014). Dental caries are associated with the presence of species Streptococcus mutans; furthermore, other bacteria, including genera Streptococcus, Veillonella, Actinomyces, Granulicatella, Leptotrichia, Thiomonas, Bifidobacterium, and Prevotella are associated with severe early childhood caries (S-ECC), and genera Atopobium, Olsenella, Pseudoramibacter, Propionibacterium, and Selenemonas are associated with root caries (RC) in adults. Characteristic shifts in bacterial communities have also been documented in periapical periodontitis, periodontal diseases such as gingivitis and periodontitis, halitosis (bad breath), and oral squamous cell carcinoma (OSCC).

### Vagina:

Provided herein are methods to treat a vaginal disease, disorder or pathological condition, the method comprising administering (e.g. locally) to the vagina a glycan preparation described herein in an amount (e.g. dose) and for a time (e.g., treatment interval) effective to treat the vaginal disease, disorder or pathological condition. In some embodiments, the vaginal disease, disorder or pathological condition, is bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, risk for a preterm birth or miscarriage.

In some embodiments, the glycan compositions described herein is administered in combination with a standard of care therapy. In one embodiment, the therapy includes, e.g., oral or vaginally-applied antibiotics (including metronidazole, clindamycin, tinidazole, and secnidazole), an antifungal, or a vaginally-applied hormone, including estradiol, e.g. in form of a cream.

In some embodiments, methods for lowering the pH in the vagina of a female subject are provided. The methods include administering to a female subject in need thereof (e.g. a subject exhibiting BV) a glycan preparation in an amount effective to lower the pH in the vagina (e.g. to a point where the pH is representative of a healthy vaginal environment). Treatment progress may be assessed, e.g. using pH and/or lactic acid as a biomarker and/or monitoring/determining the presence of Lactobacillus, e.g. species such as L. crispatus, L. iners, L. gasseri L. acidophilus and L. jensenii, or the presence or absence of pathogenic bacteria or pathobionts.

Provided herein are methods to treat a vaginal dysbiosis, the method comprising administering (e.g. locally) to the vagina a glycan preparation described herein in an amount (e.g. dose) and for a time (e.g., treatment interval) effective to treat the dysbiosis.

In some embodiments, the dysbiosis includes an increased abundance (e.g., relative to a non-dysbiotic state) of a disease-associated bacterium, pathobiont or pathogen, for example of the genera Actinomyces, Aerococcus, Atopobium, Bacteroides, Corynebacterium, Dialister, Eggerthella, Escherichia, Gardnerella, Haemophilus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, and the order Clostridiales (e.g. bacterial vaginosis-associated bacterium-1 (BVAB-1), BVAB-2, and BVAB-3).

In some embodiments, the dysbiosis includes an increased abundance (e.g., relative to a non-dysbiotic state) of a disease-associated bacterium, pathobiont or pathogen, for example of the species Gardnerella vaginalis, Prevotella species, Porphyromonas species, Peptostreptococcus species, Mycoplasma hominis, and Mobiluncus species, Fusobacterium species, Atopobium vaginae, and Enterococcus faecium.

In some embodiments, the dysbiosis includes an increased abundance (e.g., relative to a non-dysbiotic state) of a disease-associated bacterium, pathobiont or pathogen, for example of the species Aerococcus christensenii Atopobium vaginae, Bacteroides urealyticus, Corynebacterium vaginale, Dialister micraerophilus, Escherichia coli, Gardnerella vaginalis, Haemophilus influenza, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

Dysbiosis can give rise to a disease, disorder or condition, such as, e.g., bacterial vaginosis (BV), vaginal discharge, pelvic inflammatory disease, infection with vancomycin-resistant enterococci (VRE), Group B Streptococcus infection, sexually transmitted infectious diseases (including microbial, viral, and parasitic diseases), cervicitis, desquamative inflammatory vaginitis (DIV), vaginal Staphylococcus infection, risk for a preterm birth or miscarriage.

At the genus level, 218 bacteria have been characterized by 16S rRNA sequencing in the mid vagina (Zhou et al., 2013, Biogeography of the ecosystems of the healthy human body. Genome Biol. 14, R1). Vaginal microbiomes are composed of taxa that include genera Actinomyces, Corynebacterium, Bacteroides, Prevotella, Staphylococcus, Lactobacillus, Streptococcus, Anaerococcus, Finegoldia, Peptoniphilus, and Dialister (reviewed in Ma et al., 2012, The vaginal microbiome: rethinking health and diseases. Annu. Rev. Microbiol. 66, 371-389). The vaginal microbiome of most women is dominated by Lactobacillus, in particular, L. crispatus, L. iners, L. gasseri, and L. jensenii. Production of lactic acid by these bacteria lowers the pH of the vagina and is thought to contribute to the protection against pathogens. Production of hydrogen peroxide by these bacteria is also thought to contribute to the protection against pathogens. In contrast, in 20-30% of women (and more commonly in Black and Hispanic women compared with White or Asian women), the vaginal microbiome is not dominated by Lactobacillus but is instead populated with a diverse mixture of anaerobic bacteria from genera including Atopobium, Corynebacterium, Anaerococcus, Peptoniphilus, Prevotella, Gardnerella, Sneathia, Eggerthella, Mobiluncus, and Finegoldia. Bacterial vaginosis (BV) causes symptoms such as vaginal discharge and increases the risk of sexually transmitted disease, pelvic inflammatory disease, and preterm birth. BV arises from dysregulation of the vaginal microbiome.

In some embodiments, the pharmaceutical glycan composition is administered in an amount and for a time effective to result in shifted or modulated state of the subject's non-gut site. In one embodiment, the the pharmaceutical glycan composition is administered in an amount and for a time effective to result in shifted or modulated bacterial taxa (one or more, two or more, three or more, etc.). In one embodiment, the pharmaceutical glycan composition is administered in an amount and for a time effective to result in shifted or modulated microbial function (e.g., a metabolic function). In one embodiment, the pharmaceutical glycan composition is administered in an amount and for a time effective to result in a shift or modulation of the microbiome (genome), transcriptome, metabolome, or proteome of the microbiota.

In some embodiments, administration of the pharmaceutical glycan compositions improves the overall health of the host and/or the health of a specific niche, such as a non-gut site, e.g. by modulating (e.g. increasing or decreasing) the growth or abundance of one or more members of the microbial community (such as resident commensal bacteria and/or acquired pathogens or pathobionts) in the niche, e.g., the nasal cavity, the oral cavity or the vagina.

The glycan preparations described herein when administered to a subject in an effective amount may modulate the production of one or more microbial metabolites in the non-gut site. The glycan preparations when administered to a subject in an effective amount may modulate (e.g., increase or decrease) the production or level of one or more microbial metabolites listed in Table 8. In some embodiments, glycan preparations are administered to modulate short chain fatty acid (SCFA) production of commensal bacteria at the non-gut site.

In some embodiments, glycan preparations are administered to induce systemic effects, e.g. of SCFAs and other microbially produced immunomodulatory molecules or metabolites to modulate the inflammatory state of distal sites.

In some embodiments, methods of selecting a subject for a treatment (e.g., for treatment with a pharmaceutical glycan composition) are provided. The methods include: (a) identifying a subject who has a disease, disorder or pathological condition at a non-gut site (e.g., nasal cavity, oral cavity or vagina), and (b) selecting the identified subject for treatment with a glycan preparation described herein. In some embodiments, the subject is further selected for treatment with a second drug or therapy. In some embodiments, methods of selecting a subject for a treatment include selecting a subject that is treatment naïve (e.g., a subject that is treatment naïve with respect to an antimicrobial therapy).

In some embodiments, methods of selecting a subject for a treatment include selecting the glycan therapeutic preparation on the basis that it will provide therapeutic benefit to the subject. In some embodiments, methods of selecting a subject for a treatment include selecting the subject on the basis that the subject will or is expected to benefit from administration of the glycan preparation.

In some embodiments, the selection methods include assessing the subject's non-gut site microbiota, e.g., before, during and/or after the treatment. In one embodiment, the subject's non-gut site microbiota is assessed before starting treatment. In some embodiments, the results of the assessment are used to select the subject for treatment. Alternatively or in addition, assessment is used to identify a dosage or dosage regimen for the treatment.

In some embodiments, subjects are identified and selected that respond to a glycan preparation for initial and/or continued treatment. Responders may be identified using one or more suitable parameter as determined by a physician or other healthcare provider. The parameters include one or more of: a) a physiological treatment effect (e.g. reduction of a fever, increased well-being, increased energy, etc.), b) a desired change in a (host) biomarker (e.g. an inflammatory marker, etc.), c) a microbial taxa shift (e.g., in relative abundance, change in diversity, etc.), d) a functional shift of the microbiota (e.g. a shift in metabolic output, microbial signaling, microbial gene expression, microbial protein expression), e) absence or presence of a desired bacterial taxa (in the host microbiota), etc. In some embodiments, non-responders are identified and selected.

In one embodiment, treatment methods include rendering the non-responder responsive to the treatment. In some embodiments, this may include administering to the non-responder one or more bacterial taxa (e.g. one or more commensals) that are responsive to glycan (and/or second agent) treatment.

In some embodiments, methods of evaluating a subject, e.g., to evaluate suitability for glycan treatment, responsiveness to glycan treatment, or glycan treatment progression, are provided.

Optionally, the glycan treatment is in combination with another treatment or therapy (e.g., a drug treatment). Changes in a variety of suitable biomarkers may be assessed. In some embodiments, changes in the microbiota are assessed or corresponding values are acquired. In some embodiments, changes in microbial metabolism (e.g. metabolite input and/or output) are assessed or corresponding values are acquired. In some embodiments, changes in the microbiome (e.g. changes on the genome or transcriptome level) are assessed or corresponding values are acquired. In some embodiments, changes in the microbial proteome are assessed or corresponding values are acquired. In some embodiments, changes in the host are assessed or corresponding values are acquired. In some embodiments, changes in the host proteome (e.g. protein synthesis), metabolome, transcriptome (e.g. gene transcription/expression), cell signaling, etc. are assessed or corresponding values are acquired. In some embodiments, the methods include a) acquiring a value for a parameter related to the level of a biomarker modulated by a glycan preparation (and/or the drug or therapy in a combination treatment); b) responsive to the value, classifying the subject, selecting a treatment for the subject, or administering the treatment to the subject, thereby evaluating a subject.

Treatment responsiveness and/or progression may be assessed or evaluated using one or more biomarker. Suitable biomarkers may be determined by a physician. The glycan treatment may result in increases or decreases of one or more biomarkers that can be determined by methods known in the art. An investigator can determine at which point or points during treatment the biomarker(s) should be measured, e.g. prior to treatment, at various intervals during treatment and/or after treatment. Any suitable sample, e.g. a non-gut site sample such as, e.g. a (mucosal) tissue sample or biopsy, a swab, etc. may be drawn from the subject and the sample may be analyzed by suitable methods known in the art. In some embodiments, a substantial increase or decrease in a biomarker may be detected to assess treatment progression.

In some embodiments, treatment with the glycan preparation results in the release of short-chain fatty acids such as butyrate, acetate, and propionate and other metabolites (e.g. bile acids, and lactate) by the microbiota.

### Identification of Bacterial Constituents

In some embodiments, the pharmaceutical glycan compositions described herein are administered to a subject to increase the growth of beneficial bacteria and/or to decrease the growth of pathogens in the non-gut site. In some embodiments, the microbial community is shifted by the glycan preparation toward that of a healthy state. The microbial changes occurring in the non-gut site can be analyzed using any number of methods known in the art and described herein.

As one quantitative method for determining whether a glycan preparation results in a shift of the community of bacteria in the non-gut site, quantitative PCR (qPCR) can be performed. Genomic DNA can be extracted from samples using commercially-available kits, such as the Mo Bio Powersoil®-htp 96 Well Soil DNA Isolation Kit (Mo Bio Laboratories, Carlsbad, Calif.), the Mo Bio Powersoil® DNA Isolation Kit (Mo Bio Laboratories, Carlsbad, Calif.), or the QIAamp DNA Stool Mini Kit (QIAGEN, Valencia, Calif.) according to the manufacturer's instructions, or by other standard methods known to those skilled in the art.

In some embodiments, qPCR can be conducted using HotMasterMix (5PRIME, Gaithersburg, Md.) and primers specific for certain (e.g. beneficial or desired) bacteria and may be conducted on a MicroAmp® Fast Optical 96-well Reaction Plate with Barcode (0.1 mL) (Life Technologies, Grand Island, N.Y.) and performed on a BioRad C1000™ Thermal Cycler equipped with a CFX96™ Real-Time System (BioRad, Hercules, Calif.), with fluorescent readings of the FAM and ROX channels. The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples. The skilled artisan may employ alternative qPCR modes.

In some embodiments, the microbial constituents are identified by characterizing the DNA sequence of microbial 16S small subunit ribosomal RNA gene (16S rRNA gene). 16S rRNA gene is approximately 1,500 nucleotides in length, and in general is highly conserved across organisms, but contain specific variable and hypervariable regions (V1-V9) that harbor sufficient nucleotide diversity to differentiate species- and strain-level taxa of most organisms. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (See, e.g., Brosius et al., Complete nucleotide sequence of a 16S ribosomal RNA gene from Escherichia coli, PNAS 75(10):4801-4805 (1978)).

Composition of a microbial community can be deduced by sequencing full 16S rRNA gene, or at least one of the VI, V2, V3, V4, V5, V6, V7, V8, and V9 regions of this gene or by sequencing of any combination of variable regions from this gene (e.g. V1-3 or V3-5). In one embodiment, the VI, V2, and V3 regions are used to characterize a microbiota. In another embodiment, the V3, V4, and V5 regions are used to characterize a microbiota. In another embodiment, the V4 region is used to characterize a microbiota.

Sequences that are at least 97% identical to each other are grouped into Operational Taxonomic Units (OTUs). OTUs that contain sequences with 97% similarity correspond to approximately species level taxa. At least one representative sequence from each OTU is chosen, and is used to obtain a taxonomic assignment for an OTU by comparison to a reference database of highly curated 16S rRNA gene sequences (such as Greengenes or SILVA databases). Relationship between OTUs in a microbial community could be deduces by constructing a phylogenetic tree from representative sequences from each OTU.

Using known techniques, in order to determine the full 16S sequence or the sequence of any variable region of the 16S sequence, genomic DNA is extracted from a bacterial sample, the 16S rRNA (full region or specific variable regions) amplified using polymerase chain reaction (PCR), the PCR products are cleaned, and nucleotide sequences delineated to determine the genetic composition of 16S rRNA gene or a variable region of the gene. If full 16S sequencing is performed, the sequencing method used may be, but is not limited to, Sanger sequencing. If one or more variable regions is used, such as the V4 region, the sequencing can be, but is not limited to being performed using the Sanger method or using a next-generation sequencing method, such as an Illumina method. Primers designed to anneal to conserved regions of 16S rRNA genes (e.g., the 515F and 805R primers for amplification of the V4 region) could contain unique barcode sequences to allow characterizing multiple microbial communities simultaneously.

As another method to identify microbial composition is characterization of nucleotide markers or genes, in particular highly conserved genes (e.g., “house-keeping” genes), or a combination thereof, or whole genome shotgun sequence (WGS). Using defined methods, DNA extracted from a bacterial sample will have specific genomic regions amplified using PCR and sequenced to determine the nucleotide sequence of the amplified products. In the WGS method, extracted DNA will be fragmented into pieces of various lengths (from 300 to about 40,000 nucleotides) and directly sequenced without amplification. Sequence data can be generated using any sequencing technology including, but not limited to Sanger, Illumina, 454 Life Sciences, Ion Torrent, ABI, Pacific Biosciences, and/or Oxford Nanopore.

In addition to the 16S rRNA gene, a selected set of genes that are known to be marker genes for a given species or taxonomic group is analyzed to assess the composition of a microbial community. These genes are alternatively assayed using a PCR-based screening strategy. For example, various strains of pathogenic Escherichia coli are distinguished using genes that encode heat-labile (LTI, LTIIa, and LTIIb) and heat-stable (STI and STII) toxins, verotoxin types 1, 2, and 2e (VT1, VT2, and VT2e, respectively), cytotoxic necrotizing factors (CNF1 and CNF2), attaching and effacing mechanisms (eaeA), enteroaggregative mechanisms (Eagg), and enteroinvasive mechanisms (Einv). The optimal genes to utilize to determine the taxonomic composition of a microbial community by use of marker genes are familiar to one with ordinary skill in the art of sequence based taxonomic identification.

Sequencing libraries for microbial whole-genome sequencing (WGS) may be prepared from bacterial genomic DNA. For genomic DNA that has been isolated from a human or laboratory animal sample, the DNA may optionally enriched for bacterial DNA using commercially available kits, for example, the NEBNext Microbiome DNA Enrichment Kit (New England Biolabs, Ipswich, Mass.) or other enrichment kit. Sequencing libraries may be prepared from the genomic DNA using commercially available kits as well, such as the Nextera Mate-Pair Sample Preparation Kit, TruSeq DNA PCR-Free or TruSeq Nano DNA, or the Nextera XT Sample Preparation Kit (Illumina, San Diego, Calif.) according to the manufacturer's instructions.

Alternatively, libraries can be prepared using other kits compatible with the Illumina sequencing platform, such as the NEBNext DNA Library Construction Kit (New England Biolabs, Ipswich, Mass.). Libraries may then be sequenced using standard sequencing technology including, but not limited to, a MiSeq, HiSeq or NextSeq sequencer (Illumina, San Diego, Calif.).

Alternatively, a whole-genome shotgun fragment library prepared using standard methods in the art. For example, the shotgun fragment library could be constructed using the GS FLX Titanium Rapid Library Preparation Kit (454 Life Sciences, Branford, Conn.), amplified using a GS FLX Titanium emPCR Kit (454 Life Sciences, Branford, Conn.), and sequenced following standard 454 pyrosequencing protocols on a 454 sequencer (454 Life Sciences, Branford, Conn.).

Bacterial RNA may be isolated from microbial cultures or samples that contain bacteria by commercially available kits, such as the RiboPure Bacterial RNA Purification Kit (Life Technologies, Carlsbad, Calif.). Another method for isolation of bacterial RNA may involve enrichment of mRNA in purified samples of bacterial RNA through remove of tRNA.

Alternatively, RNA may be converted to cDNA, which used to generate sequencing libraries using standard methods such as the Nextera XT Sample Preparation Kit (Illumina, San Diego, Calif.).

Nucleic acid sequences are analyzed to define taxonomic assignments using sequence similarity and phylogenetic placement methods or a combination of the two strategies. A similar approach is used to annotate protein names, protein function, transcription factor names, and any other classification schema for nucleic acid sequences. Sequence similarity based methods include BLAST, BLASTx, tBLASTn, tBLASTx, RDP-classifier, DNAclust, RapSearch2, DIAMOND, USEARCH, and various implementations of these algorithms such as QIIME or Mothur. These methods map a sequence read to a reference database and select the best match. Common databases include KEGG, MetaCyc, NCBI non-redundant database, Greengenes, RDP, and Silva for taxonomic assignments. For functional assignments, reads are mapped to various functional databases such as COG, KEGG, BioCyc, MetaCyc, and the Carbohydrate-Active Enzymes (CAZy) database. Microbial clades are assigned using software including MetaPhlAn.

### Proteomic Analysis of Microbial Populations

Glycan preparations may be selected based on their ability to increase the expression of microbial proteins associated with healthy states or to decrease the expression of microbial proteins associated with diseased states. Proteomic analysis of microbial populations can be performed following protocols known to one skilled in the art (e.g., Cordwell, Exploring and exploiting bacterial proteomes, Methods in Molecular Biology, 2004, 266:115). To identify differentially expressed proteins (for example, to identify changes in protein expression upon treatment of microbial populations with glycan therapeutics), proteomic analysis can be performed as described, e.g., in Juste et al. (Bacterial protein signals are associated with Crohn's disease, Gut, 2014, 63:1566). For example, the protein is isolated from the microbial lysates of two samples (for example, an untreated microbial population and a population that has been treated with glycan therapeutics). Each protein sample is labeled (e.g., with a fluorescent dye, e.g., Cy3 or Cy5 CyDye DIGE Fluor minimal dye, GE Healthcare) and analyzed by two-dimensional differential gel electrophoresis (2D-DIGE). Gels are stained and protein spots identified as being significantly different between the two samples are excised, digested, and analyzed by liquid chromatography-tandem mass spectrometry (LC-MS/MS). X!TandemPipeline (http://pappso.inra.fr/bioinfo/xtandempipeline/) can be used to identify differentially expressed proteins.

Glycan preparations may also be selected for administration to a human subject based on their effect on the presence of microbial fermentation products. For example, glycan preparations can be selected for their ability to induce or promote growth of bacteria that produce short chain fatty acids such as propionate (propionic acid), acetate, and/or butyrate (butyric acid). Similarly, glycan preparations can be selected for their ability to induce or promote growth of bacteria that produce lactic acid, which can modulate the growth of other bacteria by producing an acidic environment. Such analysis may also be used to pair probiotic bacteria with glycan preparations such that the glycan preparation is a substrate for the production of the desired fermentation products.

The metabolites that are present in fresh or spent culture media or in biological samples collected from humans may be determined using methods described herein. Unbiased methods that may be used to determine the relative concentration of metabolites in a sample and are known to one skilled in the art, such as gas or liquid chromatography combined with mass spectrometry or 1H-NMR. These measurements may be validated by running metabolite standards through the same analytical systems.

In the case of gas chromatography-mass spectrometry (GC-MS) or liquid-chromatography-mass spectrometry (LC-MS) analysis, polar metabolites and fatty acids could be extracted using monophasic or biphasic systems of organic solvents and an aqueous sample and derivatized (Fendt et al., Reductive glutamine metabolism is a function of the α-ketoglutarate to citrate ratio in cells, Nat Commun, 2013, 4:2236; Fendt et al., Metformin decreases glucose oxidation and increases the dependency of prostate cancer cells on reductive glutamine metabolism, Cancer Res, 2013, 73:4429; Metallo et al., Reductive glutamine metabolism by IDH1 mediates lipogenesis under hypoxia, Nature, 2011, 481:380). An exemplary protocol for derivatization of polar metabolites involves formation of methoxime-tBDMS derivatives through incubation of the metabolites with 2% methoxylamine hydrochloride in pyridine followed by addition of N-tert-butyldimethylsilyl-N-methyltrifluoroacetamide (MTBSTFA) with 1% tert-butyldimethylchlorosilane (t-BDMCS). Non-polar fractions, including triacylglycerides and phospholipids, may be saponified to free fatty acids and esterified to form fatty acid methyl esters, for example, either by incubation with 2% H₂SO₄ in methanol or by using Methyl-8 reagent (Thermo Scientific). Derivatized samples may then be analyzed by GC-MS using standard LC-MS methods, for example, a DB-35MS column (30 m×0.25 mm i.d.×0.25 μm, Agilent J&W Scientific) installed on a gas chromatograph (GC) interfaced with an mass spectrometer (MS). Mass isotopomer distributions may be determined by integrating metabolite ion fragments and corrected for natural abundance using standard algorithms, such as those adapted from Fernandez et al. (Fernandez et al., Correction of 13C mass isotopomer distributions for natural stable isotope abundance, J Mass Spectrom, 1996, 31:255). In the case of liquid chromatography-mass spectrometry (LC-MS), polar metabolites may be analyzed using a standard benchtop LC-MS/MS equipped with a column, such as a SeQuant ZIC-pHILIC Polymeric column (2.1×150 mm; EMD Millipore). Exemplary mobile phases used for separation could include buffers and organic solvents adjusted to a specific pH value.

In combination or in the alternative, extracted samples may be analyzed by ¹H-nuclear magnetic resonance (H-NMR). Samples may be combined with isotopically enriched solvents such as D2O, optionally in the presence of a buffered solution (e.g., Na₂HPO₄, NaH₂PO₄ in D₂O, pH 7.4). Samples may also be supplemented with a reference standard for calibration and chemical shift determination (e.g., 5 mM 2,2-dimethyl-2-silapentane-5-sulfonate sodium salt (DSS-d₆, Isotec, USA)). Prior to analysis, the solution may be filtered or centrifuged to remove any sediment or precipitates, and then transferred to a suitable NMR tube or vessel for analysis (e.g., a 5 mm NMR tube). ¹H-NMR spectra may be acquired on a standard NMR spectrometer, such as an Avance II+500 Bruker spectrometer (500 MHz) (Bruker, DE), equipped with a 5 mm QXI-Z C/N/P probe-head) and analyzed with spectra integration software (such as Chenomx NMR Suite 7.1; Chenomx Inc., Edmonton, AB). (Duarte et al., IH-NMR protocol for exometabolome analysis of cultured mammalian cells, Methods Mol Biol, 2014:237-47). Alternatively, ¹H-NMR may be performed following other published protocols known in the art (Chassaing et al., Lack of soluble fiber drives diet-induced adiposity in mice, Am J Physiol Gastrointest Liver Physiol, 2015; Bai et al., Comparison of Storage Conditions for Human Vaginal Microbiome Studies, PLoS ONE, 2012:e36934).

Collection of Microbial Samples from Human Mucosa-Containing Sites and Titer Determination

For example, to collect vaginal microbial samples for nucleic acid extraction and analysis, a sterile Catch-All Sample Collection Swab (Epicentre Biotechnologies) is placed at the vaginal introitus posterior to the hymenal ring/tissue and rotated five times. The swab is then immediately swirled in 750 μL of MoBio buffer in a specimen collection tube and pressed against the wall of the tube multiple times for 20 seconds. A Pederson speculum is introduced to the vaginal cavity to enable sampling of the posterior fornix and vaginal midpoint in a similar manner, using separate collection swabs for each site. The samples are kept on ice until processing. (McInnes & Cutting, Manual of Procedures for Human Microbiome Project: Core Microbiome Sampling Protocol A, v12.0, 2010, http://hmpdacc.org/doc/HMP_MOP_Version12_0_072910.pdf) (Aagaard et al., A Metagenomic Approach to Characterization of the Vaginal Microbiome Signature in Pregnancy, 2012, PLoS ONE, 7: e36466). To collect vaginal microbial samples for culture, the APTIMA Vaginal Swab Specimen Collection Kit (Hologic) is used according to the manufacturer's instructions. Sampling is performed in a similar manner to the protocol described above, but after sampling the swab is collected into a transport tube containing 2.9 mL of transport medium. The pH at the vaginal introitus and posterior fornix is determined at the time of sampling using a microelectrode pH meter (Waterproof BigDisplay pH Spear, Oakton pH meter).

For example, to prepare for the collection of samples from the oral cavity, subjects are asked to rinse their mouth with water for 1 min. Five minutes after the oral rinse, subjects are asked to spit into a sterile 50 mL conical tube (Falcon) until 2-5 mL of saliva are collected (Henson & Wong, Collection, storage, and processing of saliva samples for downstream molecular applications, 2010, Methods Mol Biol, 666:21-30). Saliva samples are prepared for downstream analysis of nucleic acids by centrifuging the Falcon tubes containing the saliva at 2600xg for 15 minutes to sediment solids and then transferring the supernatant to a new 2 mL tube containing MoBio buffer (McInnes & Cutting). Soft tissue sites in the oral cavity—including the tongue, hard palate, buccal mucosa, keratinized (attached) gingiva, palatine tonsils, and throat—are sampled by swabbing the site for 5-10 seconds with a Catch-All Sample Collection Swab (Epicentre Biotechnologies). Hard tissue sites in the oral cavity—including the supragingival and subgingival plaques from multiple teeth—are sampled by gently scraping plaque the site with a sterile Gracey curette. If samples are collected for downstream analysis of nucleic acids, swabs and plaque are collected into MoBio buffer and stored on ice until processing. If samples are collected for culturing, swabs and plaque are collected into transport media, as described in Hoover & Newbrun (Survival of Bacteria from Human Dental Plaque Under Various Transport Conditions, 1977, J Clin Microbiol, 6:212-218).

For example, to collect microbial samples from the nasal cavity for nucleic acid extraction and analysis, a sterile Catch-All Sample Collection Swab (Epicentre Biotechnologies) is used to gently rub the mucosal surfaces of the anterior nares. Both the left and right nares are sampled and pooled together. The swab is then immediately swirled in 750 μL of MoBio buffer in a specimen collection tube and pressed against the wall of the tube multiple times for 20 seconds. The samples are kept on ice until processing. (McInnes & Cutting). To collect microbial samples from the nasal cavity for culture, the BD CultureSwab Specimen Collection and Transport System (Becton, Dickinson and Company) is used according to the manufacturer's instructions. Sampling is performed in a similar manner to the protocol described above, but after sampling the swab is collected into Amies media (included as part of the BD CultureSwab Specimen Collection and Transport System).

In one example, to determine the titer of common vaginal bacteria, including Lactobacillus and Gardnerella, samples containing vaginal bacteria are collected and prepared as a suspension in 5 mL of sterile phosphate buffered saline (PBS). Ten-fold serial dilutions are prepared in sterile PBS and plated (100 μL per dilution) to Lactobacilli MRS Agar (Anaerobe Systems) or Gardnerella Selective Agar with 5% Human Blood (BD). Plates are incubated at 37° C. under anaerobic conditions. After 48 hours, colonies are counted and used to back-calculate the concentration of viable cells in the original sample.

In another example, to determine the titer of common bacteria in the oral cavity, samples containing bacteria from the oral cavity are collected and prepared as a suspension in 5 mL of sterile phosphate buffered saline (PBS). Ten-fold serial dilutions are prepared in sterile PBS and plated (100 μL per dilution) to Tryptic Soy Serum Bacitracin Vancomycin Agar (Anaerobe Systems; to titer Aggregatibacter actinomycetemcomitans, which is associated with periodontitis), Mitis Salivarius Agar with Tellurite (Anaerobe Systems; to titer Streptococci and Enterococci), or Fusobacterium Selective Agar (Anaerobe Systems; to titer Fusobacterium, which is associated with periodontitis). For overall titers of Gram positive bacteria, dilutions are plated to Mannitol Salt Agar (BD). For overall titers of Gram negative bacteria, dilutions are plated to Eosin Methylene Blue Agar (BD) or MacConkey Agar (BD). Plates are incubated at 37° C. under aerobic or anaerobic conditions as appropriate for the target species. After 48 hours, colonies are counted and used to back-calculate the concentration of viable cells in the original sample.

In another example, to determine the titer of common bacteria in the nasal cavity, samples containing bacteria from the nasal cavity are collected and prepared as a suspension in 5 mL of sterile phosphate buffered saline (PBS). Ten-fold serial dilutions are prepared in sterile PBS and plated (100 μL per dilution) to Crystal Violet-Nalidixic Acid-Gentamicin Agar (to titer Streptococcus pnueumoniae), Mannitol Salt Agar (BD; to titer Staphylococcus species), or Chocolate Agar (Anaerobe Systems; to titer Haemophilus and Neisseria species). Alternatively, dilutions are plated to Brain Heart Infusion Agar (Anaerobe Systems) or Luria-Bertani Agar (BD) to non-selectively grow nasal bacteria including Corynebacterium, Staphylococcus, and Propionibacterium. Plates are incubated at 37° C. under aerobic or anaerobic conditions as appropriate for the target species. After 48 hours, colonies are counted and used to back-calculate the concentration of viable cells in the original sample.

To non-selectively culture samples containing bacteria collected from a human or animal, a rich media or agar such as Brucella Blood Agar (Anaerobe Systems), Brain Heart Infusion Agar (Anaerobe Systems), or Chopped Meat Broth (Anaerobe Systems) is used. A minimal media formulation such as M9 (Life Technologies) supplemented with amino acids, carbon sources, or other nutrients as needed is used to non-selectively culture bacteria during in vitro assays testing the effects of glycan preparations or other compounds on bacterial populations. Alternatively, other minimal media formulations known to one skilled in the art are used, for example, as reported in Martens et al. (Mucosal Glycan Foraging Enhances Fitness and Transmission of a Saccharolytic Human Gut Bacterial Symbiont, 2008, Cell Host & Microbe, 4:447-457).

All publications, patents, and patent applications cited or referenced in this specification are herein incorporated by reference to the same extent as if each independent publication or patent publication was specifically and individually indicated to be incorporated by reference.

### EXAMPLES

The invention is further illustrated by the following examples. The examples are provided for illustrative purposes only, and are not to be construed as limiting the scope or content of the invention in any way. The practice of the present invention will employ, unless otherwise indicated, conventional methods of protein chemistry, biochemistry, recombinant DNA techniques and pharmnacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1. Preparation of Glycans

To a round bottom flask equipped with an overhead stirrer and a jacketed short-path condenser was added one or more mono- or disaccharides along with 3-20% by dry weight of one or more of the catalysts described in U.S. Pat. No. 8,466,242 and WO 2014/031956, which are incorporated herein by reference in their entirety. Portions of exemplary catalysts are depicted in FIGS. 1A-1B. Water (0.25 equiv by weight) was added to the dry mixture and the slurry was combined at approximately 100 rpm using a paddle sized to match the contours of the selected round bottom flask as closely as possible. The mixture was then heated to 80-155° C., typically between 135-155° C. Once the solids achieved a molten state, the vessel was placed under 10-1000 mbar vacuum pressure, typically between 300-600 mbar. The reaction was stirred for 30 minutes to 8 hours, typically for 1.5-4 hours, constantly removing water from the reaction. Reaction progress was monitored by HPLC. When sufficient polymerization had occurred, the stirrer was shut off, the reaction was cooled to room temperature and vented to atmospheric pressure, and the solid mass was dissolved in a volume of water sufficient to create a solution of approximately 50 Brix (grams sugar per 100 g solution). Once dissolution was complete, solid catalyst was removed by filtration and the solution containing glycans was concentrated to approximately 65-75 Brix by rotary evaporation.

About 35 distinct glycan preparations were made, many in several batches (e.g., between 2 and 10 batches), including the following 15 glycan preparations that were made in multiple batches and tested in various assays described herein: Single glycan unit (homo-glycan preparations): xyl100, ara100, gal100, glu100, and man100.

Two glycan units (hetero-glycan preparations): xyl75ara25, glu80man20, glu60man40, man60glu40, man80glu20, man80gal20, man66gal33, and glu50gal50.

Three glycan units (hetero-glycan preparations): glu33ga133fuc33 and man52glu29gal19. Additional glycan preparations and the making thereof are described, e.g., in WO/2016/122889 GLYCAN THERAPEUTICS AND RELATED METHODS THEREOF, Example 1, incorporated herein, and include: a) homo-glycan preparations: rha100, fuc100, and fru100, b) hetero-glycan preparations: ara50gal50, ara80xyl20, ara60xyl40, ara50xyl50, ga175xyl25, man62glu38, the hybrid glycans glu90sor10 and glu90glyl0, and c) hetero-glycan preparations: xyl75glu12gal12, xyl33glu33ga133.

Glycans are described by a three-letter code representing the monomeric sugar component followed by a number out of one hundred reflecting the percentage of the material that monomer constitutes. Thus, ‘glu100’ is ascribed to a glycan generated from a100% D-glucose (glycan unit) input and ‘glu50gal50’ is ascribed to a glycan generated from 50% D-glucose and 50% D-galactose (glycan units) input or, alternatively from a lactose dimer (glycan unit) input. As used herein: xyl=D-xylose; ara=D-arabinose or L-arabinose; gal=D-galactose; glu=D-glucose; rha=L-rhamnose; fuc=L-fucose; man=D-mannose; sor=D-sorbitol; gly=D-glycerol.

### Example 2. Purification of Glycans

Glycans (e.g. oligo- and polysaccharides) synthesized as in Example 1 were dissolved in deionized water to a final concentration of 25-50 Brix. The material was then exposed to at least 2 mass equivalents of Dowex Monosphere 88 ion exchange resin by elution through a wet slurry packed column as long as the residence time is sufficient for the solution to achieve a final pH between 3 and 5, typically at 2-3 bed volumes per hour. The process was repeated with Dowex Monosphere 77 ion exchange resin in an analogous fashion until the solution pH was above 5.5. Finally the solution was exposed to Dowex Optipore SD-2 Adsorbent decolorizing resin until the solution was sufficiently clarified and filtered through a 0.2 micron filter to remove residual resin and resin fines. The final solution for all 35 glycan preparations made was then concentrated to 50-85 Brix by rotary evaporation or to a solid by lyophilization.

### Example 3: Modification of Glycans by Removal of Low Molecular Weight Components

Glycans prepared and purified as in Examples 1 and 2 were optionally modified so as to remove low molecular weight components. The separation was achieved by osmotic separation. Approximately 45 cm of 1.0 kD MWCO Biotech CE dialysis tubing (31 mm flat width) from Spectrum Labs was placed into deionized water and soaked for 10 minutes, then one end was sealed with a dialysis tubing clip. A 25 Brix solution of 8 grams dry glycan preparation was sterile filtered and sealed into the tube with a second clip along with a few mL of air to permit the tube to float. The filled tube was then placed in a 3 gallon tank of deionized water which was stirred with sufficient force to induce slow swirling of the sealed tubes. After 8 hours, the water in the tank was replaced and the tube was allowed to stir for an additional 16 hours. Once the dialysis was complete and the material had a DP2+ yield between 80% and 95% and a DP3+ yield between 75% and 90%, with the endpoint as desired, the dilute solution was sterile filtered and concentrated in vacuo to a final concentration of approximately 65 Brix or lyophilized to a solid with a residual moisture between 1 and 10%. Alternatively, the separation was achieved by tangential flow filtration (TFF). In this case, 100 mL of 25 Brix glycan preparation dissolved in deionized water and sterile filtered was placed into the feed bottle of a Spectrum Labs KrosFlo Research IIi TFF system that was prepared according to the manufacturer's recommendation. The glycan preparation was then diafiltered through a 1 kD mPES MidiKros hollow-fiber filter at a transmembrane pressure of 25 psig. HPLC samples of the feed stock taken every 0.5 diafiltration volumes were used to determine when the material had a DP2+ yield between 80% and 95% and a DP3+ yield between 75% and 90%, with the endpoint as desired, at which point the solution was sterile filtered and concentrated in vacuo to a 65 Brix syrup or lyophilized to a solid with residual water content of 1-10% by mass. Low molecular weight components (such as monomers or dimers or other low molecular oligomers, e.g. trimers and tetramers) can also be removed by precipitation with 70% ethanol, as described in Gras, et al. Food Chem. 2001, 128, 773-777. Glycans can also be fractionated into pools with different average molecular weights by activated charcoal chromatography as in Sanz, et al. Chromatographia 2006, 64, 233-236.

### Example 4: Methods for Analyzing Preparations of Glycans

### Measurement of Glycan Content by Liquid Refractometry

The amount of glycan in any given aqueous solution was determined for all glycan preparations that were made. A Mettler-Toledo Refracto 30GS portable sugar refractometer was calibrated using high-purity reverse-osmosis deionized water. Several drops of the glycan solution were filtered through a 0.2 micron syringe filter directly onto the lens of the refractometer. The measurement was taken at room temperature and reported as Brix. The glycan preparations were routinely concentrated to between 60 and 75 Brix without obvious solidification or crystallization at 23° C. Brix can then be converted to solubility assuming a specific density of water equal to 1.0 g/mL. Thus, 75 Brix (100 grams of solution consisting of 75 grams of glycan and 25 grams of water) equals an aqueous solubility of 3.0 g/mL. As a comparison, the aqueous solubility of D-glucose is reported to be 0.909 g/mL (48 Brix) at 25° C. by Sigma-Aldrich.

### Molecular Weight Distribution by Size-Exclusion Chromatography (SEC)

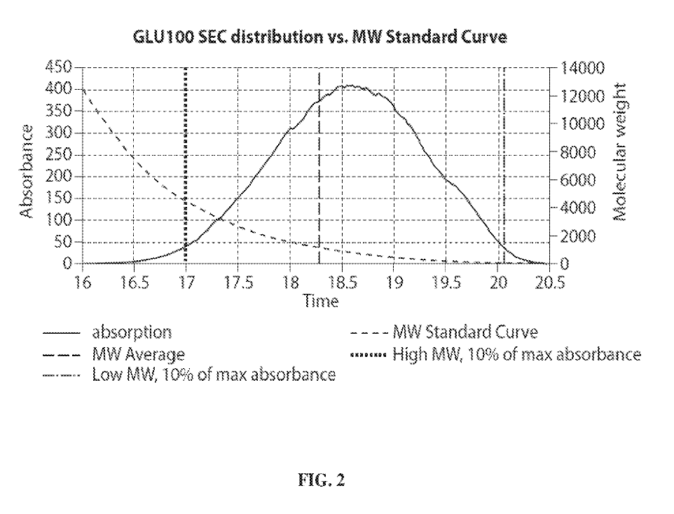

The distribution of molecular weights within a given glycan preparation was quantified. The measurement was made by HPLC using the method described in Monograph of United States Pharmacopeia, 38(6) In-Process Revision: Heparin Sodium (USP37-NF32). Separations were achieved on an Agilent 1200 HPLC system via a GE superpose 12 column using 50 mM ammonium acetate as an eluent at 1.0 mL/min flow rate and an ELSD detector. The column temperature was set at 30° C. and dextran (1 kD, 5 kD, 10 kD weight) were used to draw a standard curve. A 2 mg/ml solution of the sample glycan preparation was prepared and passed through a 0.45 μm spin filter, followed by 40 l injections into the HPLC. A third-order polynomial curve was constructed based on the logarithmic molecular weights and elution volumes of the listed standards. The weight-average molecular weight (Mw), the number average molecular weight (Mn), and the polydispersity index (PDI) for the sample were calculated by comparison to the standard curve. FIG. 2 shows an exemplary curve generated during the SEC evaluation of a glu100 sample in which the average molecular weight was determined to be 1212 g/mol or approximately DP7. The upper end of molecular weight of the material as defined by the point of the curve at 10% of maximum absorption leading the curve was determined to be 4559 g/mol or approximately DP28. The condensation reaction under the continuous withdrawal of water (Example 1) generally produces glycan preparations with an upper end of molecular weight of the material typically at about DP30. The lower end of molecular weight of the material as defined by 10% of the maximum absorption trailing the curve was determined to be 200 g/mol or approximately DP1. The data for 15 exemplary glycan preparations are shown in Table 1. The polymerization (or condensation) process can be controlled to produce glycan preparations with average DPs ranging from small, e.g., DP2.4 (low Mw man100) to large, e.g., DP18.86 (high Mw gal100).

### Molecular Weight Distribution by Ion-Affinity Chromatography (IAC)

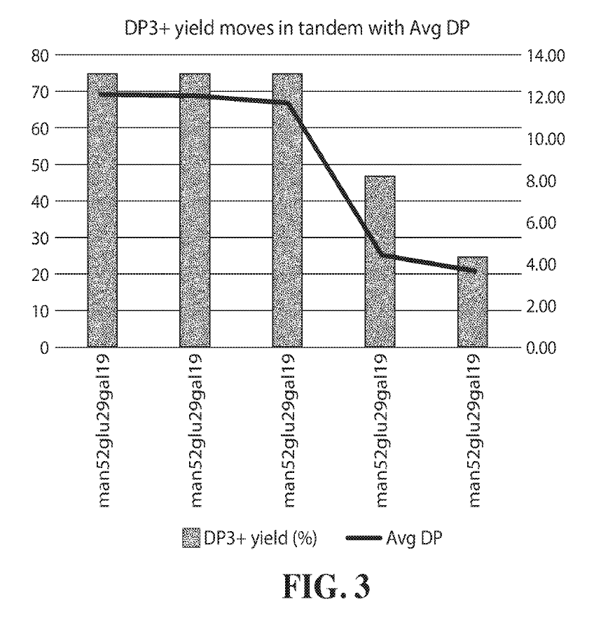

The proportion of glycans with DP greater than or equal to 2 (DP2+) and 3 (DP3+) was determined by ion-affinity chromatography. A sample glycan preparation was diluted out to 50-100 mg/mL and 10 μL of this solution was injected onto an Agilent 1260 BioPure HPLC equipped with a 7.8×300 mm BioRad Aminex HPX-42A column and RI detector. Using pure HPLC-grade water as an eluent, the sample glycan preparation was eluted at 0.6 mL/min through an 80° C. column and an RI detector maintained at 50° C. The peaks representing DP1-6 are assigned by comparison to reference standards and integrated using the Agilent ChemStation software. Peaks are typically integrated as DP1, DP2, DP3, DP4-7, and DP8+. The DP3+ yield expressed as a percentage was used to monitor the progress of the reaction. FIG. 3 shows that the DP3+ yield moves in tandem with average DP, such as shown for 5 different preparations of man52glu29gal19. An increase in Avg DP suggests that smaller glycans such as those of DP2 and DP3 are being polymerized into larger glycans with higher DP measurements. The five batches of the same man52glu29gal19 glycan preparation also demonstrate consistency of batches (columns 1-3) as well as control of Avg DP and DP3+ yield across a range of values (columns 3-5). The data shown in Table 1 for 15 exemplary glycan preparations can be achieved using the controlled process described herein that produces glycan preparations as desired, with DP3+ from, e.g., 25% (low Mw man52glu29gal19) to, e.g., 87% (man80glu20) and DP2+ from, e.g., 54 (low Mw man52glu29gal19) to, e.g., 93% (man80glu20).

### Alpha-/Beta-Distribution by 2D NMR

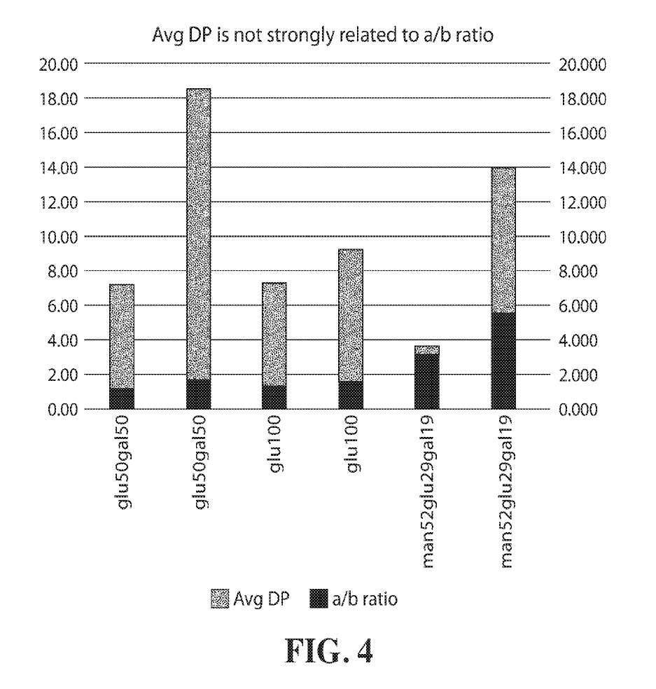

The ratio of alpha- and beta-glycosidic bonds within a given glycan preparation was determined by two-dimensional NMR. Approximately 150 mg of 65 Brix glycan solution was dried to stable mass in a vacuum oven at 45-95° C. under 400 mbar pressure. The sample glycan preparation was subjected to two cycles of dissolution in D₂O and drying to remove residual H₂O. Once dried, the sample glycan preparation was dissolved in 750 μL D₂O with 0.1% acetone, placed into a 3 mm NMR tube, and analyzed in a Bruker Avance-III operating at 500.13 MHz 1H (125.77 MHz 13C) equipped with a Bruker BBFO probe operating at 21.1° C. The sample glycan preparation was analyzed using a heteroatomic single quantum coherence pulse sequence (HSQC) using the standard Bruker pulse sequence. Anomeric protons between 4-6 ppm (1H) and 80-120 ppm (13C) were assigned by analogy to glucose as reported in Roslund, et al. (2008) Carbohydrate Res. 343:101-112. Spectra were referenced to the internal acetone signal: 1H—2.22 ppm; 13C—30.89 ppm. Isomers were quantitated by integration of their respective peaks using the MNova software package from Mestrelab Research (Santiago de Compostela, Spain). FIG. 4 shows that the alpha-/beta-ratio of two exemplary glycan preparations of gal50glu50 and glu100 does not significantly change despite shifts in the Avg DP, while a man52glu29gal19 preparation has a distinctly higher alpha-/beta-ratio even in preparations with low Avg DP. This ratio does not significantly increase as the Avg DP of man52glu29gal19 preparation rises. While the alpha-/beta-ratio and Avg DP are independent properties, they can be independently controlled. For example, the alpha-/beta-ratio may be controlled by selection of monomers with an inherent preference for one configuration over the other. The data in Table 1 for 15 exemplary glycan preparations show that the processes described herein can be controlled to produce glycans with alpha-/beta-ratios from about 1 (1.136, glu50gal50) to about 5 (5.556, man80glu20).

### Branching Analysis

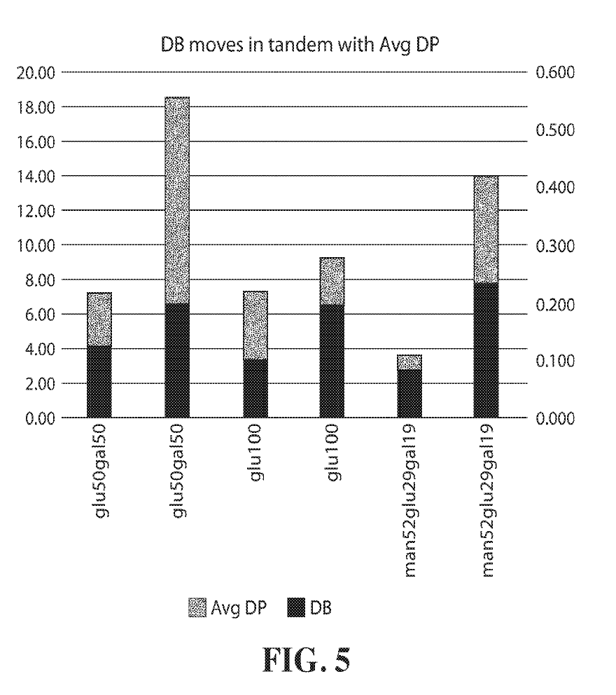

The distribution of glycosidic regioisomers (branching) within a given glycan was quantified. For glycosyl linkage analysis, the sample glycan preparation was permethylated, depolymerized, reduced, and acetylated; and the resultant partially methylated alditol acetates (PMAAs) analyzed by gas chromatography-mass spectrometry (GC-MS) as described by Heiss et al (2009) Carbohydr. Res. 344:915. The sample glycan preparation was suspended in 200 μl of dimethyl sulfoxide and left to stir for 1 day. Permethylation was effected by two rounds of treatment with sodium hydroxide (15 min) and methyl iodide (45 min). The aqueous solution was hydrolyzed by addition of 2M trifluoroacetic acid and heating to 121° C. for 2 hours. Solids were isolated in vacuo and acetylated in acetic acid/trifluoroacetic acid. The resulting PMAAs were analyzed on an Agilent 7890A GC interfaced to a 5975C MSD (mass selective detector, electron impact ionization mode); separation was performed on a 30 m Supelco SP-2331 bonded phase fused silica capillary column. Degree of branching (DB) is calculated by adding the percentages of each type of branched monomer and dividing by 100. The average DB can be controlled. FIG. 5 shows that average DB moves together with Avg DP. Glycans with DP<4 cannot branch. As glycan oligomers lengthen, the statistical likelihood that the chain elongates along the side of the backbone rather than at the end of the backbone increases. The date in Table 1 of 15 exemplary glycan preparations show that the process can be controlled to produce glycans with an average DB ranging from about 0.05 (e.g., 0.084, low Mw man52glu29gal19) to about 0.6 (e.g., 0.632, high Mw xyl100).

### Solubility

All glycan preparations were analyzed for two benchmarks of solubility: at 10% and 75% w/w in water. To determine solubility at 10% w/w in water, glycan preparations were isolated in dry form by lyophilization or other means, an accurate weight was obtained, and 9x weight of water was added to the glycan. Glycans were deemed soluble if a clear solution could be obtained using no solubilization techniques beyond vortexing, sonication, or heating to 45° C. with a temperature controlled heat gun or water bath. Glycans were deemed insoluble if after solubilization treatment the solution remained cloudy, had significant particulate matter, had an experimentally significant shift in concentration after sterile filtration, or formed a visible gel or suspension on cooling. To determine solubility at 75% w/w in water, glycan preparations were fully dissolved in water, then the water was removed from the solution using a rotary evaporator until the concentration reached 75 Brix as measured with a sugar refractometer. Glycan preparations were deemed soluble if the syrup remained clear and precipitate-free after 24 hours of storage at 4° C. Glycan preparations were deemed insoluble if the solids formed in the syrup before reaching 75 Brix or precipitates formed during cold storage. All of the glycans made were soluble in solutions of 60 Brix and up to 75 Brix.

TABLE 1
Characterization of 15 exemplary glycan preparations.

| | | DP2 + | DP3 + | | | | | | |
|---|---|---|---|---|---|---|---|---|---|
| | | yield | yield | | | | Avg | | a/b |
| Abbreviation | | (%) | (%) | Mw | Mn | PDI | DP | DB | ratio |
| xyl100 | high | 97 | 92 | 2433 | 1178 | 2.07 | 18.28 | 0.632 | 1.852 |
| | high | 89 | 79 | 1710 | 417 | 4.10 | 12.81 | | |
| | med | 88 | 75 | 1095 | 379 | 2.89 | 8.15 | | |
| | low | 82 | 63 | 606 | 273 | 2.22 | 4.45 | | |
| glu33gal33fuc33 | high | 87 | 79 | 1585 | 593 | 2.67 | 10.10 | 0.408 | 1.961 |
| | med | 84 | 74 | 1244 | 458 | 2.72 | 7.90 | | |
| | low | 77 | 70 | 839 | 385 | 2.18 | 5.29 | | |
| ara100 | high | 88 | 80 | 2229 | 619 | 3.60 | 16.74 | | |
| | med | 92 | 85 | 1503 | 536 | 2.80 | 11.24 | 0.166 | 2.041 |
| | low | 71 | 58 | 761 | 308 | 2.47 | 5.62 | | |
| gal100 | high | 90 | 86 | 3073 | 689 | 4.46 | 18.86 | 0.222 | 2.632 |
| | med | 89 | 83 | 1780 | 564 | 3.16 | 10.88 | | |
| | low | 85 | 78 | 1488 | 518 | 2.87 | 9.07 | | |
| glu80man20 | | 90 | 81 | 1182 | 517 | 2.29 | 7.19 | 0.161 | 2.273 |
| glu60man40 | | 80 | 73 | 1584 | 615 | 2.58 | 9.67 | 0.164 | 3.030 |
| man80glu20 | | 93 | 87 | 2443 | 844 | 2.89 | 14.97 | 0.186 | 5.556 |
| man60glu40 | | 89 | 81 | 1772 | 647 | 2.74 | 10.83 | 0.200 | 4.545 |
| xyl75ara25 | high | 85 | 76 | 1683 | 431 | 3.90 | 12.60 | | |
| | med | 89 | 80 | 1225 | 470 | 2.61 | 9.14 | | |
| | low | 84 | 72 | 1032 | 403 | 2.56 | 7.68 | | |
| Man80gal20 | | 90 | 80 | 1785 | 645 | 2.77 | 10.91 | | |
| Man66gal33 | | 90 | 85 | 1441 | 585 | 2.46 | 8.78 | | |
| glu100 | high | 91 | 83 | 1581 | 500 | 3.16 | 9.65 | | |
| | med | 88 | 77 | 1339 | 614 | 2.18 | 8.15 | | |
| | low | 86 | 71 | 794 | 452 | 1.76 | 4.79 | | |
| glu50gal50 | high | 99 | 98 | 3024 | 1755 | 1.72 | 18.56 | 0.200 | 1.695 |
| | med | 91 | 82 | 1573 | 674 | 2.33 | 9.60 | | |
| | low | 89 | 80 | 1195 | 729 | 1.64 | 7.27 | 0.126 | 1.136 |
| man100 | high | 88 | 84 | 1868 | 616 | 3.03 | 11.42 | | |
| | med | 78 | 63 | 1066 | 371 | 2.87 | 6.47 | | |
| | low | 37 | 16 | 406 | 224 | 1.81 | 2.40 | | |
| man52glu29gal19 | high | 85 | 75 | 1977 | 530 | 3.73 | 12.09 | | |
| | med | 67 | 47 | 734 | 338 | 2.17 | 4.42 | | |
| | low | 54 | 25 | 615 | 289 | 2.13 | 3.69 | 0.084 | 3.167 |

Additional exemplary glycan preparations were characterized (e.g. by refractometry, GC-MS, SEC, IAC, 2D NMR/HSQC spectra, and permethylation), e.g., in WO/2016/122889 GLYCAN THERAPEUTICS AND RELATED METHODS THEREOF, Example 5, incorporated herein.

### Example 5: Glycan Preparations Modulate Bacterial Communities from Nasal and Oral Human Samples Propagated In Vitro

An ex vivo assays were performed to assess growth and shifts in the relative abundance of bacterial taxa in microbial communities from the anterior nares (nasal cavity) and saliva (oral cavity) of healthy human volunteers upon exposure to different glycan preparations. The assays were designed to assess the ability of different glycan preparations to differentially modulate the bacterial microbiota associated with two exemplary mucosal sites of humans, the oral and the nasal cavity. 15 exemplary glycan preparations: glu80man20, glu60man40, man80gal20, glu100, man66gal33, glu50gal50, man100, man52glu29gal19, man60glu40, man80glu20, glu33gal33fuc33, xyl75ara25, ara100, gal100, xyl100 and a commercially available control, fructo-oligosaccharide, FOS (Nutraflora FOS; NOW Foods, Bloomingdale Ill.), were prepared at 5% w/v in water, filter-sterilized and added to Costar 3370 96-well microplates for a final concentration of 0.5% w/v in the assay, with each glycan preparation assayed in triplicate and dextrose and water supplied as positive and negative controls.

### Human Nasal Bacterial Communities

Nasal microbial communities were obtained from healthy human volunteers by inserting a sterile swab into the naris approximately half an inch and rubbing the swab along the circumference inside the naris 3 times. From each nasal sample, an inoculum was prepared agitating the swab for 15 seconds in 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L soidum bicarbonate, 0.125 mg/L biotin, 1 mg/L pyridoxine, 1 m/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot C M et al. Nat Commun. 2014; 5:3114), supplemented with 1% (v/v) final Chopped Meat Glucose broth (Anaerobe Systems).

### Human Oral Bacterial Communities

Oral microbial communities were obtained by human volunteers drooling saliva into sterile collection tubes. From each saliva sample, an inoculum was prepared by adding saliva to 1% v/v final in 100 mM potassium phosphate buffer (pH 7.2), 15 mM sodium chloride, 8.5 mM ammonium sulfate, 4 mM L-cysteine, 1.9 μM hematin, 200 μM L-histidine, 100 μM magnesium chloride, 1.4 μM iron sulfate heptahydrate, 50 μM calcium chloride, 1 μg/mL vitamin K3 and 5 ng/mL vitamin B12 (Martens E C et al. Cell Host & Microbe 2008; 4, 447-457). Inocula were added to the assay plates with final test volumes of 200 uL per well and final test concentrations of glycans and dextrose of 0.5% w/v, and incubated at 370° C. aerobically for 4 days. OD₆₀₀ measurements at the end of the incubation period were obtained using a Biotek Synergy2 reader with Gen5 2.0 software according to manufacturer's specifications.

### 16s Sequencing

To determine the composition of microbial communities, genomic DNA was extracted from 200 uL of cultures harvested at 0, 9, 12, 18.5 and 48 hours, using MoBio Soil DNA extraction. V4 region of 16S rRNA gene was amplified using 515 Forward and 806 Reverse primers as described in Caporaso J G, Lauber C L, Walters W A, Berg-Lyons D, Huntley J, Fierer N, Owens S M, Betley J, Fraser L, Bauer M, Gormley N, Gilbert J A, Smith G, Knight R. 2012. Ultra-high-throughput microbial community analysis on the Illumina HiSeq and MiSeq platforms. ISME J. Amplicons were sequenced using Illumina MiSeq instrument with 250 bp long reads using paired end chemistry. Operational Taxonomic Units (OTUs) were analyzed using 97% sequence identity. Representation of OTUs was compared across different mucosal sites and glycan preparations. Most abundant bacterial taxa for the two mucosal sites (nasal and oral cavity) in the ex vivo propagated communities from human donors are summarized in Table 2. For the nasal cavity, the most abundant bacterial genera included Corynebacterium, Alloiococcus, and Staphylococcus. For the oral cavity, the most abundant bacterial genera included Prevotella, Oribacterium, Bifidobacterium, and Moryella.

TABLE 2
Most abundant bacterial taxa in the ex vivo communities
for nasal and oral cavity, respectively. All
genera that comprise on average >5%.

| | | Average |
|---|---|---|
| | | Relative |
| Site | Genera | Abundance |
| Nasal | Corynebacterium | 68.53% |
| Nasal | Staphylococcus | 13.57% |
| Nasal | Alloiococcus | 16.83% |
| Oral | Bifidobacterium | 11.47% |
| Oral | Prevotella | 42.68% |
| Oral | Moryella | 5.03% |
| Oral | Oribacterium | 19.05% |

Modulation of Nasal Bacterial Taxa and Association with Disease and Pathogenic Conditions

In the nasal ex vivo assay, the relative abundance of the genera Corynebacterium and Staphylococcus were differentially modulated by at least 8 glycan preparations. Relative to glucose (baseline), Ara100, Xyl100, Man80gal20, Ga1100, xyl75ara25 and Man66gal33 tended to promote the growth of Corynebacterium over Staphylococcus and shifted the balance in favor of Corynebacterium, with more Corynebacterium and less Staphylococcus present in the ex vivo cultures.

The nasal cavity serves as a reservoir for species Staphylococcus aureus, and carriage of S. aureus is a significant risk factor for nosocomial S. aureus bacteraemia (Wertheim, H. F. L., et al. (2004). Risk and outcome of nosocomial Staphylococcus aureus bacteraemia in nasal carriers versus non-carriers. Lancet Lond. Engl. 364, 703-705). The nasal microbiome is also thought to play a role in the pathogenesis of chronic rhinosinusitis (CRS). Although the total bacterial burden is similar in CRS patients and healthy controls, CRS patients tend to have less diverse microbiomes and higher prevalence of S. aureus than controls (Wilson, M. T., and Hamilos, D. L. (2014). The nasal and sinus microbiome in health and disease. Curr. Allergy Asthma Rep. 14, 485).

In the anterior nares, an increase in Moraxella, an opportunistic pathogen associated with otitis media and sinusitis, is associated with a decrease in genera including Staphylococcus, Corynebacterium and Propionibacterium. As shown in Table 2, glycan preparations in the ex vivo nasal assays supported the growth of microbial communities with average relative abundances exceeding 70% for Corynebacterium and Staphylococcus, being two of the most abundant taxa. Glycan preparations may thus be administered to promote the growth of genera such as Staphylococcus and Corynebacterium for therapeutic benefit, including modulating the bacterial balance (e.g., relative abundances in the niche, such as the nasal cavity) in such way as to disfavor the growth or propagation of opportunistic pathogens such as Moraxella.

Modulation of Oral Bacterial Taxa and Association with Disease and Pathogenic Conditions

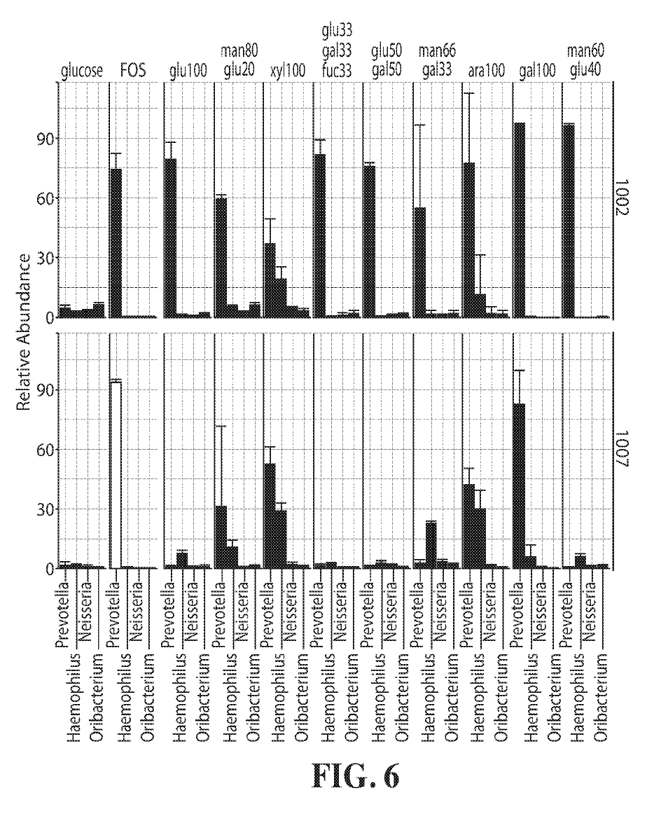

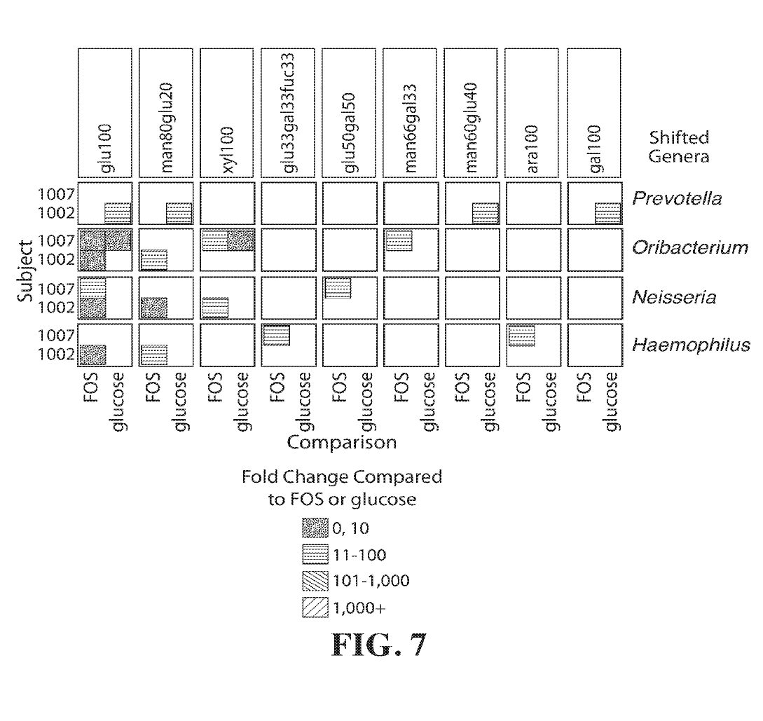

In the oral ex vivo assay, the relative abundance of the genera Prevotella, Oribacterium, Neisseria and Haemophilus with samples from two subjects were differentially modulated by at least 9 glycan preparations at 20 hours as summarized in FIG. 6. The glycan preparations resulted in different relative abundances than FOS in assays of oral microbiome samples from one or two of the subjects. In assays with samples from both subjects, FOS resulted in high relative abundance of mainly Prevotella. In the sample from subject 1007, the relative abundance of Haeomphilus was greater than Prevotella on glu100, man66gal33 and man60glu40. In the sample from subject 1002 on man80glu20 and xyl100, Prevotella was most abundant, and the relative abundances of Haemophilus, Neisseria and Oribacterium were generally at least 5%. Additionally, Prevotella, Oribacterium, Neisseria and Haemophilus were each modulated by at least 4 glycan preparations relative to FOS and/or glucose in the assay with samples from one or both subjects, as summarized in FIG. 7. Prevotella relative abundance was increased by glu100, man80glu20, man60glu40 and gal100 in the assay relative to FOS or glucose. Oribacterium was increased in the assay with glu100, man80glu20, xyl100 and man66glu33 relative to FOS or glucose. Neisseria, including the OTU for the beneficial bacterial species Neisseria subflava, was increased in the assay with glu100, glu80man20, xyl100 and glu50gal50 relative to glucose or FOS. Haemophilus was increased in the assay with glu100, glu80man20, glu33gal33fuc33 and ara100 relative to FOS or glucose. The relative abundance of 8 additional genera, Bifidobacterium, Abiotrophia, Clostridiales, Catonella, Moryella, Leptotrichia, Eikenella and Aggregatibacter were significantly increased by at least one glycan preparation in this assay relative to FOS or glucose. The genera Prevotella, Oribacterium, Neisseria and Haemophilus and the species Neisseria subflava have been associated with good oral health and low dental plaque (Pereira et al, Braz Dent J 2012). The modulation of the oral microbiota by glycan preparations in the assay supports the idea that glycan preparations may be administered to modulate the oral microbiota and promote the growth of beneficial bacteria to improve or maintain good oral health.

### Example 6: Glycan Preparations Differentially Modulate Bacterial Strains from Human Nasal, Oral, and Vaginal Sites In Vitro

An in vitro assay was performed to assess the ability of various bacterial strains, including commensals of non-gut sites harboring mucosal tissue such as the nares (nasal cavity), the vagina and the oral cavity, to utilize different glycan preparations as growth substrates. This assay was designed to assess the ability of selected glycan preparations to differentially modulate the growth of bacteria associated with various non-gut mucosal sites. Bacterial isolates were handled aerobically or anaerobically in the assay. For anaerobic cultures, strains were handled at all steps in an anaerobic chamber (AS-580, Anaerobe Systems) featuring a palladium catalyst. The chamber was made anaerobic initially by purging with an anaerobic gas mixture of 5% hydrogen, 5% carbon dioxide and 90% nitrogen and subsequently maintained in an anaerobic state using this same anaerobic gas mixture. Anaerobicity of the chamber was confirmed daily using Oxoid anaerobic indicator strips that change color in the presence of oxygen. All culture media, assay plates, other reagents and plastic consumables used in testing anaerobic cultures were pre-reduced in the anaerobic chamber for 24-48 hours prior to contact with bacteria. 14 exemplary glycan preparations: glu80man20, glu60man40, man80gal20, glu100, man66gal33, glu50gal50, man100, man52glu29gal19, man60glu40, man80glu20, glu33gal33fuc33, xyl75ara25, gal1100, xyl1100, and a commercially available control, FOS (Nutraflora FOS; NOW Foods, Bloomingdale Ill.), were prepared at 5% w/v in water, filter-sterilized and added to Costar 3370 assay plates for a final concentration of 0.5% w/v in the assay, with each glycan assayed in triplicate and dextrose (0.5% w/v final) and water supplied as positive and negative controls. Bacterial isolates were obtained from the American Type Culture Collection (ATCC, Manassas, Va.). Cultures of Staphylococcus epidermidis (ATCC 14990, “SEP.55”) and S. hominis (ATCC 27844, “SHO.56”) were grown aerobically in Brain Heart Infusion broth (BHI, Teknova), a rich infusion medium including brain infusion, heart infusion, peptone, glucose, sodium chloride and disodium phosphate, at 370° C. for 18-24 hours. Cultures of Lactobacillus crispatus (ATCC 33820, “LCR.43”), L. gasseri (ATCC 33323, “LGA.44”), L. iners (ATCC 55195, “LCR.45”), Propionibacterium acnes (ATCC 6919, “PAC.48”), S. aureus (ATCC 12600, “SAU.54”) and Streptococcus oralis (ATCC 35037, “SOR.60”) were grown anaerobically on Brucella Blood Agar (Anaerobe Systems, Morgan Hill, Calif.), an enriched medium supplemented with vitamin K, hemin and sheep's blood, for 18-48 hours at 370° C. Colonies were scraped from the Brucella Blood Agar and suspended in Chopped Meat Glucose broth, (CMG, Anaerobe Systems), a pre-reduced enriched medium including lean ground beef, enzymatic digest of casein, yeast extract, potassium phosphate, dextrose, cysteine, hemin and Vitamin K1. Inocula were prepared by determining the optical density of each liquid culture or cell suspension at 600 nM (OD₆₀₀) in a Costar 3370 polystyrene 96-well flat-bottom assay plate using a Biotek Synergy 2 plate reader with Gen5 2.0 All-In-One Microplate Reader Software according to manufacturer's protocol, and diluting the cells to OD₆₀₀ 0.01 final in defined and semi-defined media that were prepared without sugars. P. acnes and S. oralis isolates were tested in 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L soidum bicarbonate, 0.125 mg/L biotin, 1 mg/L pyridoxine, 1 m/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot C M et al. Nat Commun. 2014; 5:3114), supplemented with 0-3.5% (v/v) CMG. S. epidermidis and S. hominis were tested in 100 mM potassium phosphate buffer (pH 7.2), 15 mM sodium chloride, 8.5 mM ammonium sulfate, 4 mM L-cysteine, 1.9 μM hematin, 200 μM L-histidine, 100 μM magnesium chloride, 1.4 μM iron sulfate heptahydrate, 50 μM calcium chloride, 1 μg/mL vitamin K3 and 5 ng/mL vitamin B12 (Martens E C et al. Cell Host & Microbe 2008; 4, 447-457), supplemented with 0-5% glucose-free BHI. S. aureus, L. crispatus, L. gasseri and L. iners were tested in 10 g/L tryptone peptone, 5 g/L yeast extract, 0.5 g/L L-cysteine hydrochloride, 0.1 M potassium phosphate buffer pH 7.2, 1 μg/mL vitamin K3, 0.08% w/v calcium chloride, 0.4 μg/mL iron sulfate heptahydrate, 1 μg/mL resazurin, 1.2 μg/mL hematin, 0.2 mM histidine, 0.05% Tween 80, 0.5% meat extract (Sigma), 1% trace mineral supplement (ATCC), 1% vitamin supplement (ATCC), 0.017% v/v acetic acid, 0.001% v/v isovaleric acid, 0.2% v/v propionic acid and 0.2% v/v N-butyric acid (Romano K A et al. mBio 2015; 6(2):e02481-14). L. gasseri, S. hominis and S. epidermidis were tested aerobically, and P. acnes, S. aureus, S. oralis, L. crispatus and L. iners were tested anaerobically. Bacteria were exposed to the 14 glycan preparations glu80man20, glu60man40, man80gal20, glu100, man66gal33, glu50gal50, man100, man52glu29gal19, man60glu40, man80glu20, glu33gal33fuc33, xyl75ara25, gal100, xyl100 and FOS and dextrose at a final concentration of 0.5% w/v in 96-well microplates, 200 μL final volume per well, at 370° C. for 18-48 hours. OD₆₀₀ measurements for each isolate at the end of the incubation period were obtained using a Biotek Synergy2 reader with Gen5 2.0 software according to manufacturer's specifications. Normalized Growth Values (NGVs) were obtained by dividing the OD₆₀₀ readings of the isolate on test glycan preparations by the average OD₆₀₀ of the isolate in medium supplemented with 0.5% w/v dextrose to facilitate comparison of glycan utilization by strains that grow within significantly different OD₆₀₀ ranges. Table 3 summarizes the results obtained.

TABLE 3
Glycan preparations differentially supported growth of bacteria associated with non-gut
mucosal sites: Nasal, oral, and vaginal.

| | Bacterial Isolates from Various Mucosal Sites, Average Normalized Growth | | | | | | | |
|---|---|---|---|---|---|---|---|---|
| | vaginal | | | nasal | | | | oral |
| Glycan | LCR. | LGA. | LIN. | SEP. | SHO. | PAC. | SAU. | SOR. |
| # | 43 | 44 | 45 | 55 | 56 | 48 | 54 | 60 |
| 1 | +++ | ++ | + | + | + | + | ++ | − |
| 2 | +++ | ++ | + | + | ++ | + | + | − |
| 3 | ++ | ++ | + | + | − | + | + | − |
| 4 | + | + | − | + | − | + | ND | + |
| 5 | + | + | − | + | − | + | + | + |
| 6 | + | + | − | + | − | + | + | + |
| 7 | + | + | + | + | ++ | − | − | − |
| 8 | + | + | − | + | − | + | + | − |
| 9 | + | + | − | + | − | + | + | − |
| 10 | + | + | − | + | − | + | + | − |
| 11 | − | − | − | − | + | − | − | − |
| 12 | − | − | − | − | + | − | − | − |
| 13 | − | − | − | − | + | − | − | − |
| 14 | − | − | − | − | + | − | − | − |
| FOS | + | − | − | + | ++ | − | ND | − |

Key to Glycans

| glycan # | glycan composition |
|---|---|
| 1 | glu80man20 |
| 2 | glu100 |
| 3 | glu60man40 |
| 4 | man80gal20 |
| 5 | man66gal33 |
| 6 | man100 |
| 7 | glu33gal33fuc33 |
| 8 | man80glu20 |
| 9 | man60glu40 |
| 10 | man52glu29gal19 |
| 11 | gal100 |
| 12 | xyl75ara25 |
| 13 | glu50gal50 |
| 14 | xyl100 |

Key to Growth

| symbol | NGV |
|---|---|
| − | <0.1 |
| + | 0.1-0.3 |
| ++ | 0.3-0.7 |
| +++ | >0.7 |
| ND | Not |
| | Determined |

In the assay, glycan preparations differentially modulated the growth of bacterial strains associated with mucosal sites.

### Vaginal

Glycan preparations glu80man20, glu100 and glu60man40 promoted the strongest growth of vaginally-associated Lactobacilli, with normalized growth values of 0.3→0.7 for LCR.43 and LGA.44 and 0.1-0.3 for LIN.45 in the assay. Glu33gal33fuc33 supported growth of the 3 vaginally-associated Lactobacilli with normalized growth of at least 0.1 in the assay. 10 of 14 glycans supported growth of at least 2 Lactobacilli in the assay with normalized growth values of at least 0.1, while the commercially available comparator FOS supported growth of only 1 Lactobacillus isolate, LCR.43.

### Nasal

Glu33gal33fuc33 also supported the growth of the nasal commensals S. epidermidis (SEP.55) and S. hominis (SHO.56) with normalized growth values >0.1 in the assay, but not P. acnes (PAC.48) or S. aureus (SAU.54). S. hominis (SHO.56) was the only strain in the assay with normalized growth >0.1 on gal100, xyl75ara25, glu50gal50 and xyl100.

### Oral

In the assay, mannose-galactose containing heteroglycan preparations, e.g., man80gal20, man66gal33, and man100 promoted growth of orally-associated S. oralis SOR.60 with normalized growth of at least 0.1.

Thus glycans appear to differentially promote growth of bacterial isolates associated with various human mucosal sites. Glycan preparations may be administered to selectively promote members of the microbiota that have antagonistic relationships with or are inversely correlated with bacterial species associated with disease or dysbiotic states.

Modulation of Vaginal Bacterial Taxa and Diversity and Association with Disease and Pathogenic Conditions

Lactobacilli including L. crispatus, L. gasseri and L. iners are associated with healthy human vaginal flora and are thought to contribute to protection against pathogens by lowering vaginal pH through lactic acid production. Vaginal bacterial communities dominated by Lactobacilli are negatively correlated with bacterial vaginosis (Ravel et al, PNAS 2011 vol. 108). Hydrogen peroxide production by some Lactobacilli is also thought to contribute to protection against pathogens and maintenance of vaginal health, and the abundance of Lactobacilli, especially hydrogen-peroxide producing species, has been found to be significantly reduced in women with bacterial vaginosis (Mijac et al, European Journal of Obstetrics & Gynecology and Reproductive Biol, 2006). Administration of glycan preparations to selectively promote the growth of Lactobacilli (and that may in turn lower bacterial diversity in the vaginal site) may have therapeutic benefit in maintenance or restoration of a vaginal microbiota associated with health and may be beneficial to treat or prevent conditions associated with microbiome dysbiosis, such as bacterial vaginosis.

Modulation of Nasal Bacterial Taxa and Association with Disease and Pathogenic Conditions

Administration of glycan preparations to selectively promote growth of certain commensals may have therapeutic benefit in the nares (and nasal cavity). Staphylococcus strains represent a significant component of the nasal microbiome, and were present at an average abundance of 13% in the nasal ex vivo assays. The single strain growth assay data show differential modulation of different species of Staphylococci, and the selective promotion of growth of one species, such as S. epidermidis by glu33gal33fuc33, may modulate the growth or activity of other species, such as S. aureus. Protease secretion by S. epidermidis strains, commonly found in the nose and pharynx, has been shown to inhibit S. aureus biofilm formation and nasal colonization (Iwase et al, Nature, 2010). Decolonization of methicillin-resistant S. aureus (MRSA) in patients in surgical intensive care units with chlorhexidine baths and intranasal administration of the antibiotic mupirocin has been associated with reduced rates of MRSA infection; however, it has also been associated with significantly increased resistance to mupirocin (Cho et al, Am J Infect Control, 2016). Administration of glycan preparations that selectively promote growth of S. epidermidis may be beneficial in MRSA decolonization without promoting mupirocin resistance.

Modulation of Oral Bacterial Taxa and Association with Disease and Pathogenic Conditions

The oral commensal species S. oralis is associated with healthy human oral microbiota and is considered beneficial to oral health. It has been found to inhibit the growth of oral pathogens via production of hydrogen peroxide (Herrero et al, Antimicrobial effects of commensal oral species are regulated by environmental factors J. Dent 2016). The glycans man80gal20, man66gal33 and man100 support the growth of S. oralis in the assay with normalized growth values >0.1. Glycans may be administered to support the growth of S. oralis or other beneficial bacteria to maintain or improve oral health.

These data show that at least 35 distinct glycan preparations were made, which included preparations made from one, two, or three different monomers (Example 1), of which at least 15 glycan preparations or various batches where characterized for at least 6 properties, selected from: DP2+ (dimer and above) yield, DP3+ (trimer and above) yield, weight-average molecular weight (Mw), number average molecular weight (Mn), polydispersity index (PDI), average degree of polymerization (DP), average degree of branching (DB), and alpha-to-beta glycosidic bond ratio (a/b-ratio) (Example 4).

At least 15 characterized glycan preparations were tested in 2 ex vivo assays (Example 5) of bacterial communities derived from human samples of sites containing mucosal tissues (the nasal cavity and oral cavity). At least 12 glycan preparations modulated at least one common bacterial constituent of the oral cavity. At least 9 glycan preparations modulated at least one bacterial constituent that is thought to be associated with oral health. At least 8 glycan preparations modulated at least two of the most abundant bacterial constituents in the ex vivo nasal cavity community.

At least 14 characterized glycan preparations were tested on a panel of 8 bacterial strains in vitro (Example 6) which are representative members of 3 sites containing mucosal tissues (the nasal cavity, oral cavity and the vagina). All 14 glycan preparations modulated the growth of at least one bacterial strain, at least 10 glycan preparations modulated at least 5 of the bacterial strains tested (across at least 2 different sites).

### Example 7. Measurement of Metabolites with Mass Spectrometry (MS) or ¹H-Nuclear Magnetic Resonance (¹H-NMR)

Glycan preparations are selected for administration to an animal or human subject based on their effect on the presence of microbial fermentation products. For example, populations of glycan preparations are selected for their ability to induce or promote growth of bacteria that produce short chain fatty acids such as propionate (propionic acid), acetate, and/or butyrate (butyric acid). Similarly, populations of glycan preparations are selected for their ability to induce or promote growth of bacteria that produce lactic acid, which can modulate the growth of other bacteria by producing an acidic environment. Such analysis may also be also used to pair probiotic bacteria with a glycan preparation such that the glycan preparation is a substrate for the production of the desired fermentation products.

The metabolites that are present in fresh or spent culture media or in biological samples collected from humans or animals are determined using methods described herein. Unbiased methods are used to determine the relative concentration of metabolites in a sample and are known to one skilled in the art. Gas or liquid chromatography combined with mass spectrometry is used to determine the amounts and identities of various metabolites in the aforementioned samples. Alternatively, ¹H-NMR is used for unbiased metabolomic profiling. These measurements are validated by running metabolite standards through the same analytical systems.

### Gas Chromatography-Mass Spectrometry (GC-MS)

Polar metabolites and fatty acids are extracted using monophasic or biphasic systems of organic solvents and an aqueous sample and derivatized (Fendt et al., Reductive glutamine metabolism is a function of the α-ketoglutarate to citrate ratio in cells, Nat Commun, 2013, 4:2236) (Fendt et al., Metformin decreases glucose oxidation and increases the dependency of prostate cancer cells on reductive glutamine metabolism, Cancer Res, 2013, 73:4429) (Metallo et al., Reductive glutamine metabolism by IDH1 mediates lipogenesis under hypoxia, Nature, 2011, 481:380). Briefly, polar metabolites are derivatized to form methoxime-tBDMS derivatives by incubation with 2% methoxylamine hydrochloride (MP Biomedicals) in pyridine (or MOX reagent (Thermo Scientific)) followed by addition of N-tert-butyldimethylsilyl-N-methyltrifluoroacetamide (MTBSTFA) with 1% tert-butyldimethylchlorosilane (t-BDMCS) (Regis Technologies). Non-polar fractions, including triacylglycerides and phospholipids, are saponified to free fatty acids and esterified to form fatty acid methyl esters either by incubation with 2% H₂SO₄ in methanol or by using Methyl-8 reagent (Thermo Scientific). Derivatized samples are analyzed by GC-MS using a DB-35MS column (30 m×0.25 mm i.d.×0.25 μm, Agilent J&W Scientific) installed in an Agilent 7890A gas chromatograph (GC) interfaced with an Agilent 5975C mass spectrometer (MS). Mass isotopomer distributions are determined by integrating metabolite ion fragments and corrected for natural abundance using algorithms adapted from Fernandez et al. (Fernandez et al., Correction of 13C mass isotopomer distributions for natural stable isotope abundance, J Mass Spectrom, 1996, 31:255).

### Liquid Chromatography-Mass Spectrometry (LC-MS) of Polar Metabolites

After extraction, samples are transferred to a polypropylene vial and samples are analyzed using a Q Exactive Benchtop LC-MS/MS (Thermo Fisher Scientific). Chromatographic separation is achieved by injecting 2 μL of sample on a SeQuant ZIC-pHILIC Polymeric column (2.1×150 mm; EMD Millipore). Flow rate is set to 100 μL/min, column compartment is set to 25° C., and autosampler sample tray is set to 4° C. Mobile Phase A consists of 20 mM ammonium carbonate and 0.1% ammonium hydroxide in water. Mobile Phase B is 100% acetonitrile. The mobile phase gradient (% B) is as follows: 0 min 80%, 5 min 80%, 30 min 20%, 31 min 80%, and 42 min 80%. All mobile phase is introduced into the Ion Max source equipped with a HESI II probe set with the following parameters: Sheath Gas=40, Aux Gas=15, Sweep Gas=1, Spray Voltage=3.1 kV, Capillary Temperature=275° C., S-lens RF level=40, Heater Temp=350° C. Metabolites are monitored in negative or positive mode using full scan or a targeted selected ion monitoring (tSIM) method. For tSIM methods, raw counts are corrected for quadrupole bias by measuring the quadrupole bias experimentally in a set of adjacent runs of samples at natural abundance. Quadrupole bias is measured for all species by monitoring the measured versus theoretical m1/m0 ratio at natural abundance of all species with m−1, m0, m1, and m2 centered scans. Quadrupole bias-corrected counts are additionally corrected for natural abundance to obtain the final mass isotopomer distribution for each compound in each sample.

¹H-Nuclear Magnetic Resonance (¹H-NMR)

To prepare the extracted sample for analysis by ¹H-NMR, 400 μL of the sample is combined with 200 μL of 50 mM phosphate buffer (prepared with Na₂HPO₄, NaH₂PO₄ in D₂O, pH 7.4) supplemented with 5 mM 2,2-dimethyl-2-silapentane-5-sulfonate sodium salt (DSS-d₆, standard and reference for chemical shift; Isotec, USA) and vortexed briefly. The solution is centrifuged at 1000 g for 1 min and then 500 μL is transferred to a 5 mm NMR tube (VWR). ¹H-NMR spectra are acquired on an NMR spectrometer (Avance II+500 Bruker spectrometer (500 MHz) (Bruker, DE), equipped with a 5 mm QXI-Z C/N/P probe-head) and analyzed with spectra integration software (Chenomx NMR Suite 7.1; Chenomx Inc., Edmonton, AB). (Duarte et al., ¹H-NMR protocol for exometabolome analysis of cultured mammalian cells, Methods Mol Biol, 2014:237-47). Alternatively, ¹H-NMR is performed following other published protocols. (Chassaing et al., Lack of soluble fiber drives diet-induced adiposity in mice, Am J Physiol Gastrointest Liver Physiol, 2015) (Bai et al., Comparison of Storage Conditions for Human Vaginal Microbiome Studies, PLoS ONE, 2012:e36934)

### Example 8. Effect of Glycan Preparations on the Nasal Microbiome

Glycan preparations are formulated in such a way that they are administered directly in the nasal cavity by a spray or topically-applied gel. Alternatively, glycan preparations are administered orally by capsule or tablet form such that they provide indirect effects on the nasal microbiome via metabolites formed by the gut microbiota or other modulation of the host by the gut microbiota. Samples of the nasal microbiota are obtained both before and after application of the glycan preparation formulations by swab. Microbial population shifts are then investigated by 16S rRNA gene sequencing, whole-genome sequencing, or RNA-Seq to determine the effect of the administered glycan preparations. Shifts in microbial metabolites are measured, e.g., as described in Example 7. The post-treatment samples are compared with pre-treatment samples. In this example, the effects of glycan preparations are assessed in human subjects who are known to have nasal carriage of Staphylococcus aureus or methicillin-resistant S. aureus (MRSA). Determination of carriage is performed by culture of a nasal swab which is incubated overnight in a tryptone-based broth containing 7.5% sodium chloride and 1% mannitol (Difco m Staphylococcus broth; Becton Dickinson) and subcultured onto mannitol-salt agar supplemented with oxacillin (2 mg/mL; Quelab). MRSA is identified using standard methods, including the latex agglutination test for detection of penicillin-binding protein 2a (MRSA-Screen; Denka Seiken). Glycan preparations are applied intra-nasally or orally as described above. Administration of glycan preparations is used in the absence of a co-treatment as determined by a physician. Alternatively, glycan preparations are administered prior to, concurrent with or post treatment with standard-of-care treatments to eliminate S. aureus from the nasal cavity, including topical mupirocin application or oral antibiotics such as rifampin and doxycycline; glycan preparations may also be administered in conjunction with beneficial bacteria. Within a suitable treatment period a reduction of S. aureus or MRSA in the nasal cavity is observed. Additionally, for glycan preparations that are ingested and generate systemic effects leading to the resolution of a disease state in the nasal cavity, the following changes are observed: shifts in the gut microbiota away from a disease state and towards a healthy state or an increase in short chain fatty acids.

### Example 9. Effect of Glycan Preparations in an Animal Model of Nasal Colonization with Staphylococcus aureus

Glycan preparations are tested for their ability to reduce or abolish nasal colonization with Staphylococcus aureus using established animal models. Such models exist in the cotton rat (Methods Mol Biol. 2008; 431:241-54 The Cotton Rat as a Model for Staphylococcus aureus nasal colonization in humans: cotton rat S. aureus nasal colonization model), pig (Szab6, Istvin et al. Colonization Kinetics of Different Methicillin-Resistant Staphylococcus aureus Sequence Types in Pigs and Host Susceptibilities. Applied and Environmental Microbiology 78.2 (2012): 541-548) and mouse (Holtfreter, Silva et al. Characterization of a Mouse-Adapted Staphylococcus aureus Strain. PLoS ONE 8.9 (2013): e71142; Park, Bonggoo, Tadayuki Iwase, and George Y. Liu. Intranasal Application of S. Epidermidis Prevents Colonization by Methicillin-Resistant Staphylococcus Aureus in Mice. PLoS ONE 6.10 (2011): e25880). For the mouse model, S. aureus strains (either human pathogenic strain, such as MRSA USA500, or mouse-adapted strain JSNZ) are rendered streptomycin-resistant so that native bacterial flora from nasal tissue can be eliminated from plate counting, using streptomycin-containing media. Bacteria are generally grown overnight and inoculated directly into the nares of naïve mice. At various times post-inoculation mice are euthanized and the nasal tissue dissected out and homogenized. Dilutions of the homogenate are then applied to TSA agar plates with and without streptomycin. Colonies are then enumerated to determine the level of colonization with S. aureus. Dosing of glycan preparations is instituted following several regimens, including exclusively prior to S. aureus inoculation, post-inoculation of S. aureus, or throughout the entire study. Glycan preparations are dosed by instillation of a liquid solution directly into the nares or orally. The effects of glycan preparations versus placebo (control) are determined by enumeration of bacteria as described above. In addition, the effects of glycan preparations on the native microbiota are determined by subjecting nasal homogenates to DNA or RNA isolation and 16S or transcriptome analysis.

### Example 10: Formulation and Efficacy of a Nasal Spray for the Treatment of Chronic Rhinosinusitis

This study is carried out to determine the effectiveness of administering exemplary glycan preparations (e.g., as described herein) in combination with fluticasone propionate for the treatment of chronic rhinosinusitis. An aqueous suspension comprising up to 75% (e.g. between 50% and 75%) of a glycan preparation, microfine fluticasone propionate (50 mcg, optionally between 10 mcg and 100 mcg), and optionally one or more of: microcrystalline cellulose, carboxymethylcellulose sodium, dextrose, benzalkonium chloride, polysorbate 80, and phenylethyl alcohol (0.25% w/w) is prepared and loaded into a metering, atomizing spray pump. Subjects suffering from chronic rhinosinusitis are administered the nasal spray and instructed to apply the spray in the nostril one (or more, e.g. 2-5) time daily. After one week, subjects are examined for overall improvement in symptoms as compared to subjects that have followed the same regimen using a nasal spray that does not contain the glycan preparation.

### Example 11: Formulation and Efficacy of an Inhalation Treatment for Nasal Vestibulitis

This study is conducted to determine the efficacy of administering exemplary glycan preparations (e.g., as described herein) in combination with mupirocin for the treatment of nasal vestibulitis. An ointment comprising up to 75% (e.g. between 50% and 75%) of a glycan preparation, mupirocin (2%, optionally between 1% and 5%), and optionally PEG 400 and PEG 3350 is prepared in a bland water miscible ointment base, and is dosed in single use tubes. Subjects with nasal vestibulitis are instructed to apply the entire contents from a single use tube topically to the nostril once (or more, e.g. 2-5) a day for five days, massaging the nasal passages after each application for one minute. After one week, subjects are examined for overall improvement in symptoms as compared to subjects that have followed the same regimen using an ointment that does not contain the glycan preparation.

### Example 12. Effect of Glycan Preparations on the Oral Microbiome

Glycan preparations are formulated in a liquid, lozenge, sublingual film, paste, or gum such that they are administered directly in the oral cavity. Liquid application includes a “rinse and spit” topical application. In addition, glycan preparations administered for ingestion in a liquid, capsule or tablet form can provide indirect effects on the oral microbiome via metabolites formed by the gut microbiota or other modulation of the host by the gut microbiota. Samples of the oral microbiota are obtained both before and after application of the glycan formulations by swab, scrape, or collection of drool. Microbial population shifts are then investigated by 16S rRNA gene sequencing, whole-genome sequencing, or RNA-Seq as described to determine the effect of the administered glycan preparations. Shifts in microbial metabolites are measured as described in Example 7. The post-treatment samples are compared with pre-treatment samples. In this example, the effects of glycan preparations are assessed in human subjects that have an oral disease or condition. For example, otherwise healthy subjects diagnosed with periodontal disease are recruited or included in a clinical study. Diagnosis and severity are determined by using standard measures, such as gingival probing depth, measured with a calibrated probe, clinical attachment level and gum bleeding upon probing scores. Such subjects include those with varying levels of periodontal disease ranging from mild to moderate inflammation of the gums to oral bone loss. Administration of glycan preparations is used in the absence of a co-treatment as determined by a physician. Alternatively, a population of glycan preparations is administered prior to, concurrent with or post treatment with standard-of-care treatments, including antibiotics, physical methods to remove plaque, and probiotics. Within a suitable treatment period, the following changes are observed: a resolution of periodontal symptoms (assessed according to the diagnostic criteria listed above), a reduction in dental plaque, a decrease in the levels of pathogenic oral bacteria such as Streptococcus mutans, and/or an increase in the levels of bacteria associated with a healthy oral microbiome. Additionally, for glycan preparations that are ingested and generate systemic effects leading to the resolution of the symptoms of periodontal disease, the following changes areobserved: shifts in the gut microbiota away from a disease state and towards a healthy state or an increase in short chain fatty acids.

### Example 13. Effect of Glycan Preparations in an Animal Model of Periodontitis

Glycan preparations are tested in a variety of animal models of periodontitis. Models exist in rodents, rabbits, pigs, dogs, and nonhuman primates (Oz, Helieh S., and David A. Puleo. “Animal Models for Periodontal Disease.” Journal of Biomedicine and Biotechnology 2011 Article ID: 754857). The swamp rice rat is particularly susceptible to a rapid development of diet-induced periodontitis (Leonard, E. P. “Periodontitis. Animal Model: Periodontitis in the Rice Rat (Oryzomys Palustris).” The American Journal of Pathology 96.2 (1979): 643-646).

In one rat model, animals are anesthetized and sterile, 3-0 black braided nylon thread (surgilon; USS/DG, Norwalk, Conn., USA) is placed around the cervical margins of the bilateral lower first molars and knotted mesially. The area around the ligature becomes prone to biofilm formation, and particular species of disease-causing bacteria are introduced (e.g., Porphyromonas gingivalis) to augment biofilm formation and pathogenesis. Rats are killed under anesthesia 7 d after ligature. One side of the mandible is used for routine histological processing to paraffin wax and the other side is used for bacterial analysis after plaque/bacterial samples are taken from the tissue adjacent to the ligature. Alternatively, sampling occurs over time by swab sampling of the affected area. Dosing of populations of glycan preparations is instituted following several regimens, including exclusively prior to the initiating event (ligature placement, or inoculation of disease-causing bacteria), post-initiation, or throughout the entire study. Glycan preparations are dosed by instillation of a liquid solution directly into the oral cavity, or orally. Alternatively, glycan preparations are incorporated into food or water. The effect of glycan preparations versus placebo (control) is determined by enumeration of bacteria. In addition, the effects of glycan therapeutics on the native microbiota are determined by subjecting oral swab samples or tissue homogenates to DNA or RNA isolation and 16S or transcriptome analysis. Histopathological analysis is also performed to determine the extent of the disease state.

### Example 14. Effect of Glycan Preparations on Dental Biofilms and Tooth Decay

Dental biofilms (plaques) form on the surface of teeth and consist of multiple microbial species and their associated extracellular matrices. Bacteria growing in biofilms can display distinct metabolic and phenotypic properties compared with their planktonic (free floating) counterparts. The formation and microbial composition of biofilms on the surface of teeth have important implications for dental health; for example, plaques containing an overabundance of bacteria or an overabundance of acid-producing bacteria can result in the formation of dental caries (cavities). To identify beneficial populations of glycan preparations, in vitro models of dental biofilms are grown in the presence of glycan preparations and assayed for their cariogenic properties, growth, community composition, production of metabolites, and phenotypic or transcriptomic properties. Glycan preparations are selected based on their ability to elicit desired properties within the dental biofilm. This assay is followed by a step to ensure that the selected glycan preparations promote the growth of the healthy-state microbiota and/or the microbe(s) comprising a therapeutic composition without augmenting the growth of microbes associated with a disease state (e.g, Streptococcus mnutans, which is associated with dental caries). By testing glycan preparations against a panel of biofilm bacteria (individually or in groups) that are over- or underrepresented in a selected disease state, glycan preparations that selectively enhance the growth of healthy-state bacteria over disease-state bacteria are selected.

Dental biofilms are grown on solid supports coated with hydroxyapatite (to mimic the tooth surface) using the Calgary Biofilm Device (MBEC Assay; Innovotech) following the manufacturer's protocols. Alternatively, flow cell biofilm models or continuous biofilm models such as the artificial mouth model (AMM) are constructed according to established protocols (Salli & Ouwehand, The use of in vitro model systems to study dental biofilms associated with caries: a short review, 2015, Journal of Oral Microbiology, 7:26149) (Edlund et al., An in vitro biofilm model system maintaining a highly reproducible species and metabolic diversity approaching that of the human oral microbiome, 2013, Microbiome, 1:25). Alternatively, a model is used in which biofilms are grown on enamel slabs obtained from animal or human teeth (Steiner-Oliveira et al., An in vitro microbial model for producing caries-like lesions on enamel, 2007, Braz J Oral Sci, 6:1392). Microbial cultures used in the models include monocultures, mixed cultures, cultures isolated from humans or animals, cultures isolated from a human or animal and spiked with an isolate or collection of isolates, or cultures isolated from a human or animal and depleted of a collection of species (for example, by application of an antibiotic). Cultures include microbial species commonly used in in vitro dental biofilm models, such as Streptococcus oralis, Streptococcus sobrinus, Actinomyces naeslundii, Veillonella dispar, Fusobacterium nucleatum, and Candida albicans (Zurich biofilm model) and may also incorporate species associated with the formation of dental caries (S. mutans). Glycan preparations are prepared as concentrated stock solutions in sterile phosphate buffered saline (PBS) and added to the medium contacting the dental biofilms to achieve the desired working concentration. After a suitable incubation period at 37° C., the composition and properties of the dental biofilms are quantified using standard protocols. For in vitro models employing enamel slabs from animal or human teeth, the slabs are examined for caries-like lesions at the conclusion of the assay. In addition, this assay is performed in the presence of antibiotics or other test compounds. In addition, this assay is performed under conditions that simulate a cariogenic challenge by including 1% sucrose in the growth media (Koo et al., Exopolysaccharides Produced by Streptococcus mutans Glucosyltransferases Modulate the Establishment of Microcolonies within Multispecies Biofilms, 2010, Journal of Bacteriology, 192:3024).

### Example 15: Formulation and Efficacy of a Mouthwash for Treatment of Dental Caries and Periodontitis

This study is carried out to determine the effectiveness of administering exemplary glycan preparations (e.g., as described herein) in combination with fluoride and probiotics (e.g. beneficial bacteria) for the treatment of dental caries and/or periodontitis. A mouthwash solution comprising 1% of a glycan composition, sodium fluoride (0.05%, 0.02% w/v fluoride ion), probiotic strains (e.g., Lactobacillus casei, at 7.0×10⁹ viable cells per 50 mL solution), sorbitol, propylene glycol, methyl salicylate, flavoring agents (menthol), coloring agents (green 3, yellow 5), and preservatives (sodium benzoate, ethylenediaminetetraacetic acid, and cetylpyridinium chloride) in water is prepared and provided in 10 mL doses to 5 subjects experiencing dental caries and/or periodontitis. Five subjects will alternatively receive a vehicle, in which a similar mouthwash is prepared that comprises all of the above components except for the exemplary glycan composition. Subjects are instructed to swirl the mouthwash in the mouth twice daily for 30 seconds to achieve even distribution across the teeth and gums, then to eject the mouthwash without swallowing. Routine toothbrushing and oral care is encouraged over the course of the study. After 6 months, subjects will be examined for overall improvement in dental health, including progression of dental caries and periodontitis symptoms.

### Example 16: Formulation and Efficacy of a Lozenge for the Treatment of Periodontitis

This study will be conducted in order to evaluate the efficacy of a hard lozenge comprising exemplary glycan preparations (e.g., as described herein) to treat periodontitis. A thick syrup is prepared comprising up to 85% (e.g. between 50% and 85%)glycan composition, and optionally one or more of: additional thickeners, additional sweeteners, propylene glycol, pH adjusting agents (calcium carbonate, magnesium trisilicate), coloring agents (green 3, yellow 5), and preservatives (sodium benzoate, ethylenediamine, and cetylpyridinium chloride). The mixture is boiled and compounded to form individual hard lozenges using standard techniques. One (or more, e.g. 2-5) lozenge is provided per day to each of the subjects having periodontitis, while additional subjects each receive a vehicle with no glycan composition. Subjects are instructed to allow the lozenge to dissolve in the mouth and then not eat or drink anything for up to 30 minutes afterwards. Routine toothbrushing and oral care is encouraged over the course of the study. After 6 months, subjects will be examined for overall improvement in dental health, including progression of periodontitis symptoms.

### Example 17. Effect of Glycan Preparation on the Vaginal Microbiome

Glycan preparations are formulated in a liquid solution such that they are administered by a douche applicator or similar delivery device. Alternatively, glycan preparations are prepared in tablets, suppositories, or tampons such that they are introduced into the vagina. Sustained release of glycan preparations is achieved through their inclusion in a vaginal ring. Alternatively, oral delivery of glycan preparations in a liquid, tablet or capsule form provides indirect effects on the vaginal flora via metabolites formed by the gut microbiota or other modulation of the host by the gut microbiota. Subsequent to exposure of the vagina to glycan preparations, samples of vaginal fluid are collected under direct visualization from the posterior vaginal fornix using a sterile swab. The fluid is then analyzed for specific metabolites or microbiota population shifts by 16S rRNA gene sequencing, whole-genome sequencing, or RNA-Seq to determine the effect of the administered glycan preparations. The post-treatment samples are compared with pre-treatment samples. In this example, the effects of glycan preparations are assessed in human subjects. Otherwise healthy subjects diagnosed with bacterial vaginosis (BV) are recruited or included in a clinical study. Such subjects include those with newly diagnosed BV, recurrent BV, or BV associated with pregnancy. BV is diagnosed by satisfying three of four clinical (Amsel) criteria (vaginal pH >4.5, clue cells on saline microscopy >20% of epithelial cells, amine odor on addition of potassium hydroxide, and homogeneous vaginal discharge) present in vaginal fluid samples and Gram stain of vaginal fluid to confirm abnormal flora (Nugent score >3). Administration of glycan preparations is used in the absence of a co-treatment as determined by a physician. Alternatively, a glycan preparation is administered prior to, concurrent with or post treatment with standard-of-care treatments, as prescribed by a physician, such as oral or vaginally-applied antibiotics (including metronidazole, clindamycin, tinidazole, and secnidazole), an antifungal, or a vaginally-applied hormone, including estradiol, and probiotics. Within a suitable treatment period, the following changes are observed: resolution of symptoms of BV (assessed according to the standard diagnostic criteria listed above), an increase in the levels of Lactobacillus spp. in the vagina, and/or a decrease in total anaerobes, total Gram-negatives, Gardnerella vaginalis, Atopobium vaginae, or other bacteria associated with the disease state in the vagina.

### Example 18. Effect of Glycan Preparations in an Animal Model of Bacterial Vaginosis

Glycan preparations are tested in a mouse model of bacterial vaginosis elicited by Gardnerella. In this model (Gilbert N M, Lewis W G, Lewis A L (2013) Clinical Features of Bacterial Vaginosis in a Murine Model of Vaginal Infection with Gardnerella vaginalis. PLoS ONE 8(3): e59539), female C57/B16 mice are injected intraperitoneally with 0.5 mg β-estradiol in 100 μL filter-sterilized sesame oil three days prior to and on the day of inoculation in order to synchronize their estrus cycles. Mice are then inoculated vaginally with G. vaginalis in 20 μL sterile PBS. A streptomycin-resistant strain of G. vaginalis is used. For enumeration of G. vaginalis, vaginal washes are collected at various time points by flushing vaginas with 50 μL sterile PBS. G. vaginalis titers are determined from washes by preparing 10-fold serial dilutions in PBS (in the anaerobic chamber) and spotting 5 μL of each dilution in quadruplicate onto 1 mg/mL streptomycin selection plates (either Gardnerella semi-selective media or NYC-III agar). Colonies are then enumerated and reported as colony forming units (CFU) per mL of vaginal fluid. Enumeration of G. vaginalis is also assessed in the vaginal tissue and uterine horns. One uterine horn and half of the vagina (bisected longitudinally) from each mouse is homogenized followed by serial dilution and plating as for vaginal washes. Colonies are enumerated and reported as CFU per gram of tissue. Dosing of glycan preparations in mice in this model is performed by intravaginal administration of a liquid formulation, oral administration by either gavage or by inclusion of the glycan in the animals' drinking water or diet. Dosing frequency is variable, such as throughout the course of the study (i.e., during the estradiol treatment through to end of study) or in a “treatment” paradigm, where mice are administered glycan preparations only after G. gardnerella colonization has been established in the animals. Endpoints of the study include effects of glycan preparations versus placebo (control) on Gardnerella CFU counts in vaginal washes and tissues. Sialidase activity in the vaginal washes of the mice, a hallmark of the human disease, is also tested. High levels of sialidase is indicative of high colonization with Gardnerella.

### Example 19: Formulation and Efficacy of a Liquid Vaginal Spray for Treatment of Bacterial Vaginosis

A liquid spray for use in the treatment of bacterial vaginosis is formulated and used to treat females exhibiting symptoms of bacterial vaginosis. The following components are dissolved in mixture of water (8 mL) and benzyl alcohol (2 mL): an exemplary glycan composition (at a concentration of up to 3 g/ml, e.g., between 0.5 g/ml and 3 g/ml, alternatively up to 4 g/ml), lactic acid (4 mg), PEG 400 (126 mg), diethylene glycol monoethyl ether (80 mg), glycofurol (80 mg) and ethyl cellulose (8 mg). Each of the female patients are provided the liquid dosage form in a small spray bottle and are instructed to administer the spray every evening (or twice, three times or four times a day) for up to 2 weeks. Efficacy is determined by the suppression of symptoms after three to five days.

### Example 20: Formulation and Efficacy of a Vaginal Pessary for Treatment of Desquamative Inflammatory Vaginitis

This study is conducted in order to evaluate the efficacy of a vaginal pessary formulation comprising exemplary glycan preparations (e.g., as described herein) and optionally probiotics to treat desquamative inflammatory vaginitis. A pessary mixture is prepared comprising up to 75% (e.g. between 50% and 75%)glycan composition, optionally probiotic (beneficial) strains (e.g., Lactobacillus acidophilus, 1×10⁸ viable cells per 5 mL dose), and optionally one or more of: PEG-12, PEG-150, garlic bulb powder, and a scenting agent (rose flower oil) and filled into a soluble wax mold. Each of the subjects experiencing symptoms of desquamative inflammatory vaginitis is provided with a pessary and instructed to insert into the vagina daily and to leave it undisturbed overnight. Additional subjects each receive a vehicle with no glycan composition. Subjects are instructed to use the pessary once a day for 6 days total, and are evaluated for overall improvement in symptoms.

### Example 21. In Vitro Co-Culture Models to Test the Effect of Glycan Preparations on Host Responses to Bacterial Communities at Nasal, Oral, and Vaginal Sites

Bacteria can elicit both pro- and anti-inflammatory responses from host (mammalian) cells, and different bacterial species can elicit different host responses. Glycan preparations are used to alter the bacterial population to elicit a desired host response. An in vitro co-culture model is used to measure the host responses elicited by bacterial populations grown in the presence of glycan preparations. Glycan preparations that promote bacterial populations that elicit beneficial host responses or minimize detrimental host responses are selected using this assay.

Nasal: Primary nasal epithelial cells are obtained from human subjects by superficial nasal scrape biopsy and expanded (Müller et al., Culturing of Human Nasal Epithelial Cells at the Air Liquid Interface, 2013, Journal of Visualized Experiments, 80:e50646) (Comer et al., Comparison of Nasal and Bronchial Epithelial Cells Obtained from Patients with COPD, 2012, PLoS ONE, 7:e32924). Alternatively, cryopreserved human nasal epithelial cells are obtained from a vendor (for example, PromoCell) and cultured following the vendor-supplied protocols. Separately, bacterial cultures are grown in the presence of glycan preparations.

Oral: Primary human gingival epithelial cells (HGEC) are used in a co-culture assay (Guggenheim et al., In vitro modeling of host-parasite interactions: the ‘subgingival’ biofilm challenge of primary human epithelial cells, 2009, BMC Microbiology, 9:280). HGEC are isolated from gingival tissue biopsies obtained from human subjects undergoing periodontal procedures. HGEC are seeded on plastic tissue culture plates coated with type-I collagen (BD Biocoat) and maintained in KSFM media (Invitrogen). Alternatively, normal human oral keratinocytes (NHOK) with a buccal phenotype (EpiOral Tissue Model; MatTek Corporation, Ashland, Mass.) are cultured in antibiotic-free medium. Separately, bacterial cultures are grown in the presence of glycan preparations.

Vaginal: Epithelial cell lines or tissues from the female reproductive tract are used in a co-culture model (Fichorova et al., Novel Vaginal Microflora Colonization Model Providing New Insight into Microbicide Mechanism of Action, 2011, mBio, 2(6):e00168-11) (Anahtar et al., Cervicovaginal Bacteria Are a Major Modulator of Host Inflammatory Responses in the Female Genital Tract, 2015, Immunity, 42:965-976). Human immortalized endocervical (Endl/E6E7), ectocervical (Ectl/E6E7), and vaginal (Vk2/E6E7) epithelial cell lines are grown as monolayers in antibiotic-free keratinocyte serum-free medium (KSFM) (Invitrogen, Carlsbad, Calif.) supplemented with bovine pituitary extract, epidermal growth factor, and calcium chloride.

Alternatively, polarized tissues derived from primary human ectocervical epithelial cells grown on a permeable-membrane support (VEC-100; MatTek Corporation, Ashland, Mass.) are cultured in antibiotic-free medium. Separately, bacterial cultures are grown in the presence of populations of glycan preparations.

In all cases, after 16-24 hours of growth in the presence of glycan preparations, the bacterial suspensions are prepared in antibiotic-free media and added at 10⁴-10⁷ CFU/cm² to the human cell cultures. The co-cultures are incubated under aerobic conditions at 37° C. for 24 hours.

At the conclusion of the co-incubation period, the supernatant is collected and analyzed for inflammatory and immunomodulatory cytokines including IL-1α, IL-1β, TNF, IL-8, RANTES, IL-10, TGF-β, IFN-γ, IL-4, IL-6, IL-12, IL-17, and IL-23. This analysis is performed by enzyme linked immunosorbent assay (ELISA) or other comparable quantification technique (e.g., Luminex Assay; Life Technologies, Carlsbad, Calif.) following standard protocols. To analyze a broader range of responses, gene expression (e.g., by microarray) or transcriptomic (e.g., by RNA-Seq) analysis is performed by lysing the cells, purifying RNA, and following standard protocols. This procedure is used to analyze the expression of genes encoding inflammatory cytokines, immunomodulatory cytokines, antimicrobial peptides, and other relevant host responses.

### Example 22. Effect of Glycan Preparations on Gene Expression in a Mouse Model

The trial is conducted with two groups of mice. To the control group of mice, vehicle only is administered to either the nasal cavity, oral cavity, or vagina daily. To the treatment group of mice, vehicle containing the glycan preparation is administered to the nasal cavity, oral cavity, or vagina daily twice daily, daily, or 1-7 times per week. After 1-30 days the mice are sacrificed and the nasal tissue, components of the oral cavity (including, for example, the tongue, cheeks, and palate), and vaginal tissue is extracted and stored at −80° C. RNA is isolated from the tissues and converted to cDNA. The GeneChip Mouse Genome 430 2.0 Array (Affymetrix) is used to analyze the differential expression of approximately 14,000 murine genes. The experimental protocol and raw data analysis are performed according to the manufacturer's instructions and standard protocols. The biological function of the differentially expressed genes and their involvement in various processes are obtained from the following databases: RefGene (Reference for genes, proteins and antibodies; http://refgene.com/), CTD (Comparative Toxicogenomics Database; http://ctd.mdibl.org/), MGI (Mouse Genomics Informatics; http://www.informatics.jax.org/), KEGG (Kyoto Encyclopedia of Genes and Genomes; http://www.genome.jp/kegg/genes.html). This procedure is used to identify the differential expression of genes encoding inflammatory cytokines, immunomodulatory cytokines, antimicrobial peptides, and other relevant effector molecules.

TABLE 4
Genus level Microbial Constituents of the Nasal communities (nasal cavity/nares).

| Phylum | Class | Genus | Species |
|---|---|---|---|
| Actinobacteria | Actinobacteria | Corynebacterium, | Corynebacterium accolens, |
| | | Propionibacterium, | Corynebacterium |
| | | Tomitella | tuberculostearicum, |
| | | | Corynebacterium |
| | | | pseudodiphtericum, |
| | | | Corynebacterium mucifaciens, |
| | | | Mycobacterium fallax, |
| | | | Propionibacterium acnes |
| Firmicutes | Bacilli | Dolosigranulum, | Dolosigranulum pigrum, |
| | | Staphylococcus | Staphylococcus epidermidis, |
| | | | Staphylococcus aureus |
| | Clostridia | Anaerococcus, | Finegoldia magna |
| | | Finegoldia, | |
| | | Peptoniphilus | |
| | Gammaproteobacteria | Moraxella | Moraxella catarrhalis |

TABLE 5
Genus level Microbial Constituents of the Teeth (oral cavity)

| Phylum | Class | Genus |
|---|---|---|
| Actinobacteria | Actinobacteria | Actinomyces, Alloscardovia, Arthrobacter, |
| | | Atopobium, Bifidobacterium, Cellulomonas, |
| | | Collinsella, Corynebacterium, Gardnerella, |
| | | Microbacterium, Mobiluncus, Mycobacterium, |
| | | Olsenella, Parascardovia, Propionibacterium, |
| | | Pseudonocardia, Renibacterium, Rhodococcus, |
| | | Rothia, Scardovia, Slackia |
| Bacteroidetes | Bacteroidia | Alistipes, Bacteroides, Dysgonomonas, |
| | | Odoribacter, Parabacteroides, Porphyromonas, |
| | | Prevotella, Tannerella |
| | Flavobacteria | Capnocytophaga, Chryseobacterium, |
| | | Flavobacterium, Gillisia, Haloanella |
| | Sphingobacteria | Segetibacter, Sphingobacterium |
| Chloroflexi | Anaerolineae | SHD-231 |
| Firmicutes | Bacilli | Abiotrophia, Alicyclobacillus, Anoxybacillus, |
| | | Bacillus, Brevibacillus, Enterococcus, Gemella, |
| | | Geobacillus, Granulicatella, Jeotgalicoccus, |
| | | Lactobacillus, Lactococcus, Paenibacillus, |
| | | Staphylococcus, Streptococcus, Weissella |
| | Clostridia | Anaerococcus, Anaeroglobus, Anaerotruncus, |
| | | Anaerovorax, Bacteroides, Blautia, Butyrivibrio, |
| | | Catonella, Clostridium, Coprococcus, Dialister, |
| | | Dorea, Eubacterium, Faecalibacterium, Filifactor, |
| | | Finegoldia, Johnsonella, Lachnobacterium, |
| | | Lachnospira, Megamonas, Megasphaera, |
| | | Mitsuokella, Mogibacterium, Moryella, |
| | | Oribacterium, Oscillospira, Peptococcus, |
| | | Peptoniphilus, Peptostreptococcus, |
| | | Phascolarctobacterium, Pseudobutyrivibrio, |
| | | Pseudoramibacter, Roseburia, Ruminococcus, |
| | | Selenomonas, Shuttleworthia, Subdoligranulum, |
| | | Veillonella |
| Fusobacteria | Fusobacteria | Fusobacterium, Leptotrichia, Streptobacillus |
| Proteobacteria | Alphaproteobacteria | Afipia, Agrobacterium, Bradyrhizobium, |
| | | Brevundimonas, Erythrobacter, |
| | | Methylobacterium, Novosphingobium, |
| | | Paracoccus, Phenylobacterium, Phyllobacterium, |
| | | Rhodobacter, Rhodoplanes, Rubellimicrobium, |
| | | Sphingobium, Sphingomonas |
| | Betaproteobacteria | Acidovorax, Aquabacterium, Azohydromonas, |
| | | Brachymonas, Burkholderia, Comamonas, |
| | | Delftia, Diaphorobacter, Eikenella, |
| | | Herbaspirillum, Hydrogenophilus, Kingella, |
| | | Lautropia, Massilia, Methyloversatilis, Neisseria, |
| | | Paucibacter, Polaromonas, Ralstonia, |
| | | Rhodocyclus, Roseateles, Simonsiella, Sutterella, |
| | | Tepidimonas, Variovorax |
| | Deltaproteobacteria | Desulfobulbus, Desulfovibrio |
| | Epsilonproteobacteria | Campylobacter, Helicobacter, Wolinella |
| | Gammaproteobacteria | Acinetobacter, Actinobacillus, Aggregatibacter, |
| | | Cardiobacterium, Citrobacter, Dichelobacter, |
| | | Erwinia, Escherichia, Haemophilus, Klebsiella, |
| | | Luteibacter, Marinomonas, Moraxella, Nevskia, |
| | | Providencia, Pseudomonas, Raoultella, Serratia, |
| | | Stenotrophomonas, Thermomonas, Trabulsiella |
| Spirochaetes | Spirochaetes | Treponema |
| Synergistetes | Synergistetia | TG5 |
| Tenericutes | Erysipelotrichi | Bulleidia, Clostridium, Coprobacillus, |
| | | Holdemania, Sharpea |
| | Mollicutes | Asteroleplasma, Mycoplasma |
| Thermi | Deinococci | Deinococcus |
| Verrucomicrobia | Verrucomicrobiae | Akkermansia |
| Euryarchaeota | Methanobacteria | Methanobrevibacter |

TABLE 6
Genus level Microbial Constituents of the Mouth (oral cavity)

| Phylum | Class | Genus |
|---|---|---|
| Actinobacteria | Actinobacteria | Actinomyces, Adlercreutzia, Alloscardovia, |
| | | Arthrobacter, Atopobium, Bifidobacterium, |
| | | Collinsella, Corynebacterium, Demequina, |
| | | Eggerthella, Gardnerella, Geodermatophilus, |
| | | Microbacterium, Micrococcus, Mobiluncus, |
| | | Mycobacterium, Nesterenkonia, Olsenella, |
| | | Propionibacterium, Rathayibacter, |
| | | Renibacterium, Rhodococcus, Rothia, Scardovia, |
| | | Slackia |
| Bacteroidetes | Bacteroidia | Alistipes, Bacteroides, Dysgonomonas, |
| | | Odoribacter, Parabacteroides, Porphyromonas, |
| | | Prevotella, Tannerella |
| | Flavobacteria | Capnocytophaga, Chryseobacterium, |
| | | Elizabethkingia, Flavobacterium, Haloanella, |
| | | Wautersiella |
| | Sphingobacteria | Sphingobacterium |
| Chloroflexi | Anaerolineae | SHD-231 |
| Cyanobacteria | Oscillatoriophycideae | Chroococcidiopsis |
| Firmicutes | Bacilli | Abiotrophia, Aerococcus, Alicyclobacillus, |
| | | Anoxybacillus, Bacillus, Brochothrix, |
| | | Carnobacterium, Enterococcus, Gemella, |
| | | Geobacillus, Granulicatella, Jeotgalicoccus, |
| | | Lactobacillus, Lactococcus, Leuconostoc, |
| | | Listeria, Melissococcus, Paenibacillus, |
| | | Planomicrobium, Staphylococcus, Streptococcus, |
| | | Thermicanus, Turicibacter, Weissella |
| | Clostridia | Acidaminococcus, Anaerococcus, Anaeroglobus, |
| | | Anaerostipes, Anaerovorax, Bacteroides, |
| | | Bacteroides, Blautia, Butyrivibrio, Catonella, |
| | | Clostridium (families Clostridiaceae, |
| | | Lachnospiraceae, Ruminococcaceae), |
| | | Coprococcus, Dialister, Dorea, Eubacterium |
| | | (families ClostridialesFamilyXIII.IncertaeSedis, |
| | | Eubacteriaceae, Lachnospiraceae, |
| | | Ruminococcaceae), Faecalibacterium, Filifactor, |
| | | Finegoldia, Johnsonella, Lachnobacterium, |
| | | Lachnospira, Megamonas, Megasphaera, |
| | | Mitsuokella, Mogibacterium, Moryella, |
| | | Oribacterium, Oscillospira, Peptococcus, |
| | | Peptoniphilus, Peptostreptococcus, |
| | | Phascolarctobacterium, Propionispora, |
| | | Pseudoramibacter, Roseburia, Ruminococcus |
| | | (families Lachnospiraceae, Ruminococcaceae), |
| | | Selenomonas, Shuttleworthia, Subdoligranulum, |
| | | Veillonella |
| Fusobacteria | Fusobacteria | Fusobacterium, Leptotrichia, Sneathia, |
| | | Streptobacillus |
| Proteobacteria | Alphaproteobacteria | Afipia, Agrobacterium, Bosea, Bradyrhizobium, |
| | | Brevundimonas, Hyphomicrobium, |
| | | Mesorhizobium, Methylobacterium, |
| | | Novosphingobium, Paracoccus, |
| | | Phenylobacterium, Phyllobacterium, |
| | | Rhodobacter, Roseomonas, Rubellimicrobium, |
| | | Skermanella, Sphingobium, Sphingomonas |
| | Betaproteobacteria | Achromobacter, Acidovorax, Aquabacterium, |
| | | Azospira, Brachymonas, Burkholderia, |
| | | Comamonas, Cupriavidus, Delftia, |
| | | Diaphorobacter, Eikenella, Herbaspirillum, |
| | | Janthinobacterium, Kingella, Lautropia, Massilia, |
| | | Methylophilus, Methyloversatilis, Neisseria, |
| | | Paucibacter, Ralstonia, Rhodocyclus, Roseateles, |
| | | Simonsiella, Stenoxybacter, Sutterella, |
| | | Tepidimonas, Thauera, Variovorax, Zoogloea |
| | Deltaproteobacteria | Bilophila, Desulfobulbus |
| | Epsilonproteobacteria | Arcobacter, Campylobacter, Helicobacter, |
| | | Wolinella |
| | Gammaproteobacteria | Acinetobacter, Actinobacillus, Aggregatibacter, |
| | | Cardiobacterium, Citrobacter, Dichelobacter, |
| | | Escherichia, Haemophilus, Halomonas, |
| | | Klebsiella, Luteibacter, Moraxella, Nevskia, |
| | | Pantoea, Proteus, Providencia, Pseudomonas, |
| | | Pseudoxanthomonas, Psychrobacter, Raoultella, |
| | | Serratia, Shewanella, Stenotrophomonas, |
| | | Succinivibrio, Tolumonas, Trabulsiella |
| Spirochaetes | Spirochaetes | Treponema |
| Synergistetes | Synergistetia | TG5 |
| Tenericutes | Erysipelotrichi | Bulleidia, Catenibacterium, Clostridium, |
| | | Coprobacillus, Erysipelothrix, Holdemania, |
| | | RFN20, Sharpea |
| | Mollicutes | Asteroleplasma, Mycoplasma, Ureaplasma |
| Thermi | Deinococci | Deinococcus, Meiothermus, Thermus |
| Verrucomicrobia | Verrucomicrobiae | Akkermansia |
| Euryarchaeota | Methanobacteria | Methanobrevibacter |

TABLE 7
Genus level Microbial Constituents of the Vaginal communities

| Phylum | Class | Genus |
|---|---|---|
| Actinobacteria | Actinobacteria | Actinobaculum, Actinomyces, Actinoplanes, |
| | | Adlercreutzia, Alloscardovia, Arcanobacterium, |
| | | Atopobium, Bifidobacterium, Brachybacterium, |
| | | Brevibacterium, Collinsella, Corynebacterium, |
| | | Dermabacter, Dietzia, Eggerthella, Gardnerella, |
| | | Kocuria, Microbacterium, Micrococcus, |
| | | Mobiluncus, Mycobacterium, Phycicoccus, |
| | | Propionibacterium, Pseudoclavibacter, |
| | | Renibacterium, Rhodococcus, Rothia, Slackia, |
| | | Tessaracoccus, Varibaculum, Williamsia |
| Bacteroidetes | Bacteroidia | Alistipes, Bacteroides, Dysgonomonas, |
| | | Odoribacter, Parabacteroides, Porphyromonas, |
| | | Prevotella, Tannerella |
| | Flavobacteria | Bergeyella, Capnocytophaga, Chryseobacterium, |
| | | Elizabethkingia, Flavobacterium, Wautersiella |
| | Sphingobacteria | Pedobacter, Sphingobacterium |
| | Bacilli | Abiotrophia, Aerococcus, Alicyclobacillus, |
| | | Bacillus, Enterococcus, Facklamia, Gemella, |
| | | Granulicatella, Lactobacillus, Listeria, |
| | | Melissococcus, Planomicrobium, |
| | | Staphylococcus, Streptococcus, Thermicanus, |
| | | Turicibacter |
| Firmicutes | Clostridia | Acidaminococcus, Anaerococcus, Anaeroglobus, |
| | | Anaerotruncus, Anaerovorax, Bacteroides, |
| | | Blautia, Catonella, Clostridium (families |
| | | Clostridiaceae, Lachnospiraceae, |
| | | Ruminococcaceae), Coprococcus, |
| | | Dehalobacterium, Dialister, Dorea, Eubacterium |
| | | (families Clostridiales FamilyXIII Incertae Sedis, |
| | | Eubacteriaceae, Lachnospiraceae, |
| | | Ruminococcaceae), Faecalibacterium, Finegoldia, |
| | | Helcococcus, Lachnobacterium, Lachnospira, |
| | | Megamonas, Megasphaera, Mitsuokella, |
| | | Mogibacterium, Moryella, Oribacterium, |
| | | Oscillospira, Peptococcus, Peptoniphilus, |
| | | Peptostreptococcus, Phascolarctobacterium, |
| | | Pseudoramibacter, Roseburia, Ruminococcus |
| | | (families Lachnospiraceae, Ruminococcaceae), |
| | | Selenomonas, Shuttleworthia, Subdoligranulum, |
| | | Veillonella |
| Fusobacteria | Fusobacteria | Fusobacterium, Leptotrichia, Sneathia, |
| | | Streptobacillus |
| Proteobacteria | Alphaproteobacteria | Afipia, Bosea, Bradyrhizobium, Brevundimonas, |
| | | CandidatusOdyssella, Kaistobacter, |
| | | Methylobacterium, Novosphingobium, |
| | | Paracoccus, Phenylobacterium, Phyllobacterium, |
| | | Rhodobacter, Rhodoplanes, Roseomonas, |
| | | Sphingobium, Sphingomonas, Sphingopyxis |
| | Betaproteobacteria | Achromobacter, Acidovorax, Aquabacterium, |
| | | Burkholderia, Comamonas, Cupriavidus, Delftia, |
| | | Diaphorobacter, Eikenella, Herbaspirillum, |
| | | Hydrogenophilus, Kingella, Lautropia, Massilia, |
| | | Methylophilus, Methyloversatilis, Neisseria, |
| | | Oligella, Pandoraea, Paucibacter, Ralstonia, |
| | | Rhodocyclus, Roseateles, Sutterella, Variovorax, |
| | | Zoogloea |
| | Deltaproteobacteria | Bilophila, Desulfovibrio, Geobacter |
| | Epsilonproteobacteria | Campylobacter, Helicobacter |
| | Gammaproteobacteria | Acinetobacter, Actinobacillus, Aeromonas, |
| | | Aggregatibacter, Cardiobacterium, Citrobacter, |
| | | Erwinia, Escherichia, Haemophilus, Klebsiella, |
| | | Luteibacter, Moraxella, Nevskia, Photobacterium, |
| | | Proteus, Providencia, Pseudomonas, |
| | | Psychrobacter, Raoultella, Serratia, |
| | | Stenotrophomonas, Trabulsiella, Xanthomonas |
| Spirochaetes | Spirochaetes | Treponema |
| Synergistetes | Synergistetia | Jonquetella, Pyramidobacter, TG5 |
| Tenericutes | Erysipelotrichi | Bulleidia, Catenibacterium, Clostridium, |
| | | Coprobacillus, Holdemania |
| | Mollicutes | Asteroleplasma, Mycoplasma, Ureaplasma |
| Thermi | Deinococci | Deinococcus, Thermus |
| Verrucomicrobia | Verrucomicrobiae | Akkermansia |

TABLE 8
Microbial Metabolites

2-hydroxyisobutyrate, 3-hydroxyisovalerate, 3-methyl-crotonylglycine, 3-
methylcrotonylglycine, allantoin, betaine, formate, mannitol, p-cresol glucuronide,
phenylacetylglycine, sarcosine, taurine, acetic acid, acetylaldehyde, ascorbic acid, butanedione,
butyric acid, deoxycholic acid, ethylphenyl sulfate, formic acid/formate, indole, isobutyric acid,
isovaleric acid, propionic acid, serotonin, succinic acid/succinate, TMAO, tryptophan, valeric
acid, ursodeoxycholic acid, lactate, lactic acid, hydrogen peroxide

TABLE 9
Polyphenols

| Polyphenol Sub- | |
|---|---|
| Class | Compound Name |
| Anthocyanins | Malvidin 3-O-(6″-p-coumaroyl-glucoside), Cyanidin, total, Delphinidin 3-O- |
| | (6″-acetyl-galactoside), Cyanidin 3-O-(6″-acetyl-galactoside), Malvidin, |
| | Cyanidin 3-O-galactoside, Cyanidin 3-O-glucoside, Cyanidin 3-O-rutinoside, |
| | Cyanidin 3-O-sophoroside, Pelargonidin 3-O-glucoside, Cyanidin 3-O-(6″- |
| | malonyl-glucoside), Peonidin, Peonidin 3-O-glucoside, Peonidin 3-O- |
| | rutinoside, Pelargonidin 3-O-rutinoside, Pelargonidin, Cyanidin, Malvidin |
| | 3,5-O-diglucoside, Cyanidin 3-O-glucosyl-rutinoside, Pelargonidin 3-O- |
| | sophoroside, Pelargonidin 3-O-glucosyl-rutinoside, Cyanidin 3-O-(6″- |
| | succinyl-glucoside), Pelargonidin 3-O-(6″-succinyl-glucoside), Delphinidin, |
| | Delphinidin 3-O-galactoside, Delphinidin 3-O-glucoside, Delphinidin 3-O- |
| | arabinoside, Petunidin, Petunidin 3-O-galactoside, Cyanidin 3-O-arabinoside, |
| | Petunidin 3-O-glucoside, Peonidin 3-O-galactoside, Petunidin 3-O- |
| | arabinoside, Malvidin 3-O-glucoside, Malvidin 3-O-arabinoside, Cyanidin 3- |
| | O-(6″-acetyl-arabinoside), Delphinidin 3-O-(6″-acetyl-glucoside), Petunidin |
| | 3-O-(6″-acetyl-galactoside), Peonidin 3-O-(6″-acetyl-galactoside), Cyanidin |
| | 3-O-(6″-acetyl-glucoside), Malvidin 3-O-(6″-acetyl-galactoside), Petunidin 3- |
| | O-(6″-acetyl-glucoside), Polymeric anthocyanins, total, Malvidin 3-O-(6″- |
| | acetyl-glucoside), Peonidin 3-O-(6″-acetyl-glucoside), Pelargonidin 3-O- |
| | arabinoside, Delphinidin 3-O-rutinoside, Cyanidin 3-O-sambubioside, |
| | Pelargonidin 3-O-(6″-malonyl-glucoside), Peonidin 3-O-(6″-p-coumaroyl- |
| | glucoside), Cyanidin 3-O-xyloside, Malvidin 3-O-galactoside, Peonidin 3-O- |
| | arabinoside, Petunidin 3-O-rutinoside, Delphinidin 3-O-xyloside, Petunidin |
| | 3-O-(6″-p-coumaroyl-glucoside), Pelargonidin 3-O-galactoside, Pelargonidin |
| | 3-O-sambubioside, Delphinidin 3-O-sambubioside, Cyanidin 3-O-xylosyl- |
| | rutinoside, Vitisin A, Delphinidin 3-O-(6″-p-coumaroyl-glucoside), Pigment |
| | A, p-Coumaroyl vitisin A, Acetyl vitisin A, Cyanidin 3-O-(6″-p-coumaroyl- |
| | glucoside), Cyanidin 3-O-sambubioside 5-O-glucoside, Cyanidin 3-O-(6″- |
| | caffeoyl-glucoside), Cyanidin 3,5-O-diglucoside, Pinotin A, Delphinidin 3,5- |
| | O-diglucoside, Pelargonidin 3,5-O-diglucoside, Malvidin 3-O-(6″-caffeoyl- |
| | glucoside), Cyanidin 3-O-(6″-dioxalyl-glucoside), Cyanidin 3-O- |
| | laminaribioside, Cyanidin 3-O-(3″-malonyl-glucoside), Peonidin 3-O-(6″- |
| | malonyl-glucoside), Cyanidin 3-O-(6″-malonyl-laminaribioside), Cyanidin 3- |
| | O-dimalonyl-laminaribioside, Cyanidin 3-O-(6″-malonyl-arabinoside), |
| | Delphinidin 3-O-glucosyl-glucoside, Cyanidin 3-O-(6″-malonyl-3″-glucosyl- |
| | glucoside), Cyanidin 3-O-(2″-xylosyl-6″-glucosyl-galactoside), Cyanidin 3- |
| | O-(2″-xylosyl-6″-(6″′-caffeoyl-glucosyl)-galactoside), Cyanidin 3-O-(2″- |
| | xylosyl-galactoside), Cyanidin 3-O-(2″-xylosyl-6″-(6″′-p-hydroxybenzoyl- |
| | glucosyl)-galactoside), Cyanidin 3-O-(2″-xylosyl-6″-(6″′-sinapoyl-glucosyl)- |
| | galactoside), Cyanidin 3-O-(2″-xylosyl-6″-(6″′-feruloyl-glucosyl)- |
| | galactoside), Cyanidin 3-O-(2″-xylosyl-6″-(6″′-p-coumaroyl-glucosyl)- |
| | galactoside), Delphinidin 3-O-(6″-malonyl-glucoside), Malvidin 3-O- |
| | rutinoside, Luteolinidin 3-O-glucoside, Delphinidin 3-O-feruloyl-glucoside, |
| | Petunidin 3,5-O-diglucoside, Petunidin 3-O-rhamnoside, Luteolinidin, Vitisin |
| | A aglycone, Pigment A aglycone, Pinotin A aglycone, 4-O-Methylcyanidin |
| | 3-O-galactoside, Malvidin 3-O-(6″-O-acetyl)-glucoside, Cyanidin 3-O- |
| | diglucoside-5-O-glucoside, Peonidin 3-O-diglucoside-5-O-glucoside, |
| | Peonidin 3,5-O-diglucoside, Peonidin 3-O-(2-O-(6-O-(E)-caffeoyl-D- |
| | glucosyl)-D-glucoside)-5-O-D-glucoside, Peonidin 3-O-sophoroside, |
| | Peonidin 3-O-sambubioside, Peonidin 3-O-sambubioside-5-O-glucoside, |
| | Peonidin 3-O-xyloside, 4′-O-Methylcyanidin 3-O-D-glucoside, Cyanidin 3- |
| | O-glucuronide, Cyanidin 3-O-(3″,6″-O-dimalonyl-glucoside), Cyanidin 3- |
| | sulfate, 4-O-Methyldelphinidin 3-O-L-arabinoside, 4-O-Methyldelphinidin 3- |
| | O-D-glucoside, Isopeonidin 3-O-arabinoside, Isopeonidin 3-O-galactoside, |
| | Isopeonidin 3-O-glucoside, Isopeonidin 3-O-rutinoside, Isopeonidin 3-O- |
| | sambubioside, Isopeonidin 3-O-xyloside, 4-O-Methylpetunidin 3-O-D- |
| | galactoside, 4-O-Methylpetunidin 3-O-D-glucoside, Cyanidin 3-O-(2-O-(6- |
| | O-(E)-caffeoyl-D glucoside)-D-glucoside)-5-O-D-glucoside, 4′-O- |
| | Methyldelphinidin 3-O-rutinoside, Pelargonidin 3-O-(6″-acetyl-glucoside) |
| Chalcones | Chalconaringenin, total, Butein, Xanthohumol, Chalconaringenin, |
| | Chalconaringenin 2′-O-glucuronide, Chalconaringenin 4′-O-glucuronide, |
| | Chalconaringenin 7-O-glucuronide |
| _Dihydro- | Phloretin, Phloridzin, Phloretin xylosyl-galactoside, Phloretin 2′-O-xylosyl- |
| chalcones | glucoside, 3-Hydroxyphloretin 2′-O-xylosyl-glucoside, 3-Hydroxyphloretin |
| | 2′-O-glucoside, Phloridzin, total, 3-Hydroxyphloretin, Phloretin 2′-O- |
| | glucuronide, 3-Methoxyphloretin 3′-O-glucoside, 3-Hydroxy-4-O- |
| | methylphloretin 3′-O-glucoside, 3-Hydroxyphloretin 3′-O-glucoside |
| Dihydro- | Dihydroquercetin 3-O-rhamnoside, Dihydroquercetin, Engeletin, |
| flavonols | Dihydromyricetin 3-O-rhamnoside, Dihydroquercetin 3-O-glucoside, |
| | Dihydromyricetin, Dihydrokaempferol |
| Flavanols | (+)-Catechin, (−)-Epicatechin, (+)-Gallocatechin, (−)-Epigallocatechin, (−)- |
| | Epicatechin 3-O-gallate, (−)-Epigallocatechin 3-O-gallate, Catechins, total, |
| | Theaflavins, total, Thearubigins, total, Theaflavin, Theaflavin 3-O-gallate, |
| | Theaflavin 3′-O-gallate, Theaflavin 3,3′-O-digallate, (+)-Gallocatechin 3-O- |
| | gallate, (−)-Catechin, (+)-Catechin 3-O-gallate, Theaflavic acid, Epitheaflavic |
| | acid, Epitheaflavic acid 3′-O-gallate, Isoneotheaflavin 3-O-gallate, (−)- |
| | Gallocatechin 3-O-gallate, (−)-Gallocatechin, (−)-Catechin 3-O-gallate, (+)- |
| | Epicatechin, (−)-Epicatechin 8-C-galactoside, Isoneotheaflavin, Procyanidin |
| | dimer B1, Procyanidin dimer B2, Procyanidin dimer B3, Procyanidin dimer |
| | B4, Procyanidin dimer B5, Procyanidin dimer B7, Prodelphinidin dimer B3, |
| | Procyanidin trimer C1, Procyanidin tetramer T4, 02 mers, Procyanidins, total, |
| | Procyanidin trimer EEC, 01 mers, Polymers (>10 mers), 03 mers, 04-06 |
| | mers, 07-10 mers, Procyanidin dimer B6, Procyanidin trimer T2, Procyanidin |
| | trimer C2, Procyanidin dimer B2 3-O-gallate, Procyanidin dimer B2 3′-O- |
| | gallate, Procyanidin dimer B1 3-O-gallate, Prodelphinidin trimer GC-GC-C, |
| | Procyanidin trimer T3, 04 mers, Procyanidin dimer A2, 05 mers, 06 mers, 07 |
| | mers, 08 mers, 09 mers, 10 mers, 02-03 mers, (+)-Epicatechin-(2a-7)(4a-8)- |
| | catechin 3-O-arabinoside, Cinnamtannin B1 3-O-galactoside, (+)- |
| | Epicatechin-(2a-7)(4a-8)-epicatechin 3-O-arabinoside, Cinnamtannin B1 3- |
| | O-arabinoside, Procyanidin dimer A1, Cinnamtannin B1, Proanthocyanidins, |
| | total, Prodelphinidin trimer GC-C-C, Prodelphinidin trimer C-GC-C, (+)- |
| | Epicatechin-(2a-7)(4a-8)-catechin, (+)-Epicatechin-(2a-7)(4a-8)-epicatechin, |
| | (−)-Epicatechin-(2a-7)(4a-8)-epicatechin 3-O-galactoside, Cinnamtannin A2, |
| | Bis-8,8′-Catechinylmethane, Cinnamtannin A3, (+)-Catechin 3-O-glucose, 3′- |
| | O-Methylepicatechin, 4′-O-Methyl-(−)-epicatechin 3′-O-glucuronide, |
| | Epicatechin 3′-O-glucuronide, Epigallocatechin 3-O-gallate-4″-O- |
| | glucuronide, 3′-O-Methylcatechin, 3′-O-Methyl-(−)-epicatechin 3-O-gallate, |
| | 4′,4″-O-Dimethylepigallocatechin 3-O-gallate, 4′-O-Methylepigallocatechin, |
| | 4″-O-Methylepigallocatechin 3-O-gallate, 4′-O-Methylepicatechin, |
| | Epigallocatechin 3-O-gallate-7-O-glucoside-4″-O-glucuronide, Theasinensin |
| | A, 3-O-Methylepigallocatechin, 3′,4″-Dimethyl-(−)-epicatechin 3-O-gallate, (−)- |
| | Epigallocatechin 3-O-glucuronide, 3′-O-Methyl-(−)-epigallocatechin 3-O- |
| | gallate, 3″-O-Methyl-(−)-epigallocatechin 3-O-gallate, 3′,3″-O-Dimethyl-(−)- |
| | epigallocatechin 3-O-gallate, 3′-O-Methyl-(−)-epicatechin 7-O-glucuronide, |
| | Epicatechin 7-O-glucuronide, (−)-Epigallocatechin 3′-O-glucuronide, (−)- |
| | Epigallocatechin 7-O-glucuronide, 4′-O-Methyl-(−)-epigallocatechin 3′-O- |
| | glucuronide, 4′-O-Methyl-(−)-epigallocatechin 7-O-glucuronide, 4′-O-Methyl- |
| | (−)-epigallocatechin 3′-sulfate |
| Flavanones | Naringenin, Eriodictyol, Hesperetin, Hesperetin, total, Naringenin, total, |
| | Eriocitrin, Hesperidin, Naringin, Narirutin, Neoeriocitrin, Neohesperidin, |
| | Isosakuranetin 7-O-rutinoside, Poncirin, Didymin, Narirutin 4′-O-glucoside, |
| | Naringin 4′-O-glucoside, Naringin 6′-malonate, Isosakuranetin, Naringenin 7- |
| | O-glucoside, Pinocembrin, 8-Prenylnaringenin, 6-Prenylnaringenin, 6- |
| | Geranylnaringenin, Isoxanthohumol, Eriodictyol 7-O-glucoside, Sakuranetin, |
| | Hesperetin 3′-O-glucuronide, Hesperetin 7-O-glucuronide, Hesperetin 3′- |
| | sulfate, Hesperetin 7-sulfate, Homoeriodictyol, Naringenin 4′-O-glucuronide, |
| | Naringenin 5-O-glucuronide, Naringenin 7-O-glucuronide, Hesperetin 3′,7-O- |
| | diglucuronide, Hesperetin 5,7-O-diglucuronide, Pinobanksin, 5-O- |
| | Methylpinobanksin |
| Flavones | Apigenin, Luteolin, Apigenin, total, Luteolin, total, Diosmin, Isorhoifolin, |
| | Neodiosmin, Rhoifolin, Sinensetin, Nobiletin, Tangeretin, Luteolin 7-O- |
| | diglucuronide, Chrysin, Diosmetin, Acacetin, Luteolin 7-O-rutinoside, |
| | Tetramethylscutellarein, Luteolin 7-O-glucoside, Apigenin 7-O-glucoside, |
| | Apigenin 6,8-di-C-glucoside, Sinensetin, total, Apigenin 6,8-C-arabinoside- |
| | C-glucoside, Apigenin 6,8-C-galactoside-C-arabinoside, Luteolin 7-O- |
| | glucuronide, Apigenin 7-O-glucuronide, Luteolin 7-O-malonyl-glucoside, |
| | Luteolin 6-C-glucoside, Luteolin 8-C-glucoside, Luteolin 6-C-glucoside 8-C- |
| | arabinoside, Luteolin 7-O-(2-apiosyl-glucoside), Luteolin 7-O-(2-apiosyl-4- |
| | glucosyl-6-malonyl)-glucoside, Apigenin 6-C-glucoside 8-C-arabinoside, |
| | Luteolin 7-O-(2-apiosyl-6-malonyl)-glucoside, Apigenin 7-O-apiosyl- |
| | glucoside, Apigenin 8-C-glucoside, 7,3′,4′-Trihydroxyflavone, 7,4′- |
| | Dihydroxyflavone, Geraldone, Baicalein, Apigenin 6-C-glucoside, |
| | Hispidulin, Cirsimaritin, Luteolin 4′-O-glucoside, 5,6-Dihydroxy-7,8,3′,4′- |
| | tetramethoxyflavone, Pebrellin, Gardenin B, Nepetin, Jaceosidin, Cirsilineol, |
| | Eupatorin, 6-Hydroxyluteolin, 6-Hydroxyluteolin 7-O-rhamnoside, |
| | Scutellarein, Apigenin 7-O-(6″-malonyl-apiosyl-glucoside), Chrysoeriol, |
| | Chrysoeriol 7-O-apiosyl-glucoside, Chrysoeriol 7-O-(6″-malonyl-apiosyl- |
| | glucoside), Chrysoeriol 7-O-glucoside, Chrysoeriol 7-O-(6″-malonyl- |
| | glucoside), Apigenin 7-O-diglucuronide, Rhoifolin 4′-O-glucoside, 3′-O- |
| | Demethylnobiletin, 4′-O-Demethylnobiletin, 6-O-Demethyleupatilin, 6-O- |
| | Methylscutellarin, Apigenin 4′-O-glucuronide, Apigenin 5-O-glucuronide, |
| | Eupatilin, Isoscutellarein, Scutellarein 4′-O-glucuronide, Scutellarein 5-O- |
| | glucuronide, Scutellarein 6,7-O-diglucuronide, Scutellarein 6-O-glucuronide, |
| | Scutellarein 7-sulfate, Scutellarein 7-O-glucuronide, Tricin, 6-O- |
| | Methylscutellarein |
| Flavonols | Kaempferol, Quercetin, Quercetin 3-O-galactoside, Quercetin 3-O-glucoside, |
| | Quercetin 3-O-xyloside, Quercetin 3-O-rhamnoside, Quercetin 3-O- |
| | rutinoside, Quercetin 3-O-sophoroside, Quercetin 3-O-arabinoside, Quercetin |
| | 3-O-xylosyl-glucuronide, Quercetin, total, Kaempferol, total, Myricetin, total, |
| | Isorhamnetin 3-O-glucoside 7-O-rhamnoside, Isorhamnetin 3-O-rutinoside, |
| | Kaempferol 3-O-glucuronide, Isorhamnetin 7-O-rhamnoside, Quercetin 3,4′- |
| | O-diglucoside, Myricetin 3-O-rutinoside, Myricetin, Morin, Kaempferide, |
| | Myricetin 3-O-galactoside, Myricetin 3-O-glucoside, Quercetin 3-O- |
| | glucosyl-xyloside, Quercetin 3-O-acetyl-rhamnoside, Kaempferol 3-O- |
| | galactoside, Galangin, Isorhamnetin, Kaempferol 3-O-glucoside, Kaempferol |
| | 3-O-rutinoside, Kaempferol 3-O-glucosyl-rhamnosyl-galactoside, |
| | Kaempferol 3-O-glucosyl-rhamnosyl-glucoside, Quercetin 3-O-glucosyl- |
| | rhamnosyl-galactoside, Quercetin 3-O-glucosyl-rhamnosyl-glucoside, |
| | Rhamnetin, Isorhamnetin 3-O-glucoside, Myricetin 3-O-rhamnoside, |
| | Quercetin 3-O-rhamnosyl-galactoside, Kaempferol 3-O-arabinoside, |
| | Quercetin 3-O-glucuronide, Isorhamnetin 3-O-glucuronide, Myricetin 3-O- |
| | arabinoside, Quercetin 3,7,4′-O-triglucoside, Quercetin 7,4′-O-diglucoside, |
| | Quercetin 4′-O-glucoside, Isorhamnetin 4′-O-glucoside, 3,7- |
| | Dimethylquercetin, Kaempferol 3-O-sophoroside, Kaempferol 3,7-O- |
| | diglucoside, Quercetin 3-O-diglucoside, Kaempferol 3-O-sophoroside 7-O- |
| | glucoside, Kaempferol 3-O-sophorotrioside 7-O-sophoroside, Kaempferol 3- |
| | O-sinapoyl-caffeoyl-sophoroside 7-O-glucoside, Kaempferol 3-O-feruloyl- |
| | caffeoyl-sophoroside 7-O-glucoside, Kaempferol 3-O-feruloyl- |
| | sophorotrioside, Kaempferol 3-O-sinapoyl-sophoroside 7-O-glucoside, |
| | Kaempferol 3-O-caffeoyl-sophoroside 7-O-glucoside, Kaempferol 3-O- |
| | feruloyl-sophoroside 7-O-glucoside, Quercetin 3-O-(6″-malonyl-glucoside), |
| | Kaempferol 3-O-(6″-malonyl-glucoside), Kaempferol 3-O-rhamnoside, |
| | Quercetin 3-O-(6″-malonyl-glucoside) 7-O-glucoside, Patuletin, |
| | Quercetagetin, Spinacetin, Patuletin 3-O-glucosyl-(1−>6)-[apiosyl(1−>2)]- |
| | glucoside, Spinacetin 3-O-glucosyl-(1−>6)-[apiosyl(1−>2)]-glucoside, |
| | Patuletin 3-O-(2″-feruloylglucosyl)(1−>6)-[apiosyl(1−>2)]-glucoside, |
| | Spinacetin 3-O-(2″-p-coumaroylglucosyl)(1−>6)-[apiosyl(1−>2)]-glucoside, |
| | Spinacetin 3-O-(2″-feruloylglucosyl)(1−>6)-[apiosyl(1−>2)]-glucoside, |
| | Spinacetin 3-O-glucosyl-(1−>6)-glucoside, Jaceidin 4′-O-glucuronide, 5,3′,4′- |
| | Trihydroxy-3-methoxy-6:7-methylenedioxyflavone 4′-O-glucuronide, 5,4′- |
| | Dihydroxy-3,3′-dimethoxy-6:7-methylenedioxyflavone 4′-O-glucuronide, |
| | Spinatoside, Spinatoside 4′-O-glucuronide, Kaempferol 3-O-xylosyl- |
| | glucoside, Kaempferol 3-O-acetyl-glucoside, Quercetin 3-O-xylosyl- |
| | rutinoside, Kaempferol 3-O-xylosyl-rutinoside, Quercetin 3-O-glucosyl- |
| | glucoside, Quercetin 7-O-glucoside, Quercetin 3-O-(6″-acetyl-glucoside), |
| | Kaempferol 3-O-robinoside 7-O-rhamnoside, Kaempferol 7-O-glucoside, |
| | Kaempferol 3-O-galactoside 7-O-rhamnoside, Kaempferol 3-O-(6″-acetyl- |
| | galactoside) 7-O-rhamnoside, Quercetin 3-O-galactoside 7-O-rhamnoside, |
| | Quercetin 3-O-(6″-acetyl-galactoside) 7-O-rhamnoside, Kaempferol 3-O-(2″- |
| | rhamnosyl-galactoside) 7-O-rhamnoside, Kaempferol 3-O-(2″-rhamnosyl-6″- |
| | acetyl-galactoside) 7-O-rhamnoside, 6,8-Dihydroxykaempferol, Isorhamnetin |
| | 3-O-galactoside, Quercetin 3-O-rhamnosyl-rhamnosyl-glucoside, Kaempferol |
| | 3-O-rhamnosyl-rhamnosyl-glucoside, Methylgalangin, Kaempferol 3,7,4′-O- |
| | triglucoside, 5,3′,4′-Trihydroxy-3-methoxy-6:7-methylenedioxyflavone, 5,4′- |
| | Dihydroxy-3,3′-dimethoxy-6:7-methylenedioxyflavone, Jaceidin, |
| | Natsudaidain, 3-Methoxynobiletin, 3-Methoxysinensetin, Quercetin 3′-O- |
| | glucuronide, Quercetin 3′-sulfate, Quercetin 4′-O-glucuronide, Isorhamnetin |
| | 4′-O-glucuronide, Tamarixetin, Quercetin 3-O-glucosyl-rutinoside |
| Isoflavonoids | Daidzein, Formononetin, Genistein, Biochanin A, Glycitein, Glycitin, 6″-O- |
| | Acetyldaidzin, 6″-O-Malonylgenistin, Daidzin, Genistin, 6″-O- |
| | Acetylgenistin, 6″-O-Acetylglycitin, 6″-O-Malonyldaidzin, 6″-O- |
| | Malonylglycitin, 2′,7-Dihydroxy-4′,5′-dimethoxyisoflavone, 2-Dehydro-O- |
| | desmethylangolensin, 2′-Hydroxyformononetin, 3′,4′,7-Trihydroxyisoflavan, |
| | 3′,4′,7-Trihydroxyisoflavanone, 3′,7-Dihydroxyisoflavan, 3′- |
| | Hydroxydaidzein, 3′-Hydroxy-O-desmethylangolensin, 4′,6,7- |
| | Trihydroxyisoflavanone, 4′,7,8-Trihydroxyisoflavanone, 4′,7-Dihydroxy-3′- |
| | methoxyisoflavan, 4′,7-Dihydroxy-6-methoxyisoflavan, 4-Hydroxyequol, 4′- |
| | O-Methylequol, 5,6,7,3′,4′-Pentahydroxyisoflavone, 5,6,7,4′- |
| | Tetrahydroxyisoflavone, 5,7,8,3′,4′-Pentahydroxyisoflavone, 5,7,8,4′- |
| | Tetrahydroxyisoflavone, 5′-Hydroxy-O-desmethylangolensin, 5′-Methoxy-O- |
| | desmethylangolensin, 6,7,3′,4′-Tetrahydroxyisoflavone, 6,7,4′- |
| | Trihydroxyisoflavone, 6′-Hydroxyangolensin, 6′-Hydroxy-O- |
| | desmethylangolensin, 7,3′,4′-Trihydroxy-6-methoxyisoflavone, 7,3′,4′- |
| | Trihydroxyisoflavone, 7,8,3′,4′-Tetrahydroxyisoflavone, 7,8,4′- |
| | Trihydroxyisoflavone, Angolensin, Calycosin, Daidzein 4′,7-O- |
| | diglucuronide, Daidzein 4′,7-disulfate, Daidzein 4′-O-glucuronide, Daidzein |
| | 4′-sulfate, Daidzein 7-O-glucuronide, Dihydrobiochanin A, Dihydrodaidzein, |
| | Dihydrodaidzein 7-O-glucuronide, Dihydroformononetin, Dihydrogenistein, |
| | Dihydroglycitein, Equol, Formononetin 7-O-glucuronide, Formononetin 7- |
| | sulfate, Genistein 4′,7-O-diglucuronide, Genistein 4′,7-disulfate, Genistein 4′- |
| | O-glucuronide, Genistein 4′-sulfate, Genistein 5-O-glucuronide, Genistein 7- |
| | O-glucuronide, Genistein 7-sulfate, Glycitein 4′-O-glucuronide, Glycitein 7- |
| | O-glucuronide, Koparin, O-Desmethylangolensin, Orobol, Prunetin, |
| | Pseudobaptigenin, Puerarin, Daidzin 4′-O-glucuronide, Irisolidone 7-O- |
| | glucuronide, Tectorigenin 7-sulfate, Tectorigenin 4′-sulfate, Irisolidone, |
| | Tectorigenin, Tectoridin, 5,7-Dihydroxy-8,4′-dimethoxyisoflavone, |
| | Isotectorigenin, Equol 7-O-glucuronide, Equol 4′-O-glucuronide, 8- |
| | Hydroxydaidzein, Daidzein 7-sulfate, Daidzein 4′-O-sulfo-7-O-glucuronide, |
| | Daidzein 7-O-sulfo-4′-O-glucuronide, Equol 4′-sulfate, 3′,4′,5,7- |
| | Tetrahydroxyisoflavanone, 3′-O-Methylequol, 6-O-Methylequol, 3′- |
| | Hydroxygenistein, 3′-Hydroxydihydrodaidzein, 6-Hydroxydihydrodaidzein, |
| | 3′-Hydroxyequol, cis-4-Hydroxyequol, 4′-Methoxy-2′,3,7- |
| | trihydroxyisoflavanone, Irilone, Vestitone, Sativanone, Butin, 3′- |
| | Hydroxymelanettin, Liquiritigenin, Melanettin, Stevenin, Violanone, |
| | Isoliquiritigenin, Dalbergin, 3′-O-Methylviolanone, 8- |
| | Hydroxydihydrodaidzein |
| Lignans | Secoisolariciresinol, Matairesinol, Lariciresinol, Pinoresinol, Syringaresinol, |
| | Isolariciresinol, Arctigenin, Trachelogenin, Medioresinol, 1- |
| | Acetoxypinoresinol, Secoisolariciresinol di-O-glucoside, Sesamin, |
| | Sesamolin, Sesamolinol, Sesaminol, Sesaminol 2′-O-b-D-glucosyl (1−>2)-O- |
| | [b-D-glucosyl (1−>6)]-b-D-glucoside, Sesaminol 2′-O-b-D-glucosyl (1−>6)- |
| | O-b-D-glucoside, Sesaminol 2′-O-b-D-glucoside, Sesamol, Sesamolinol 4′-O- |
| | b-D-glucosyl (1−>6)-O-b-D-glucoside, 7-Hydroxymatairesinol, |
| | Isohydroxymatairesinol, Secoisolariciresinol-sesquilignan, Cyclolariciresinol, |
| | 7-Oxomatairesinol, Todolactol A, Conidendrin, 7- |
| | Hydroxysecoisolariciresinol, Nortrachelogenin, Lariciresinol-sesquilignan, |
| | Anhydro-secoisolariciresinol, Dimethylmatairesinol, Episesamin, |
| | Episesaminol, Sesaminol 2′-O-b-D-glucosyl (1−>2)-O-b-D-glucoside, |
| | Enterodiol, Enterolactone, Sesaminol 2-O-triglucoside, Schisandrin, Gomisin |
| | D, Schisandrol B, Tigloylgomicin H, Schisanhenol, Schisantherin A, Gomisin |
| | M2, Deoxyschisandrin, Schisandrin B, Schisandrin C, 2-Hydroxyenterodiol, |
| | 4-Hydroxyenterodiol, 6-Hydroxyenterodiol, 2-Hydroxyenterolactone, 4- |
| | Hydroxyenterolactone, 6-Hydroxyenterolactone, 2′-Hydroxyenterolactone, 4′- |
| | Hydroxyenterolactone, 6′-Hydroxyenterolactone, 5-Hydroxyenterolactone, 7- |
| | Hydroxyenterolactone |
| Non-phenolic | 4-Ethylbenzoic acid, Glycine, 1,3,5-Trimethoxybenzene, Vanilloylglycine |
| metabolites | |
| Alkylmethoxy- | 4-Vinylguaiacol, 4-Ethylguaiacol, 4-Vinylsyringol |
| phenols | |
| Alkylphenols | 5-Heneicosenylresorcinol, 5-Heneicosylresorcinol, 5-Heptadecylresorcinol, |
| | 5-Nonadecenylresorcinol, 5-Nonadecylresorcinol, 5-Pentacosenylresorcinol, |
| | 5-Pentacosylresorcinol, 5-Pentadecylresorcinol, 5-Tricosenylresorcinol, 5- |
| | Tricosylresorcinol, Alk(en)ylresorcinols, total, Alkenylresorcinols, total, |
| | Alkylresorcinols, total, 3-Methylcatechol, 4-Methylcatechol, 4-Ethylcatechol, |
| | 4-Vinylphenol, 4-Ethylphenol |
| Betacyanins | Betanin, Isobetanin, Betanidin, Isobetanidin |
| Capsaicinoids | Capsaicin |
| Curcuminoids | Curcumin, Demethoxycurcumin, Bisdemethoxycurcumin |
| Dihydro- | Dihydrocapsaicin, Nordihydrocapsaicin |
| capsaicins | |
| Furano- | Bergapten, Psoralen, Xanthotoxin, Isopimpinellin, Angelicin |
| coumarins | |
| Hydroxy- | Syringaldehyde, Protocatechuic aldehyde, Vanillin, 4-Hydroxybenzaldehyde, |
| benzaldehydes | Gallic aldehyde, p-Anisaldehyde, Ethyl vanillin, Vanillin 4-sulfate |
| Hydroxy- | 3-Methoxyacetophenone, 2,3-Dihydroxy-1-guaiacylpropanone, Paeonol, 2,4- |
| benzoketones | Dihydroxyacetophenone 5-sulfate, 2-Hydroxy-4-methoxyacetophenone 5- |
| | sulfate, Resacetophenone, Norathyriol |
| Hydroxycinnamal- | Ferulaldehyde, Sinapaldehyde |
| dehydes | |
| Hydroxy- | Coumarin, Isocoumarin, Mellein, Scopoletin, Esculetin, Esculin, |
| coumarins | Umbelliferone, 4-Hydroxycoumarin, Urolithin D, Urolithin B 3-sulfate, |
| | Urolithin A 3,8-O-diglucuronide, Urolithin A 3,8-disulfate, Urolithin A, |
| | Urolithin B, Urolithin B 3-O-glucuronide, Urolithin C |
| Hydroxyphenyl- | Homovanillyl alcohol |
| alcohols | |
| Hydroxy- | 2-Methoxy-5-prop-l-enylphenol, Anethole, Eugenol, Acetyl eugenol, [6]- |
| phenylpropenes | Gingerol, Estragole |
| Methoxyphenols | Guaiacol, p-Anisidine |
| Naphtoquinones | Juglone, 1,4-Naphtoquinone |
| Phenolic | Carnosic acid, Rosmanol, Carnosol, Epirosmanol, Rosmadial, Thymol, |
| terpenes | Carvacrol |
| Tyrosols | Hydroxytyrosol, 3,4-DHPEA-AC, p-HPEA-AC, Oleuropein, |
| | Demethyloleuropein, 3,4-DHPEA-EA, Ligstroside, 3,4-DHPEA-EDA, |
| | Hydroxytyrosol 4-O-glucoside, Oleoside dimethylester, Oleoside 11- |
| | methylester, Hydroxytyrosol 1′-O-glucoside, p-HPEA-EDA, p-HPEA-EA, |
| | Oleuropein-aglycone, Ligstroside-aglycone, Elenolic acid, Tyrosol 4-O- |
| | glucuronide, Tyrosol 4-sulfate, Hydroxytyrosol, total |
| Other | Coumestrol, Catechol, Pyrogallol, Phlorin, Phenol, Phloroglucinol, Arbutin, |
| polyphenols | Hydroquinone, 3,4-Dihydroxyphenylglycol, 5,5′,6,6′-Tetrahydroxy-3,3′- |
| | biindolyl, Resorcinol, 1-Phenyl-6,7-dihydroxy-isochroman, 1-(3-methoxy-4- |
| | hydroxy)-phenyl-6,7-dihydroxy-isochroman, Lithospermic acid, |
| | Lithospermic acid B, Salvianolic acid B, Salvianolic acid C, Salvianolic acid |
| | D, Salvianolic acid G, Isopropyl 3-(3,4-dihydroxyphenyl)-2- |
| | hydroxypropanoate |
| Hydroxybenzoic | Ellagic acid glucoside, Protocatechuic acid, Gallic acid, Vanillic acid, Ellagic |
| acids | acid, total, Gentisic acid, Ellagic acid, 4-Hydroxybenzoic acid, 3,4- |
| | Dimethoxybenzoic acid, Syringic acid, 5-O-Galloylquinic acid, Ellagic acid |
| | arabinoside, Ellagic acid acetyl-xyloside, Ellagic acid acetyl-arabinoside, 4- |
| | Methoxybenzoic acid, Gallic acid, total, Benzoic acid, 2-Hydroxybenzoic |
| | acid, 3-Hydroxybenzoic acid, 2,3-Dihydroxybenzoic acid, 2,4- |
| | Dihydroxybenzoic acid, 1-O-Galloyl glucose, 4-Hydroxybenzoic acid 4-O- |
| | glucoside, Protocatechuic acid 4-O-glucoside, Gallic acid 4-O-glucoside, 3,5- |
| | Dihydroxybenzoic acid, 2,6-Dihydroxybenzoic acid, Gallic acid 3-O-gallate, |
| | Gallic acid ethyl ester, Valoneic acid dilactone, 2,6-Dimethoxybenzoic acid, |
| | 2-Hydroxy-4-methoxybenzoic acid, Sanguisorbic acid dilactone, Galloyl |
| | glucose, Lambertianin C, Sanguiin H-6, Sanguiin H-10, Ellagitannins, total, |
| | Punicalagin, Gallagic acid, Tannic acid, Hydrolysable tannins, total, 3-O- |
| | Methylgallic acid, 4-O-Methylgallic acid, 3,4-O-Dimethylgallic acid, |
| | Punicalin, 4-Hydroxyhippuric acid, 3-Hydroxyhippuric acid, 2- |
| | Hydroxyhippuric acid, Hippuric acid, Paeoniflorin, Vanillic acid 4-sulfate, |
| | 2,3,4-Trihydroxybenzoic acid |
| Hydroxy- | p-Coumaric acid, 5-p-Coumaroylquinic acid, 4-p-Coumaroylquinic acid, |
| cinnamic acids | Caffeic acid, Feruloyl glucose, Ferulic acid, Caffeoyl tartaric acid, |
| | Rosmarinic acid, o-Coumaric acid, m-Coumaric acid, Sinapic acid, p- |
| | Coumaroyl glucose, p-Coumaroylquinic acid, 3-Caffeoylquinic acid, |
| | Verbascoside, 4-Caffeoylquinic acid, p-Coumaroyl tartaric acid, 2,5-di-S- |
| | Glutathionyl caftaric acid, Feruloyl tartaric acid, Caffeic acid ethyl ester, |
| | Cinnamoyl glucose, 5-Caffeoylquinic acid, 3-p-Coumaroylquinic acid, 2-S- |
| | Glutathionyl caftaric acid, 5-Feruloylquinic acid, 4-Feruloylquinic acid, 3- |
| | Feruloylquinic acid, 5-Sinapoylquinic acid, 4-Sinapoylquinic acid, 3- |
| | Sinapoylquinic acid, 3,5-Dicaffeoylquinic acid, Isoferulic acid, Caffeoyl |
| | glucose, p-Coumaric acid 4-O-glucoside, Caffeic acid 4-O-glucoside, Ferulic |
| | acid 4-O-glucoside, p-Coumaroyl tartaric acid glucosidic ester, p-Coumaric |
| | acid ethyl ester, Trans-Caffeoyl tartaric acid, Cis-Caffeoyl tartaric acid, |
| | Trans-p-Coumaroyl tartaric acid, Cis-p-Coumaroyl tartaric acid, Trans- |
| | Caffeic acid, Cis-Caffeic acid, Trans-p-Coumaric acid, Trans-Ferulic acid, |
| | Cis-p-Coumaric acid, Cis-Ferulic acid, 3,4-Dimethoxycinnamic acid, |
| | Hydroxycaffeic acid, Caffeic acid, total, Sinapic acid, total, Chicoric acid, 5- |
| | 5′-Dehydrodiferulic acid, 5-8′-Dehydrodiferulic acid, 1,2- |
| | Disinapoylgentiobiose, 1-Sinapoyl-2-feruloylgentiobiose, 1,2- |
| | Diferuloylgentiobiose, 1,2,2′-Trisinapoylgentiobiose, 1,2′-Disinapoyl-2- |
| | feruloylgentiobiose, 1-Sinapoyl-2,2′-diferuloylgentiobiose, 1,2,2′- |
| | Triferuloylgentiobiose, 8-O-4′-Dehydrodiferulic acid, 8-8′-Dehydrodiferulic |
| | acid, 5-8′-Benzofuran dehydrodiferulic acid, Cis-3-Caffeoylquinic acid, 3,4- |
| | Dicaffeoylquinic acid, Cis-5-Caffeoylquinic acid, 3,4-Diferuloylquinic acid, |
| | 3,5-Diferuloylquinic acid, 1-Caffeoylquinic acid, 1,3-Dicaffeoylquinic acid, |
| | 1,5-Dicaffeoylquinic acid, 4,5-Dicaffeoylquinic acid, Dicaffeoylquinic acid, |
| | b-D-fructosyl-a-D-(6-O-(E))-feruloylglucoside, Avenanthramide 1p, |
| | Avenanthramide 1f, Avenanthramide 2p, Avenanthramide 2c, |
| | Avenanthramide 2f, Avenanthramide 1c, Avenanthramide 1s, |
| | Avenanthramide 2s, Sinapoyl glucose, p-Coumaroyl malic acid, p-Coumaroyl |
| | glycolic acid, 3-Caffeoyl-1,5-quinolactone, 4-Caffeoyl-1,5-quinolactone, |
| | Quinic acid esters, total, 3-Feruloyl-1,5-quinolactone, 4-Feruloyl-1,5- |
| | quinolactone, 3,4-Dicaffeoyl-1,5-quinolactone, 3-p-Coumaroyl-1,5- |
| | quinolactone, 4-p-Coumaroyl-1,5-quinolactone, Cinnamic acid, Caffeoyl 3- |
| | hydroxytyrosine, Caffeoyl aspartic acid, p-Coumaroyl aspartic acid, p- |
| | Coumaroyl tyrosine, Caffeoyl tyrosine, p-Coumaroyl 3-hydroxytyrosine, |
| | Isoverbascoside, Sinapine, Avenanthramide A2, Avenanthramide K, |
| | Campesteryl ferulate, Sitostanyl ferulate, 4-O-8′,5′-5″-Dehydrotriferulic acid, |
| | 24-Methylcholestanol ferulate, 24-Methylcholesterol ferulate, 24- |
| | Methyllathosterol ferulate, Stigmastanol ferulate, Sitosterol ferulate, |
| | Schottenol ferulate, 24-Methylenecholestanol ferulate, Trans-5- |
| | Caffeoylquinic acid, Trans-3-Caffeoylquinic acid, 3-O-Methylrosmarinic |
| | acid, Sinapic acid 4-O-glucuronide, Sinapic acid 4-sulfate, Feruloylglycine 4- |
| | sulfate, Feruloylglycine, Isoferulic acid 3-O-glucuronide, Isoferulic acid 3- |
| | sulfate, Ferulic acid 4-sulfate, Ferulic acid 4-O-glucuronide, Caffeic acid 4- |
| | sulfate, Caffeic acid 3-sulfate, p-Coumaric acid 4-sulfate, Feruloyl C1- |
| | glucuronide, Isoferuloyl C1-glucuronide, Caffeic acid 3-O-glucuronide, |
| | Caffeic acid 4-O-glucuronide, Caffeoyl C1-glucuronide, Chlorogenic acid, |
| | total, 1,5-Diferuloylquinic acid, 1-Caffeoyl-5-feruloylquinic acid, 1-Feruloyl- |
| | 5-caffeoylquinic acid |
| Hydroxy- | 3,4-Dihydroxyphenylacetic acid, 4-Hydroxyphenylacetic acid, Homovanillic |
| phenylacetic | acid, Homoveratric acid, Methoxyphenylacetic acid, 3-Hydroxyphenylacetic |
| acids | acid, 2-Hydroxyphenylacetic acid, 4-Methoxyphenylacetic acid, |
| | Phenacetylglycine, Phenylacetic acid, 4-Hydroxymandelic acid, 2-Hydroxy- |
| | 2-phenylacetic acid, Homovanillic acid 4-sulfate, 4-Hydroxyphenyllactic acid |
| Hydroxy- | Dihydro-p-coumaric acid, Dihydrocaffeic acid, 3,4-Dihydroxyphenyl-2- |
| phenylpropanoic | oxypropanoic acid, 3-Hydroxy-3-(3-hydroxyphenyl)propionic acid, 3-(3,4- |
| acids | Dihydroxyphenyl)-2-methoxypropionic acid, 3 -Hydroxyphenylpropionic |
| | acid, Dihydroferulic acid 4-sulfate, Dihydroisoferulic acid 3-O-glucuronide, |
| | Dihydrocaffeic acid 3-O-glucuronide, Dihydrocaffeic acid 3-sulfate, |
| | Dihydroferulic acid, Dihydroferulic acid 4-O-glucuronide, Dihydrosinapic |
| | acid, Dihydroferuloylglycine 4-sulfate, Dihydroferuloylglycine, Danshensu, |
| | 3-Methoxy-4-hydroxyphenyllactic acid, 3,4-Dihydroxyphenyllactic acid |
| | methyl ester, Hydroxydanshensu, 3-Phenylpropionic acid, 3-Hydroxy-4- |
| | methoxyphenyllactic acid, Dihydroferulic acid 3-sulfate, 4-Hydroxyphenyl-2- |
| | propionic acid |
| Hydroxy- | 5-(3′-Methoxy-4′-hydroxyphenyl)-γ-valerolactone, 5-(3′-Methoxy-4′- |
| phenylpentanoic | hydroxyphenyl)-γ-valerolactone 4′-O-glucuronide, 4-Hydroxy-(3′,4′- |
| acids | dihydroxyphenyl)valeric acid, 5-(3′,4′-dihydroxyphenyl)-valeric acid, 5- |
| | (3′,4′,-dihydroxyphenyl)-γ-valerolactone, 5-(3′,4′,5′-trihydroxyphenyl)-γ- |
| | valerolactone, 5-(3′,5′-dihydroxyphenyl)-γ-valerolactone, 5-Hydroxyphenyl- |
| | γ-valerolactone, 3-Hydroxyphenylvaleric acid, 5-(3′,5′-dihydroxyphenyl)-γ- |
| | valerolactone 3-O-glucuronide |
| Stilbenes | Trans-Resveratrol, Trans-Resveratrol 3-O-glucoside, Piceatannol, Cis- |
| | Resveratrol, e-Viniferin, Pterostilbene, d-Viniferin, Cis-Resveratrol 3-O- |
| | glucoside, Pallidol, Piceatannol 3-O-glucoside, Pinosylvin, Resveratrol 5-O- |
| | glucoside, Resveratrol, Resveratrol 3-O-glucoside, 3,4,5,4′- |
| | Tetramethoxystilbene, 3′-Hydroxy-3,4,5,4′-tetramethoxystilbene, 3-Hydroxy- |
| | 4,5,4′-trimethoxystilbene, 4,4′-Dihydroxy-3,5-dimethoxystilbene, 4′- |
| | Hydroxy-3,4,5-trimethoxystilbene, 4-Hydroxy-3,5,4′-trimethoxystilbene, cis- |
| | Resveratrol 3-O-glucuronide, cis-Resveratrol 3-sulfate, cis-Resveratrol 4′-O- |
| | glucuronide, cis-Resveratrol 4′-sulfate, Resveratrol 3-O-glucuronide, |
| | Resveratrol 3-sulfate, Resveratrol 4′-O-glucuronide, trans-Resveratrol 3,5- |
| | disulfate, trans-Resveratrol 3,4′-disulfate, trans-Resveratrol 3-O-glucuronide, |
| | trans-Resveratrol 3-sulfate, trans-Resveratrol 4′-O-glucuronide, trans- |
| | Resveratrol 4′-sulfate, Dihydroresveratrol |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A method of modulating the abundance of at least a first and a second bacterial taxa in a subject's vaginal microbiota, the method comprising: administering to a subject in need thereof a composition comprising a population of glycan therapeutics in an amount effective to modulate the abundance of at least the first and second bacterial taxa, wherein at least 60% of the glycan population has a degree of polymerization (DP) of at least 5 and less than 30 glycan units, at least 10% of glycosidic bonds are beta-glycosidic bonds, at least 10% of glycosidic bonds are alpha glycosidic bonds, and optionally wherein the glycan population has an average degree of branching (DB, branching points per residue) of at least 0.01, 0.05, or at least 0.1.

2. The method of claim 1, wherein the abundance of each the first and second bacterial taxa in a human subject's vaginal microbiota is independently increased by at least 5%, 10%, 25% 50%, 75%, 100%, 250%, 500%, 750%, or by at least 1000%.

3. The method of claim 1, wherein the abundance of each the first and second bacterial taxa in a human subject's vaginal microbiota independently is decreased by at least 5%, 10%, 25% 50%, 75%, 85%, 90%, 95%, 96%, 97%, 98%, 99%, or by at least 99.9%.

4. The method of claim 2, wherein the first and second bacterial taxa are commensal bacterial taxa.

5. The method of claim 3, wherein the first and/or second bacterial taxa are pathogenic bacterial taxa.

6. The method of any one of claims 1-5, wherein at least 60% of the glycan population has a DP of at least 8 and less than 30 glycan units.

7. The method of any one of claims 1-5, wherein at least 60% of the glycan population has a DP of at least 10 and less than 30 glycan units.

8. The method of any one of claims 1-7, wherein the DB is between about 0.01 and 0.2 or at least about 0.4.

9. The method of any one of the preceding claims, wherein at least one glycan unit is in L-form.

10. The method of any one of the preceding claims, wherein at least one glycan unit is in D-form.

11. The method of any one of the preceding claims, wherein at least one glycan unit is a furanose sugar.

12. The method of any one of the preceding claims, wherein at least one glycan unit is a pyranose sugar.

13. The method of any one of the preceding claims, wherein at least one glycan unit is a tetrose, a pentose, a hexose, or a heptose.

14. The method of any one of the preceding claims, wherein at least one glycan unit is selected from the group consisting of a glucose, a galactose, an arabinose, a mannose, a fructose, a xylose, a fucose, and a rhamnose.

15. The method of any one of the preceding claims, wherein at least 5% of glycosidic bonds are 1→2 glycosidic bonds.

16. The method of any one of the preceding claims, wherein at least 5% of glycosidic bonds are 1→3 glycosidic bonds.

17. The method of any one of the preceding claims, wherein at least 5% of glycosidic bonds are 1→4 glycosidic bonds.

18. The method of any one of the preceding claims, wherein at least 5% of glycosidic bonds are 1→6 glycosidic bonds.

19. The method of any one of the preceding claims, wherein at least 5% each of glycosidic bonds are 1→2, 1→3, 1→4, and 1→6 glycosidic bonds.

20. The method of any one of claims 15-19, wherein at least 1% of glycosidic bonds are further selected from the group consisting of alpha 1→4, alpha 2→1, alpha 2→6, alpha 2→3, alpha 2→4, beta 1→4, beta 2→1, beta 2→6, beta 2→3, and beta 2→4 glycosidic bonds.

21. The method of claim 1, wherein the alpha:beta glycosidic bond ratio is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or 10:1.

22. The method of any one of the preceeding claims, wherein the population of glycan therapeutics does not comprise a detectable repeating unit of a DP of at least 3 glycan units.

23. The method of any one of the preceding claims, wherein the composition is insoluble in deionized water at 20° C.

24. The method of any one of claims 1-22, wherein the composition is soluble in deionized water and has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L at 20° C.

25. The method of any one of the preceding claims, wherein the population of glycan therapeutics is synthetic and not isolated from a natural oligo- or polysaccharide source.

26. The method of any one on the preceding claims, wherein the first and second bacterial taxa is individually selected from the group consisting of the genus Actinomyces, Aerococcus, Anaerococcus, Atopobium, Bacteroides, Corynebacterium, Dialister, Eggerthella, Escherichia, Finegoldia, Fusobacterium, Gardnerella, Haemophilus, Lactobacillus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, of the order Clostridiales, including. Bacterial vaginosis-associated bacterium-1 (BVAB-1), BVAB-2, and BVAB-3), and of the species Aerococcus christensenii Atopobium vaginae, Bacteroides urcalyticus, Corynebacterium vaginale, Dialister micraerophilus, Escherichia coli, Enterococcus faecium, Gardnerella vaginalis, Haemophilus influenza, Lactobacillus coleohominis, Lactobacillus crispatus, Lactobacillus gasseri, Lactobacillus iners, Lactobacillus jensenii, Lactobacillus vaginalis, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

27. The method of any one of the preceding claims, wherein the abundance of at least 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 150, 200, 250, or 500 bacterial taxa in the vagina is modulated, optionally selected from the taxa listed in Table 1.

28. The method of any one of the preceeding claims, wherein modulating is increasing, or, wherein modulating is decreasing the abundance of the bacterial taxa.

29. The method of any one on the preceding claims, wherein the subject has or is suspected of having a disease, disorder or condition related to a disturbed microbiota in the vagina.

30. The method of claim 29, wherein the disease, disorder or condition is bacterial vaginosis (BV), vaginal discharge, pelvic inflammatory disease, infection with vancomycin-resistant enterococci (VRE), Group B Streptococcus infection, sexually transmitted infectious disease, cervicitis, desquamative inflammatory vaginitis (DIV), vaginal Staphylococcus infection, risk for a preterm birth or risk of miscarriage.

31. The method of claim 29, the method further comprising selecting a subject for administration of the pharmaceutical composition, wherein the selection encompasses diagnosing the disease, disorder or condition of claim 30.

32. The method of any one of the preceding claims, wherein the composition further comprises one or more of: an excipient, a prebiotic, a probiotic, and a therapeutic agent.

33. The method of claim 32, wherein the therapeutic agent is an oral or vaginally-applied antibiotic, including metronidazole, clindamycin, tinidazole, and secnidazole, a vaginally-applied hormone, including estradiol, an antifungal, or a probiotic.

34. The method of any one of the preceding claims, wherein the composition is a pharmaceutical composition formulated for vaginal or oral administration.

35. The method of claim 34, whercin the pharmaceutical composition is formulated as a liquid, semi-solid or solid selected from an oral or vaginal tablet, a capsule, a lozenge, a vaginal cream or gel, a douche, a vaginal suppository, an intravaginal implant or pessary, tampon, or a vaginal ring.

36. The method of any one of the preceeding claims, wherein modulating the abundance of at least the first and the second bacterial taxa in a subject's vaginal microbiota further modulates the abundance of one or more microbial metabolite in the vagina of the subject.

37. The method of claim 36, wherein the microbial metabolite is selected from the group consisting of: formic acid, acetic acid, propionic acid, butryic acid, isobutyric acid, valeric acid, isovaleric acid, acorbic acid, tryptophan, serotonin, indole, succinic acid, trimethylamine (TMA), trimethylamine N-oxide (TMAO), deoxy cholic acid, ethyphenyl sulfate, acetylaldehyde, lactic acid, hydrogen peroxide and butanedione.

38. The method of claim 37, wherein the abundance of the metabolite is increased.

39. The method of claim 37, wherein the abundance of the metabolite is decreased.

40. A method for screening and selecting a population of glycan therapeutics, the method comprising:
providing a plurality of populations of glycan therapeutics, wherein at least 60% of the glycan population has a degree of polymerization (DP) of at least 5 and less than 30 glycan units, at least 10% of glycosidic bonds are beta-glycosidic bonds, at least 10% of glycosidic bonds are alpha glycosidic bonds, and optionally wherein the glycan population has an average degree of branching (DB, branching points per residue) of at least 0.01, 0.05, or at least 0.1,
subjecting the populations to one or more selection screens,
selecting a population of glycan therapeutics based on the selection screens, and
optionally isolating the selected population of glycan therapeutics.

41. The method of claim 40, wherein at least 60% of the glycan population has a DP of at least 8 and less than 30 glycan units.

42. The method of claim 40, wherein at least 60% of the glycan population has a DP of at least 10 and less than 30 glycan units.

43. The method of claim 40, wherein the DB is between about 0.01 and 0.2.

44. The method of claim 40, wherein the DB is at least about 0.4.

45. The method of any one of claims 40-44, wherein at least one glycan unit is in L-form.

46. The method of any one of claims 40-44, wherein at least one glycan unit is in D-form.

47. The method of any one of claims 40-44, wherein at least one glycan unit is a furanose sugar.

48. The method of any one of claims 40-44, wherein at least one glycan unit is a pyranose sugar.

49. The method of any one of claims 40-44, wherein at least one glycan unit is a tetrose, a pentose, a hexose, or a heptose.

50. The method of any one of claims 40-44, wherein at least one glycan unit is selected from the group consisting of a glucose, a galactose, an arabinose, a mannose, a fructose, a xylose, a fucose, and a rhamnose.

51. The method of any one of claims 40-50, wherein at least 5% of glycosidic bonds are 1→2 glycosidic bonds.

52. The method of any one of claims 40-50, wherein at least 5% of glycosidic bonds are 1→3 glycosidic bonds.

53. The method of any one of claims 40-50, wherein at least 5% of glycosidic bonds are 1→4 glycosidic bonds.

54. The method of any one of claims 40-50, wherein at least 5% of glycosidic bonds are 1→6 glycosidic bonds.

55. The method of any one of claims 40-50, wherein at least 5% each of glycosidic bonds are 1→2, 1→3, 1→4, and 1→6 glycosidic bonds.

56. The method of any one of claims 51-55, wherein at least 1% of glycosidic bonds are further selected from the group consisting of alpha 1→4, alpha 2→1, alpha 2→6, alpha 2→3, alpha 2→4, beta 1→4, beta 2→1, beta 2→6, beta 2→3, and beta 2→4 glycosidic bonds.

57. The method of claim 40, wherein the alpha:beta glycosidic bond ratio is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or 10:1.

58. The method of any one of claims 40-57, wherein the population of glycan therapeutics does not comprise a detectable repeating unit of a DP of at least 3 glycan units.

59. The method of any one of claims 40-58, wherein the composition is insoluble in deionized water at 20° C.

60. The method of any one of claims 40-58, wherein the composition is soluble in deionized water and has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L at 20° C.

61. The method of any one of claims 40-60, wherein the population of glycan therapeutics is synthetic and not isolated from a natural oligo- or polysaccharide source.

62. The method of any one of claims 40-61, wherein the selection screen is an in vitro assay in which two or more bacterial taxa are grown in a growth medium and the growth is monitored in the presence of the population of glycan therapeutics and compared to growth in the absence of the population of glycan therapeutics.

63. The method of claim 62, wherein at least 3, 4, 5, 6, 7, 8, 9, 10, 15, 20, 30, 40, or 50 taxa are grown in a growth medium.

64. The method of claim 62 or 63, wherein selection comprises selecting the population of glycan therapeutics that is capable of modulating the growth of at least one of the two or more bacterial taxa.

65. The method of claim 62 or 63, wherein selection comprises selecting the population of glycan therapeutics that is capable of modulating the growth of at least 3, 4, 5, 6, 7, 8, 9, 10, 15, or at least 20 bacterial taxa.

66. The method of any one of claims 62-65, wherein the bacterial taxa is selected from the group consisting of the genus Actinomyces, Aerococcus, Anaerococcus, Atopobium, Bacteroides, Corynebacterium, Dialister, Eggerthella, Escherichia, Finegoldia, Fusobacterium, Gardnerella, Haemophilus, Lactobacillus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, of the order Clostridiales, including. Bacterial vaginosis-associated bacterium-1 (BVAB-1), BVAB-2, and BVAB-3), and of the species Acrococcus christensenii Atopobium vaginae, Bacteroides urealyticus, Corynebacterium vaginale, Dialister micraerophilus, Escherichia coli, Enterococcus faecium, Gardnerella vaginalis, Haemophilus influenza, Lactobacillus coleohominis, Lactobacillus crispatus, Lactobacillus gasseri, Lactobacillus iners, Lactobacillus jensenii, Lactobacillus vaginalis, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

67. The method of any one of claims 64-66, wherein modulating the growth is increasing the abundance of the bacterial taxa.

68. The method of any one of claims 64-66, wherein modulating the growth is decreasing the abundance of the bacterial taxa.

69. The method of claim 67, wherein the abundance is increased by at least 5%, 10%, 25% 50%, 75%, 100%, 250%, 500%, 750%, or by at least 1000%.

70. The method of claim 68, wherein the abundance is decreased by at least 5%, 10%, 25% 50%, 75%, 85%, 90%, 95%, 96%, 97%, 98%, 99%, or by at least 99.9%.

71. The method of any one of claim 40-61, wherein the method is carried out in vivo using a laboratory animal.

72. The method of claim 71, wherein the population of glycan therapeutics is administered to the animal, after a period of time a sample is taken from the animal's vagina and analyzed for growth of bacterial taxa.

73. The method of claim 72, wherein selection of the population of glycan therapeutics comprises selecting the population that is capable of modulating the growth of bacterial taxa in the animal, wherein the animal is optionally contacted with the bacterial taxa prior to or concommitant with the administration of the therapeutic glycan.

74. The method of claim 72, wherein selection of the population of glycan therapeutics comprises selecting the population that is capable of modulating the growth of at least 2, 3, 4, 5, 6, 7, 8, 9, 10, 15, or at least 20 bacterial taxa.

75. The method of claim 74, wherein the bacterial taxa is selected from the group consisting of the genus Actinomyces, Acrococcus, Anaerococcus, Atopobium, Bacteroides, Corynebacterium, Dialister, Eggerthella, Escherichia, Finegoldia, Fusobacterium, Gardnerella, Haemophilus, Lactobacillus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, of the order Clostridiales, including. Bacterial vaginosis-associated bacterium-1 (BVAB-1), BVAB-2, and BVAB-3), and of the species Aerococcus christensenii Atopobium vaginae, Bacteroides urealyticus, Corynebacterium vaginale, Dialister micraerophilus, Escherichia coli, Enterococcus faecium, Gardnerella vaginalis, Haemophilus influenza, Lactobacillus coleohominis, Lactobacillus crispatus, Lactobacillus gasseri, Lactobacillus iners, Lactobacillus jensenii, Lactobacillus vaginalis, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

76. The method of any one of claims 73-75, wherein modulating the growth is increasing the abundance of the bacterial taxa.

77. The method of any one of claims 73-75, wherein modulating the growth is decreasing the abundance of the bacterial taxa.

78. The method of claim 76, wherein the abundance is increased by at least 5%, 10%, 25% 50%, 75%, 100%, 250%, 500%, 750%, or by at least 1000%.

79. The method of claim 77, wherein the abundance is decreased by at least 5%, 10%, 25% 50%, 75%, 85%, 90%, 95%, 96%, 97%, 98%, 99%, or by at least 99.9%.

80. A population of glycan therapeutics produced according to a method comprising: contacting a starting material comprising at least one glycan unit with a polymer catalyst under conditions that promote the formation of one or more glycosidic bond between glycan units, thereby producing a population of glycan therapeutics, wherein the glycan unit is a monosaccharide and wherein the polymer catalyst comprises acidic monomers and ionic monomers that are connected to form a polymeric backbone, wherein each acidic monomer has at least one Bronsted-Lowry acid, and each ionic monomer independently has at least one nitrogen-containing cationic group or phosphorous-containing cationic group.

81. The population of glycan therapeutics of claim 80, wherein each acidic monomer of the polymer catalyst has one Bronsted-Lowry acid.

82. The population of glycan therapeutics of claim 80, wherein at least one acidic monomer of the polymer catalyst has a distinct Bronsted-Lowry acid.

83. The population of glycan therapeutics of claim 80 wherein each ionic monomer of the polymer catalyst has one nitrogen-containing cationic group or phosphorous-containing cationic group.

84. The population of glycan therapeutics of claim 80, wherein at least one ionic monomer of the polymer catalyst has two nitrogen-containing cationic groups or phosphorous-containing cationic groups.

85. The population of glycan therapeutics of any one of claims 80-84, wherein at least 60% of the population has a degree of polymerization (DP) of at least 5 and less than 30 glycan units, at least 10% of glycosidic bonds are beta-glycosidic bonds, at least 10% of glycosidic bonds are alpha glycosidic bonds, and optionally wherein the glycan population has an average degree of branching (DB, branching points per residue) of at least 0.01, 0.05, or at least 0.1.

86. The population of glycan therapeutics of claim 85, wherein at least 60% of the population has a DP of at least 8 and less than 30 glycan units.

87. The population of glycan therapeutics of claim 85, wherein at least 60% of the population has a DP of at least 10 and less than 30 glycan units.

88. The population of glycan therapeutics of claim 85, wherein the DB is between about 0.01 and 0.2.

89. The population of glycan therapeutics of claim 85, wherein the DB is at least about 0.4.

90. The population of glycan therapeutics of any one of claims 80-89, wherein at least one glycan unit is in L-form.

91. The population of glycan therapeutics of any one of claims 80-89, wherein at least one glycan unit is in D-form.

92. The population of glycan therapeutics of any one of claims 80-89, wherein at least one glycan unit is a furanose sugar.

93. The population of glycan therapeutics of any one of claims 80-89, whcrcin at least one glycan unit is a pyranose sugar.

94. The population of glycan therapeutics of any one of claims 80-89, wherein at least one glycan unit is a tetrose, a pentose, a hexose, or a heptose.

95. The population of glycan therapeutics of any one of claims 80-89, wherein at least one glycan unit is selected from the group consisting of a glucose, a galactose, an arabinose, a mannose, a fructose, a xylose, a fucose, and a rhamnose.

96. The population of glycan therapeutics of any one of claims 80-95, wherein at least 5% of glycosidic bonds are 1→2 glycosidic bonds.

97. The population of glycan therapeutics of any one of claims 80-95, wherein at least 5% of glycosidic bonds are 1→3 glycosidic bonds.

98. The population of glycan therapeutics of any one of claims 80-95, wherein at least 5% of glycosidic bonds are 1→4 glycosidic bonds.

99. The population of glycan therapeutics of any one of claims 80-95, wherein at least 5% of glycosidic bonds are 1→6 glycosidic bonds.

100. The population of glycan therapeutics of any one of claims 80-95, wherein at least 5% each of glycosidic bonds are 1→2, 1→3, 1→4, and 1→6 glycosidic bonds.

101. The population of glycan therapeutics of any one of claims 96-100, wherein at least 1% of glycosidic bonds are further selected from the group consisting of alpha 1→4, alpha 2→1, alpha 2→6, alpha 2→3, alpha 2→4, beta 1→4, beta 2→1, beta 2→6, beta 2→3, and beta 2→4 glycosidic bonds.

102. The population of glycan therapeutics of any one of claims 80-101, wherein the alpha:beta glycosidic bond ratio is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or 10:1.

103. The population of glycan therapeutics of any one of claims 80-102, wherein the population does not comprise a detectable repeating unit of a DP of at least 3 glycan units.

104. The population of glycan therapeutics of any one of claims 80-103, wherein the composition is insoluble in deionized water at 20° C.

105. The population of glycan therapeutics of any one of claims 80-103, wherein the composition is soluble in deionized water and has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L at 20° C.

106. The population of glycan therapeutics of any one of claims 80-105, wherein the population modulates the abundance of one or more bacterial taxa in a vagina of a subject when administered to the vagina.

107. The population of glycan therapeutics of any one of claims 80-105, wherein the population modulates the abundance of one or more bacterial taxa when subjected to an in vitro bacterial growth assay comprising bacteria associated with the vagina.

108. The population of glycan therapeutics of claim 106 or 107, wherein the population modulates the abundance of at least 2, 3, 4, 5, 6, 7, 8, 9, 10, 15, or at least 20 bacterial taxa.

109. The population of glycan therapeutics of claim 108, wherein the bacterial taxa is selected from the group consisting of the genus Actinomyces, Aerococcus, Anaerococcus, Atopobium, Bacteroides, Corynebacterium, Dialister, Eggerthella, Escherichia, Finegoldia, Fusobacterium, Gardnerella, Haemophilus, Lactobacillus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, of the order Clostridiales, including. Bacterial vaginosis-associated bacterium-1 (BVAB-1), BVAB-2, and BVAB-3), and of the species Aerococcus christensenii Atopobium vaginae, Bacteroides urealyticus, Corynebacterium vaginale, Dialister micraerophilus, Escherichia coli, Enterococcus faecium, Gardnerella vaginalis, Haemophilus influenza, Lactobacillus coleohominis, Lactobacillus crispatus, Lactobacillus gasseri, Lactobacillus iners, Lactobacillus jensenii, Lactobacillus vaginalis, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

110. The population of glycan therapeutics of any one of claims 106-109, wherein modulating the growth is increasing the abundance of the bacterial taxa.

111. The population of glycan therapeutics of any one of claims 106-109, wherein modulating the growth is decreasing the abundance of the bacterial taxa.

112. The population of glycan therapeutics of claim 110, wherein the abundance is increased by at least 5%, 10%, 25% 50%, 75%, 100%, 250%, 500%, 750%, or by at least 1000%.

113. The population of glycan therapeutics of claim 111, wherein the abundance is decreased by at least 5%, 10%, 25% 50%, 75%, 85%, 90%, 95%, 96%, 97%, 98%, 99%, or by at least 99.9%.

114. A method of modulating microbial diversity in a subject's vagina, the method comprising: administering to a subject in need thereof a pharmaceutical composition comprising a population of glycan therapeutics, in an amount effective to modulate microbial diversity in the subject's vagina, wherein at least 60% of the glycan population has a degree of polymerization (DP) of at least 5 and less than 30 glycan units, at least 10% of glycosidic bonds are beta-glycosidic bonds, at least 10% of glycosidic bonds are alpha glycosidic bonds, and optionally wherein the glycan population has an average degree of branching (DB, branching points per residue) of at least 0.01, 0.05, or at least 0.1.

115. The method of claim 114, wherein modulating microbial diversity increases the Shannon Diversity of the microbial community in the vagina.

116. The method of claim 114, wherein modulating microbiome diversity decreases the Shannon Diversity of the microbial community in the vagina.

117. The method of claim 115, wherein the increase in Shannon Diversity is at least 0.1%, 0.5%, 1%, 2%, 3%, 4%, 5%, or at least 10%.

118. The method of claim 116, wherein the decrease in Shannon Diversity is at least 0.1%, 0.5%, 1%, 2%, 3%, 4%, 5%, or at least 10%.

119. The method of any one of claims 114-118, wherein at least 60% of the glycan population has a DP of at least 8 and less than 30 glycan units.

120. The method of any one of claims 114-118, wherein at least 60% of the glycan population has a DP of at least 10 and less than 30 glycan units.

121. The method of any one of claims 114-120, wherein the DB is between about 0.01 and 0.2 or at least about 0.4.

122. The method of any one of claims 114-121, wherein at least one glycan unit is in L-form.

123. The method of any one of claims 114-121, wherein at least one glycan unit is in D-form.

124. The method of any one of claims 114-121, wherein at least one glycan unit is a furanose sugar.

125. The method of any one of claims 114-121, wherein at least one glycan unit is a pyranose sugar.

126. The method of any one of claims 114-121, wherein at least one glycan unit is a tetrose, a pentose, a hexose, or a heptose.

127. The method of any one of claims 114-121, wherein at least one glycan unit is selected from the group consisting of a glucose, a galactose, an arabinose, a mannose, a fructose, a xylose, a fucose, and a rhamnose.

128. The method of any one of claims 114-127, wherein at least 5% of glycosidic bonds are 1→2 glycosidic bonds.

129. The method of any one of claims 114-127, wherein at least 5% of glycosidic bonds are 1→3 glycosidic bonds.

130. The method of any one of claims 114-127, wherein at least 5% of glycosidic bonds are 1→4 glycosidic bonds.

131. The method of any one of claims 114-127, wherein at least 5% of glycosidic bonds are 1→6 glycosidic bonds.

132. The method of any one of claims 114-127, wherein at least 5% each of glycosidic bonds are 1→2, 1→3, 1→4, and 1→6 glycosidic bonds.

133. The method of any one of claims 128-132, wherein at least 1% of glycosidic bonds are further selected from the group consisting of alpha 1→4, alpha 2→1, alpha 2→6, alpha 2→3, alpha 2→4, beta 1→4, beta 2→1, beta 2→6, beta 2→3, and beta 2→4 glycosidic bonds.

134. The method of any one of claims 114-133, wherein the alpha:beta glycosidic bond ratio is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or 10:1.

135. The method of any one of claims 114-134, wherein the population of glycan therapeutics does not comprise a detectable repeating unit of a DP of at least 3 glycan units.

136. The method of any one of claims 114-136, wherein the composition is insoluble in deionized water at 20° C.

137. The method of any one of claims 114-136, wherein the composition is soluble in deionized water and has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L at 20° C.

138. The method of any one of claims 114-137, wherein the population of glycan therapeutics is synthetic and not isolated from a natural oligo- or polysaccharide source.

139. The method of any one of claims 114-138, wherein the subject has or is suspected of having a disease, disorder or condition related to a disturbed microbiota in the vagina.

140. The method of claim 139, wherein the disease, disorder or condition is bacterial vaginosis (BV), vaginal discharge, pelvic inflammatory disease, infection with vancomycin-resistant enterococci (VRE), Group B Streptococcus infection, sexually transmitted infectious disease, cervicitis, desquamative inflammatory vaginitis (DIV), vaginal Staphylococcus infection, risk for a preterm birth or risk of miscarriage.

141. A population of therapeutic glycans, wherein at least 60% of the glycan population has a degree of polymerization (DP) of at least 5 and less than 30 glycan units, at least 10% of glycosidic bonds are beta-glycosidic bonds, at least 10% of glycosidic bonds are alpha glycosidic bonds, and optionally wherein the glycan population has an average degree of branching (DB, branching points per residue) of at least 0.01, 0.05, or at least 0.1.

142. The population of claim 141, wherein at least 60% of the glycan population has a DP of at least 8 and less than 30 glycan units.

143. The population of claim 141, wherein at least 60% of the glycan population has a DP of at least 10 and less than 30 glycan units.

144. The population of claim 141, wherein the DB is between about 0.01 and 0.2 or at least about 0.4.

145. The population of any one of claims 141-144, wherein at least one glycan unit is in L-form.

146. The population of any one of claims 141-144, wherein at least one glycan unit is in D-form.

147. The population of any one of claims 141-144, wherein at least one glycan unit is a furanose sugar.

148. The population of any one of claims 141-144, wherein at least one glycan unit is a pyranose sugar.

149. The population of any one of claims 141-144, wherein at least one glycan unit is a tetrose, a pentose, a hexose, or a heptose.

150. The population of any one of claims 141-144, wherein at least one glycan unit is selected from the group consisting of a glucose, a galactose, an arabinose, a mannose, a fructose, a xylose, a fucose, and a rhamnose.

151. The population of any one of claims 141-150, wherein at least 5% of glycosidic bonds are 1→2 glycosidic bonds.

152. The population of any one of claims 141-150, wherein at least 5% of glycosidic bonds are 1→3 glycosidic bonds.

153. The population of any one of claims 141-150, wherein at least 5% of glycosidic bonds are 1→4 glycosidic bonds.

154. The population of any one of claims 141-150, wherein at least 5% of glycosidic bonds are 1→6 glycosidic bonds.

155. The population of any one of claims 141-150, wherein at least 5% each of glycosidic bonds are 1→2, 1→3, 1→4, and 1→6 glycosidic bonds.

156. The population of any one of claims 151-155, wherein at least 1% of glycosidic bonds are further selected from the group consisting of alpha 1→4, alpha 2→1, alpha 2→6, alpha 2→3, alpha 2→4, beta 1→4, beta 2→1, beta 2→6, beta 2→3, and beta 2→4 glycosidic bonds.

157. The population of any one of claims 141-156, wherein the alpha:beta glycosidic bond ratio is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or 10:1.

158. The population of any one of claims 141-157, wherein the population of glycan therapeutics does not comprise a detectable repeating unit of a DP of at least 3 glycan units.

159. The population of any one of claims 141-145 comprising at least 2 glycan units selected from the group consisting glucose and galactose, glucose and arabinose, glucose and mannose, glucose and fructose, glucose and lactose, glucose and xylose, glucose and fucose, glucose and rhamnose, galactose and arabinose, galactose and mannose, galactose and fructose, galactose and lactose, galactose and xylose, galactose and fucose, and galactose and rhamnose, arabinose and mannose, arabinose and fructose, arabinose and lactose, arabinose and xylose, arabinose and fucose, and arabinose and rhamnose, mannose and fructose, mannose and lactose, mannose and xylose, mannose and fucose, and mannose and rhamnose, fructose and lactose, fructose and xylose, fructose and fucose, and fructose and rhamnose, lactose and xylose, lactose and fucose, and lactose and rhamnose, xylose and fucose, and xylose and rhamnose, and fucose and rhamnose

160. The population of any one of claims 141-145 comprising at least 2 glycosidic bonds selected from the group consisting of alpha 1→2 and alpha 1→3, alpha 1→2 and alpha 1→4, alpha 1→2 and alpha 1→6, alpha 1→2 and beta 1→2, alpha 1→2 and beta 1→3, alpha 1→2 and beta 1→4, alpha 1→2 and beta 1→6, alpha 1→3 and alpha 1→4, alpha 1→3 and alpha 1→6, alpha 1→3 and beta 1→2, alpha 1→3 and beta 1→3, alpha 1→3 and beta 1→4, alpha 1→3 and beta 1→6, alpha 1→4 and alpha 1→6, alpha 1→4 and beta 1→2, alpha 1→4 and beta 1→3, alpha 1→4 and beta 1→4, alpha 1→4 and beta 1→6, alpha 1→6 and beta 1→2, alpha 1→6 and beta 1→3, alpha 1→6 and beta 1→4, alpha 1→6 and beta 1→6, beta 1→2 and beta 1→3, beta 1→2 and beta 1→4, beta 1→2 and beta 1→6, beta 1→3 and beta 1→4, beta 1→3 and beta 1→6, and beta 1→4 and beta 1→6.

161. The population of claim 160 further comprising one or more of an alpha 2→1, alpha 2→6, alpha 2→3, alpha 2→4, beta 2→1, beta 2→6, beta 2→4, or beta 2→3 glycosidic bond.

162. The population of any one of claims 141-161, wherein the composition is insoluble in deionized water at 20° C.

163. The population of any one of claims 141-161, wherein the composition is soluble in deionized water and has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L at 20° C.

164. The population of any one of claims 141-163, wherein the glycan population modulates the abundance of one or more bacterial taxa in a vagina of a subject when administered to the vagina.

165. The population of any one of claims 141-163, wherein the glycan population modulates the abundance of one or more bacterial taxa when subjected to an in vitro bacterial growth assay comprising bacteria associated with the vagina.

166. The population of claim 164 or 165, wherein the glycan population modulates the abundance of at least 2, 3, 4, 5, 6, 7, 8, 9, 10, 15, or at least 20 bacterial taxa.

167. The population of any one of claims 164-166, wherein the bacterial taxa is selected from the genus Actinomyces, Aerococcus, Anaerococcus, Atopobium, Bacteroides, Corynebacterium, Dialister, Eggerthella, Escherichia, Finegoldia, Fusobacterium, Gardnerella, Haemophilus, Lactobacillus, Leptotrichia, Listeria, Megasphaera, Mycoplasma, Mobiluncus, Neisseria, Peptoniphilus, Peptostreptococcus, Porphyromonas, Prevotella, Sneathia, Staphylococcus, Streptococcus, and Ureaplasma, of the order Clostridiales, including. Bacterial vaginosis-associated bacterium-1 (BVAB-1), BVAB-2, and BVAB-3), and of the species Aerococcus christensenii Atopobium vaginae, Bacteroides urealyticus, Corynebacterium vaginale, Dialister micraerophilus, Escherichia coli, Enterococcus faecium, Gardnerella vaginalis, Haemophilus influenza, Lactobacillus coleohominis, Lactobacillus crispatus, Lactobacillus gasseri, Lactobacillus iners, Lactobacillus jensenii, Lactobacillus vaginalis, Leptotrichia amnionii, Listeria monocytogenes, Mycoplasma hominis, Neisseria gonorrhoeae, Peptoniphilus lacrimalis, Porphyromonas asaccharolytica, Prevotella timonensis, Sneathia sanguinegens, Staphylococcus aureus, Streptococcus agalactiae, Streptococcus pneumonia, and Ureaplasma urealyticum.

168. The population of any one of claims 164-167, wherein modulating the growth is increasing the abundance of the bacterial taxa.

169. The population of any one of claims 164-167, wherein modulating the growth is decreasing the abundance of the bacterial taxa.

170. The population of claim 168, wherein the abundance is increased by at least 5%, 10%, 25% 50%, 75%, 100%, 250%, 500%, 750%, or by at least 1000%.

171. The population of claim 169, wherein the abundance is decreased by at least 5%, 10%, 25% 50%, 75%, 85%, 90%, 95%, 96%, 97%, 98%, 99%, or by at least 99.9%.

172. The population of claim 141, wherein:
a) at least 65%, 70%, 75%, 80%, 85%, 90%, or at least 95% of the glycan population has a DP of at least 5 and less than 30 glycan units, at least 8 and less than 30 glycan units, or at least 10 and less than 30 glycan units,
b) at least 10%, 15%, 20%, 25%, 30%, 35%, 40%, 45%, 50%, 55%, 60%, 65%, 70%, 75%, 80%, or at least 85% of glycosidic bonds are beta-glycosidic bonds,
c) at least 10%, 15%, 20%, 25%, 30%, 35%, 40%, 45%, 50%, 55%, 60%, 65%, 70%, 75%, 80%, or at least 85% of glycosidic bonds are alpha glycosidic bonds, and wherein b) and c) do not exceed 100%.

173. The glycan population of any one of claims 141-172, wherein the glycan population does not comprise N-acetylgalactosamine, N-acetylglucosamine, and/or sialic acid.

174. The glycan population of any one of claims 141-173, wherein the glycan population does not comprise a lipid and fatty acid.

175. The glycan population of any one of claims 141-174, wherein the glycan population does not comprise an amino acid.

176. The glycan population of any one of claims 141-175, wherein when the glycan population is contacted with one or more bacterium selected from the group consisting of the bacteria listed in Table 1 under conditions effective for reducing the DP of the glycan population, the DP is reduced in the population of glycans by at least 10%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or at least 95% after 1 hour, 6 hours, 12 hours, 18 hours or 24 hours of contacting.

177. The glycan population of claim 176, wherein the glycan population is contacted with two or more, three or more, four or more, five or more, six or more, seven or more, eight or more, nine or more, 10 or more, 11 or more, or 12 or more bacteria selected from the group consisting of the bacteria listed in Table 1.

178. The glycan population of any one of claims 141-175, wherein when the glycan population is contacted with one or more bacterium selected from the group consisting of the bacteria listed in Table 1 under conditions effective for reducing the DP of the glycan population, the degree of polymerization is reduced in the population of glycans by no more than 5%, 4%, 3%, 2%, 1%, 0.5%, 0.25%, 0.1%, or no more than 0.05% after 1 hour, 6 hours, 12 hours, 18 hours or 24 hours of contacting.

179. The glycan population of claim 178, wherein the glycan population is contacted with two or more, three or more, four or more, five or more, six or more, seven or more, eight or more, nine or more, 10 or more, 11 or more, or 12 or more bacteria selected from the group consisting of the bacteria listed in Table 1.

180. The glycan population of any one of claims 141-175, wherein the glycan population exhibits a decrease in the percentage of one or more glycosidic bond selected from the group consisting of alpha 1→2, alpha 1→3, alpha 1→4, alpha 1→6, alpha 2→1, alpha 2→6, alpha 2→3, alpha 2→4, beta 1→2, beta 1→3, beta 1→4, beta 1→6, beta 2→1, beta 2→6, beta 2→3, and beta 2→4, wherein the percentage decrease is at least 1%, 2%, 3%, 4%, 5%, 6%, 7%, 8%,9%, 10%, 15%, 20%, 25%, 30%, 35%, 40%, 45%, 50%, 55%, 60%, 65%, 70%, 75%, 80%, 85%, 90%, or at least 95% after 1 hour, 6 hours, 12 hours, 18 hours or 24 hours of contacting.

181. The glycan population of any one of claims 141-175, wherein the glycan population exhibits an increase in the percentage of one or more glycosidic bond selected from the group consisting of alpha 1→2, alpha 1→3, alpha 1→4, alpha 1→6, alpha 2→1, alpha 2→6, alpha 2→3, alpha 2→4, beta 1→2, beta 1→3, beta 1→4, beta 1→6, beta 2→1, beta 2→6, beta 2→3, and beta 2→4, wherein the percentage increase is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or at least 95% after 1 hour, 6 hours, 12 hours, 18 hours or 24 hours of contacting.

182. The glycan population of any one of claims 141-175, wherein when the glycan population is contacted with one or more bacterium selected from the group consisting of the bacteria listed in Table 1 under conditions effective for bacterial growth, the glycan population modulates the growth of the one or more bacterium.

183. The glycan population of claim 182, wherein the glycan population is contacted with two or more, three or more, four or more, five or more, six or more, seven or more, eight or more, nine or more, 10 or more, 11 or more, or 12 or more bacteria selected from the group consisting of the bacteria listed in Table 1.

184. The glycan population of claim 182 or 183, wherein the growth of the one or more bacterium is increased by at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 110%, 120%, 130%, 140%, 150%, 160%, 170%, 180%, 190%,200%,250%, 300%, 350%, 400%, 450%, 500%, 600%, 700%, 800%, 900%, or by at least 1000% after 1 hour, 6 hours, 12 hours, 18 hours or 24 hours of contacting.

185. The glycan population of claim 182 or 183, wherein the growth of the one or more bacterium is decreased by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or by at least 99.9% after 1 hour, 6 hours, 12 hours, 18 hours or 24 hours of contacting.

186. The glycan population of any one of claims 141-175, wherein when the glycan population is contacted with one or more bacterium selected from the group consisting of the bacteria listed in Table 1 under conditions effective for bacterial growth, the glycan population modulates the concentration of one or more metabolite selected from the group consisting of the metabolites listed in Table 2.

187. The glycan population of claim 186, wherein the glycan population is contacted with two or more, three or more, four or more, five or more, six or more, seven or more, eight or more, nine or more, 10 or more, 11 or more, or 12 or more bacteria selected from the group consisting of the bacteria listed in Table 1.

188. The glycan population of claim 187, wherein the metabolite concentration is increased by at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 100%, 110%, 120%, 130%, 140%, 150%, 160%, 170%, 180%, 190%, 200%, 250%, 300%, 350%, 400%, 450%, 500%, 600%, 700%, 800%, 900%, or by at least 1000% after 1 hour, 6 hours, 12 hours, 18 hours or 24 hours of contacting.

189. The glycan population of claim 182 or 183, wherein the metabolite concentration is decreased by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or by at least 99.9% after 1 hour, 6 hours, 12 hours, 18 hours or 24 hours of contacting.

190. A pharmaceutical composition comprising the glycan population of any one of claims 141-189 and a pharmaceutically acceptable excipient.

191. A dosage form formulated for vaginal or oral administration comprising the glycan population of any one of claims 141-189.

192. The dosage form of claim 191 formulated as a liquid, semi-solid or solid selected from an oral or vaginal tablet, a capsule, a lozenge, a vaginal cream or gel, a douche, a vaginal suppository, an intravaginal implant or pessary, tampon, or a vaginal ring.

193. A kit comprising the pharmaceutical composition of claim 190 or the dosage form of claim 191 and a therapeutic agent and/or dietary component.

194. The kit of claim 193, wherein the therapeutic agent is an oral or vaginally-applied antibiotic, including metronidazole, clindamycin, tinidazole, and secnidazole, a vaginally-applied hormone, including estradiol, an antifungal, or a probiotic.

195. The kit of claim 193, wherein the dietary component is a prebiotic or probiotic.
